SEQUENCE LISTING (255 sequences)

SEQ ID NO: 1 - DNA, 1514 bp, unidentified
  misc_feature 1..1514: note = F01
  source 1..1514: mol_type = other DNA
agggttcgattctggctcaggatgaacgctggcggcgtgcttaacacatgcaagtcgaacgggatccatcaggctttgcttggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatacggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcctaaccccttgtgggatggagccgtctaaggtgaggctcgtgattgggactaagtcgtaacaaggtagccgtaccggaaggtgcggctggatcacct

SEQ ID NO: 2 - DNA, 1517 bp, unidentified
  misc_feature 1..1517: note = F02
  source 1..1517: mol_type = other DNA
agagtttgatcctggctcaggatgaacgctagctacaggcttaacacatgcaagtcgaggggcagcattttagtttgcttgcaaactaaagatggcgaccggcgcacgggtgagtaacacgtatccaacctgccgataactcggggatagcctttcgaaagaaagattaatatccgatagtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacct

SEQ ID NO: 3 - DNA, 1517 bp, unidentified
  misc_feature 1..1517: note = F03
  source 1..1517: mol_type = other DNA
agagtttgatcctggctcaggatgaacgctagctacaggcttaacacatgcaagtcgaggggcatcaggaagaaagcttgctttctttgctggcgaccggcgcacgggtgagtaacacgtatccaacctgccctttactcggggatagcctttcgaaagaaagattaatacccgatggtataattattccgcatggtttgattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgatggcctgaaccagccaagtagcgtgaaggatgaaggctctatgggtcgtaaacttcttttatattagaataaagtgcagtatgtatactgttttgtatgtataatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatctttagttactaacaggtcatgctgaggactctggagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaaggagcgtcctagggtaaaactggtgactggggctaagtcgtaacaaggtagccgtaccggaaggtgcggctggaacacct

SEQ ID NO: 4 - DNA, 1515 bp, unidentified
  misc_feature 1..1515: note = F04
  source 1..1515: mol_type = other DNA
agagtttgatcctggctcaggatgaacgctagctacaggcttaacacatgcaagtcgaggggcagcatgaacttagcttgctaagtttgatggcgaccggcgcacgggtgagtaacacgtatccaacctgccgatgactcggggatagcctttcgaaagaaagattaatacccgatggcatagttcttccgcatggtagaactattaaagaatttcggtcatcgatggggatgcgttccattaggttgttggcggggtaacggcccaccaagccttcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagtgaggcacgtgtgcctttttgtatgtaccgtatgaataaggatcggctaactccgtgccagcagccgcggtaatacggaggatccgagcgttatccggatttattgggtttaaagggagcgtaggcggacgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttgaattgcaactgaatgatgtggagacatgtcagccgcaaggcagttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcaaggagcgccctagggtaaaactggtgattggggctaagtcgtaacaaggtagccgtaccggaaggtgcggctggaacacct

SEQ ID NO: 5 - DNA, 1517 bp, unidentified
  misc_feature 1..1517: note = F05
  source 1..1517: mol_type = other DNA
agagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatacccgatggtataatcagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacct

SEQ ID NO: 6 - DNA, 1515 bp, unidentified; identical to SEQ ID NO: 4
  misc_feature 1..1515: note = F06
  source 1..1515: mol_type = other DNA

SEQ ID NO: 7 - DNA, 1518 bp, unidentified
  misc_feature 1..1518: note = F07
  source 1..1518: mol_type = other DNA
agagtttgatcctggctcaggatgaacgctagctacaggcttaacacatgcaagtcgaggggcagcatttcagtttgcttgcaaactggagatggcgaccggcgcacgggtgagtaacacgtatccaacctgccgataactcggggatagcctttcgaaagaaagattaatatccgatggtatatttctcccgcatgagagagatattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaaatgttccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactggtcactgacactgaggctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatagtggaaacattatagccgcaaggcaaatgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggccgctacctggtgacaggatgccaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaaggagcgtcctagggtaaaactggtaattggggctaagtcgtaacaaggtagccgtaccggaaggtgcggctggaacacct

SEQ ID NO: 8 - DNA, 1517 bp, unidentified; identical to SEQ ID NO: 3
  misc_feature 1..1517: note = F08
  source 1..1517: mol_type = other DNA

SEQ ID NO: 9 - DNA, 1515 bp, unidentified
  misc_feature 1..1515: note = F09
  source 1..1515: mol_type = other DNA
agagtttgatcctggctcaggatgaacgctagcgacaggcttaacacatgcaagtcgaggggcagcacgatgtagcaatacattggtggcgaccggcgcacgggtgagtaacgcgtatgcaacctacctatcagaggggaataacccggcgaaagtcggactaataccgcataaaacaggggttccacatggaaatatttgttaaagaattatcgctgatagatgggcatgcgttccattagatagttggtgaggtaacggctcaccaagtccacgatggataggggttctgagaggaaggtcccccacactggtactgagacacggaccagactcctacgggaggcagcagtgaggaatattggtcaatgggcgagagcctgaaccagccaagtcgcgtgaaggatgaaggatctatggtttgtaaacttcttttatatgggaataaagtgaggaacgtgttcctttttgtatgtaccatatgaataagcatcggctaactccgtgccagcagccgcggtaatacggaggatgcgagcgttatccggatttattgggtttaaagggtgcgtaggtggttaattaagtcagcggtgaaagtttgtggctcaaccataaaattgccgttgaaactggttgacttgagtatatttgaggtaggcggaatgcgtggtgtagcggtgaaatgcatagatatcacgcagaactccgattgcgaaggcagcttactaaactataactgacactgaagcacgaaagcgtggggatcaaacaggattagataccctggtagtccacgcagtaaacgatgattactagctgtttgcgatacacagtaagcggcacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgcattcggaccggagtggaaacacttcttctagcaatagccgtttgcgaggtgctgcatggttgtcgtcagctcgtgccgtgaggtgtcggcttaagtgccataacgagcgcaacccttatcactagttactaacaggtcatgctgaggactctagtgagactgccagcgtaagctgtgaggaaggtggggatgacgtcaaatcagcacggcccttacatccggggcgacacacgtgttacaatggtggggacaaagggcagctaccgtgtgagcggatgctaatctccaaaccccatctcagttcggatcgaagtctgcaacccgacttcgtgaagctggattcgctagtaatcgcgcatcagccatggcgcggtgaatacgttcccgggccttgtacacaccgcccgtcaagccatgggagttgggggtacctaaagtccgtaaccgcaaggatcggcctagggtaaaaccgatgactggggctaagtcgtaacaaggtagccgtaccggaaggtgcggctggaacacct

SEQ ID NO: 10 - DNA, 1519 bp, unidentified
  misc_feature 1..1519: note = F10
  source 1..1519: mol_type = other DNA
agagtttgatcctggctcaggatgaacgctggcggcgtgcttaacacatgcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacct

SEQ ID NO: 11 - DNA, 1519 bp, unidentified
  misc_feature 1..1519: note = F11
  source 1..1519: mol_type = other DNA
agagtttgatcctggctcaggatgaacgctggcggcgtgcttaacacatgcaagtcgaacgggaatcacttcattgagacttcggtggatttgatttgattctagtggcggacgggtgagtaacgcgtgggtaacctgccttatacagggggataacagtcagaaatgactgctaataccgcataagcgcacaggaccgcatggtccggtgtgaaaaactccggtggtataagatggacccgcgttggattagcttgttggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggaggggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcatggccattcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccgactcttaaccgagtctttccttcgggacagaggagacaggtggtgcatggttgtcgtcagctcgtgtcgtgagatgttgggttaagtcccgcaacgagcgcaacccctatcctcagtagccagcaagttaagttgggcactctgtggagactgccagggataacctggaggaaggcggggatgacgtcaaatcatcatgccccttatgatttgggctacacacgtgctacaatggcgtaaacaaagggaagcgagattgtgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacctaactgcaaagaaggagctgccgaaggcgggaccgatgactggggtgaagtcgtaacaaggtagccgtatcggaaggtgcggctggatcacct

SEQ ID NO: 12 - DNA, 1519 bp, unidentified
  misc_feature 1..1519: note = F12
  source 1..1519: mol_type = other DNA
agagtttgatcctggctcaggatgaacgctggcggcgtgcttaacacatgcaagtcgaacgaagcacttgaatggaattcttcggaaggaagctcaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaatggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccgctctttaatcggagctttccttcgggacagaggagacaggtggtgcatggttgtcgtcagctcgtgtcgtgagatgttgggttaagtcccgcaacgagcgcaacccctatcttcagtagccagcggcaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacct

SEQ ID NO: 13 - DNA, 1518 bp, unidentified
  misc_feature 1..1518: note = F13
  source 1..1518: mol_type = other DNA
agagtttgatcctggctcaggatgaacgctggcggcgtgcctaacacatgcaagtcgagcgaagcggtctggaggaagttttcggatggaatccggattgactgagcggcggacgggtgagtaacgcgtgggtaacctgcctcatacagggggataacagttagaaatggctgctaataccgcataagcgcacagcttcgcatggagcagtgtgaaaaactccggtggtatgagatggacccgcgtctgattagctggttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatagcaagtctggagtgaaagcccggggctcaaccccggtactgctttggaaactgttaagctagagtgctggagaggtaagtggaattcctagtgtagcggtgaaatgcgtagatattaggaggaacaccagtggcgaaggcggcttactggacagtaactgacgttgaggctcgaaagcgtggggagcaaacaggattagataccctggtagtccacgccgtaaacgatgaatactaggtgttggtgggcaaagcccatcggtgccgccgcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatcggaatgaccgggaagtaatgttcccttctctacggagcattctagacaggtggtgcatggttgtcgtcagctcgtgtcgtgagatgttgggttaagtcccgcaacgagcgcaacccttatccttagtagccagcagtaagatgggcactctagggagactgccagggataacctggaggaaggtggggatgacgtcaaatcatcatgccccttatgatttgggctacacacgtgctacaatggcgtaaacaaagagaggcgagcctgcgagggggagcgaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgactcaaccgaaaggggagagctgccgaaggcggggcaggtaactggggtgaagtcgtaacaaggtagccgtatcggaaggtgcggctggatcacct

SEQ ID NO: 14 - DNA, 1519 bp, unidentified
  misc_feature 1..1519: note = F14
  source 1..1519: mol_type = other DNA
agagtttgatcctggctcaggatgaacgctggcggcgtgcttaacacatgcaagtcgaacgaagcgcttaaacggatttcttcggattgaagtttttgtgactgagtggcggacgggtgagtaacgcgtgggtaacctgcctcatacagggggataacagttagaaatgactgctaataccgcataagcgcacagtgctgcatggcacagtgtgaaaaactccggtggtatgagatggacccgcgtctgattagctagttggtggggtaacggcctaccaaggcgacgatcagtagccggcctgagagggtgaacggccacattgggactgagacacggcccaaactcctacgggaggcagcagtggggaatattgcacaatgggggaaaccctgatgcagcgacgccgcgtgagcgaagaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggttgtgtaagtctgatgtgaaagcccggggctcaaccccgggactgcattggaaactatgtaactagagtgtcggagaggtaagcggaattcctagtgtagcggtgaaatgcgtagatattaggaggaacaccagtggcgaaggcggcttactggacgatcactgacgttgaggctcgaaagcgtggggagcaaacaggattagataccctggtagtccacgccgtaaacgatgactactaggtgtcggggagcaaagctcttcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatcccggtgaccggcaagtaatgttgcctttccttcgggacaccggtgacaggtggtgcatggttgtcgtcagctcgtgtcgtgagatgttgggttaagtcccgcaacgagcgcaacccctatcttcagtagccagcatttaaggtgggcactctggagagactgccagggataacctggaggaaggtggggatgacgtcaaatcatcatgccccttatgaccagggctacacacgtgctacaatggcgtaaacaaagggaagcgaacctgtgaggggaagcaaatctcaaaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagtcagtaacgcccgaagtcagtgacccaaccgtaaggagggagctgccgaaggtgggaccgataactggggtgaagtcgtaacaaggtagccgtatcggaaggtgcggctggatcacct

SEQ ID NO: 15 - DNA, 1519 bp, unidentified; identical to SEQ ID NO: 10
  misc_feature 1..1519: note = F15
  source 1..1519: mol_type = other DNA

SEQ ID NO: 16 - DNA, 1521 bp, unidentified
  misc_feature 1..1521: note = F16
  source 1..1521: mol_type = other DNA
agagtttgatcctggctcaggatgaacgctggcggcgtgcttaacacatgcaagtcgaacgaaacaccttatttgattttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactagagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagtgacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgtaaggagggagctgccgaaggcgggaccgataactggggtgaagtcgtaacaaggtagccgtatcggaaggtgcggctggatcacct

SEQ ID NO: 17 - DNA, 1518 bp, unidentified
  misc_feature 1..1518: note = F17
  source 1..1518: mol_type = other DNA
agagtttgatcctggctcaggatgaacgctggcggcgtgcttaacacatgcaagtcgaacggggaacattttatggaagcttcggtggaaatagcttgttcctagtggcggacgggtgagtaacgcgtgggtaacctgcctcacactgggggataacagtcagaaatgactgctaataccgcataagcgcacgggattgcatgatccagtgtgaaaaactccggtggtgtgagatggacccgcgttggattagccagttggcagggtaacggcctaccaaagcgacgatccatagccggcctgagagggtggacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaggcgggggctcaacccccggactgcattggaaactgttgatcttgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggtgagtaacgtcacctttccttcgggacagaggagacaggtggtgcatggttgtcgtcagctcgtgtcgtgagatgttgggttaagtcccgcaacgagcgcaacccctatccttagtagccagcggtttggccgggcactctgaggagactgccagggataacctggaggaaggcggggatgacgtcaaatcatcatgccccttatgatttgggctacacacgtgctacaatggcgtaaacaaagggaagcgagagtgtgagcttaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgaccgaaccgaaaggacggagctgccgaaggcgggacggatgactggggtgaagtcgtaacaaggtagccgtatcggaaggtgcggctggatcacct

SEQ ID NO: 18 - DNA, 1522 bp, unidentified
  misc_feature 1..1522: note = F18
  source 1..1522: mol_type = other DNA
agagtttgatcctggctcaggatgaacgctggcggcgtgcctaacacatgcaagtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtagagctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggagctgccgaaggcggggcaggtaactggggtgaagtcgtaacaaggtagccgtatcggaaggtgcggctggatcacct

SEQ ID NO: 19 - DNA, 1520 bp, unidentified
  misc_feature 1..1520: note = F19
  source 1..1520: mol_type = other DNA
agagtttgatcctggctcaggatgaacgctggcggcgtgcttaacacatgcaagtcgaacggggattattttgacagagacttcggttgaagtcgttataatcctagtggcggacgggtgagtaacgcgtgggtaacctgcctcacactgggggataacagtcagaaatgactgctaataccgcataagcgcacgggattgcatgatccagtgtgaaaaactccggtggtgtgagatggacccgcgttggattagccagttggcagggtaacggcctaccaaagcgacgatccatagccggcctgagagggtggacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggagcagcaagtctgatgtgaaaggcgggggctcaacccccggactgcattggaaactgttgatcttgagtaccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcagagccattcggtgccgcagcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccctctgaccggtgagtaacgtcacctttccttcgggacagaggagacaggtggtgcatggttgtcgtcagctcgtgtcgtgagatgttgggttaagtcccgcaacgagcgcaacccctatccttagtagccagcggtttggccgggcactctgaggagactgccagggataacctggaggaaggcggggatgacgtcaaatcatcatgccccttatgatttgggctacacacgtgctacaatggcgtaaacaaagggaagcgagagtgtgagcttaagcaaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgaccgaaccgaaaggacggagctgccgaaggcgggacggatgactggggtgaagtcgtaacaaggtagccgtatcggaaggtgcggctggatcacct

SEQ ID NO: 20 - DNA, 1528 bp, unidentified
  misc_feature 1..1528: note = F20
  source 1..1528: mol_type = other DNA
agagtttgatcctggctcaggatgaacgctggcggcatgcctaatacatgcaagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggaaacaaataccctagagatagggggataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgagccgtcgaaggtaggaccgatgactggggttaagtcgtaacaaggtatccctacgggaacgtggggatggatcacct

SEQ ID NO: 21 - DNA, 1521 bp, unidentified
  misc_feature 1..1521: note = F21
  source 1..1521: mol_type = other DNA
agagtttgatcctggctcaggatgaacgctggcggcgtgcttaacacatgcaagtcgagcgaagcacttaagtggatctcttcggattgaaacttatttgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttataggagggagctgccgaaggcgggaccgataactggggtgaagtcgtaacaaggtagccgtatcggaaggtgcggctggatcacct

SEQ ID NO: 22 - DNA, 1518 bp, unidentified
  misc_feature 1..1518: note = F22
  source 1..1518: mol_type = other DNA
agagtttgatcctggctcaggatgaacgctggcggcgtgcctaacacatgcaagtcgaacgaagcattttagatgaagttttcggatggattctgagatgactgagtggcggacgggtgagtaacacgtggataacctgcctcacactgggggacaacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagataatgacggtacctgactaagaagccccggctaactacgtgccagcagccgcggtaatacgtagggggcaagcgttatccggatttactgggtgtaaagggagcgtagacggcatggcaagtctgaagtgaaaacccagggctcaaccctgggactgctttggaaactgtcaagctagagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgagtgctaggtgttggggggcaaagcccttcggtgccgtcgcaaacgcaataagcactccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgcctttccttcgggacaagagagacaggtggtgcatggttgtcgtcagctcgtgtcgtgagatgttgggttaagtcccgcaacgagcgcaacccttatccttagtagccagcattaagatgggcactctagggagactgccagggacaacctggaggaaggtggggatgacgtcaaatcatcatgccccttatgatttgggctacacacgtgctacaatggcgtaaacaaagggaagcgaccctgcgaaggtgagcaaatctcaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggagggagctgccgaaggcggggcaggtaactggggtgaagtcgtaacaaggtagccgtatcggaaggtgcggctggatcacct

SEQ ID NO: 23 - DNA, 1519 bp, unidentified
  misc_feature 1..1519: note = F23
  source 1..1519: mol_type = other DNA
agagtttgatcctggctcaggatgaacgctggcggcgtgcttaacacatgcaagtcgaacgaagcactttaccggatttcttcgggatgaaagttttgtgactgagtggcggacgggtgagtaacgcgtgggtaacctgcctcatacagggggataacagttagaaatgactgctaataccgcataagaccacaggattgcatgatccggtggtaaaaactccggtggtatgagatggacccgcgtctgattaggtagttggtggggtaacggctcaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagaggcaagtctgatgtgaaaacccggggctcaaccccgggactgcattggaaactgtttttctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggaggcaaagcctttcggtgccgcagcaaacgcaataagcagtccacctggggagtacgttcgcaagaatgaaactcaaaggaattgacggggacccgcacaagcggtggagcatgtggtttaattcgaagcaacgcgaagaaccttacctgcccttgacatccggctgaccggcgagtaatgtcgcctttccttcgggacagccgagacaggtggtgcatggttgtcgtcagctcgtgtcgtgagatgttgggttaagtcccgcaacgagcgcaacccttatctttagtagccagcatttcggatgggcactctagagagactgccagggataacctggaggaaggtggggatgacgtcaaatcatcatgccccttatgggcagggctacacacgtgctacaatggcgtaaacaaagggaggcaagcctgcgagggtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagttggtaacgcccgaagtcagtgacccaaccgtaaggagggagctgccgaaggtgggaccgataactggggtgaagtcgtaacaaggtagccgtatcggaaggtgcggctggatcacct

SEQ ID NO: 24 - DNA, 1520 bp, unidentified
  misc_feature 1..1520: note = F24
  source 1..1520: mol_type = other DNA
agagtttgatcctggctcaggatgaacgctggcggcgtgcctaacacatgcaagtcgaacgaagttacacagaggaagttttcggatggaatcggtataacttagtggcggacgggtgagtaacgcgtgggaaacctgccctgtaccgggggataacacttagaaataggtgctaataccgcataagcgcacggaaccgcatggttgtgtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaaacccagggctcaaccttgggattgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctggggagtacgttcgcaagaatgaaactcaaaggaattgacggggacccgcacaagcggtggagcatgtggtttaattcgaagcaacgcgaagaaccttaccaagtcttgacatcccattgacgtgcctgtaaagaggcattcccttcggggcaatggagacaggtggtgcatggttgtcgtcagctcgtgtcgtgagatgttgggttaagtcccgcaacgagcgcaacccttattcttagtagccagcaggtaaagctgggcactctaaggagactgccggggataacccggaggaaggcggggatgacgtcaaatcatcatgccccttatgatttgggctacacacgtgctacaatggcgtaaacaaagggaagcgagacagtgatgtggagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagtctgtgacctaaccgaaagggaggagcagccgaaggcaggtctgataactggggtgaagtcgtaacaaggtagccgtatcggaaggtgcggctggatcacct

SEQ ID NO: 25 - DNA, 1519 bp, unidentified; identical to SEQ ID NO: 23
  misc_feature 1..1519: note = F25
  source 1..1519: mol_type = other DNA

SEQ ID NO: 26 - DNA, 1520 bp, unidentified
  misc_feature 1..1520: note = F26
  source 1..1520: mol_type = other DNA
agagtttgatcctggctcaggatgaacgctggcggcgtgcttaacacatgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgtatcggaaggtgcggctggatcacct

SEQ ID NO: 27 - DNA, 1519 bp, unidentified; identical to SEQ ID NO: 11
  misc_feature 1..1519: note = F27
  source 1..1519: mol_type = other DNA

SEQ ID NO: 28 - DNA, 1491 bp, unidentified
  misc_feature 1..1491: note = F28
  source 1..1491: mol_type = other DNA
agagtttgatcctggctcaggatgaacgctggcggcgtgcctaacacatgcaagtcgagcgattctcttcggagaagagcggcggacgggtgagtaacgcgtgggtaacctgccctgtacacacggataacataccgaaaggtatgctaatacgggataacataagaaattcgcatgtttttcttatcaaagctccggcggtacaggatggacccgcgtctgattagctagttggtgaggtaacggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtcttttaagtcaggagtgaaaggctacggctcaaccgtagtaagctcttgaaactggaggacttgagtgcaggagaggagagtggaattcctagtgtagcggtgaaatgcgtagatattaggaggaacaccagtagcgaaggcggctctctggactgtaactgacgctgaggcacgaaagcgtggggagcaaacaggattagataccctggtagtccacgccgtaaacgatgagtactagctgtcggaggttacccccttcggtggcgcagctaacgcattaagtactccgcctggggagtacgctcgcaagagtgaaactcaaaggaattgacggggacccgcacaagtagcggagcatgtggtttaattcgaagcaacgcgaagaaccttacctaagcttgacatccttttgaccgatgcctaatcgcatctttcccttcggggacagaagtgacaggtggtgcatggttgtcgtcagctcgtgtcgtgagatgttgggttaagtcccgcaacgagcgcaacccttgcctttagttgccatcattaagttgggcactctagagggactgccagggataacctggaggaaggtggggatgacgtcaaatcatcatgccccttatgcttagggctacacacgtgctacaatgggtggtacagagggcagcgaagtcgtgaggccaagctaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttgggggcgcccgaagccggctagctaaccttttggaagcggtcgtcgaaggtgaaaccaataactggggtgaagtcgtaacaaggtagccgtatcggaaggtgcggctggatcacct

SEQ ID NO: 29 - DNA, 1519 bp, unidentified
  misc_feature 1..1519: note = F29
  source 1..1519: mol_type = other DNA
agagtttgatcctggctcaggatgaacgctggcggcgtgcttaacacatgcaagtcgagcgaagcacttttgcagatttcttcggattgaagcaattgtgactgagcggcggacgggtgagtaacgcgtgggtaacctgcctcatacagggggataacagttggaaacggctgctaataccgcataagcgcacagtaccgcatggtaccgtgtgaaaaactccggtggtatgagatggacccgcgtctgattagctagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccttatctttagtagccagcatttaaggtgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacct

SEQ ID NO: 30 - DNA, 1512 bp, unidentified
  misc_feature 1..1512: note = F30
  source 1..1512: mol_type = other DNA
agagtttgatcctggctcaggatgaacgctggcggcgtgcctaatacatgcaagtcggacgcaatgcttcggcattgagtggcgaacgggtgagtaagacataagcaacctgcccctgtgagggggataactgctggaaacggcagctaagaccgcataggcatagaggacgcatgtcgactatgttaaatatcccacgggatagcacagggatgggcttatgacgcattagccagctggtgaggtaacggctcaccagggcgacgatgcgtagccggcctgagagggtggacggccacactgggactgagacacggcccagactcctacgggaggcagcagtagggaattttcggcaatgggcgaaagcctgaccgagcaacgccgcgtgaaggaagaagtcattcgtgatgtaaacttctgttatgaaggaagaacggcagatggagggaatgccatgtgcgtgacggtacttcatgaggaagccacggctaactacgtgccagcagccgcggtaatacgtaggtggcgagcgttatccggaatcattgggcgtaaagagggagcaggcggcagtgcaggtctgcggtgaaagaccggagctaaacttcggtaagccgtggaaaccgcacagctagagagcatcagaggatcgcggaattccatgtgtagcggtgaaatgcgtagatatatggaggaacaccagtggcgaaggcggcggtctggggtgcagctgacgctcagtcccgaaagcgtggggagcaaataggattagataccctagtagtccacgccgtaaacgatgagtgctaagtgttgggggtcagacctcagtgctggagttaacgcaataagcactccgcctgagtagtacgttcgcaagaatgaaactcaaaggaattgacgggggcccgcacaagcggtggagcatgtggtttaattcgaagcaacgcgaagaaccttaccaggtcttgacatggagataaaggccctggagacagggagatagatatatctcacacaggtggtgcatggttgtcgtcagctcgtgtcgtgagatgttgggttaagtcccgcaacgagcgcaacccctgttgccagttgccagcattaggttggggactctggcgagactgcctctgcaaggaggaggaaggcggggatgacgtcaaatcatcatgccccttatgacctgggctacacacgtgctacaatggacggatcagagggaggcgaagccgcgaggtggagcgaaacccagaaacccgttcacagttcggactgcagtctgcaactcgactgcacgaagctggaatcgctagtaatcgcgaatcagcatgtcgcggtgaatacgttctcgggccttgtacacaccgcccgtcacaccatgagagttggtaacacccgaagccggtggcccaaccgcaaggagggagctgtctaaggtgggactgatgattggggtgaagtcgtaacaaggtatccctacgggaacgtggggatggatcacct

SEQ ID NO: 31 - DNA, 1523 bp, unidentified
  misc_feature 1..1523: note = F31
  source 1..1523: mol_type = other DNA
agagtttgatcctggctcaggatgaacgctggcggcgtgcttaacacatgcaagtcgaacgaagcattttggaaggaagttttcggatggaattccttaatgactgagtggcggacgggtgagtaacgcgtggggaacctgccctatacagggggataacagctggaaacggctgctaataccgcataagcgcacagaatcgcatgattcggtgtgaaaagctccggcagtataggatggtcccgcgtctgattagctggttggcggggtaacggcccaccaaggcgacgatcagtagccggcttgagagagtggacggccacattgggactgagacacggcccaaactcctacgggaggcagcagtggggaatattgcacaatggggggaaccctgatgcagcgacgccgcgtgagtgaagaagtatttcggtatgtaaagctctatcagcagggaagaaaaaagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattcgaagcaacgcgaagaaccttacctggtcttgacatctaactgaccggttcgtaatgggacctttccttcgggacagttaagacaggtggtgcatggttgtcgtcagctcgtgtcgtgagatgttgggttaagtcccgcaacgagcgcaacccctatctttagtagccagcatataaggtgggcactctagagagactgccagggataacctggaggaaggtggggacgacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccttataggagggagctgccgaaggtgggaccgataactggggtgaagtcgtaacaaggtagccgtatcggaaggtgcggctggatcacct

SEQ ID NO: 32 - DNA, 1520 bp, unidentified
  misc_feature 1..1520: note = F32
  source 1..1520: mol_type = other DNA
agagtttgatcctggctcaggatgaacgctggcggcgtgcttaacacatgcaagtcgagcgaagcgctaagacggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggagctgccgaaggcgggaccgataactggggtgaagtcgtaacaaggtagccgtatcggaaggtgcggctggatcacct

SEQ ID NO: 33 - DNA, 1515 bp, unidentified
  misc_feature 1..1515: note = F33
  source 1..1515: mol_type = other DNA
agagtttgatcctggctcaggatgaacgctggcggcgtgcctaatacatgcaagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaaggaaggagctgtctaaggtgggattgatgattggggtgaagtcgtaacaaggtatccctacgggaacgtggggatggatcacct

SEQ ID NO: 34 - DNA, 1552 bp, unidentified
  misc_feature 1..1552: note = F34
  source 1..1552: mol_type = other DNA
agagtttgatcctggctcaggacgaacgctggcggcgtgcttaacacatgcaagtcgaacgaagagcgatggaagcttgcttctatcaatcttagtggcgaacgggtgagtaacgcgtaatcaacctgcccttcagagggggacaacagttggaaacgactgctaataccgcatacgatctaatctcggcatcgaggatagatgaaaggtggcctctacatgtaagctatcactgaaggaggggattgcgtctgattagctagttggaggggtaacggcccaccaaggcgatgatcagtagccggtctgagaggatgaacggccacattgggactgagacacggcccagactcctacgggaggcagcagtggggaatcttccgcaatggacgaaagtctgacggagcaacgccgcgtgagtgatgacggccttcgggttgtaaagctctgttaatcgggacgaaaggccttcttgcgaatagtgagaaggattgacggtaccggaatagaaagccacggctaactacgtgccagcagccgcggtaatacgtaggtggcaagcgttgtccggaattattgggcgtaaagcgcgcgcaggcggataggtcagtctgtcttaaaagttcggggcttaaccccgtgatgggatggaaactgccaatctagagtatcggagaggaaagtggaattcctagtgtagcggtgaaatgcgtagatattaggaagaacaccagtggcgaaggcgactttctggacgaaaactgacgctgaggcgcgaaagccaggggagcgaacgggattagataccccggtagtcctggccgtaaacgatgggtactaggtgtaggaggtatcgaccccttctgtgccggagttaacgcaataagtaccccgcctggggagtacgaccgcaaggttgaaactcaaaggaattgacgggggcccgcacaagcggtggagtatgtggtttaattcgacgcaacgcgaagaaccttaccaggtcttgacattgatggacagaaccagagatggttcctcttcttcggaagccagaaaacaggtggtgcacggttgtcgtcagctcgtgtcgtgagatgttgggttaagtcccgcaacgagcgcaacccctatcttatgttgccagcacgttagggtgggaactcatgagagactgccgcagacaatgcggaggaaggcggggatgacgtcaaatcatcatgccccttatgacctgggctacacacgtactacaatgggagttaatagacggaagcgagatcgcgagatggagcaaacccgagaaacactctctcagttcggatcgtaggctgcaactcgcctacgtgaagtcggaatcgctagtaatcgcaggtcagcatactgcggtgaatacgttcccgggccttgtacacaccgcccgtcacaccacgaaagtcggaagtgcccaaagccggtggggtaaccttcgggagccagccgtctaaggtaaagtcgatgattggggtgaagtcgtaacaaggtagccgtatcggaaggtgcggctggatcacct

SEQ ID NO: 35 - DNA, 1496 bp, unidentified
  misc_feature 1..1496: note = F35
  source 1..1496: mol_type = other DNA
agagtttgatcctggctcaggatgaacgctgacagaatgcttaacacatgcaagtctacttgatccttcgggtgaaggtggcggacgggtgagtaacgcgtaaagaacttgccttacagactgggacaacatttggaaacgaatgctaataccggatattatgattgggtcgcatgatctggttatgaaagctatatgcgctgtgagagagctttgcgtcccattagttagttggtgaggtaacggctcaccaagacgatgatgggtagccggcctgagagggtgaacggccacaaggggactgagacacggcccttactcctacgggaggcagcagtggggaatattggacaatggaccaaaagtctgatccagcaattctgtgtgcacgatgaagtttttcggaatgtaaagtgctttcagttgggaagaagtcagtgacggtaccaacagaagaagcgacggctaaatacgtgccagcagccgcggtaatacgtatgtcgcaagcgttatccggatttattgggcgtaaagcgcgtctaggcggcttagtaagtctgatgtgaaaatgcggggctcaaccccgtattgcgttggaaactgctaaactagagtactggagaggtaggcggaactacaagtgtagaggtgaaattcgtagatatttgtaggaatgccgatggggaagccagcctactggacagatactgacgctaaagcgcgaaagcgtgggtagcaaacaggattagataccctggtagtccacgccgtaaacgatgattactaggtgttgggggtcgaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcccaagaagttaacagagatgttttcgtgcctcttcggaggaacttggtgacaggtggtgcatggctgtcgtcagctcgtgtcgtgagatgttgggttaagtcccgcaacgagcgcaacccctttcgtatgttaccatcattaagttggggactcatgcgagactgcctgcgatgagcaggaggaaggtggggatgacgtcaagtcatcatgccccttatacgctgggctacacacgtgctacaatgggtagtacagagagctgcaaacctgcgagggtaagctaatctcataaaactattcttagttcggattgtactctgcaactcgagtacatgaagttggaatcgctagtaatcgcaaatcagctatgttgcggtgaatacgttctcgggtcttgtacacaccgcccgtcacaccacgagagttggttgcacctgaagtaacaggcctaaccgtaaggagggatgttccgagggtgtgattagcgattggggtgaagtcgtaacaaggtatccgtacgggaacgtgcggatggatcacct

SEQ ID NO: 36 - DNA, 1496 bp, unidentified; identical to SEQ ID NO: 35
  misc_feature 1..1496: note = F36
  source 1..1496: mol_type = other DNA

SEQ ID NO: 37 - DNA, 1530 bp, unidentified
  misc_feature 1..1530: note = F37
  source 1..1530: mol_type = other DNA
agagtttgatcatggctcagattgaacgctggcggcaggcctaacacatgcaagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacct

SEQ ID NO: 38 - DNA, 20 bp, synthetic construct
  source 1..20: mol_type = other DNA
agctttcgcggtatgggatg

SEQ ID NO: 39 - DNA, 20 bp, synthetic construct
  source 1..20: mol_type = other DNA
gccgtatctcagtcccaatg

SEQ ID NO: 40 - DNA, 21 bp, synthetic construct
  source 1..21: mol_type = other DNA
acacgtgatcgcatgatcgag

SEQ ID NO: 41 - DNA, 20 bp, synthetic construct
  source 1..20: mol_type = other DNA
ccaactagctaatcagacgc

SEQ ID NO: 42 - DNA, 22 bp, synthetic construct
  source 1..22: mol_type = other DNA
atggaagcttcggtggaaatag

SEQ ID NO: 43 - DNA, 20 bp, synthetic construct
  source 1..20: mol_type = other DNA
cacactgaatcatgcagtcc

SEQ ID NO: 44 - DNA, 20 bp, synthetic construct
  source 1..20: mol_type = other DNA
gcgaagcaagtctgaagtga

SEQ ID NO: 45 - DNA, 20 bp, synthetic construct
  source 1..20: mol_type = other DNA
ctccgacactctagcaaaac

SEQ ID NO: 46 - DNA, 21 bp, synthetic construct
  source 1..21: mol_type = other DNA
cggttgaagtcgttataatcc

SEQ ID NO: 47 - DNA, 21 bp, synthetic construct
  source 1..21: mol_type = other DNA
ccatgcagtcccgtgcgctta

SEQ ID NO: 48 - DNA, 21 bp, synthetic construct
  source 1..21: mol_type = other DNA
cttgcttccaaagagacttag

SEQ ID NO: 49 - DNA, 20 bp, synthetic construct
  source 1..20: mol_type = other DNA
gcagttatcccggacacatg

SEQ ID NO: 50 - DNA, 20 bp, synthetic construct
  source 1..20: mol_type = other DNA
aagtggatctcttcggattg

SEQ ID NO: 51 - DNA, 20 bp, synthetic construct
  source 1..20: mol_type = other DNA
ttatgcggtattagcagcca

SEQ ID NO: 52 - DNA, 20 bp, synthetic construct
  source 1..20: mol_type = other DNA
gattctgagatgactgagtg

SEQ ID NO: 53 - DNA, 20 bp, synthetic construct
  source 1..20: mol_type = other DNA
gcagtcatttctaactgttg

SEQ ID NO: 54 - DNA, 20 bp, synthetic construct
  source 1..20: mol_type = other DNA
tcggtataacttagtggcgg

SEQ ID NO: 55 - DNA, 20 bp, synthetic construct
  source 1..20: mol_type = other DNA
acaaccatgcggttccgtgc

SEQ ID NO: 56 - DNA, 20 bp, synthetic construct
  source 1..20: mol_type = other DNA
taccggatttcttcgggatg

SEQ ID NO: 57 - DNA, 20 bp, synthetic construct
  source 1..20: mol_type = other DNA
gatcatgcaatcctgtggtc

SEQ ID NO: 58 - DNA, 20 bp, synthetic construct
  source 1..20: mol_type = other DNA
tttatcgctctgagatggcc

SEQ ID NO: 59 - DNA, 20 bp, synthetic construct
  source 1..20: mol_type = other DNA
tcaacctctcagtccggcta

SEQ ID NO: 60 - DNA, 20 bp, synthetic construct
  source 1..20: mol_type = other DNA
gtacacacggataacatacc

SEQ ID NO: 61 - DNA, 20 bp, synthetic construct
  source 1..20: mol_type = other DNA
accaactagctaatcagacg

SEQ ID NO: 62 - DNA, 20 bp, synthetic construct
  source 1..20: mol_type = other DNA
tatcccacgggatagcacag

SEQ ID NO: 63 - DNA, 20 bp, synthetic construct
  source 1..20: mol_type = other DNA
tctcaggccggctacgcatc

SEQ ID NO: 64 - DNA, 20 bp, synthetic construct
  source 1..20: mol_type = other DNA
ttccttaatgactgagtggc

SEQ ID NO: 65 - DNA, 20 bp, synthetic construct
  source 1..20: mol_type = other DNA
cacaccgaatcatgcgattc

SEQ ID NO: 66 - DNA, 20 bp, synthetic construct
  source 1..20: mol_type = other DNA
tgaagtctttgtgactgagc

SEQ ID NO: 67 - DNA, 18 bp, synthetic construct
  source 1..18: mol_type = other DNA
atgcggtattagcagtca

SEQ ID NO: 68 - DNA, 20 bp, synthetic construct
  source 1..20: mol_type = other DNA
gtgcctcaaagcactggtag

SEQ ID NO: 69 - DNA, 20 bp, synthetic construct
  source 1..20: mol_type = other DNA
tctcaggtcggctacgcatc

SEQ ID NO: 70 - DNA, 20 bp, synthetic construct
  source 1..20: mol_type = other DNA
acttgatccttcgggtgaag

SEQ ID NO: 71 - DNA, 20 bp, synthetic construct
  source 1..20: mol_type = other DNA
atgttgtcccagtctgtaag

SEQ ID NO: 72 - DNA, 20 bp, synthetic construct
  source 1..20: mol_type = other DNA
gctgctttgctgacgagtgg

SEQ ID NO: 73 - DNA, 20 bp, synthetic construct
  source 1..20: mol_type = other DNA
ttggtcttgcgacgttatgc

SEQ ID NO: 74 - DNA, 20 bp, synthetic construct
  source 1..20: mol_type = other DNA
agcactagcggctgtggtat

SEQ ID NO: 75 - DNA, 20 bp, synthetic construct
  source 1..20: mol_type = other DNA
acttactcgggcccttgatt

SEQ ID NO: 76 - protein, 121 aa, unidentified
MAAGHPLTDEDRWPWLDTIAAWIDERVAAGEPAVVTCSALKKIYRDKLRRQGVVFVYMQGTFDEVMERLSHRQGHFMKPSMLQSQSDILEEPGDDEIHVNVHIGQADPEHEAMAVAGALGL

SEQ ID NO: 77 - protein, 175 aa, unidentified
MSTTNHDHHIYVLMGVSGSGKSAVASEVAHQLHAAFLDGDFLHPRCNIEKMASGEPLNDDDRKPWLQALNDAAFAMQRTNKVSLIVCSALKKHYRDLLREGNPNLSFIYLKGDFDVIESRLKARKGHFFKTQMLVTQFETLQEPGADETDVLVVDIDQPLEGVVASTIEVIKKGK

SEQ ID NO: 78 - protein, 178 aa, unidentified
MSIHVVVMGVAGCGKSTVAEAIHERLGYVYAEGDDFHPQANIDKMSAGIPLTDEDRWPWLKVINTWMVAREALDENTVVSSSALKRSYREVLAQNVPTFFVHLTGSQELIQQRLNERKGHFMPPALLPSQFAILEPLQPDENGVEVSIEGSVEEMVERAVAAVNEYAAKVASQSQQAE

SEQ ID NO: 79 - protein, 183 aa, unidentified
MSIHVVVMGVAGCGKSTVAEAIHERLGYAYAEGDDFHPQANIDKMSAGIPLTDEDRWPWLNVINSWMVAREALGENTVVSSSALKRSYREALAKDVPTFFIHLNGSHELIQQRLSERKGHFMPPALLPSQFAILEPLAPEENGVEISIEGSVDEMADRAIEALNAYAATVAATVAAQTATQAA

SEQ ID NO: 80 - protein, 121 aa, unidentified; identical to SEQ ID NO: 76

SEQ ID NO: 81 - protein, 121 aa, unidentified; identical to SEQ ID NO: 76

SEQ ID NO: 82 - protein, 519 aa, unidentified
MNTITPMDSANQEVWIGVDVGTTGVRAIAYTEDGTNVCSSEAFYPLLTPHPDWAEESPLQILEAVQEVISKTAAQLRYKNKELAGIALSTVMHSFAGLDEGKEPLMDMQTWADSRSASIVREMKKDEGLCRSFYERTGCPIHACYPLAKIIWLRQNQPELFRQMRYVGSLKDYLFYHLTGQWVIDKSAASTSGMYNERTLDWDDEILAYAGVTKDQLPPVVSTTYSQGLCEDAAKILRLPAGLPVVIGATDGVLVNVGIGAVEPGQLSGTIGTSGALRMLTRQPKTDPQMRTWCYNLTDDMWVAGGAINNGGMILRWVRDKICHYGGSALETLDIDPYDLMTMKAEHVDAGADGLICLPYFTGERAPYWNSELRGMFFGFSLNHSRSHMIRAVMEGICYSLNSVMAALKEFGDIKDIRVSGSFTKSKLWLQILSDVLNQPITLPDNSEGAAFGAAVLGFISSGKLKSIADTADLVHAKKIYTPIEENVAVYQQLYDIFVRLYHNLQGEFADITAYQQKL

SEQ ID NO: 83 - protein, 520 aa, unidentified
MNTITPMDSANQEVWIGVDVGTTGVRAIAYTEEGINVCSSEAFYPLLTPRPDWAEESPLQILEAVQDVIRKTTSQLRYKNKELAGIALSTAMHSFAALDENFEPLHDMQTWADSRSAAIVREMKKDEALCRSFYERTGCPIHACYPLAKIIWLRQNQPELFRQMRFAGSLKDYLYYHLTGEWLVDKSTASTTGMYNEHTLAWDDEIIAYAGITKDQLPPVVSTTYSHGLSEKAAKTLRLPEGLPVVIGATDGVLVNVGIGAVEPGQISGTIGTSGALRMLTRQPKIDPQMRTWCYNLTDDMWVAGGAINNGGMILRWVRDKICHYGGNALEMLDIDPYDLMTMKAEHVDAGADGLICLPCFTGERAPHWNSELRGMFFGFSLNHSRSHMIRAVMEGICYSLNSVMAALREFGDVKDIRVSGSFTKSKLWLQIMSDVFNEPITLPDNSEGAAFGAAVLGFISSGKLKSIADTANLVHAKKVYTPIEENAAVYRELYDIFTRLYQKLQGEFADIAAYQQKSK

SEQ ID NO: 84 - protein, 175 aa, unidentified; identical to SEQ ID NO: 77

SEQ ID NO: 85 - protein, 176 aa, unidentified
MSTTNHDHHVYVLMGVSGSGKSAVASEVAHQLHAAFLDGDFLHPRSNITKMASGEPLNDDDRTPWLQALNDAAFAMQRTNKVSLIVCSALKKSYRDILRKGNPNLSFIYLKGDFDVIESRLKARKGHFFKTQMLVTQFETLQEPGADESDVLIVDIDQPLEGVVASTIEVINKGSH

SEQ ID NO: 86 - protein, 573 aa, unidentified
MELTSQKLRKIAPELDPLRLGTGWKLEDLSKPQIMIESTFGDSHPGSGHLLDLVEEARQGVAEAGGFGARYFCTDICDGEAQGHDGINYSLPSRDMMANMIEIHANATPFDGGVFIASCDKGMPAHLMAVGRINIPSIIVTGGVMDAGPDLLTLEQIGKYNAMCERGEITKEKMEFYKHNACPSCGACSFMGTASTMQVMAEALGLMLPGSALMPATSKDLREFAKKAGKQAVWLAEHNLTPDKIVTMKSFENAIMVHAAISGSTNSLLHIPAIAKEFGIEIDGDTFDRLHRGAHYLLNIRPAGEWPAQFFYYAGGVPTIMEEIKDMLHLDVMTVTGKTLGENLEELKKNGFYDKCQSYLDQWNLKREDVIRPFDRPFGTDGSIAILKGNLAPDGAVVKHTVVPKEMFEAVLRARPFDCEEDAIHAVLTHEIQPGDAVIIRYEGPKGSGMPEMFYTTEAIASDATLGASIALLTDGRFSGASKGPAIGHISPEAAQGGPIALVEENDLIEISISNRVLRIVGVNGERKSEEEMEAILKERKSRWQPRENKYTKGVLKQFAEKAVSPMKGGYME

SEQ ID NO: 87 - protein, 573 aa, unidentified
MELNSQRVRALAPENDPLKMGMGWTVEDLDKPQILVESTFGDSHPGSAHLNQFVDEAMRGISDAGARGARYFTTDICDGIAQGHDGINYSLAHRDMIANMIEIHGNSTGFDGGVFIASCDKSVPACLMGLARLDMPSIVITGGVMDAGPDLLTLEQIGMYSAMCQRGEITDEKLTYYKHHACPSCGACSFMGTASTMQIMAEALGLMLPGSALMPATCEDLKEMAYKAGKQITELVKKGIKASDIVTEKAFENAIMVHAAISGSTNSLLHIPAMAHELGIEIDADTFDRMHRGAHYLLDIRPAGKWPAQFFYYAGGVPAVMEEIKSMLHLDVMTVTGKTLGENLEDLKKNGFYDKCEGYLKKWGLKRTDVIRTFDEPIGDDGTVGILRGNLAPEGSVVKHSAVPKEMFKAVLKARPFDCEEDAIAAVLSHRIQPGDAVFIRYEGPKGSGMPEMFYTTEAISSDPELGKTIALITDGRFSGASKGPAIGHVSPEAAEGGPIALVEEGDLIRIDIPERVLEIVGIHGQEMSREEVDKVLAERKAAWKPKAPKYTKGVLKIYSEHAVSPMKGGYMV

SEQ ID NO: 88 - protein, 580 aa, unidentified
MKLVSQEMRKIAPELDPLRIGTGWKPEDLSKPQVIIESTFGDSHPGSGHLNILVEEVRKGIEKAGGHGARYFCTDICDGESQGTDGINYSLASREMIANMIEIHANATPFDAGVYLASCDKGMPGNLMGLARADVPSVVVPGGTMNAGPEMLTLEQLGMYSAKYERGEIDEEKLEWAKCNACPSCGACSFIGTASTMQIMAEALGLALPGSALMPATSPDLLEYARQAGEQAVKLATMPGMRPSEIVTEKSFENAILVHAAVSGSTNCLLHIPAIAHEFGIEITGDTFDRLHRNARYLLDVRPAGRWPAECFYYAGGVPAIMEEIKEHLHLDVMTVTGKTLGENLEELRRNGFYEKCEKWLQEFNRRYNVNLTREDIIRSYDNAIGTDGSIAVLRGNLAPEGAVIKHTACPKEMFRAVLNARPFDSEEECLDAVLHHRVQKGDAVFIRYEGPKGSGMPEMFYTSEAISSDKELGRSIALITDGRFSGASTGPVIGHCSPEAVEGGPIALVEEGDLIEIDVPARKLNIIGVKGQRRSMEEIDEILEERRKNWQPREPKYKKGVLRLFSQHAVSPMKGAYLE

SEQ ID NO: 89 - protein, 573 aa, unidentified
MELNSQRVRALAPENDPLKIGMGWKVEDLDKPQIMVESTFGDSHPGSAHLDQFVNEAMRGIADAGGKGARYFTTDICDGIAQGHDGINYSLAHRDMIANMIEIHGNSTGFDGGVFIASCDKSVPACLMGLARLDMPSIVVTGGVMDAGPDLLTLEQIGAYSAMCQRGEISEEKLTYYKHNACPSCGACSFMGTASTMQIMAEALGLMLPGSALMPATCEDLKEMAYKAGLQVMELARKGLKVSDIVTMKSFENAIMVHAAISGSTNSLLHIPAIAHELGLEIDADTFDRMHRGAHYLLDIRPAGKWPAQFFYYAGGVPAVMEEIKSMLHLDVMTVTGKTLGENLEELKAGGFYDKCDGYLKKWGLKRTDVIRTFEEPIGTDGTVAILRGNLAPEGSVVKHSAVPKEMFKAVLKAKPFDCEEDAIEAVISRKIQPGDAVFIRYEGPKGSGMPEMFYTTEAISSDPELGKTIALITDGRFSGASKGPAIGHVSPEAADGGPIALVEEDDLIRIDIPERVLEIIGVKGEELPKEEVERILAERRKAWKPREAKYKKGVLKIYSEHAVSPMKGGYMV

SEQ ID NO: 90 - protein, 478 aa, unidentified
MKLVSQEMRKIAPELDPLRIGTGWKPEDLSKPQVIIESTFGDSHPGSGHLNILVEEVRKGIEKAGGHGARYFCTDICDGESQGTDGINYSLASREMIANMIEIHANATPFDAGVYLASCDKGMPGNLMGLARADVPSVVVPGGTMNAGPEMLTLEQLGMYSAKYERGEIDEEKLEWAKCNACPSCGACSFIGTASTMQIMAEALGLALPGSALMPATSPDLLEYARQAGEQAVKLATMPGMRPSEIVTEKSFENAILVHAAVSGSTNCLLHIPAIAHEFGIEITGDTFDRLHRNARYLLDVRPAGRWPAECFYYAGGVPAIMEEIKEHLHLDVMTVTGKTLGENLEELRRNGFYEKCERWLHEFNRRYNVNLTREDIIRSYDNAIGTDGSIAVLRGNLAPEGAVIKHTACPKEMFRAVLNARPFDSEEECLDAVLHHRVQKGDAVFIRYEGPKGSGMPECFIPLRRSAAIKNLEEALP

SEQ ID NO: 91 - protein, 573 aa, unidentified
MKLRSQELRKIAPELDPLRLGTGWKLEDLSKPQIMIESTFGDSHPGSGHLLELVEEARAGIAEAGGHGARYFTTDICDGEAQGHDGINYSLASRDMIANMIEIHANATPFDGGVFVASCDKGMPAHLMGVGRVNIPSIIVTGGVMDAGPDLLTLEQIGKYNAMCERGEITQEKLTFYKQNACPSCGACSFMGTASTMQIMAEALGLMLPGSALMPATSKDLRVVAREAGKQSVWLAEHDLTPDKIVTMKSFENAIMVHAAISGSTNSLLHLPAIAREFGIEIDGDTFDRLHRGAHYLLNIRPAGEWPAQFFYYAGGVPTIMEEIKDMLHLDVMTVTGKTLGENLEELKKNGFYDKCDSYLEKVGLKREDVIRPFDKPFGTDGSIAILYGNLAPEGAVVKHTVVPKEMFEATLKARPFDCEEDAIHAVLTHEIKPGDAVIIRYEGPKGSGMPEMFYTTEAIASDAELGASIALLTDGRFSGASKGPAIGHISPEAAEGGPIALVEEGDLIEVNIPGRVLRIVGIAGEKKTQEEIETVLKERRSKWQPKPVKYEKGVLKIFSERAVSPMKGGYME

SEQ ID NO: 92 - protein, 572 aa, unidentified
MYRSEEVRKLAPEMDPLRLGMGWKRSDLDKPKIMIESTMGDSHPGSAHLMEFVEQAAKGVHDAGGKAARYYTTDICDGQAQGHDGINYSLASRDMIANMVEIHENATPFDGGVFIASCDKGMPGLLMGIGRCNIPSIMVTGGVMEAGPDMLTLEEIGKYSAMCQRGEITGEQFLFYKHHACPSCGACSFMGTACTMQVMAEALGLMLPGTSVMPATCADLKRAAYDAGLQAMKLAEMDLKPRDMVTLKSFENAIMVHAAISGSTNALLHLPAIAHEFGITIDADTFERIHKSAHYLLNVRPAGEWPAAYFYYAGGVPAVMEEIRHSLHLDVMTVTGKPLGENLEELKKNGFYENCARLLEPTGLKKEQIIYPYDKPIGDKGTIAILKGNLAPEGAVVKHSAVPKEMHVAMLNARPFDCEEDAIAAVLSRAIRPGDAVFIRYEGPKGSGMPEMFYTTEAIASDEELGKSIALITDGRFSGASKGPAIGHVSPEAAEGGPIALVEEGDLIEIDIPRRVLRIAGIKGEAKSEEEILEVLEQRRKTWQPKPARYQSGVLKIYSEHAVGPMKGGYMK

SEQ ID NO: 93 - protein, 574 aa, unidentified
MKELHSQEVRKLAPENDPLKIGMGWTVEDLEKPQILVESTFGDSHPGSAHLDLFVERAVEGISKEGGKGARYFATDICDGIAQGHDGINYSLASRDMIANMIEIHGNATGFDAGVFIASCDKSVPAILMGIGRLNLPAVVVTGGVMEAGPDLLTLEQIGMYSAMYQRGEISEDKFLYYKHHACPSCGACSFMGTASTMQIMAEALGLMIPGTALMPATCDELKTAAYQAGELAVKLARSGLTARDIVTKDSFENAIMVHAAISGSTNSLLHIPAIAHEFGIEIDADTFDRMHRNAHYLLDIRPAGKWPAQFFYYAGGVPRIMEEIKSMLHLDARTINGRTVGENLEKLKKDGFYEKCESYLEPWGLKATDVIRTFEKPIGTNGTVAILRGNLAPDGAVVKHSAVPKEMFHAVLQARPFDSEEEAIEAVLTHKIRPGDAVIIRYEGPVGSGMPEMFYTTEAIASDESLAKSIALITDGRFSGASKGPAIGHVSPEAAAGGPIAFLETDDLILLDIEERKLEIIGVKGQKLAEEEIEKILEERKRAWIPKEEKYKSGVLKIYSEHAVSPMKGGYME

SEQ ID NO: 94 - protein, 574 aa, unidentified
MKEWNSQRVRALAPENDPLKLGMGWTAADLEKPQIMVESTFGDSHPGSAHLNQFVEEAMRGIKDAGGRGARYYTTDICDGIAQGHDGINYSLAHRDMIANMIEIHGNSTGFDGGVFIASCDKSVPACLMGLARLDMPSIVITGGVMDAGPDLLTLEQIGAYSAMYQRGEISEEKLTYYKHNACPSCGACSFMGTASTMQIMAEALGLMLPGSALMPAVCPELKEMAYKAGLQVMELAKQGLKVSDIVTMKSFENAIMVHAAISGSTNSLLHIPAMAHELGFEIDGDTFDRMHRGAHYLLDIRPAGKWPAQFFYYAGGVPAVMEEIKSMLHLDVMTVTGKTLGENLEELKQNGFYETCHSYLEKWNLKPTDVIRSFDNPIGTDGTVAVLRGNLAPEGSVVKHSAVPREMFQAVLKARPFDCEEDAIAAVLSGSIHPGDAVFIRYEGPKGSGMPEMFYTTEAISSDPELGKSIALITDGRFSGASKGPAIGHVSPEAAQGGPIALVEEGDLIHIDIPARILEIIGIKGQELSAKEVEQILEERRKKLQPREPKYKKGVLKIYSEHAVSPMRGGYME

SEQ ID NO: 95 - protein, 573 aa, unidentified
MELTSQKLRKIAPELDPLRLGTGWKLEDLSKPQIIIESTFGDSHPGSGHLLDLVEEARQGVAEAGGFGARYFCTDICDGEAQGHDGINYSLPSRDMMANMIEIHANATPFDGGVFIASCDKGMPAHLMAVGRINIPSIIVTGGVMDAGPDLLTLEQIGKYNAMCERGEITKEKMEFYKHNACPSCGACSFMGTASTMQVMAEALGLMLPGSALMPATSKDLREFAKKAGKQAVWLAEHNLTPDKIVTMKSFENAIMVHAAISGSTNSLLHIPAIAREFGIEIDADTFDRLHRGAHYLLNIRPAGEWPAQFFYYAGGVPTIMEEIKDMLHLDVMTVTGKTLGENLEELKKNGFYDKCQSYLDQWNLKREDVIRPFDRPFGTDGSIAILKGNLAPDGAVVKHTVVPKEMFEAVLRARPFDCEEDAIHAVLTHEIQPGDAVIIRYEGPKGSGMPEMFYTTEAIASDATLGASIALLTDGRFSGASKGPAIGHISPEAAQGGPIALVEENDLIEISISNRVLRIVGVNGERKSEEEMEAILKERKSRWQPRENKYTKGVLKQFAEKAVSPMKGGYME

SEQ ID NO: 96 - protein, 572 aa, unidentified
MLKSQKLREMAPELDPLRLGTGWKLEDLSKPQIIIESTFGDSHPGSGHLLELVEEARKGIDEAGGYGARYFTTDICDGEAQGHDGINYSLASRDMIANMIEIHANATPFDGGVFIASCDKGMPAHLMGLGRVNIPSIVVTGGVMDAGPDLLTLEQIGMYSAQYERGEISKEKFEFYKHHACPSCGACSFMGTASTMQIMAEALGLTLPGSALLPATSKDLREFAYRAGKQAVWLAKHDLKPRDIVTMKSFENAIMVHAAISGSTNSLLHLPAIAKEFGIEIDSDTFDRLHRGAHYLLDIRPAGRWPAQFFYYAGGVPMIMEQIKSMLHLDVMTVTGKTLGENLEELKQTGYYEKCFKNLEKYGIQREDVIRSFDNAFGTDGSIAILRGNLAPEGAVVKHTVVPKEMFEATLIARPFDCEEDAIAAVLSHKIQPGDAVFIRYEGPQGSGMPEMFYTTEAIASDEKLGASIALITDGRFSGASKGPAIGHVSPEAAQGGPIALVEEGDLIEINIPQRILRICGIHGQKMSEEDIQKVLEERRKHLKPFVSKYQDGVLKIFSEHAVSAMKGGYMK

SEQ ID NO: 97 - protein, 573 aa, unidentified
MKLRSQELRKIAPELDPLRLGTGWKLEDLSKPQIMIESTFGDSHPGSGHLLELVEEARAGIAEAGGHGARYFTTDICDGEAQGHDGINYSLASRDMIANMIEIHANATPFDGGVFVASCDKGMPAHLMGVGRVNIPSIIVTGGVMDAGPDLLTLEQIGMYNAMCERGEITEEKLTFYKHNACPSCGACSFMGTASTMQIMAEALGLMLPGSALMPATSKDLRTVAREAGKQSVWLAEHDLTPDKIVTMKSFENAIMVHAAISGSTNSLLHLPAIAREFGIEIDGDTFDRLHRGAHYLLNIRPAGEWPAQFFYYAGGVPTIMEEIKDMLHLDVMTVTGKTLGENLEELKKNGFYDKCDSYLEKVGLKREDVIRPFDKPFGTDGSIGILYGNLAPDGAVVKHTVVPKEMFEATLKARPFDCEEDAIHAVLTHEIKPGDAVIIRYEGPKGSGMPEMFYTTEAIASDAELGASIALLTDGRFSGASKGPAIGHISPEAAEGGPIALVEEGDLIEVNIPNRVLRIVGIAGEKKTQEEIEAVLKERRSKWQPKPVKYEKGVLKIFSERAVSPMKGGYME

SEQ ID NO: 98 - protein, 572 aa, unidentified
MKSQELRKIAPEIDPLRIGTGWSVADLSKPQIMIESTYGDSHPGSVGLLDLVYEVEKGINDHGAKASKFFTTDICDGEAQGHDGINYSLASRDMIAGMIEIHNNATPFDGSVFVASCDKGMPAHLMGAGRINNPCIFVTGGVMEAGPDMLTLEQIGKYSAMYLRGEISKEQFEYYQHHACPSCGACSFMGTASTMQIMTEALGLMLPGTALMPATCKDLKEQAYKAGKQIIKLAKMDLTARDIVTMKSFENAIMVHAAISGSTNSLLHLPAIASEFGIDLNADLFDQMHQNAHYLLDIRPAGKYPAEYFYYAGGVPAVMEAIKENLNLDVMTVTGKTLGENLEELKNNGYYENCQTFLDKINLKSTDIIRPYDDPIGKDGTIAILKGNLAPDGAVIKHSACPQEMMQAVLRARPFDSEEAAIDAIIKKQIKPGDAVFIRYEGPKGSGMPEMFYTGEAISSDQELAKSIALITDGRFSGASKGPVIGHVSPEASEGGPIALIEENDLIKIDVPQRRLEIIGIEGKQRTSQEITKILKERHAKWKPKEPKYKNGILSIYTKLATSPMNGGKMKG

SEQ ID NO: 99 - protein, 574 aa, unidentified
MKSQQLRKLAPELDSLRLGSGWKIDELSKPQIIVESSYGHSHPGSAHLDKLVDEAGIGIKEKGGRAANYFVTDICDGEAQGHDGMNYSLVSRDIMAAMMEIHVKATPFDAGVFIASCDKSVPAHLMAIARLDMPAIFMPGGIMKAGPNLLTLEQIGTYSAQYERKEITEEQFMVYKRDACPDCGACSFMGTASTMQVMAEALGIALPGSALIPAHLPELKETARKAGEHALGLAKEELKPSDIMTIQAFENAIMVHAAIAGSSNSLLHLPAIAHELGIQLSPDLFDKIHRKIPYLLNIRPSGFWPGEYFWYAGGVPAIMEEIKEFLHLDVMTVTGKTLEENLKDLQLNGFYENCHKYLPTLGVKVGDIIKPLHTPIKAQGAIAILKGNLAPEGAVVKHSAISPRLMQVTLKARVFDCEENAIEAVLQKKIHPGEAVFIRYEGPKGSGMPEMFYTTEAIASDPELVETIALITDGRFSGATRGPAIGHVSPEASEGGPIALVENDDLIRIDIPARTLDIIGTDSIERSVEEIEKILSERRAHWIKPESQYKKGILDIYTHNSVSPMKGAYMEFFM

SEQ ID NO: 100 - protein, 595 aa, unidentified
MELRSQAVRKLAPENDPLKIGMGWKVDDLAKPQIMVESTFGDSHPGSAHLDQFVKEAVQAVNTHGGKAARYFATDMCDGIAQGHDGINYSLPHRDAIVNLVEAQANATVYDGGVFIASCDKSMPAMLMSIGRLKDMSAIVVTGGVMEAHTLPKKYVVNDPACAINDLLTLEQIGKFDAWEKTGVIPNEQLDYYKHNACPSCGACSFMGTASTMQIMAEALGLMLPGTALMPATAPELKQAAYDAGVQVMKLVAEGIKAKDIVTMKSFENAIMVHAAISGSTNATMHLPAIAHEFGFEIDADTFDRMHRGAHYLLNIRPSGDWPAQYFYYAGGVPRVMEEIKSMLHLDVMTVTGKTLGENLEDLKKNGFYEHCDEILQEKTKGLGIKVTREDIIHSFDNAKGTDGSIAILKGNLAPEGSVIKHTACPKNMFHATLRARPFDSEEECISAVLNKQVQPGDAIFIRYEGPRGSGMPEMFYTGEAICADPKLASSVALITDGRFSGASRGPVIGHVSPEAAVGGPIALVEEGDLIRIDVPNRQLAIVGVNGEEKTPEEMEAILAQRRANWKPKAPKYTKGLLKLYSQHAVSPMKGAYME

SEQ ID NO: 101 - protein, 595 aa, unidentified; identical to SEQ ID NO: 100

SEQ ID NO: 102 - protein, 581 aa, unidentified
MQLKSQEMRKLAPELDPLRIGTGWKKEDLGKVQVMVESTYGDSHPGSGHLNILVEEVRKGIAEEGGFGARYHCTDICDGESQGTDGINYSLASREMIANMIEIHANATPFDAGVYLSSCDKGVPGNLMGLARVNIPSVFVPGGTMNAGPEMLTLEQLGMYSAKFERGEIDEEKLDWAKCNACPSCGACSFIGTASTMQIMAEALGLALPGTALMPSTSPDLLAFAREAGRQAVRIAQMENMRPSDIVTMDSFENAILVHAAISGSTNCLLHLPAIAHEFGIEITGDTFDRLHRNARYLLDVRPAGRWPAECFYYAGGVPAIMEEIKEHLHLDVMTVTGKTLGENLEELKNNGFYEKCEKWLQEFNKRYNVNLTKEDIIRPYDKAIGTDGSIAILRGNLAPEGAVIKHTACPKEMFKSVLRARPFDSEEECLDAVLKHKVQKGDAVFIRYEGPKGSGMPEMFYTSEAISSDKELGKSIALITDGRFSGASTGPVIGHCSPEAVDGGPIALVEEGDLIEIDVMERKLNIIGIAGERKSMEEIDEILAERRKNWKPREAKYKKGVLRLFSQHAASPMKGAYLEY

SEQ ID NO: 103 - protein, 581 aa, unidentified
MELKSQEMRKLAPELDPLRIGTGWKKEDLDKVQVMVESTYGDSHPGSGHLNILVEEVRAGVAEEGGFGARYFCTDICDGESQGTDGINYSLASREMIANMIEIHANATPFDAGVYLSSCDKGVPGNLMGLARVNIPSVVVPGGTMNAGPEMLTLEQLGMYSAKYERGEIDQEKLDWAKCNACPSCGACSFIGTASTMQIMAEALGLALPGSALMPATSPDLLAFAREAGRQAVRLATMEHMLPSDIVTMDSFENAILVHAAISGSTNCLLHIPAIAHEFGIEITGDTFDRLHRNARYLLDIRPAGKWPAECFYYAGGVPAIMEEIKEHLHLDVMTVTGKTLGENLEELKKNGFYEKCDKWLQEFNKRYNVNVIKEDIIRPYDKAIGTDGSIAVLRGNLAPEGAVIKHTACPKEMFKSVLRARPFDSEEECLDAVLKHKVQKGDAVFIRYEGPKGSGMPEMFYTSEAISSDAELGRSIALITDGRFSGASTGPVIGHCSPEAVEGGPIALVEEGDLIEIDVMERKLNIIGVAGERKAMEEIDEILKERRKNWKPKEMKYKTGVLRMFSQHATSPMKGAYLDF

SEQ ID NO: 104 - protein, 595 aa, unidentified
MELRSQAVRKLAPENDPLKIGMGWKVDDLAKPQIMVESTFGDSHPGSAHLDQFVKEAVQAVNTHGGKAARYFATDMCDGIAQGHDGINYSLPHRDAIVNLVEAQANATVYDGGVFIASCDKSMPAMLMSIGRLKDMSAIVVTGGVMEAHTLPKKYVVNDPACAINDLLTLEQIGKFDAWEKTGVIPNEQLDYYKHNACPSCGACSFMGTASTMQIMAEALGLMLPGTALMPATAPELKQAAYDAGVQVMKLVAEGIKAKDIVTMKSFENAIMVHAAISGSTNATMHLPAIAHEFGFEIDADTFDRMHRGAHYLLNIRPSGDWPAQYFYYAGGVPRVMEEIKSMLHLDVMTVTGKTLGENLEDLKKSGFYEHCDEILQEKTKGLGIKVTREDIIHSFNNAKGTDGSIAILKGNLAPEGSVIKHTACPKNMFHATLRAKPFDSEEECISAVLNKQVQPGDAIFIRYEGPRGSGMPEMFYTGEAICADPKLASSVALITDGRFSGASRGPVIGHVSPEAAVGGPIALVEEGDLIRIDVPNRQLAIVGVNGEEKTPEEMEAILAQRRANWKPKAPKYTKGLLKLYSQHAVSPMKGAYME

SEQ ID NO: 105 - protein, 573 aa, unidentified; identical to SEQ ID NO: 97

SEQ ID NO: 106 - protein, 573 aa, unidentified
MKLRSQELRKIAPELDPLRLGTGWKLEDLSKPQIMIESTFGDSHPGSGHLLELVEEVRAGVAEAGGHGARYFTTDICDGEAQGHDGINYSLASRDMIANMIEIHANATPFDGGVFVASCDKGMPAHLMGVGRVNIPSIIVTGGVMDAGPDLLTLEQIGMYNAMCERGEITQEKLTFYKQNACPSCGACSFMGTASTMQIMAEALGLMLPGSALMPATSKDLRTVAREAGKQSVWLAEHDLTPDKIVTMKSFENAIMVHAAISGSTNSLLHLPAIAREFGIEIDGDTFDRLHRGAHYLLNIRPAGEWPAQFFYYAGGVPTIMEEIKDMLHLDVMTVTGKTLGENLEELKKNGFYNKCDKYLEQVGLKREDVIRPFDKPFGTDGSIAILYGNLAPEGAVVKHTVVPKEMFEATLKARPFDCEEDAIHAVLTHEIKPGDAVIIRYEGPKGSGMPEMFYTTEAIASDAELGASIALLTDGRFSGASKGPAIGHISPEAAEGGPIALVEEGDLIEVNIPNRVLRIVGIAGEKKTPEEIETVLKERRSKWQPKPVKYEKGVLKIFSERAVSPMKGGYME

SEQ ID NO: 107 - protein, 573 aa, unidentified
MELNSQRVRALAPENDPLKIGMGWTVEDLDKPQILVESTFGDSHPGSAHLNLFVEEAMKGIRDSGGRGARYFTTDICDGIAQGHDGINYSLAHRDMIANMIEIHGNSTGFDGGVFIASCDKSVPACLMGLARLNMPSIVITGGVMEAGPDLLTLEQIGAYSAMCQRGEITEEKLTYYKQNACPSCGACSFMGTASTMQIMAEALGLMLPGSALMPATCPELKEMAYKAGKQVMELVKAGLKVSDIVTMEAFENAIMVHAAISGSTNSLLHIPAMAHELGLEIDADTFDRMHRGAHYLLDIRPAGKWPAQFFYYAGGVPAVMEEIKSMLHLDVMTVTGKTLGENLEELKQNGFYEKCSVSLEKWGLKRTDVIRPFDNPIGSDGTVAILRGNLAPEGSVVKHSAVPAEMFQAVLKARPFDCEEDAIAAVLSRKIRPGDAVFIRYEGPKGSGMPEMFYTTEAISSDPELGRSIALITDGRFSGASKGPAIGHVSPEAADGGPIALVEEDDLIRIDIPARVLEIIGVKGRELPPEEIDKILEERRANLKPREPKYKKGVLKIYSEHAVSPMKGGYME

SEQ ID NO: 108 - protein, 572 aa, unidentified; identical to SEQ ID NO: 98

SEQ ID NO: 109 - protein, 573 aa, unidentified
MKLKSQEVRALAPEMDPLRMGMGWKVDDLSKPQIIVESTFGDSHPGSAHLLELVEESVKGINHSGGKGARYFATDICDGMAQGHDGINYSLASRDTICNMIEIHANATTFDAGVFIASCDKAVPAHLMAIGRLKFPSIVVTGGVMDAGPDLLTLEQIGMYSAKCQRGEITEDQLTWYKQHACPSCGACSFMGTASTMQVMAEALGLMLPGTALMPATCEDLKKAAYDAGVQAVKLAEAGLKSTDIVTMKSFENAIMVHAAISGSSNSLLHIPAAAHEFGYELDADYFDRMHRNAHYLLDIRPAGKWPAQYFYYAGGVPRIMEEIKSMLHLDVLTVTGKTLGENLEDLKKNGYYEKCDEYLAKVGRKRTDIIRSFQNPIGDNGTIAILRGNLAPEGAEVKHSAVPKEMFQAVLKAKPFDCEEEAIAAVLDKTIRPGDAVIIRYEGPKGSGMPEMFYTTEAISSDEELSKSIALITDGRFSGASRGPAIGHVSPEAAEGGPIALIEEDDLIEIDIPARILRIIGVRGEKKTPEEMDAILAVRRKNWMPKPAKYTSGVLKIFSEHAVSPMKGGYME

SEQ ID NO: 110 - protein, 573 aa, unidentified
MELNSQRVRALAPENDPLKIGMGWKVEDLDKPQIMVESTFGDSHPGSAHLDQLVNEAMRGIADAGGKGARYFTTDICDGIAQGHDGINYSLAHRDMIANMIEIHGNSTGFDGGVFIASCDKSVPACLMGLARLDMPSIVVTGGVMDAGPDLLTLEQIGAYSAMCQRGEITEEKLTYYKHNACPSCGACSFMGTASTMQIMAESLGLMLPGSALMPATCADLREMAYKAGLQVMELAGKGLKVSDIVTMKSFENAIMVHAAISGSTNSLLHIPAIAHELGLEIDADTFDRMHRGAHYLLDIRPAGKWPAQFFYYAGGVPAVMEEIKSMLHLDVMTVTGKTLGENLEELKANGFYDKCDGYLKKWGLKRTDVIRTFEEPIGTDGTVAILRGNLAPEGSVVKHSAVPREMFKAVLRAKPFDCEEDAIEAVISRKIQPGDAVFIRYEGPKGSGMPEMFYTTEAISSDPELGKTIALITDGRFSGASKGPAIGHVSPEAADGGPIALVEEDDLIRIDIPERVLEIIGVKGKELPGEEVERILAERRKAWKPREAKYKKGVLKIYSEHVVSPMKGGYMV

SEQ ID NO: 111 - protein, 574 aa, unidentified
MMELNSQRVRGLAPENDPLKIGMGWTVEDLDKPQIMVESTFGDSHPGSAHLDLFVNEALRGIADAGGKGARYFTTDICDGIAQGHDGINYSLAHRDMIANMIEIHGNSTGFDGGIFIASCDKSVPACLMGLARLDMPSIVVTGGVMDAGPDLLTLEQIGAYSAMCQRGEITEEKLTYYKHNACPSCGSCSFMGTASTMQVMAESLGLMLPGSALMPAACGDLKEVAYKAGLQVMELARKGIKVSDIVTMKSFENAIMVHAAISGSTNSLLHIPAIAHEMGFEIDGDTFDRMHRGAHYLLDIRPAGKWPAQFFYYAGGVPAVMEEIKSMLHLDVMTVTGRTLGENLEELKSNGFYDKCGGYLEKWGLKRTDVIRTFEEPIGTDGTVGILRGNLAPEGSVVKHSAVPKEMFRAILKAKPFDCEEDAIEAVISRKIQPGDAVFIRYEGPKGSGMPEMFYTTEAISSDPELGKSIALITDGRFSGASKGPAIGHVSPEAAEGGPIALVEEDDLIRIDIPGRVLEIIGVKGRELPAGEVERILAERRKAWKPREAKYKRGVLKIFSEHAVSPMKGGYMV

SEQ ID NO: 112 - protein, 573 aa, unidentified; identical to SEQ ID NO: 86

SEQ ID NO: 113 - protein, 573 aa, unidentified; identical to SEQ ID NO: 86

SEQ ID NO: 114 - protein, 573 aa, unidentified; identical to SEQ ID NO: 91

SEQ ID NO: 115 - protein, 573 aa, unidentified
MELNSQRVRALAPENDPLKIGMGWKVEDLDKPQIMVESTFGDSHPGSAHLDRFVNEAMRGIADAGGKGARYFTTDICDGIAQGHDGINYSLAHRDMIANMIEIHGNSTGFDGGVFIASCDKSVPACLMGLARLDMPSIVVTGGVMDAGPDLLTLEQIGAYSAMCQRGEISEEKLTYYKHNACPSCGACSFMGTASTMQIMAEALGLMLPGSALMPATCEDLKEMAYKAGLQVMELARKGLKVSDIVTMKSFENAIMVHAAISGSTNSLLHIPAIAHELGLEIDADTFDRMHRGAHYLLDIRPAGKWPAQFFYYAGGVPAVMEEIKSMLHLDVMTVTGKTLGENLEELKAGGFYDKCDGYLKKWGLKRTDVIRTFEEPIGTDGTVAILRGNLAPEGSVVKHSAVPEEMFKAVLKAKPFDCEEDAIEAVISRKIQPGDAVFIRYEGPKGSGMPEMFYTTEAISSDPELGKTIALITDGRFSGASKGPAIGHVSPEAADGGPIALVEEDDLIRIDIPERVLEIIGVKGEELPKEEVERILAERRKAWKPREAKYKKGVLKIYSEHAVSPMKGGYMV

SEQ ID NO: 116 - protein, 571 aa, unidentified
MISQEIRKLAPEQDPLRMGMGWTVEDLSKPQIMIESTFGDSHPGSAHLLELVDRAADGVKQAGGRPARYFATDICDGMAQGHDGINYSLASRDTICSMIEIHAGATPFDGGIFVASCDKAVPAHLMGIGRVNMPSILVTGGVMDAGPDLLTLEQIGKYSAMCQRGEITEEQLTYYKQHACPSCGACSFMGTASTMQVMGEALGLMLPGSALMPATCEDLRDVARRAGEQVMRLAQAGLKPRDIVTRESFENAIMVHAAISGSTNSLIHMPAIAHEFGIELDAELFDRMHRGAHYLLDIRPAGKWPAQYFYYAGGVPRIMEEIKSMLHLDAMTVTGKTLGENLEELRANGFYDKCDEYCAAHGVTRDDVIHPFDKAIGTDGAIAILKGNLAPEGAVIKHTACPKEMFQATLRARPFDSEEEAIDAVLTHKIQPGDAVFIRYEGPKGSGMPEMFYTSEAISSDKELGRSIALITDGRFSGASTGPSIGHVSPEAASGGPIALVEEGDLIRIDIPARVLAIVGVNGEEKTPAEMEAILATRRAKWQPKPAKYTSGVLKIFSEHAVSPMQGGYMK

SEQ ID NO: 117 - protein, 573 aa, unidentified
MKMRSQELRKAAPELDPLRLGTGWKLEDLSKPQIMIESTFGDSHPGSGHLLELVEEAREGVAEAGGFGARYFTTDICDGEAQGHDGINYSLASRDMIANMIEIHANATPFDGGVFIASCDKGMPAHLMGVGRVNIPSIVVTGGVMDAGPDLLTLEQIGKYNAMCERGEITEEKLTYYKHHACPSCGACSFMGTASTMQIMAEALGLMLPGSALMPATSRDLRTMAREAGRQAVWLAEHDLTPDKIVTMKSFENAIMVHAAISGSTNSLLHLPAIAREFGIEIDGDTFDRLHRGAHYLLNIRPAGEWPAQYFYYAGGVPTIMEEIKDMLHLDVMTVTGKTLGENLEDLKKNGFYDKCDEYLAAAGLKREDVIRPFDKPFGTDGSIAILYGNLCPDGAVVKHTVVPKEMFRATLNARPFDCEEDAIHAVLTHDIKPGDAVFIRYEGPKGSGMPEMFYTTEAIASDAELGASIALLTDGRFSGASKGPAIGHISPEAADGGPIALVEEGDLIEVNIPERILQIVGIAGERKTPEEIDAALKERKSKWSPKPSKYKKGVLKIFSERAVSPMKGGYME

SEQ ID NO: 118 - protein, 451 aa, unidentified
MTEPVFYADPMRLLIASVAGILLLLFLIIKLKLHPVISMMISAVLIGIGAGMPLHMIGETVEKGVGKTLQGIALLIGLGSMFGGILEVSGGAQQMAQTLIRRFGQKKAGWALGLTGLVLGTTVFFEAGVVILIPLAFNVAKQTKKSTLYYAIPLLAGLASGYAFVPPSAGSVLVANALGVNLGTMMAVGIPVALISTVVAGIIWGKIIGNKIYTKLPANVEEIADIDKNLPSFGIVFTIILIPLILILFSTISGYLPIPESAQSVLQFLGEPFVALTIATLTAMYMLGTRMGYNRDQLKKILDHSLRPVGMILLVIASGGVIRWMLQDSGLGNIIGPILEKSGMPLIVIAFLIAFLVRASVGSSIVAMTMASGIMASMPAMADSSTLYRAAMCCAICGGATALSHVNDAGFWLVSTFLEIDEKTTLKSWTIMETLIGITGLILGLVISIFA

SEQ ID NO: 119 - protein, 451 aa, unidentified
MTETTFAAEPVRLLIAAAAGIVCLLVLIIKLKLHPVISMMISAIVIGIGAGMPLAMISDTVEKGVGETLQGIALLVGLGSMFGGVLESSGGARRIAQTLVGKFGQEKAGWALGLTGLVIGTTVFFEAGVVVLIPLVFSVAKQTKKSTLYYAIPLLSGLASGYAFVPPSAASVLAANALEVNLGTMIMVGVPMAILCMVTGGIVWGRWIGTKIFTYLPKNIEEIHDEDETLPSFGLVFFVILVPLVLILLSTVSAYLPVAEHIKSIMEFLGEPFLALTIATLLAIYFLGIRRGFSKKRIKEILEHSLRPVGMILLVIASGGVIRWMLQDSGLGNVIGPLLENSDMPLILIAFLVALMVRASVGSSIVAITMASGIMASMPAVMGESMVYRAVMCCAICGGATALSHVNDAGFWLVGTFLEIDEKTTLKSWTIMETVIGLTSLAAGLIISMFV

SEQ ID NO: 120 - protein, 452 aa, unidentified
MSMEGTALNPTRLVAAALIGLVILLVLIIKFKIQAMVAILIGAISIGLIAGMPFENIVTAVNDGIGNTLKGIALLVGLGSMFGAILEVSGGAQTLAVTMVRRFGDKKAAWALGITGLVIAMPVFFDAGLIILIPLAFSLAKRTRRSSLFYAIPLLAGLAVGHAFIPPTPGPVLVATMLNVDLGWVIMVGIFCGVFAMIAAGPVWGAICGKKYMIPVPEHVAEQQDIDESKLPNFWTIVGIILIPLVLIIMDSVCGVVPALSALAPVFSFLGEPFVALLIATVAAMFILGIRHGYTTEELEKVMTKSLEPTGLILLVTACGGVLRYMLQYSGLGEVIGDAVSSANLPIVVVAFVVAALVRISVGSSTVAMTMAAGIVAALPGIAELSPLYLACTTAAVAGGATVCSHFNDSGFWLVKSLVGMDEKTTLKTWTIMETLVGVTGFIVALIISMFA

SEQ ID NO: 121 - protein, 465 aa, unidentified
MSESTRMLLGLALGIIVMILLVMKTKVHSFMALLIAAMITGIIGGMPVSDVTLADGTTQTGLITAIQEGFGSTLKSTGIIIGLGVMMGGILEKSGAAEKMAYSFIKVIGQKNEEWALAITGWFISIPVFADSAIVIFAPLCKAMSRVTGKSVIGLALSLAVGLQLTHCLVPPTPGPLTAAGMLNVDVGTMIIGGALISIPMLIVVVFYTKWIGKQIYQIPTEDGGFDRKQFKKEYIKSMDELNAMMDAKPLPGFGVSVAPIIVPIVLILLKTVLDFVDAGNAVVDFFGSPIVALGIGTLLAIYGLVKKEDNKKVLAMMDNAIKDTGIIMLITGAGGSLGNVVKVSGIGTALGEMVTSWPIPAVLIPFIIAALMRIALGSATVAITTAASLTAPLMSVLSISPLLMALSCCVGAISFSYFNDSGFWVFNGMFGLTELKDQVKCKTAVSMIMSGVGVIELLIAQFFI

SEQ ID NO: 122 - protein, 479 aa, unidentified
MKFALLYYIKLAVFYIFHLCKENKRKELLGEKMVMVLIVVLGIAFLLLLVLKCKLHAFIALLIASIAVGVAAGMPLTDITASIQNGMGGTLGTVAIIVGLGAVFGQMLEASGGAQALAKSMIRFFGEKHAPLAFAVTGFIIAIPVFFDVGFIILLPLIYAVAYKTKRSVATFAFPLIISLCLTNAFVPPTPGPVATCSALDANVGWVIFFGIIISIPLTLVGYLYSIKYIDKHLFIPVPDYMIPEESNDENLPSFSSVILIIAVPILLILVNTGVDSLVNEGILADSFFTQAAKFLGTSYIALLIAVLLSFYILGKRRGFHKEDLYEISSKAFLPVGLIILVTGAGGVFKQILLDSGVGDVIAQSVETIGLPPIILGYLVAVFIRVSQGSGMVAMITAAGIVSPLLAVSPVSEMQRALVVLAIAAGSVAASHVNDSGFWLVCKYLNMTEGQTLKTWTFITAFLSLCSLVLVILLSLIIP

SEQ ID NO: 123 - protein, 453 aa, unidentified
MTTTTAAALDPTRLIIAALAGLVILLLLIIKFKVQAMISILVGAVAIGLIAGMPFADIISSVNEGIGSTLKGIALLVGLGSMFGSILEISGGAQTLAVTMVKKFGDKKAAWALGITGLVIAMPVFFDAGLIILIPLAFSLAKRTKRSSLFYAIPLLAGLAVGHAFIPPTPGPVLVATMLGVDLGWVIMVGIVCGIFAMIVAGPIWGTICGNKYDIPVPEHIANQEDFDESKLPKFGTIVGIILIPLVLIILNSIAKVVPALASVQPVLGFLGEPFVALTIATVVAMVLLGYRHGYSNEELEKVLTKSLEPTGLILLVTACGGVLRFMLQNSGLGDVIGNAVSSASLPIVVVAFVVAALVRISVGSATVAMTMAAGIIAAMPEIATLSPLYLACVTAAVAGGATVCSHFNDSGFWLVKSLVGLDEKTTLKTWTIMETLVGGTGFVVALIISFFA

SEQ ID NO: 124 - protein, 465 aa, unidentified
MSESTRMLLGLALGIIVMILLVMKTKVHSFMALLIAAMITGIIGGMPISDVTLADGTTQTGLITAIQDGFGSALKSTGIIIGLGVMMGGVLEKSGAAEKMAYSFIKLIGQKNEEWALAITGWFISIPVFADSAIVIFAPLCKAMSRVTGKSVIGLALSLAVGLQLTHCLVPPTPGPLTAAGMLNVDVGTMIIGGALISIPMLIVVVFYTKWIGKQIYQIPTADGGFDRKEFKKEYIKSMDELNAMMDAKPLPGFWVSVAPIIIPIVLILLKTVLDFVGVGNAFIDFFGSPIVALGIGTLLAIYGLVKKEDNKKVLGMMDEAIKDTGIIMLITGAGGSLGNVVKVSGIGTALGEMVTSWPIPAVLIPFIIAALMRIALGSATVAITTAATLTAPLMSMLSISPLLMALSCCVGAISFSYFNDSGFWVFNGMFGLTELKDQVRCKTAVSMIMSVVGVVELLIAQFFI

SEQ ID NO: 125 - protein, 346 aa, unidentified
MSIEGTALNPVRLVAAALIGLVILLVLIIKFKIQAMVAILIGAISIGLIAGLAVGHAFIPPTPGPVLVATMLNVDLGWVIIVGIFCGVFAMIVAGPVWGSICGKKYMIPVPEHVAEQNDIDESKLPNFWTIVGIILIPLVLIIMDSVCGVVPALSALAPVFSFLGEPFVALLIATVAAMFILGIRHGYTTEELEKVMTKSLEPTGLILLVTACGGVLRYMLQYSGLGEVIGDAVSSANLPIVVVAFVVAALVRISVGSSTVAMTMAAGIVAALPGIAELSPLYLACTTAAVAGGATVCSHFNDSGFWLVKSLVGMDEKTTLKTWTIMETLVGVTGFIVALIISMFA

SEQ ID NO: 126 - protein, 465 aa, unidentified
MFSGEMVTGPLLIGIFILAIVVLLLLIIKFKLNAFVALLITAFGTGVLVNMPLADVAQTVTDGFGGTLGGIGMVTGLGVMLGKFMFESGGIESISNKILGAFGEKKSPIAVALSGFITGIPVFGDVVYIMFAPMLRVLSKKTKISMVTFACAISVATTCTFALVLPTAPPLAVAEELNIEIGIFFFYALISAFVGMIVGGIFYGSILNKQDHKNNHFYTFEDLDEEEAEMSKSGKGKERAKMGAGKALSILLVPIVLILLGSFIPLAVGKDSAIVPFVTFIGNKNFAMMAGVIYAAVISRKYITKTATDIMTEAADQVGLILLITGAGGAFGKVLQATGIADYVAGSLSQFSIPILVLCFLIAQIIRCAQGSTTVALMTTAAIMSSTIASGGVSPILCAIAICAGGIGLSLPNDSGFWAISRFFKISVTDTIRGWSIGGFVAGVAILIFVSILSLFQGFLPGLMM

SEQ ID NO: 127 - protein, 452 aa, unidentified
MENFVASPERLIIGALLGFAVLLFMIIKLKVQPFLSIMLSALIIGLAVGMPFTMITDTLSEGVGTTLQTIAVLIGLGSMFGAILQVSGGVEVIAQTLLDKFGEKKAAWALGVTGLIVGMPVFFESGLLILIPIAFGIAKKTNRSIFHYAIPLLAGLAIGHAFIPPTPGPVLVAEMLNVELSYVIILGIIIGIPAMILAGPVFGKFAGDRYNFPVPSSFEQNEKKIVEGKLPSFGTVVAIILLPLFLIILNTLSSVIPFMKPLNPVLTFLGTPVIALLLSTITAMVLLGMRHGYTREELTKIMTESLSSAGNIILLIAGGGMLRFILQNSGIGEAFGNFVGGLPLPLVLVAFLIAAVVRISVGSATVSMTMAAGIMASMPAIADLSQIQLATITIAVAAGATAFSHVNDSGFWLSKSLFEVDEKTNLKTWTVMETIIGIVGVIGATIIWFIVQ

SEQ ID NO: 128 - protein, 453 aa, unidentified
MDEGLRMALGLALGLVILVLMITKTKIHVFLAVIICSIFIGLFGGMGQEDVIASITKGFGNSLSSIGIIIGFGVMLGKLLEMSGATRVMANTFIKIFGKNREEEALAISGFVTSLSIFCTSGFIILAPLMKALSKKTKKSVVALGIALAGGLVASHSLVPPAAGPIGVAGILNVNIGQFMLYGTVLSVPIILVSLIYGKYLGRKICQIPDGEGWTREYEPGQTQAEPEAAAADSGALSISPLTAFSPILFPILLILLKTGLKSAGVENGVVSLLGQPIVALALGLLIAIYGLMRRFGKSDTLNAMEEGLKASGKLMLLVGGGGALGMIIQNSGLGEYIASNIIKTSIPPLLLPFLIAALLRVAQGSGSVAMMTAASVVAPMIGTLNLNPVFAALAACIGAIPFSHFNDSYFWVINESIGISDTREQMRVWSVTTTLSGFAALAVLLVMNHFLG

SEQ ID NO: 129 - protein, 478 aa, unidentified
MSPTSFIIMLVAAIVILTVLTLKFKMHPVMTLFITAVFIGLVSGKPLVDTFNSIKNYFGSTLGSIGVMIIFGAVIASGISDTGAATSIVNFFIRLFKGKRLELAPALTGFIMSIPVFGDIAIILNAPISAILAKRKNMSMSQVAPFVNLGLTLTHGLVPPTPGVLAVAVLLQADIGTVIMWGLVCSVVSFAVCYFALAPIYAKGEFIEPLADYTEGIEAVDDGASVEKLLIAEENAPGAGMSFVPLLLPAVMIAVGSIGKLMVEEGSGAYVFFNTFGDTVLALFCGIVALGLMVFGRKDKVVKKANEATGTQELSEKSSWTEIVFGNWCGRALKIAISALLITAMGGAMGGILRENEAISVIGNAIARTNFPSLLIPFLLAAILMTVCGSMTTASMTAAALMVELVPVLGMSPVVATLAIGAGSMVGWHVNNSGFWIFTSLYGFDTKQGLKFFTTTNALGGIVAFICLAGLTMIGLVG

SEQ ID NO: 130 - protein, 457 aa, unidentified
MGLGLLLIVFAVSLVFVLVSIIKFNLHPFLSLLIGGLIMGILAGLPLTQVADGLAAGFGSTMQGIGILIILGVGLGHLLHISGCTSQIAAAMLKLTGQKRAGVAVNLTGYIVSIPVFFDAAFVILVNLVKSLSRKGKIPFVSLVTALSVGLIVTHAMVIPTPGPIAVAGTMGVNIGWFLLYSIVVSLPASLVGGVLYGKYLGGKAEYADDFANAFDDIEEEEIQESADLPSGALGIFLIFLPIIIILIGTIASMLLEPGTSAYIFFSFIGNKNIALLIGLLAAFFSLRRYVKDSFNDVVTEAATQSGIILAITGAGGAFGAIINLTGIGEKLVGGMAGMTEGAGIAMIVAAFVISQVLRAAQGSTTVALVTTSAIFAPILAGMATVSPILVSLAICAGGIGLSLPNDSGFWVINRFSKFDVPGTIKVWTVGGTIAGLTALVILVVLSMFTGVLPGLL

SEQ ID NO: 131 - protein, 451 aa, unidentified
MTEPVFYADPMRLLIASAAGILLLLFLIIKLKLHPVISMMISAVLIGIGAGMPLHMIGETVEKGVGKTLQGIALLIGLGSMFGGILEVSGGAQQMAQTLIRRFGQQKAGWALGLTGLVLGTTVFFEAGVVILIPLAFNVAKQTKKSTLYYATPLLAGLASGYAFVPPSAGSVLVANALGVNLGTMMAIGIPVALVSTVVAGIIWGKIIGNKIYTKLPANVEEIADIDKNLPSFGIVLMIILIPLVLILLSTISGYLPIPESVQSVLQFLGEPFVALTIATLTAMYMLGTRMGYNRDQLKKILDHSLRPVGMILLVIASGGVIRWMLQDSGLGNIIGPILEKSGMPLIVIAFLIAFLVRASVGSSIVAMTMASGIMASMPAMADSSTLYRAAMCCAICGGATALSHVNDAGFWLVSTFLEIDEKTTLKSWTIMETLIGITGLILGLVISIFA

SEQ ID NO: 132 - protein, 467 aa, unidentified
MFSGEVVTGPLLIGIFILAIAVLLLLIIKFKLNAFVALLITAFGTGVLVNMPLADVAQTVTDGFGGTLGGIGMVTGLGVMLGKFMFESGGIDSISNKILGAFGEKKSPIAVALSGFITGIPVFGDVVYIMFAPMLRVLSKKTKISMVTFACAISVATTCTFALVLPTAPPLAVAEELNIEIGIFFFYALISAFVGMIVGGIFYGSILNKQDHKNNHFYTFEDLDEEEAEMSKGGKGKGKEQAKMGAGKALSILLVPIILILLGSFVPLAVGKDAAIVPFVTFIGNKNFAMMAGVIYAAVISRKYITKTATDIMTEAADQVGLILLITGAGGAFGKVLQATGIADYVAGSLSQFSIPILVLCFLIAQIIRCAQGSTTVALMTTAAIMSSTIASGGVSPILCAIAICAGGIGLSLPNDSGFWAISRFFKISVTDTIRGWSIGGFVAGVAILIFVSILSLFQGFLPGLMM

SEQ ID NO: 133 - protein, 465 aa, unidentified
MGAVSSTQMLVGLAIGIGILIALSLKTKIHTFIALIIATIVTGLIGGMEVTVVMKSITTGFGNTLGSTGIIIGLGVMMGIILEKSGAAEQMAFTVIKLIGKNKEEWALGLTGYIVSIPVFSDSALVILTPIAKALSKLSGKSVVGLGLALATGLQLTHVFVPPTPGPLTVAGILGIDVGVMIMAGILCTIPVYIVSMLYCKWLGTKIYQVPSDDPACGENEFTRMDFKKEYLKSIENIEELHKARNLPSVGLSFAPVIVPLILIFINTITKFIGLKGSYMTAVELFGSPITALIIGTLISIYGLGAKMTKKEVHDAMNEAIQSTGMIMLITGAGGALGFVVRESGIGNALGNAVIYIGIPGLLIPFIIAALMRIALGSATVALTTAATLTAPLLSQLGISPVLAAMSTCAGGVAFSYFNDSGFWVFNGLYGLEDIKDQFWAKTMISFVGSGAALAVVLIASIFIK

SEQ ID NO: 134 - protein, 438 aa, unidentified
MPLVIVAIGVILLLLLMIRFKMNGFIALVLVALAVGLMQGMPLDKVIGSIKAGVGGTLGSLALIMGFGAMLGKMLADCGGAQRIATTLIAKFGKKHIQWAVVLTGFTVGFALFYEVGFVLMLPLVFTIAASANIPLLYVGVPMAAALSVTHGFLPPHPGPTAIATIFNADMGKTLLYGTILAIPTVILAGPVYARVLKGIDKPIPEGLYSAKTFSEEEMPSFGVSVWTSLVPVVLMAMRAIAEMILPKGHAFLPVAEFLGDPVMATLIAVLIAMFTFGLNRGRSMDQINDTLVSSIKIIAMMLLIIGGGGAFKQVLVDSGVDKYIASMMHETNISPLLMAWSIAAVLRIALGSATVAAITAGGIAAPLIATTGVSPELMVIAVGSGSVIFSHVNDPGFWLFKEYFNLTIGETIKSWSMLETIISVCGLVGCLLLNMVI

SEQ ID NO: 135 - protein, 446 aa, unidentified
MTTLTLVLTAVGSVLLLLFLVMKARMHAFLALMVVSMGAGLFSGMPLDKIAATMEKGMGGTLGFLAVVVALGAMFGKILHETGAVDQIAVKMLKSFGHSRVHYAIGLAGLVCALPLFFEVAIVLLISVAFSMARHTGTNLVKLVIPLFAGVAAAAAFLVPGPAPMLLASQMNADFGWMILIGLCAAIPGMIIAGPLWGNFISRYVELHIPDDISEPHLGEGKMPSFGFSLSLILLPLVLVGLKTIAARFVPEGSTAYEWFEFIGHPFTAILVACLVAIYGLAMRQGMPKDKVMEICGHALQPAGIILLVIGAGGVFKQVLVDSGVGPALGEALTGMGLPIAITCFVLAAAVRIIQGSATVACLTAVGLVMPVIEQLNYSGAQMAALSICIAGGSIVVSHVNDAGFWLFGKFTGATEAETLKTWTMMETILGTVGAIVGMIAFQLLS

SEQ ID NO: 136 - protein, 447 aa, unidentified
MHVLNILWVVFGIGLMLVLNLKFKINSMVALLVAALSVGMLAGMDLMSLLHTMKAGFGNTLGELAIIVVFGAVIGKLMVDSGAAHQIAHTLLARLGLRYVQISVIIIGLIFGLAMFYEVAFIMLAPLVIVIAAEAKIPFLKLAIPAVAAATTAHSLFPPQPGPVALVNAYGADMGMVYIYGVLVTIPSVICAGLILPKFLGNLERPTPSFLKADQPVDMNNLPSFGVSILVPLIPAIIMISTTIANIWLVKDTPAWEVVNFIGSSPIAMFIAMVVAFVLFGTARGHDMQWVMNAFESAVKSIAMVILIIGAGGVLKQTIIDTGIGDTIGMLMSHGNISPYIMAWLITVLIRLATGQGVVSAMTAAGIISAAILDPATGQLIGVNPALLVLATAAGSNTLTHINDASFWLFKGYFDLSVKDTLKTWGLLELVNSVVGLIIVLIISMVA

SEQ ID NO: 137 - protein, 442 aa, unidentified
MNLIAAAIIGIAVIVLLIAACKVHPFLSLLAGSFVMAVCAGVPFDKAFDSFTSGVGGTISNVGLLIAFGSIIGTILFKSGGADTIVDTIMAKTPLQRLPWAMALIAFIVGIPMFFEVGVVILIPVVLFAARRSKSPVVLLGIPALAGLSTLHAFVPPHPGPLTAIGALNANLGITLALGLIVAIPTVIISGPLFGRLAAKWVPIAAPENEAEAEAPKSAENRPSFGTALSVILLPVVLMLAASIVDLTGQNTTAWGRVIAFLGTPLVALLITTVFAMVALGYMQKFSRDAVNGMVGQSFGSVAGIILIVAAGGGFKQTLVDSGIGDVIANSITESAMNPLIAGWLVAVLIRLATGSATVATVTASGIMVPLAAGMSPTHLALLVLAIGAGSVFFSHLNDAGFWLVKEYFGMSVGQTLKTWSLMETILSVVGLGCVMLLSLVL

SEQ ID NO: 138 - protein, 442 aa, unidentified
MNLIAAAIIGIAIIVLLIAVCKVHPFLSLLAGSFTMAVCAGVEYDEAFGSFTSGVGSTIANVGLLIAFGSIIGTILFKSGGADTIVDTIMAKTPLQRLPWAMALIAFIVGIPMFFEVGVVILIPVVLFAARRAKAPVVLLGIPALAGLSTLHAFVPPHPGPLTAIGALNANLGITLALGLLVAIPTVIISGPIFGKLAAKWVPITAPENKAEAEAPKSAENRPSFASAIAVILLPVVLMLAASIVDLTGQSETTWGRALAFIGTPLTALLITTVFAMVALGYLQKFSRDAVNGMVGQSFSSVAGIILIVAAGGGFKQTLVDSGIGDVIANSITESAMNPLIAGWLVAVLIRLATGSATVATVTASGIMVPLATGMSPTHLALLVLAIGAGSVFFSHLNDAGFWLVKEYFGMTVGQTLKTWSLMETILSAVGLGCVMLLSLVL

SEQ ID NO: 139 - protein, 439 aa, unidentified
MSFIIVVLCLALLIVLISYFKVDAFIAFLISSILAGIALGIPLEKLPDSVNNGIGSILGGLTLIIVLGAMLGKLVAESGAAQKIASVMVSLFGTKHIQWGLMVTGFVVGIPLFYGIGFVLLVPLIFSIVYKYKLPAVYIGLPMLAALSVTHGFLPPHPSPVALVVQFNANIGLTLIYGLCLAIPAIIIAGPVFGSRLKHIKSGPVTLFEQKEDSGRYSMPGKANSFISATLPVFLLIIVTVIPYCFKDMGEMASGIVSFIGAPSIVMLIALLFATYTLGIRQGRSIKSVMAVYGEAVKDISMILLIIAGSGIFKQIMEDSGVSNELATSLEQLPVHPLILGWLISAVIRLCVGSATVAALTAAGVVMPLVTQTGANPNLMVLSLGAGSLMFSHVNDSGFWMFKEYFNLSIKDTICSWSIMETIVSVVGLIGVMILNMIV

SEQ ID NO: 140 - protein, 451 aa, unidentified
MTAVATPDAARLVFAAVAGLILLLILIIKFKVHAMISILIGAVGIGLMAGMPLSDIIGSVNEGIGNTLKGIALLVGLGSMFGAILEESGGAQTLAVTMVRKFGDEKAAWALGITGLVVAMPVFFDAGLIILIPLAFSLAKRTKRSVLYYVIPLLAGLAVGHAFIPPTPGPVLVASMLSVDLGWVILIGIFCGIFAMIAAGPIWGSICGKKYMIEVPEHVAQQADIDESKLPKFGTIVGIILIPLVLIIANSVAKVVPALAGIQPVLAFLGEPFMALLLATIAAMYLLGTRHGYNNAQLEKIMTKSLEPTGMILLVTACGGVLRYMLQNSGLGDVIGNAVANASLPLVLVAFIVAALVRISVGSSTVAMTMAAGIISAMPGISELSPLYLACVTAAIAGGSTVCSHFNDSGFWLVKSLVGMDEKTTLKTWTIMETLVGGVGFLVALIISFFA

SEQ ID NO: 141 - protein, 451 aa, unidentified; identical to SEQ ID NO: 140

SEQ ID NO: 142 - protein, 451 aa, unidentified
MEETTFAADPMRLMIAAAVGIIVLLVLIIKFKLHPVLSMMISAIIIGVGAGMPLTMISDTVEKGVGKTLQGIALLVGLGSMFGGILEVSGGAQRIAETLIGKFGQKKAGWALGLTGLVIGTTVFFEAGVVVLIPLAFSVAKQTKKSTLYYAIPLLSGLASGYAFVPPSAGSVLVANALGVNLGTMIMVGIPTALICMLVSGIIWGGFIGNKIFTELPANVGEIKDDGKELPSFGLVLGVILIPLVLILLSTLSKYMPLPAGVQDVLGFLGKPFLALTIATLAAMYFLGIRRGFSGAQLKKILDHSLRPVGMILLVIASGGVIRWMLQDSGLGNIIGPALEKSGLPLILIAFFIAILVRASVGSSIVAMTMASGIMATMPAVMDTSMLYRAAMCCAICGGATALSHVNDAGFWLVGTFLEIDEKTTLKSWTIMETLIGVTALIVSLVISIFA

SEQ ID NO: 143 - protein, 455 aa, unidentified
MSEFVASPERLIIGALLGFAVLLFLIIKVKIQPFIAIMSSALIIGLAVGMPFNMITDTIAEGVGTTLQTIAILIGLGSMFGAILQVSGGVEVIADTLLKVFGEKKAPWALGITGLIVGMPVFFESGLLILIPVAFGIARKTKKSVFCYSIPLLAGLAIGHAFVPPTPGPVLVAEMLNVELSYVIMLGIIIGIPAMILAGPVFGKFAGNKFHCEVPTSYLENEKEAHEKENGKRPNFFLIVSIILLPLFLIILNTLSSVIPFMKPVQPVLEFLGTPVIALLLSTICAMLFLGLPGGFGITEITTVMTNSLHSCGNIILLISGGGMLRFILQNSGIGEVFGNFVGSLPLPMVLVAFLVAAVVRISVGSATVSMTMAAGIIASMPMVANLTQLQLATLTIAIAAGGTAFSHVNDSGFWLSKSLFEVDEKTNLKTWTVMETIVGLVGIVGATIVWFIVS

SEQ ID NO: 144 - protein, 451 aa, unidentified
MTAVATPDAARLVFAAVAGLILLLILIIKFKVHAMISILIGAVGIGLMAGMPLSDIIGSVNEGIGNTLKGIALLVGLGSMFGAILEESGGAQTLAVTMVRKFGDEKAAWALGITGLVVAMPVFFDAGLIILIPLAFSLAKRTKRSVLYYVIPLLAGLAVGHAFIPPTPGPVLVASMLGVDLGWVILIGIFCGIFAMIAAGPIWGSICGKKYMIEVPEHVAQQADIDESKLPKFGTIVGIILIPLVLIIANSVAKVVPALAGIQPVLAFLGEPFMALLLATIAAMYLLGTRHGYNNAQLEKIMTKSLEPTGMILLVTACGGVLRYMLQNSGLGDVIGNAVANASLPLVLVAFIVAALVRISVGSSTVAMTMAAGIISAMPGISELSPLYLACVTAAIAGGSTVCSHFNDSGFWLVKSLVGMDEKTTLKTWTIMETLVGGVGFLVALIISFFA

SEQ ID NO: 145 - protein, 452 aa, unidentified
MTDAVFAADPARLLIAAAAGILLLLLLIIKFKFHPVLSLLISALVIGLGAGMPVPTLVDTVEKGAGETLQGIVLLIGLGSLFGGILEVSGGAQCVAQTLVNRFGEKKAGIALGITGLVVGTTVFFEAGVVILIPLAFGLAKKTKKSTLYYVIPLLAGLATGFAFIPPSAGSVLVANMLGVDLGVMIAVGVPTGILSLIFAGILWSKFIGTKIHTGLPATVSEVREENEANLPKFSTVIEIILVPLVLILCSTLSEYIPALKGIRPVLEFVGTPFVALIIAVLCAMYFLGKKQGYDGEQLKKIMDRSLRPTGQILLVITGGGIIRWVLQDCGMGDIIGPALEKSGLPLILVAFLIAALVRASVGAAVVAMTMAAGIMASMPAVAGLSPLYLAAMVCAITGGATAFSHVSDSGFWLVSSLLEIDEKTTLKSWTMMETIIGFTGLLCALVISLFA

SEQ ID NO: 146 - protein, 455 aa, unidentified
MASPVTLIAALVAAIAVILFCIIVLKLNAAIGMVVACILMGLAGGLDLLTTATTISSGFGSMMTSIGLPVGFGTILGQLMSDSGAASVIADKLVAAFPEKRAIWGLSLAGFILSVPVFFDVTFIILIPIGITIASKINMKMCYVTGCLTIGATTAHCVVPPTPNPLAAADIFGFDLGIMLIVGLVVGFITVLVSDFLFIKIHDMGIWNEAKDVNHSSNVTAELVSARNSQPSGKKQPSLFISLLPIIIPVVCILFGTLGSAMFEEAPVICSFLGDKLIAMLLGTLGAYLVSLPYIGRDHLEESAGEALKSAGVVLLITGAGGSFASVITATGIGDAIVGLLGTDTTSVVPAMFLAYIIGMIFRVAQGSGTVAGMTSMGIMATIAPAVGCHPVWIALACLSGGNSIGHINDSGFWVATNMSGLTITGGLKTYTLGSFLSSLCIFVLALIGALMIPL

SEQ ID NO: 147 - protein, 439 aa, unidentified
MPLVILAIGILVLFFLIVKVKLNSFLALLLVAGAVGLAEGMPIDKLIPTIEKGLGGTLGGLAIVVSFGAMLGKLMAESGGAQRIATTLINVFGREKVKWAVCLTGFIVGIALFYEIGFVLLIPLVFTIAAEAKIPLLEVGIPMAAALSVTHGFLPPHPGPTAISIIYNADIGTTLVYGAIIAIPTAIVAGPLYCTLTRDINPEIPKGMYNPKIFKDDEMPGFGISVFTALVPVILMAASAIAKLTMGADVPARHILTFFGQPDIALTISVAIAIYTFGLGRGKSMEDVMKTVQDAVLTIAMILLIVGGGGALKQVLIDSGVGTYIGNLVAGSDLSPLLLAWLVAAVIRTACGSATVAALTAGGIAAPICAVTGANPELMVLATGAGSLIFSPPNDPGFWLFKEFFGLTVKQTVRTWCALETIIAVMGLAGVLVLNLFVG

SEQ ID NO: 148 - protein, 439 aa, unidentified
MPLVILAIGILILFFLIVKVKLNSFLALLLVAGLVGLAEGMPIDQLIPTIEKGLGGTLGGLAIVVSFGAMLGKLMAESGGAQRIATTLINVFGREKVKWAVCLTGFIVGIALFYEIGFVLLIPLVFTIAAEAKIPLLEVGIPMAAALSVTHGFLPPHPGPTAIAIIYNADIGTTLLYGAAIAIPTAIVAGPLYCNLTKDINPEIPKGMYNPKIFKDDEMPGFGISVFTALVPVILMAASAISKLTMDADLPLRHVLTFFGQPDIALTISVAIAIYTFGIGRGKSMEDVMKTVQDAVLAIAMILLIVGGGGALKQVLIDSGLGLYIGELVANSDLSPLFLAWLVAAVIRTACGSATVAALTAGGIAAPIAVATGANPELMVLATGAGSLIFSPPNDPGFWLFKEFFGLTIKETVRTWCALETIIAVMGLIGVLVLNVFVG

SEQ ID NO: 149 - protein, 467 aa, unidentified
MFSGEVVTGPLLIGIFILAIAVLLLLIIKFKLNAFVALLITAFGTGVLVNMPLADVAQTVTDGFGGTLGGIGMVTGLGVMLGKFMFESGGIDSISNKILGAFGEKKSPIAVALSGFITGIPVFGDVVYIMFAPMLRVLSKKTKISMVTFACAISVATTCTFALVLPTAPPLAVAEELNIEIGIFFFYALISAFVGMIVGGIFYGSILNKQDHKNNHFYTFEDLDEEEAELSKSGKGKGKEQAKMGAGKALSILLVPIILILLGSFVPLAVGKDAAIVPFVTFIGNKNFAMMAGVIYAAVISRKYITKTATDIMTEAADQVGLILLITGAGGAFGKVLQATGIADYVAGSLSQFSIPILVLCFLIAQIIRCAQGSTTVALMTTAAIMSSTIASGGVSPILCAIAICAGGIGLSLPNDSGFWAISRFFKISVTDTIRGWSIGGFVAGVAILIFVSILSLFQGFLPGLMM

SEQ ID NO: 150 - protein, 452 aa, unidentified
MENFVASPERLIFGALLGFAVLLFMIIKLKVQPFLSIMLSALIIGLAVGMPFTMITDTLSEGVGTTLQTIAVLIGLGSMFGAILQVSGGVEVIAQTLLDRFGEKKAAWALGVTGLIVGMPVFFESGLLILIPIAFGIAKKTNRSIFHYAIPLLAGLAIGHAFIPPTPGPVLVAEMLNVELSYVILLGIIIGIPAMILAGPVFGKFAGDRYHFPVPSSFTRNEKKMAEGKLPSFGTVVAIILLPLFLIILNTLSSVIPFMRPLKPVLTFLGTPMIALLLSTITAMVLLGIRHGYTREELTKIMTESLSSAGNILLLIAGGGMLRFILQNSGIGEAFGNFVGALPLPLVLVAFLIAAAVRISVGSATVSMTMAAGIMASMPAVGDLSQIQLATITIAVAAGATAFSHVNDSGFWLSKSLFEVDEKTNLKTWTVMETIIGIVGIVGATIVWFIVR

SEQ ID NO: 151 - protein, 467 aa, unidentified
MFSGEMVTGPLLIGIFILAIAVLLLLIIKFKLNAFVALLLTAFGTGVLVNMPLADVAQTVTDGFGGTLGGIGMVTGLGVMLGKFMFESGGIESISNKILGAFGEKKSPIAVALSGFITGIPVFGDVVYIMFAPMLRVLSKKTKISMVTFACAISVATTCTFALVLPTAPPLAVAEELNIEIGIFFFYALISAFVGMIVGGIFYGSILNKQDQKNNHFYTFEDLDEEEAEMSKSGKGKGKERAKMSAGKALSILLVPIILILLGSFVPLAVGKDAAIVPFVTFIGNKNFAMMAGVIYAAVISRKYITKTATDIMTEAADQVGLILLITGAGGAFGKVLQATGIADYVAGSLSQFSIPILVLCFLIAQIIRCAQGSTTVALMTTAAIMSSTIASGGVSPILCAIAICAGGIGLSLPNDSGFWAISRFFKISVTDTIRGWSIGGFVAGVAILIFVSILSLFQGFLPGLMM

SEQ ID NO: 152 - protein, 452 aa, unidentified
MDGNLSLILAFLVSISVVLALVIKFKLHPFLSLFIGCLLMGVCSGLDPMAIIKTSCKGFGDTMGDIGIIILLGVAMGQILHESGCTREIANTMLRLCGREKSALALNLTGYIISIPVFFDAAFVILIGLIREMAREGKLALNTLVTALVVGLTCTHALVIPTPGPVAVAGNMGADIGWFIVYGVLIALPASLIGGVLYGKFLGRKSPIFLTDEEPDAALVEQALAKSDHPSGALGIAIIFLPIMLIFFANILNAFLDPASSASRVVSFFGNTNIVLLITVFVAYFSLREHLPEPFDTVVSRGAESVGSILAIIGAGGAFGAVIGASGISTAIVDVMQSWSIPVILLGFLMSQCLRIGLGSITVSIVTASAVLAPVASQLGASPVLVGLAICCGGIGLGLPNDSGFWTICKMSGLSTKQAFLVYPIPTLISGLVGLAVLLILNMFASSLPGLM

SEQ ID NO: 153 - protein, 457 aa, unidentified
MVTGGWLIVIFLIAIAILLITIIKFKVNAFIALLVTTIGTGLMVRMPVSDISKVIGNGFGNTLGSIGVIIGLGVMLGRFLYESGGIEVIADTFLKRFNGSKSRIAVAMAGFITGIPVFGDVVHIMYAPMVRVISKKTKVSIVSLCCATVCATVATGAMVIPTPNPMAVAENLQLDYGVFFLYAALAGLFGTIVGGLGYGRFLDWQDRKNHHEYAFEDIEDFEKESRTSTAGRKVPGFGVAVSILLVPIMLILFGSFGPMILPEGGTLVKILNFVGDKNIAMLIGVIYAALVSLPYLQKPVGDVMNDAAGQVGLVLLITGSGGAFGKMLQSTGIADFIATSLSQFHIPVLVLCFIIAQILRCAQGSTGVALITTSSMFAPLIAASGISPVLCGIAICAGGIGLSLPNDSGFWAINRFYKISVEDTIRAWTIGGFITGVAALVFVCILSVFQAHLPGLL

SEQ ID NO: 154 - protein, 454 aa, unidentified
MDYVANPAQLITATLVGFVVLLFLIIKLKLHALLSVLIAGVIIGVGSGMPFSLVTQAVTDGMGNTLKGIALIVGLGSMFGGILEVSGAAQAIADKLVKVFGEKNSAFALGLAGVIIGIPVFFDPAFIILMPIVYSIAKKTNKSLLYYVLPMTAGLGIGSAIPPTPAPMLITGSLPVDLGTVTILALVLVIPKYLISYAVAVPFAKKMYVPVPENAVMAPEKKEGTAGGPSFGLVISIVLLPILLILISASAGMMTAEGAGMQRFLDFCVFIGQPYLALIIANVAGIIFLCVRKGITMETVESVLAKSLQPVAMIVLVTSAGGVLRYVLDYSGMGTLIGNALQNANLSIILVAYIISTLMRIGVGSTTVALTMTLGIVASFPQISTYSPMYLACIGMSAMFGATSFSHVNDSGFWLTKEYMGIDLKTAFKSWTLYGGFCSILSFIVLYAISFIWA

SEQ ID NO: 155 - protein, 453 aa, unidentified
MTTTTAAALDPTRLIIAALTGLVILLLLIIKFKVQAMISILVGAVAIGLIAGMPFGDIISSVNEGIGSTLKGIALLVGLGSMFGSILEISGGAQTLAVTMVKKFGDKKAAWALGITGLVIAMPVFFDAGLIILIPLAFSLAKRTKRSSLFYAIPLLAGLAVGHAFIPPTPGPVLVATMLGVDLGWVIMVGIVCGIFAMIVAGPIWGAICGNKYDIPVPEHIANQEDFDESKLPKFGTIVGIILIPLVLIILNSIAKVMPALASVQPVLGFLGEPFVALTIATVVAMVLLGYRHGYSNEELEKVLTKSLEPTGLILLVTACGGVLRFMLQNSGLGDVIGNAVSSASLPIVAVAFVVAALVRISVGSATVAMTMAAGIIAAMPEIATMSPLYLACVTAAVAGGATVCSHFNDSGFWLVKSLVGLDEKTTLKTWTIMETLVGGTGFVVALIISFFA

SEQ ID NO: 156 - protein, 444 aa, unidentified
MPMEFVFIIGMAVLLFLCLKVRANAFISLLATALVMGLLSGMTGADTIGAITGGFSGTVKSIGIIIIFGIMLGNYLDAAKGTNRMALDAVRLVGQKRAGLAMAITGYIVSIPVFSDAAFVILTPLVKAIHKKTRIPLAILAVSLSAGLLATHVYVPPTPGPLAAAGLLGIDIGQAIMYGAFAAVFMTLFGWGFAEIYFRNKPSSFYSFQESGKKEEEGLDIEDETDLPGTMASLLPLVVPILLILGKTTCDMLCPEGSLLLTVTSFIGDSNIALAIGAVLAMATLGKRLGSKKVLEIMDKTLKDAGPIVFITAAGGALGQVLKVSGAGDSLAAMVVNTGLPFILIPFVISGLLKIVQGSGTVAVTTAATLCAPIAASLGLNPILIFLASGAGARACCHVNDSYFWVYTNCMGFDMETGLKTLSISNVMMSLGGLCATFICSLWL

SEQ ID NO: 157 - protein, 451 aa, unidentified; identical to SEQ ID NO: 118

SEQ ID NO: 158 - protein, 451 aa, unidentified
MTEPVFYADPMRLLIASVAGILLLLFLIIKLKLHPVISMMISAVLIGIGAGMPLHMIGETVEKGVGKTLQGIALLIGLGSMFGGILEVSGGAQQMAQTLIRRFGQKKAGWALGLTGLVLGTTVFFEAGVVILIPLAFNVAKQTKKSTLYYAIPLLAGLASGYAFVPPSAGSVLVANALGVNLGTMMAVGIPVALISTVVAGIIWGKIIGNKIYTKLPANVEEIADIDKNLPSFEIVFTIILIPLILILFSTISGYLPIPESAQSVLQFLGEPFVALTIATLTAMYMLGTRMGYNRDQLKKILDHSLRPVGMILLVIASGGVIRWMLQDSGLGNIIGPILEKSGMPLIVIAFLIAFLVRASVGSSIVAMTMASGIMASMPAMADSSTLYRAAMCCAICGGATALSHVNDAGFWLVSTFLEIDEKTTLKSWTIMETLIGITGLILGLVISIFA

SEQ ID NO: 159 - protein, 452 aa, unidentified
MENFVASPERLIIGALLGFAVLLFMIIKLKVQPFLSIMLSALIIGLAVGMPFTMITDTLSEGVGTTLQTIAVLIGLGSMFGAILQVSGGVEVIAQTLLDKFGEKKAAWALGVTGLIVGMPVFFESGLLILIPIAFGIAKKTNRSIFHYAIPLLAGLAIGHAFIPPTPGPVLVAEMLNVELSYVIILGIIIGIPAMILAGPVFGKFAGDRYNFPVPSSFEQNEKKMVEGKLPSFGTVVAIILLPLFLIILNTLSSVIPFMKPLNPVLTFLGTPVIALLLSTITAMVLLGMRHGYTREELTKIMTESLSSAGNIILLIAGGGMLRFILQNSGIGEAFGNFVGGLPLPLVLVAFLIAAVVRISVGSATVSMTMAAGIMASMPAIADLSQIQLATITIAVAAGATAFSHVNDSGFWLSKSLFEVDEKTNLKTWTVMETIIGIVGVIGATIIWFIVQ

SEQ ID NO: 160 - protein, 465 aa, unidentified; identical to SEQ ID NO: 126

SEQ ID NO: 161 - protein, 479 aa, unidentified
MKFALLYYIKLAVFYIFHLCKENKRKELLGEKMVMVLIVVLGIAFLLLLVLKCKLHAFIALLIASIAVGVAAGMPLTDITASIQNGMGGTLGTVAIIVGLGAVFGQMLEASGGAQALAKSMIRFFGEKHAPLAFAVTGFIIAIPVFFDVGFIILLPLIYAVAYKTKRSVATFAFPLIISLCLTNAFVPPTPGPVATCSALDANVGWVIFFGIIISIPLTLVGYLYSIKYIDKHLFIPVPDYMIPEESNDENLPSFSSVILIIAVPILLILVNTGVDALVNEGILADSFFTQAAKFLGTSYIALLIAVLLSFYILGKRRGFHKEDLYEISSKAFLPVGLIILVTGAGGVFKQILLDSGVGDVIAQSVETIGLPPIILGYLVAVFIRVSQGSGMVAMITAAGIVSPLLAVSPVSEMQRALVVLAIAAGSVAASHVNDSGFWLVCKYLNMTEGQTLKTWTFITAFLSLCSLVLVILLSLIIP

SEQ ID NO: 162 - protein, 453 aa, unidentified; identical to SEQ ID NO: 123

SEQ ID NO: 163 - protein, 446 aa, unidentified; identical to SEQ ID NO: 135

SEQ ID NO: 164 - protein, 438 aa, unidentified; identical to SEQ ID NO: 134

SEQ ID NO: 165 - protein, 481 aa, unidentified
MLNPVVAMLIGIVVMMVLIIFTKMHAFPSLIISAILIAVLAMPFGEVSSLTQCVSTVTTGFGNTMASIGVVIGFGCIMGIFLEKSGAAKRMALTILKAVGVKNCDVVLGLTGWVVSIPVFCDSGFVILSSLAKEFSRLTKRSMVGLGGILGMGLYITHFMVPPTPGPLAVVSTFQENGLGIDLGMFILCGIGISIPLFAFAVPLFRHFGKKYPNLVVPYEIDRSQYSEAQLRVLDRINEKYQKNEDLVTEDFADLLAAEKLPSAFMSFATLLVPIVLIFANTLVGQISSLQGTMVANVVQFLGQPVIALFISLCMGAFLLCRDYDKKTCVNLMQEACRDAGPIVFITAAGGALGAVINATGAAKMMADAIVAAGVPGILVPIIIGVIMRFPQGSGTTAMITGSAIIAPMVAAGLAVNPYLAAMALCLTTMCPSYLNDSYFHVVTNFSGMDIKTSLKTWSVGSILIPAFGSCIFIVLSFFIH

SEQ ID NO: 166 - protein, 462 aa, unidentified
MFSGEMVTGPLLIGIFILAIAVLLVLIIKFKLNAFVALLVTAFGTGVLVNMPLADVAQTVTDGFGGTLGGIGMVTGLGVMLGKFMFESGGIESISNKILGAFGEKKSPIAVALSGFITGIPVFGDVVYIMFAPMLRVLSKKTKISMVNFACAISVATTCTFALVLPTAPPLAVAEELGIEIGIFFFYALISAFVGMLVGGIVYGSFLNKQDHKNNHFYTFEDLDQEEAQMAKGDSKDKMGAGKALSILLVPIVLILLGSFVPLAVGKEAAIVPFVTFIGNKNFAMMVGVIYAAVISRKYIKKTATDIMTEAADQVGLILLITGAGGAFGKVLQATGIADYVAGTLSSFSIPILILCFLIAQIIRCAQGSTTVALMTTAAIMSSTIATSGVSPILCAIAICAGGIGLSLPNDSGFWAISRFFRISVTDTIRGWSIGGFVAGVAILIFVSILSLFQGFLPGLMI

SEQ ID NO: 167 - protein, 421 aa, unidentified
MHAFVALMVVSIGAGLFSGMPLDKIAATMEKGMGGTLGFLAIVVALGAMFGKILHETGAVDQIAVKMLKSFGHNRAHYAIGLAGLICALPLFFEVAIVLLISVAFSMARHTGTNLVKLVIPLFAGVAAAAAFLLPGPAPMLLASQMHADFGWMILIGLCAAIPGMIIAGPLWGNFISRYVELHIPDDVTEPHLGEGKMPSFGFSLALILLPLVLVGLKTIAARFVPVGSTAYEWFEFIGHPFTAILVACLVAIYGLAVRQGMAKDRVMEICGHALQPAGIILLVIGAGGVFKQVLVDSGVGPALGEALTGMGLPIAITCFVLAAAVRIIQGSATVACLTAVGLVMPVIEQLNYSGAQMAALSICIAGGSIVVSHVNDAGFWLFGKFTGASEAQTLKTWTMMETILGTTGAIVGMIAFQLLS

SEQ ID NO: 168 - DNA, 1339 bp, unidentified
  misc_feature 1..1339: note = I01
  misc_feature 139: note = n is a, c, g, or t
  misc_feature 237: note = n is a, c, g, or t
  misc_feature 248: note = n is a, c, g, or t
  misc_feature 308..309: note = n is a, c, g, or t
  misc_feature 311..313: note = n is a, c, g, or t
  misc_feature 489: note = n is a, c, g, or t
  misc_feature 1158: note = n is a, c, g, or t
  misc_feature 1284: note = n is a, c, g, or t
  misc_feature 1286: note = n is a, c, g, or t
  source 1..1339: mol_type = other DNA
ctgggtgagagtggcgaacgggtgagtaatgcgtgaccgacctgccccatacaccggaatagctcctggaaacgggtggtaatgccggatgctccagttggatgcatgtccttctgggaaagattcatcggtatggganggggtcgcgtcctatcagcttgatggcggggtaacggcccaccatggcttccacgggtagccggcctgagagggcgaccggccacattgggactgagntacggcccagnctcctacgggaggcagcagtggggaatattgcacaatgggcgcaagcctgatgcagcganncnnngtgcgggatgacggccttcgggttgtaaaccgcttttgactgggagcaagcccttcggggtgagtgtacctttcgaataagcaccggctaactacgtgccagcagccgcggtaatacgtaaggtgcaagcgttatccggaattattgggcgtaaagggctcgtaagcggttcgtcncgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgtcactgacgctgaggagcgaaagcgtggggagcgaacaggattagataccctggtagtccacgccgtaaacggtggatgctggatgtggggaccattccacggtctccgtgtcggagccaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagccgtagagatacggtttcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcganaccgcgaggtggagcggatcccttaaaaccggtctcagttcggattggagtctgcaacccgactccatgaaggcggagtcgctagtaatcgcggatcagcaacgccgcggtgaatgcgttcccggncnttgtacacaccgcccgtcaagtcatgaaagtgggtagcacccgaagccggtgg

SEQ ID NO: 169 - DNA, 1346 bp, unidentified
  misc_feature 1..1346: note = I02
  misc_feature 911..913: note = n is a, c, g, or t
  misc_feature 923: note = n is a, c, g, or t
  source 1..1346: mol_type = other DNA
ttgcttggtggtgagagtggcgaacgggtgagtaatgcgtgaccgacctgccccatacaccggaatagctcctggaaacgggtggtaatgccggatgctccgactcctcgcatggggtgtcgggaaagatttcatcggtatgggatggggtcgcgtcctatcaggtagtcggcggggtaacggcccaccgagcctacgacgggtagccggcctgagagggcgaccggccacattgggactgagatacggcccagactcctacgggaggcagcagtggggaatattgcacaatgggcgcaagcctgatgcagcgacgccgcgtgcgggatgacggccttcgggttgtaaaccgcttttgatcgggagcaagccttcgggtgagtgtacctttcgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatctgcgccgggtacgggcgggctggagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacagcnnnagagatatgncctcccttcggggcgggttcacaggtggtgcatggtcgtcgtcagctcgtgtcgtgagatgttgggttaagtcccgcaacgagcgcaaccctcgccctgtgttgccagcacgtcgtggtgggaactcacgggggaccgccggggtcaactcggaggaaggtggggatgacgtcagatcatcatgccccttacgtccagggcttcacgcatgctacaatggccggtacaacgggatgcgacacggcgacgtggagcggatccctgaaaaccggtctcagttcggattggagtctgcaacccgactccatgaaggcggagtcgctagtaatcgcggatcagcaacgccgcggtgaatgcgttcccgggccttgtacacaccgcccgtcaagtcatgaaagtgggtagcacccgaagccggtggcc

SEQ ID NO: 170 - DNA, 1353 bp, unidentified
  misc_feature 1..1353: note = I03
  source 1..1353: mol_type = other DNA
aagcttgcttggtggtgagagtggcgaacgggtgagtaatgcgtgaccgacctgccccatgctccggaatagctcctggaaacgggtggtaatgccggatgttccacatgatcgcatgtgattgtgggaaagattctatcggcgtgggatggggtcgcgtcctatcagcttgttggtgaggtaacggctcaccaaggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttgtttgggagcaagccttcgggtgagtgtacctttcgaataagcgccggctaactacgtgccagcagccgcggtaatacgtagggcgcaagcgttatccggatttattgggcgtaaagggctcgtaggcggctcgtcgcgtccggtgtgaaagtccatcgcttaacggtggatctgcgccgggtacgggcgggctggagtgcggtaggggagactggaattcccggtgtaacggtggaatgtgtagatatcgggaagaacaccgatggcgaaggcaggtctctgggccgtcactgacgctgaggagcgaaagcgtggggagcgaacaggattagataccctggtagtccacgccgtaaacggtggacgctggatgtggggcacgttccacgtgttccgtgtcggagctaacgcgttaagcgtcccgcctggggagtacggccgcaaggctaaaactcaaagaaattgacgggggcccgcacaagcggcggagcatgcggattaattcgatgcaacgcgaagaaccttacctgggcttgacatgttcccgacgacgccagagatggcgtttcccttcggggcgggttcacaggtggtgcatggtcgtcgtcagctcgtgtcgtgagatgttgggttaagtcccgcaacgagcgcaaccctcgccccgtgttgccagcacgttatggtgggaactcacgggggaccgccggggttaactcggaggaaggtggggatgacgtcagatcatcatgccccttacgtccagggcttcacgcatgctacaatggccggtacagcgggatgcgacatggcgacatggagcggatccctgaaaaccggtctcagttcggatcggagcctgcaacccggctccgtgaaggcggagtcgctagtaatcgcggatcagcaacgccgcggtgaatgcgttcccgggccttgtacacaccgcccgtcaagtcatgaaagtgggcagcacccgaagccggtggcctaa

SEQ ID NO: 171 - DNA, 1338 bp, unidentified
  misc_feature 1..1338: note = I04
  source 1..1338: mol_type = other DNA
ggtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatgcggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggcct

SEQ ID NO: 172 - DNA, 1326 bp, unidentified
  misc_feature 1..1326: note = I05
  source 1..1326: mol_type = other DNA
gatagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaaccccccgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctaggtgtggggggacgatccccccgtgccgcagccaacgcattaagcgtcccgcctggggagtacggccgcaaggctaaaactcaaaggaattgacgggggcccgcacaagcagcggagcatgtggcttaattcgaagcaacgcgaagaaccttaccagggcttgacatatgggtgaagcgggggagaccccgtggccgagaggagcccatacaggtggtgcatggctgtcgtcagctcgtgtcgtgagatgttgggttaagtcccgcaacgagcgcaacccccgccgcgtgttgccatcgggtgatgccgggaacccacgcgggaccgccgccgtcaaggcggaggagggcggggacgacgtcaagtcatcatgccccttatgccctgggctgcacacgtgctacaatggccggtacagagggatgccaccccgcgagggggagcggatcccggaaagccggccccagttcggattgggggctgcaacccgcccccatgaagtcggagttgctagtaatcgcggatcagcatgccgcggtgaatgcgttcccgggccttgtacacaccgcccgtcacaccacccgagtcgtctgcacccgaagtcgccggc

SEQ ID NO: 173 - DNA, 729 bp, unidentified
  misc_feature 1..729: note = I06
  misc_feature 13: note = n is a, c, g, or t
  source 1..729: mol_type = other DNA
acctgccttcggncagaagcgagtggcgaacggctgagtaacacgtggagaacctgccccctcccccgggatagccgcccgaaaggacgggtaataccggataccccggggtgccgcatggcaccccggctaaagccccgacgggaggggatggctccgcggcccatcaggtagacggcggggtgacggcccaccgtgccgacaacgggtagccgggttgagagaccgaccggccagattgggactgagacacggcccagactcctacgggaggcagcagtggggaatcttgcgcaatggggggaaccctgacgcagcgacgccgcgtgcgggacggaggccttcgggtcgtaaaccgctttcagcagggaagagtcaagactgtacctgcagaagaagccccggctaactacgtgccagcagccgcggtaatacgtagggggcgagcgttatccggattcattgggcgtaaagcgcgcgtaggcggcccggcaggccgggggtcgaagcggggggctcaacccctcgaagcccccggaacctccgcggcttgggtccggtaggggagggtggaacacccggtgtagcggtggaatgcgcagatatcgggtggaacaccggtggcgaaggcggccctctgggccgagaccgacgctgaggcgcgaaagctgggggagcgaacaggattagataccctggtagtcccagccgtaaacgatggacgctagg

SEQ ID NO: 174 - DNA, 1338 bp, unidentified
  misc_feature 1..1338: note = I07
  source 1..1338: mol_type = other DNA
gtggtgagagtggcgaacgggtgagtaatgcgtgaccgacctgccccatacaccggaatagctcctggaaacgggtggtaatgccggatgctccagttgatcgcatggtcttctgggaaagctttcgcggtatgggatggggtcgcgtcctatcagcttgacggcggggtaacggcccaccgtggcttcgacgggtagccggcctgagagggcgaccggccacattgggactgagatacggcccagactcctacgggaggcagcagtggggaatattgcacaatgggcgcaagcctgatgcagcgacgccgcgtgagggatggaggccttcgggttgtaaacctcttttatcggggagcaagcgagagtgagtttacccgttgaataagcaccggctaactacgtgccagcagccgcggtaatacgtagggtgcaagcgttatccggaattattgggcgtaaagggctcgtaggcggttcgtcgcgtccggtgtgaaagtccatcgcttaacggtggatccgcgccgggtacgggcgggcttgagtgcggtaggggagactggaattcccggtgtaacggtggaatgtgtagatatcgggaagaacaccaatggcgaaggcaggtctctgggccgttactgacgctgaggagcgaaagcgtggggagcgaacaggattagataccctggtagtccacgccgtaaacggtggatgctggatgtggggcccgttccacgggttccgtgtcggagctaacgcgttaagcatcccgcctggggagtacggccgcaaggctaaaactcaaagaaattgacgggggcccgcacaagcggcggagcatgcggattaattcgatgcaacgcgaagaaccttacctgggcttgacatgttcccgacggtcgtagagatgcggcttcccttcggggcgggttcacaggtggtgcatggtcgtcgtcagctcgtgtcgtgagatgttgggttaagtcccgcaacgagcgcaaccctcgccccgtgttgccagcggattatgccgggaactcacgggggaccgccggggttaactcggaggaaggtggggatgacgtcagatcatcatgccccttacgtccagggcttcacgcatgctacaatggccggtacaacgggatgcgacgcggcgacgcggagcggatccctgaaaaccggtctcagttcggatcgcagtctgcaactcgactgcgtgaaggcggagtcgctagtaatcgcgaatcagcaacgtcgcggtgaatgcgttcccgggccttgtacacaccgcccgtcaagtcatgaaagtgggcagcacccgaagccggtggccta

SEQ ID NO: 175 - DNA, 1337 bp, unidentified
  misc_feature 1..1337: note = I08
  misc_feature 105: note = n is a, c, g, or t
  misc_feature 117..119: note = n is a, c, g, or t
  source 1..1337: mol_type = other DNA
ttgatggatggcgaccggcgcacgggtgagtaacacgtatccaacctgccgacaacactgggatagcctttcgaaagaaagattaataccggatggcataattantccgcatgggannnttattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatactgtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggaatcggaaacggttctttcttcggacagttgtgaaggtgctgcatggttgtcgtcagctcgtgccgtgaggtgtcggcttaagtgccataacgagcgcaacccttacgggtagttaccatcaggttatgctggggactctacccggactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctg

SEQ ID NO: 176 - DNA, 1353 bp, unidentified
  misc_feature 1..1353: note = I09
  source 1..1353: mol_type = other DNA
ttagcttgctaaggccgatggcgaccggcgcacgggtgagtaacgcgtatccaacctgccttacactcttggacagccttctgaaagggagattaatacaagatgttatcatgagtaagcattttcgcatgattaaaggtttaccggtgtaagatggggatgcgttccattagatagtaggcggggtaacggcccacctagtcttcgatggataggggttctgagaggaaggtcccccacattggaactgagacacggtccaaactcctacgggaggcagcagtgaggaatattggtcaatggacgagagtctgaaccagccaagtagcgtgaaggatgaaggttctatggattgtaaacttcttttatacgggaataaacgaatccacgcgtggatttttgcatgtaccgtatgaataaggatcggctaactccgtgccagcagccgcggtaatacggaggatccgagcgttatccggatttattgggtttaaagggagcgtagatgggttgttaagtcagttgtgaaagtttgcggctcaaccgtaaaattgcaattgatactggcagtcttgagtacagttgaggtaggcggaattcgtggtgtagcggtgaaatgcttagatatcacgaagaactccgattgcgaaggcagcttactaacctgtaactgacattgatgctcgaaagtgtgggtatcaaacaggattagataccctggtagtccacacggtaaacgatgaatactcgctgtaggcgatatacggtctgcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaaccgaatatggcggaaacgccatagctagcaatagcggttgtgaaggtgctgcatggttgtcgtcagctcgtgccgtgaggtgtcggcttaagtgccataacgagcgcaacccttgccgatagttactaacaggttatgctgaggactctgtcgggactgccatcgtaagatgtgaggaaggtggggatgacgtcaaatcagcacggcccttacgtccggggctacacacgtgttacaatggggggtacagagggctgctaccacgcaagtggatgccaatcccaaaaacctctctcagttcggattgaagtctgcaacccgacttcatgaagctggattcgctagtaatcgcgcatcagccacggcgcggtgaatacgttcccgggccttgtacacaccgcccgtcaagccatgggagccgggggtacctgaag

SEQ ID NO: 177 - DNA, 1350 bp, unidentified
  misc_feature 1..1350: note = I10
  source 1..1350: mol_type = other DNA
cttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctga

SEQ ID NO: 178 - DNA, 1356 bp, unidentified
  misc_feature 1..1356: note = I11
  source 1..1356: mol_type = other DNA
tcttagcttgctaaggctgatggcgaccggcgcacgggtgagtaacacgtatccaacctgccgtctactcttggccagccttctgaaaggaagattaatccaggatgggatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaa

SEQ ID NO: 179 - DNA, 1353 bp, unidentified
  misc_feature 1..1353: note = I12
  misc_feature 6: note = n is a, c, g, or t
  misc_feature 8: note = n is a, c, g, or t
  source 1..1353: mol_type = other DNA
tgtagnantacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacaagcggaggaacatgtggtttaattcgatgatacgcgaggaaccttacccgggtttgaacgtagtctgaccggagtggaaacactccttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggcgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaa

SEQ ID NO: 180 - DNA, 824 bp, unidentified
  misc_feature 1..824: note = I13
  source 1..824: mol_type = other DNA
ggcgaccggcgcacgggtgagtaacgcgtatgcaacttacctatcagagggggataacccggcgaaagtcggactaataccgcatgaagcaggggccccgcatggggatatttgctaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatgggcgtaagcctgaaccagccaagtcgcgtgagggatgaaggttctatggatcgtaaacctcttttataagggaataaagtgcgggacgtgtcctgttttgtatgtaccttatgaataaggatcggctaactccgtgccagcagccgcggtaatacggaggatccgagcgttatccggatttattgggtttaaagggtgcgtaggcggccttttaagtcagcggtgaaagtctgtggctcaaccatagaattgccgttgaaactggggggcttgagtatgtttgaggcaggcggaatgcgtggtgtagcggtgaaatgcttagatatcacgcagaaccccgattgcgaaggcagcctgccaagccatgactgacgctgatgcacgaaagcgtggggatcaaacaggattagataccctggtagtccacgcagtaaacgatgatcactagctgtttgcgatacagtgtaagcggcacagcgaaagcgttaagtgatccacctggggagtacgccggcaacggtgaaactcaaaggaattgac

SEQ ID NO: 181 - DNA, 1348 bp, unidentified
  misc_feature 1..1348: note = I14
  source 1..1348: mol_type = other DNA
gatagcaatatctatggtggcgaccggcgcacgggtgcgtaacgcgtatgcaacctacctttaacagggggataacactgagaaattggtactaataccccataatatcatagaaggcatcttttatggttgaaaattccgatggttagagatgggcatgcgttgtattagctagttggtggggtaacggctcaccaaggcgacgatacatagggggactgagaggttaaccccccacactggtactgagacacggaccagactcctacgggaggcagcagtgaggaatattggtcaatggacgcaagtctgaaccagccatgccgcgtgcaggatgacggctctatgagttgtaaactgcttttgtacgagggtaaacgcagatacgtgtatctgtctgaaagtatcgtacgaataaggatcggctaactccgtgccagcagccgcggtaatacggaggattcaagcgttatccggatttattgggtttaaagggtgcgtaggcggtttgataagttagaggtgaaatttcggggctcaaccctgaacgtgcctctaatactgttgagctagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttagcgacgattcttgaaagaggatttcccttcggggcgcgaaactaggtgctgcatggttgtcgtcagctcgtgccgtgaggtgtcgggttaagtcccataacgagcgcaacccctaccgttagttgccatcaggtgaagctgggcactctggcgggactgccggtgtaagccgagaggaaggtggggatgacgtcaaatcagcacggcccttacgtccggggctacacacgtgttacaatggtaggtacagagggcagctacccagcgatgggatgcgaatctcgaaagcctatctcagttcggattggaggctgaaacccgcctccatgaagttggattcgctagtaatcgcgcatcagccatggcgcggtgaatacgttcccgggccttgtacacaccgcccgtcaagccatgggagccgggggtgcctga

SEQ ID NO: 182 - DNA, 1337 bp, unidentified
  misc_feature 1..1337: note = I15
  misc_feature 838..839: note = n is a, c, g, or t
  source 1..1337: mol_type = other DNA
ttctttgctggcgaccggcgcacgggtgagtaacacgtatccaacctgccgatgactcggggatagcctttcgaaagaaagattaatacccgatggtatatctgaaaggcatctttcagctattaaagaatttcggtcattgatggggatgcgttccattaggttgttggcggggtaacggcccaccaagccatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttgggcacgtgtgcctttttgtatgtaccgtatgaataaggatcggctaactccgtgccagcagccgcggtaatacggaggatccgagcgttatccggatttattgggtttaaagggagcgtaggcggatgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcnngcacaagcggaggaacatgtggtttaattcgatgatacgcgaggaaccttacccgggcttaaattgcaaatgaatgttctggaaacagatcagccgcaaggcatttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggcgacgtgatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctg

SEQ ID NO: 183 - DNA, 1343 bp, unidentified
  misc_feature 1..1343: note = I16
  misc_feature 5: note = n is a, c, g, or t
  misc_feature 7: note = n is a, c, g, or t
  misc_feature 110..113: note = n is a, c, g, or t
  misc_feature 337..338: note = n is a, c, g, or t
  misc_feature 1170: note = n is a, c, g, or t
  source 1..1343: mol_type = other DNA
cttgnanactgaagatggcgaccggcgcacgggtgagtaacacgtatccaacctgccgataactccggaatagcctttcgaaagaaagattaataccggatagcatacgnnnntcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatganngctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgtcactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcanctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacct

SEQ ID NO: 184 - DNA, 1335 bp, unidentified
  misc_feature 1..1335: note = I17
  source 1..1335: mol_type = other DNA
gggtggcgaccggcgcacgggtgcgtaacgcgtatgcaacctacccataacagggggataacactgagaaattggtactaataccccataacatcagaaccggcatcggttttggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaaacgcttctacgtgtaggagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaa

SEQ ID NO: 185 - DNA, 1309 bp, unidentified
  misc_feature 1..1309: note = I18
  source 1..1309: mol_type = other DNA
cgtatgcaacctgcccgataccggggtatagcccatggaaacgtggattaacaccccatagtacttttatcctgcatgggatgtgagttaaatgttcaaggtatcggatgggcatgcgtcctattagttagttggcggggtaacagcccaccaagacgatgataggtaggggttctgagaggaaggtcccccacattggaactgagacacggtccaaactcctacgggaggcagcagtgaggaatattggtcaatggacgtaagtctgaaccagccaagtcgcgtgagggaagactgccctatgggttgtaaacctcttttataagggaagaataagttctacgtgtagaatgatgcctgtaccttatgaataagcatcggctaactccgtgccagcagccgcggtaatacggaggatgcgagcgttatccggatttattgggtttaaagggtgcgtaggcggtttattaagttagtggttaaatatttgagctaaactcaattgtgccattaatactggtaaactggagtacagacgaggtaggcggaataagttaagtagcggtgaaatgcatagatataacttagaactccgatagcgaaggcagcttaccagactgtaactgacgctgatgcacgagagcgtgggtagcgaacaggattagataccctggtagtccacgccgtaaacgatgctcactggttctgtgcgatatattgtacgggattaagcgaaagtattaagtgagccacctggggagtacgtcggcaacgatgaaactcaaaggaattgacgggggcccgcacaagcggaggaacatgtggtttaattcgatgatacgcgaggaaccttacctgggtttaaatgggaaatgtcgtatttggaaacagatattctcttcggagcgtttttcaaggtgctgcatggttgtcgtcagctcgtgccgtgaggtgtcgggttaagtcccataacgagcgcaacccttaccgttagttgctagcatgtaatgatgagcactctaacgggactgccaccgtaaggtgagaggaaggcggggatgacgtcaaatcagcacggcccttacacccagggctacacacgtgttacaatggccggtacagagggccgctaccaggtgactggatgccaatctcaaaagccggtcgtagttcggattggagtctgtaacccgactccatgaagttggattcgctagtaatcgcgcatcagccatggcgcggtgaatacgttcccgggccttgtacacaccgcccgtcaagccatggaagccgggggtgcctgaagtcc

SEQ ID NO: 186 - DNA, 1339 bp, unidentified
  misc_feature 1..1339: note = I19
  misc_feature 10: note = n is a, c, g, or t
  misc_feature 124..125: note = n is a, c, g, or t
  misc_feature 183: note = n is a, c, g, or t
  misc_feature 190..191: note = n is a, c, g, or t
  misc_feature 911: note = n is a, c, g, or t
  source 1..1339: mol_type = other DNA
agagagcttnctttctcgagcgagtggcgaacgggtgagtaacgcgtgaggaacctgcctcaaagagggggacaacagttggaaacgactgctaataccgcataagcccacgggtcggcatcgnncagagggaaaaggagcaatccgctttgagatggcctcgcgtccgattagctagttggngaggtannggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacaganatagaaatatgttttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtagtc

SEQ ID NO: 187 - DNA, 1339 bp, unidentified
  misc_feature 1..1339: note = I20
  misc_feature 192: note = n is a, c, g, or t
  misc_feature 205..206: note = n is a, c, g, or t
  misc_feature 1337: note = n is a, c, g, or t
  source 1..1339: mol_type = other DNA
gagagagcttgctttctcgagcgagtggcgaacgggtgagtaacgcgtgaggaacctgcctcaaagagggggacaacagttggaaacgactgctaataccgcataagcccacgggtcggcatcgaccagagggaaaaggagtaatccgctttgagatggcctcgcgtccgattagctagttggtgaggtaanggcccaccaaggnnacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacagacatagaaatatgttttctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcaagaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtngt

SEQ ID NO: 188 - DNA, 1354 bp, unidentified
  misc_feature 1..1354: note = I21
  misc_feature 184..187: note = n is a, c, g, or t
  misc_feature 573..574: note = n is a, c, g, or t
  misc_feature 752..755: note = n is a, c, g, or t
  misc_feature 1175..1176: note = n is a, c, g, or t
  source 1..1354: mol_type = other DNA
cattgagacttcggtggatttgatctatttctagtggcggacgggtgagtaacgcgtgggtaacctgccttatacagggggataacagtcagaaatggctgctaataccgcataagcgcacagagctgcatggctcagtgtgaaaaactccggtggtataagatggacccgcgttggattagcnnnntggtggggtaacggcccaccaaggcgacgatccatagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagatagtgacggtacctgactaagaagccccggctaactacgtgccagcagccgcggtaatacgtagggggcaagcgttatccggatttactgggtgtaaagggagcgtagacggtgtggcaagtctgatgtgaaaggcatgggctcaacctgtggactgcattggaaactgtcatacttgagtgccggnngggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcggggagcnnnnctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccgcctgaccgatccttaaccggatctttccttcgggacaggcgagacaggtggtgcatggttgtcgtcagctcgtgtcgtgagatgttgggttaagtcccgcaacgagcgcaacccctatcctcagtagccagcatttaaggtgggcactctggggagactgccagggataacctggaggaaggcggggatgacgtcaaatcatcatgccccttatgatttgggctacacacgtgctacaatggcgtaaacaaagggaagcgagatnntgagatggagcaaatcccaaaaataacgtcccagttcggactgtagtctgcaacccgactacacgaagctggaatcgctagtaatcgcggatcagaatgccgcggtgaatacgttcccgggtcttgtacacaccgcccgtcacaccatgggagtcagtaacgcccgaagtcagtgacc

SEQ ID NO: 189 - DNA, 1264 bp, unidentified
  misc_feature 1..1264: note = I22
  misc_feature 144..145: note = n is a, c, g, or t
  source 1..1264: mol_type = other DNA
ttacttggatttcttcggaatgacgagtattgtgactgagcggcggacgggtgagtaacgcgtgggtaacctgcctcatacagggggataacagttagaaatgactgctaataccgcataagaccacagcaccgcatggtgcannggtaaaaactccggtggtatgagatggacccgcgtctgattagctggttggtggggtaacggcctaccaaggcgacgatcagtagccggcctgagagggcgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaactacgtgccagcagccgcggtaatacgtagggggcccgtttttctagagtgtcggagaggtaagcggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactactaggtgtcgggtggcaaagccattcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctgctcttgacatcccggtgaccgctccgtaatgggagcttttcttcggaacaccggagacaggtggtgcatggttgtcgtcagctcgtgtcgtgagatgttgggttaagtcccgcaacgagcgcaacccctatcttcagtagccagcggtttggccgggcactctggagagactgccagggataacctggaggaaggtggggatgacgtcaaatcatcatgccccttatgagcagggctacacacgtgctacaatggcgtaaacaaagggaggcgaactcgcgagggtaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgaccc

SEQ ID NO: 190 - DNA, 1340 bp, unidentified
  misc_feature 1..1340: note = I23
  source 1..1340: mol_type = other DNA
gagaagcttgcttttctgatctagtggcggacgggtgagtaacacgtgagcaatctgcctttcagagggggataccgattggaaacgatcgttaataccgcataatataattgaaccgcatgatttgattatcaaagatttatcgctgaaagatgagctcgcgtctgattagctagttggtaaggtaacggcttaccaaggcgacgatcagtagccggactgagaggttgatcggccacattgggactgagacacggcccagactcctacgggaggcagcagtggggaatattgcacaatggaggaaactctgatgcagcgatgccgcgtgagggaagaaggttttaggattgtaaacctctgtcttcagggacgaaaaatgacggtacctgaggaggaagctccggctaactacgtgccagcagccgcggtaatacgtagggagcgagcgttgtccggaattactgggtgtaaagggagcgtaggcgggatcgcaagtcagatgtgaaaactatgggcttaacccataaactgcatttgaaactgtggttcttgagtgaagtagaggtaagcggaattcctagtgtagcggtgaaatgcgtagatattaggaggaacatcagtggcgaaggcggcttactgggctttaactgacgctgaggctcgaaagcgtggggagcaaacaggattagataccctggtagtccacgccgtaaacgatgattactaggtgtggggggactgaccccttccgtgccgcagcaaacgcaataagtaatccacctggggagtacgaccgcaaggttgaaactcaaaggaattgacgggggcccgcacaagcagtggagtatgtggattaattcgaagcaacgcgaagaaccttaccaggtcttgacatcgtatgcatagctcagagatgagtgaaatctcttcggagacatatagacaggtggtgcatggttgtcgtcagctcgtgtcgtgagatgttgggttaagtcccgcaacgagcgcaacccttactgttagttgctacgcaagagcactctagcaggactgccgttgacaaaacggaggaaggtggggatgacgtcaaatcatcatgccccttatgacctgggcctcacacgtactacaatggctgtcaacagagggatgcaaagccgcgaggtggagcgaacccctaaaagcagtcttagttcggattgtaggctgcaacccgcctacatgaagtcggaattgctagtaatcgcagatcagcatgctgcggtgaatacgttcccgggccttgtacacaccgcccgtcacgccatgggagtcggtaacacccgaagcctgtagtc

SEQ ID NO: 191 - DNA, 1337 bp, unidentified
  misc_feature 1..1337: note = I24
  misc_feature 53..54: note = n is a, c, g, or t
  misc_feature 113..115: note = n is a, c, g, or t
  misc_feature 123..125: note = n is a, c, g, or t
  misc_feature 1155..1156: note = n is a, c, g, or t
  misc_feature 1336: note = n is a, c, g, or t
  source 1..1337: mol_type = other DNA
agagagcttgctttctcgagcgagtggcgaacgggtgagtaacgcgtgaggannctgcctcaaagagggggacaacagttggaaacgactgctaataccgcataagcccacgnnnccgcatgnnncagagggaaaaggagtaatccgctttgagatggcctcgcgtccgattagctagttggtgaggtaacggcccaccaaggcgacgatcggtagccggactgagaggttgaacggccacattgggactgagacacggcccagactcctacgggaggcagcagtggggaatattgcacaatgggggaaaccctgatgcagcgacgccgcgtggaggaagaaggtcttcggattgtaaactcctgttgttggggaagataatgacggtacccaacaaggaagtgacggctaactacgtgccagcagccgcggtaaaacgtaggtcacaagcgttgtccggaattactgggtgtaaagggagcgcaggcgggaagacaagttggaagtgaaatctatgggctcaacccataaactgctttcaaaactgtttttcttgagtagtgcagaggtaggcggaattcccggtgtagcggtggaatgcgtagatatcgggaggaacaccagtggcgaaggcggcctactgggcaccaactgacgctgaggctcgaaagtgtgggtagcaaacaggattagataccctggtagtccacaccgtaaacgatgattactaggtgttggaggattgaccccttcagtgccgcagttaacacaataagtaatccacctggggagtacgaccgcaaggttgaaactcaaaggaattgacgggggcccgcacaagcagtggagtatgtggtttaattcgacgcaacgcgaagaaccttaccaagtcttgacatcccttgacagacatagaaatatgtaatctcttcggagcaaggagacaggtggtgcatggttgtcgtcagctcgtgtcgtgagatgttgggttaagtcccgcaacgagcgcaacccttatggtcagttactacgcaagaggactctggccagactgccgttgacaaaacggaggaaggtggggatgacgtcaaatcatcatgccctttatgacttgggctacacacgtactacaatggcgttaaacaaagagaagcnngaccgcgaggtggagcaaaactcagaaacaacgtcccagttcggactgcaggctgcaactcgcctgcacgaagtcggaattgctagtaatcgtggatcagcatgccacggtgaatacgttcccgggccttgtacacaccgcccgtcacaccatgagagccggggggacccgaagtcggtng

SEQ ID NO: 192 - DNA, 1353 bp, unidentified
  misc_feature 1..1353: note = I25
  misc_feature 570..572: note = n is a, c, g, or t
  source 1..1353: mol_type = other DNA
gattcttcggatgaagacttttgtgactgagcggcggacgggtgagtaacgcgtgggtaacctgcctcatacagggggataacagttagaaatgactgctaataccgcataagaccacggtaccgcatggtacagtggtaaaaactccggtggtatgagatggacccgcgtctgattaggtagttggtggggtaacggcctaccaagccgacgatcagtagccgacctgagagggtgaccggccacattgggactgagacacggcccagactcctacgggaggcagcagtggggaatattgcacaatggaggaaactctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcacggcaagccagatgtgaaagcccggggctcaaccccgggactgcatttggaactgctgagctagagtgtcggnnnggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactgctaggtgtcgggtggcaaagccattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagcttttcttcggaacatcggtgacaggtggtgcatggttgtcgtcagctcgtgtcgtgagatgttgggttaagtcccgcaacgagcgcaacccctatcttcagtagccagcaggttaagctgggcactctggagagactgccagggataacctggaggaaggtggggatgacgtcaaatcatcatgccccttatgaccagggctacacacgtgctacaatggcgtaaacaaagagaagcgaactcgcgagggtaagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtgaccc

SEQ ID NO: 193 - DNA, 1361 bp, unidentified
  misc_feature 1..1361: note = I26
  misc_feature 9: note = n is a, c, g, or t
  misc_feature 939..940: note = n is a, c, g, or t
  misc_feature 1052..1053: note = n is a, c, g, or t
  misc_feature 1357: note = n is a, c, g, or t
  source 1..1361: mol_type = other DNA
cttaagttngattcttcggatgaagacttttgtgactgagcggcggacgggtgagtaacgcgtgggtaacctgcctcatacagggggataacagttagaaatggctgctaataccgcataagaccacagtactgcatggtacagtggtaaaaactccggtggtatgagatggacccgcgtctgattaggtagttggtgaggtaacggcccaccaagccgacgatcagtagccgacctgagagggtgaccggccacattgggactgagacacggcccagactcctacgggaggcagcagtggggaatattgcacaatgggcgaaagcctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggctgtgcaagtctgaagtgaaaggcatgggctcaacctgtggactgctttggaaactgtgcagctagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgactgctaggtgtcgggtagcaaagctattcggtgccgcagctaacgcaataagcagtccacctggggagtacgttcgcaagaatgaaactcaaaggaattgacggggacccgcacaagcggtggagcatgtggtttaattcgaagcaacgcgaagaaccttacctgatcttgacatcccgatgaccgcttcgtaatggaagnntttcttcggaacatcggtgacaggtggtgcatggttgtcgtcagctcgtgtcgtgagatgttgggttaagtcccgcaacgagcgcaacccttatcttcagtagccagcattnnggatgggcactctggagagactgccagggataacctggaggaaggtggggatgacgtcaaatcatcatgccccttatgaccagggctacacacgtgctacaatggcgtaaacaaagggaagcagagccgcgaggccgagcaaatctcaaaaataacgtctcagttcggattgtagtctgcaactcgactacatgaagctggaatcgctagtaatcgcagatcagaatgctgcggtgaatacgttcccgggtcttgtacacaccgcccgtcacaccatgggagtcagtaacgcccgaagtcagtnaccc

SEQ ID NO: 194 - DNA, 1345 bp, unidentified
  misc_feature 1..1345: note = I27
  source 1..1345: mol_type = other DNA
tttcttcggaactgaagatttggtgattgagtggcggacgggtgagtaacgcgtgggtaacctgccctgtacagggggataacagtcagaaatgactgctaataccgcataagaccacagcaccgcatggtgcaggggtaaaaactccggtggtacaggatggacccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggaattactgggtgtaaagggtgcgtaggtggtatggcaagtcagaagtgaaaacccagggcttaactctgggactgcttttgaaactgtcagactggagtgcaggagaggtaagcggaattcctagtgtagcggtgaaatgcgtagatattaggaggaacatcagtggcgaaggcggcttactggactgaaactgacactgaggcacgaaagcgtggggagcaaacaggattagataccctggtagtccacgccgtaaacgatgaatactaggtgtcggggccgtagaggcttcggtgccgcagccaacgcagtaagtattccacctggggagtacgttcgcaagaatgaaactcaaaggaattgacggggacccgcacaagcggtggagcatgtggtttaattcgaagcaacgcgaagaaccttacctggtcttgacatccttctgaccggtccttaaccggacctttccttcgggacaggagagacaggtggtgcatggttgtcgtcagctcgtgtcgtgagatgttgggttaagtcccgcaacgagcgcaacccctatctttagtagccagcatatcaggtgggcactctagagagactgccagggataacctggaggaaggtggggacgacgtcaaatcatcatgccccttatgaccagggctacacacgtgctacaatggcgtaaacagagggaagcagcctcgtgagagtgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtca

SEQ ID NO: 195 - DNA, 400 bp, unidentified
  misc_feature 1..400: note = I28
  misc_feature 63..68: note = n is a, c, g, or t
  misc_feature 120..121: note = n is a, c, g, or t
  source 1..400: mol_type = other DNA
gcggcggacgggtgagtaacgcgtgggtaacctgccctgtacacacggataacataccgaaannnnnnctaatacgggataacataagaaattcgcatgtttttcttatcaaagctccgnnggtacaggatggacccgcgtctgattagctagttggtgaggtaacggctcaccaaggcgacgatcagtagccgacctgagagggtgatcggccacattggaactgagacacggtccaaactcctacgggaggcagcagtggggaatattgcacaatgggcgaaagcctgatgcagcaacgccgcgtgagcaatgaaggccttcgggtcgtaaagctctgtcctcaaggaagataatgacggtacttgaggaggaagccccggctaactacgtgccagca

SEQ ID NO: 196 - DNA, 1366 bp, unidentified
  misc_feature 1..1366: note = I29
  source 1..1366: mol_type = other DNA
gggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacctgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaggcagagatgcgttaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtaaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcc

SEQ ID NO: 197 - DNA, 1358 bp, unidentified
  misc_feature 1..1358: note = I30
  source 1..1358: mol_type = other DNA
ttagaaagaggattcgtccaattgataaggttacttagtggcggacgggtgagtaacgcgtgaggaacctgcctcggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggctacacacgtactacaatggtggtaaacagagggaagcaagaccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagtc

SEQ ID NO: 198 - DNA, 1342 bp, unidentified
  misc_feature 1..1342: note = I31
  source 1..1342: mol_type = other DNA
ggcgaacgggtgagtaatacataagtaacctggcatctacagggggataactgatggaaacgtcagctaagaccgcataggtgtagagatcgcatgaactctatatgaaaagtgctacgggactggtagatgatggacttatggcgcattagctggttggtagggtaacggcctaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaagtaattcgttatgtaaacttctgtcatagaggaagaacggtggatatagggaatgatatccaagtgacggtactctataagaaagccacggctaactacgtgccagcagccgcggtaatacgtaggtggcgagcgttatccggaattattgggcgtaaagagggagcaggcggcactaagggtctgtggtgaaagatcgaagcttaacttcggtaagccatggaaaccgtagagctagagtgtgtgagaggatcgtggaattccatgtgtagcggtgaaatgcgtagatatatggaggaacaccagtggcgaaggcgacgatctggcgcataactgacgctcagtcccgaaagcgtggggagcaaataggattagataccctagtagtccacgccgtaaacgatgagtactaagtgttgggagtcaaatctcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatcgatctaaaggctccagagatggagagatagctatagagaagacaggtggtgcatggttgtcgtcagctcgtgtcgtgagatgttgggttaagtcccgcaacgagcgcaacccctgttgccagttgccagcattaagttggggactctggcgagactgccggtgacaagccggaggaaggcggggatgacgtcaaatcatcatgccccttatgacctgggctacacacgtgctacaatggacagagcagagggaagcgaagccgcgaggtggagcgaaacccataaaactgttctcagttcggactgcagtctgcaactcgactgcacgaagatggaatcgctagtaatcgcgaatcagcatgtcgcggtgaatacgttctcgggccttgtacacaccgcccgtcacaccatgagagtcggtaacacccgaagccggtggccta

SEQ ID NO: 199 - DNA, 1390 bp, unidentified
  misc_feature 1..1390: note = I32
  misc_feature 921..923: note = n is a, c, g, or t
  misc_feature 972: note = n is a, c, g, or t
  misc_feature 983: note = n is a, c, g, or t
  misc_feature 990..991: note = n is a, c, g, or t
  source 1..1390: mol_type = other DNA
atgagaagcttgcttcttattgattcgagtggcaaacgggtgagtaacgcgtaagcaacctgcccttcagatggggacaacagctggaaacggctgctaataccgaatacgttctttttgtcgcatggcagagggaagaaagggaggctcttcggagctttcgctgaaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatacgggacgaatggcgtagcggtcaatacccgttacgagtgacggtaccgtaagagaaagccacggctaactacgtgccagcagccgcggtaatacgtaggtggcaagcgttgtccggaattattgggcgtaaagggcgcgcaggcggcgtcgtaagtcggtcttaaaagtgcggggcttaaccccgtgaggggaccgaaactgcgatgctagagtatcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgaccccttctgtgccggagttaacgcaataagtatcccgcctggggagtacggccgcaaggctgaaactcaaaggaattgacgggggcccgcacaagcggtggagtatgtggtttaattcgacgcaacgcgaagnnncttaccaagccttgacattgattgctatggatagagatatccagttccncttcggagganaagaaannaggtggtgcacggctgtcgtcagctcgtgtcgtgagatgttgggttaagtcccgcaacgagcgcaacccctatcttctgttaccagcggttcggccggggactcaggagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgag

SEQ ID NO: 200 - DNA, 1373 bp, unidentified
  misc_feature 1..1373: note = I33
  source 1..1373: mol_type = other DNA
atctagtggcaaacgggtgagtaacacgtaaacaacctgccttcaggatggggacaacagacggaaacgactgctaataccgaatacgttccttaggtcgcatgactttaggaagaaagggtggcctctacttgtaagctatcgcctgaagaggggtttgcgtctgattaggtagttggtgaggtaacggcccaccaagccgacgatcagtagccggtctgagaggatgaacggccacactggaactgagacacggtccagactcctacgggaggcagcagtggggaatcttccgcaatgggcgaaagcctgacggagcaacgccgcgtgagtgatgacggccttcgggttgtaaagctctgtgatcggggacgaacggtcagcagacgaatactctgctgaagtgacggtacccgaatagcaagccacggctaactacgtgccagcagccgcggtaatacgtaggtggcaagcgttgtccggaattattgggcgtaaagcgcgcgcaggcggcttcttaagtccatcttaaaagtgcggggcttaaccccgtgatgggatggaaactgagaggctggagtatcggagaggaaagtggaattcctagtgtagcggtgaaatgcgtagagattaggaagaacaccggtggcgaaggcgactttctggacgacaactgacgctgaggcgcgaaagcgtggggagcaaacaggattagataccctggtagtccacgccgtaaacgatgaatactaggtgtaggaggtatcgaccccttctgtgccggagctaacacaataagtattccgcctgggaagtacgatcgcaagattaaaactcaaaggaattgacgggggcccgcacaagcggtggagtatgtggtttaattcgacgcaacgcgaagaaccttaccaggtcttgacattgatcgctattccaagaaattggaagttctccttcgggagacgagaaaacaggtggtgcacggctgtcgtcagctcgtgtcgtgagatgttgggttaagtcccgcaacgagcgcaacccctatcttatgttaccagcacgttatggtggggactcatgagagaccgccgcggacaacgcggaggaaggtggggatgacgtcaagtcatcatgccccttatgacctgggctacacacgtactacaatgggtgtcaacaaagagaagcgaagccgcgaggcagagcaaacctcaaaaacacacccccagttcagattgcaggctgcaacccgcctgcatgaagtaggaatcgctagtaatcgcgggtcagcataccgcggtgaatacgttcccgggccttgtacacaccgcccgtcacactatgagagtcagaaacacccgaagccggtgagg

SEQ ID NO: 201 - DNA, 1366 bp, unidentified
  misc_feature 1..1366: note = I34
  source 1..1366: mol_type = other DNA
ttcttagtggcgaacgggtgagtaacgcgtgggcaacctgccctccagttggggacaacattccgaaagggatgctaataccgaatgtgctccctcctccgcatggaggagggaggaaagatggcctctgcttgcaagctatcgctggaagatgggcccgcgtctgattagctagttggtggggtaacggctcaccaaggcgatgatcagtagccggtctgagaggatgaacggccacattgggactgagacacggcccaaactcctacgggaggcagcagtggggaatcttccgcaatggacgaaagtctgacggagcaacgccgcgtgagtgatgaaggtcttcggattgtaaaactctgttgttagggacgaaagcaccgtgttcgaacaggtcatggtgttgacggtacctaacgaggaagccacggctaactacgtgccagcagccgcggtaatacgtaggtggcaagcgttgtccggaattattgggcgtaaagagcatgtaggcgggcttttaagtctgacgtgaaaatgcggggcttaaccccgtatggcgttggatactggaagtcttgagtgcaggagaggaaaggggaattcccagtgtagcggtgaaatgcgtagatattgggaggaacaccagtggcgaaggcgcctttctggactgtgtctgacgctgagatgcgaaagccagggtagcaaacgggattagataccccggtagtcctggccgtaaacgatggatactaggtgtaggaggtatcgaccccttctgtgccggagttaacgcaataagtatcccgcctggggactacgatcgcaagattgaaactcaaaggaattgacgggggcccgcacaagcggtggagtatgtggtttaattcgacgcaacgcgaagaaccttaccaaggcttgacattgagtgaaagacctagagataggtccctcccttcggggacacgaaaacaggtggtgcatggctgtcgtcagctcgtgtcgtgagatgttgggttaagtcccgcaacgagcgcaacccctatcctatgttaccagcgcgtaatggcggggactcataggagactgccagggataacttggaggaaggcggggatgacgtcaagtcatcatgccccttatgtcttgggctacacacgtactacaatggtcggcaacaaagggcagcgaaaccgcgaggtggagcaaatcccagaaacccgaccccagttcggatcgtaggctgcaacccgcctacgtgaagttggaatcgctagtaatcgcaggtcagcatactgcggtgaatacgttcccgggccttgtacacaccgcccgtcacaccacgaaagttggtaacacccgaagccgg

SEQ ID NO: 202 - DNA, 1387 bp, unidentified
  misc_feature 1..1387: note = I35
  misc_feature 11: note = n is a, c, g, or t
  misc_feature 190: note = n is a, c, g, or t
  misc_feature 399: note = n is a, c, g, or t
  source 1..1387: mol_type = other DNA
cttgctctttnttggattctagtggcaaacgggtgagtaacacgtaaacaacctgccttcaggatggggacaacagacggaaacgactgctaataccgaataccttccaatttccgcatggagataggaagaaagggtggcctctacttgtaagctatcgcctgaagaggggtttgcgtctgattagctngttggtgaggtaacggcccaccaaggcgacgatcagtagccggtctgagaggatgaacggccacactggaactgagacacggtccagactcctacgggaggcagcagtggggaatcttccgcaatggacgaaagtctgacggagcaacgccgcgtgaacgatgaaggtcttcggattgtaaagttctgtgatccgggacgaaggcattnattgagaacattgattgatgttgacggtaccggaaaagcaagccacggctaactacgtgccagcagccgcggtaatacgtaggtggcaagcgttgtccggaattattgggcgtaaagcgcgcgcaggcggccgtgcaagtccatcttaaaagcgtggggcttaaccccatgaggggatggaaactgcatggctggagtgtcggaggggaaagtggaattcctagtgtagcggtgaaatgcgtagagattaggaagaacaccggtggcgaaggcgactttctagacgacaactgacgctgaggcgcgaaagcgtggggagcaaacaggattagataccctggtagtccacgccgtaaacgatggatactaggtgtaggaggtatcgaccccttctgtgccggagttaacgcaataagtatcccgcctgggaagtacgatcgcaagattaaaactcaaaggaattgacgggggcccgcacaagcggtggagtatgtggtttaattcgacgcaacgcgaagaaccttaccaagccttgacattgatcgcaatctgcagaaatgcggagttcctcttcggaggacgagaaaacaggtggtgcacggctgtcgtcagctcgtgtcgtgagatgttgggttaagtcccgcaacgagcgcaacccctatcttctgttgccagcacgtaaaggtgggaactcaggagagaccgccgcggacaacgcggaggaaggcggggatgacgtcaagtcatcatgccccttatggcttgggctacacacgtactacaatgggtgcaaacaaagagaagcgaagtcgcgagacggagcggacctcataaacgcactcccagttcagattgcaggctgcaacccgcctgcatgaagtaggaatcgctagtaatcgcgggtcagcataccgcggtgaatacgttcccgggccttgtacacaccgcccgtcacactatgagagtcagagacacccaaagccggtgg

SEQ ID NO: 203 - DNA, 1386 bp, unidentified
  misc_feature 1..1386: note = I36
  misc_feature 20: note = n is a, c, g, or t
  misc_feature 112..113: note = n is a, c, g, or t
  misc_feature 115..117: note = n is a, c, g, or t
  misc_feature 130: note = n is a, c, g, or t
  misc_feature 189: note = n is a, c, g, or t
  misc_feature 1200..1201: note = n is a, c, g, or t
  source 1..1386: mol_type = other DNA
gagaagcttgcttcttatcnattctagtggcaaacgggtgagtaacgcgtaagcaacctgcccttcagatggggacaacagctggaaacggctgctaataccgaatacgttnntnnngccgcatgacganatgaagaaagggaggccttcgggctttcgctggaggaggggcttgcgtctgattagctngttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatatgggacgaacaggacatcggttaatacccggtgtctttgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagaggganncgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgag

SEQ ID NO: 204 - DNA, 1364 bp, unidentified
  misc_feature 1..1364: note = I37
  misc_feature 939..940: note = n is a, c, g, or t
  source 1..1364: mol_type = other DNA
cggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgtattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacaggattagataccctggtagtccacgccctaaacgatgtctactagttgttggggacgatagtccttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaaggnntgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagac

SEQ ID NO: 205 - DNA, 1343 bp, unidentified
  misc_feature 1..1343: note = I38
  source 1..1343: mol_type = other DNA
ggaaacggattagcggcggacgggtgagtaacacgtgggtaacctgcctcatagaggggaatagcctcccgaaagggagattaataccgcataacattgcagtttcgcatgaaacagcaattaaaggagcaatccgctatgagatggacccgcggcgcattagctagttggtaaggtaatggcttaccaaggcgacgatgcgtagccgacctgagagggtgatcggccacattgggactgagacacggcccagactcctacgggaggcagcagtggggaatattgcacaatgggggaaaccctgatgcagcaacgccgcgtgagtgatgacggtcttcggattgtaaagctctgtctttggggacgataatgacggtacccaaggaggaagccacggctaactacgtgccagcagccgcggtaatacgtaggtggcgagcgttgtccggatttactgggcgtaaagggagcgtaggcggatttttaagtgggatgtgaaatacccgggctcaacctgggtgctgcattccaaactgggaatctagagtgcaggaggggagagtggaattcctagtgtagcggtgaaatgcgtagagattaggaagaacaccagtggcgaaggcgactctctggactgtaactgacgctgaggctcgaaagcgtggggagcgaacaggattagataccctggtagtccacgccgtaaacgatgaatactaggtgtaggggtttcaacacctctgtgccgccgctaacgcattaagtattccgcctggggagtacggtcgcaagattaaaactcaaaggaattgacgggggcccgcacaagtagcggagcatgtggtttaattcgaagcaacgcgaagaaccttacctagacttgacatcctctgcattacccttaatcggggaagttccttcgggaacagagtgacaggtggtgcatggttgtcgtcagctcgtgtcgtgagatgttgggttaagtcccgcaacgagcgcaacccctattgttagttgctaccattaagttgagcactctagcgagactgcctgggttaaccaggaggaaggtggggatgacgtcaaatcatcatgccccttatgtctagggctacacacgtgctacaatggcaagtacagagagatgcaataccgcgaggtggagctaaacttcaaaacttgtctcagttcggattgtaggctgaaactcgcctacatgaagctggagttactagtaatcgcgaatcagcatgtcgcggtgaatacgttcccgggccttgtacacaccgcccgtcacaccatgagagttggcaatacccaaagttcgtgagctaacgcgtaag

SEQ ID NO: 206 - DNA, 1318 bp, unidentified
  misc_feature 1..1318: note = I39
  source 1..1318: mol_type = other DNA
cttagtggcggacgggtgagtaacgcgtgagtaacctgcctttcagaggggaataacattctgaaaagaatgctaataccgcatgagatcgtagtatcgcatggtacagcgaccaaaggagcaatccgctgaaagatggactcgcgtccgattagctagttggtgagataaaggcccaccaaggcgacgatcggtagccggactgagaggttgaacggccacattgggactgagacacggcccagactcctacgggaggcagcagtgggggatattgcacaatgggggaaaccctgatgcagcaacgccgcgtgaaggaagaaggtcttcggattgtaaacttctgtcctcagggaagataatgacggtacctgaggaggaagctccggctaactacgtgccagcagccgcggtaatacgtagggagcaagcgttgtccggatttactgggtgtaaagggtgcgtaggcggatctgcaagtcagtagtgaaatcccagggcttaaccctggaactgctattgaaactgtgggtcttgagtgaggtagaggcaggcggaattcccggtgtagcggtgaaatgcgtagagatcgggaggaacaccagtggcgaaggcggcctgctgggccttaactgacgctgaggcacgaaagcatgggtagcaaacaggattagataccctggtagtccatgccgtaaacgatgattactaggtgtgggtggtctgaccccatccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatcctgctaacgaggtagagatacgttaggtgcccttcggggaaagcagagacaggtggtgcatggttgtcgtcagctcgtgtcgtgagatgttgggttaagtcccgcaacgagcgcaacccctgctattagttgctacgcaagagcactctaataggactgccgttgacaaaacggaggaaggtggggacgacgtcaaatcatcatgccccttatgacctgggctacacacgtactacaatggccgtcaacagagagaagcaaagccgcgaggtggagcaaaactctaaaaacggtcccagttcggatcgtaggctgcaacccgcctacgtgaagttggaattgctagtaatcgcggatcatcatgccgcggtgaatacgttcccgggccttgtacacaccgcccgtcacaccatgggagccggtaatacccgaagtca

SEQ ID NO: 207 - DNA, 1360 bp, unidentified
  misc_feature 1..1360: note = I40
  source 1..1360: mol_type = other DNA
tcatgacagaggattcgtccaatggagtgagttacttagtggcggacgggtgagtaacgcgtgagtaacctgccttggagtggggaataacaggtggaaacatctgctaataccgcatgatgcagttgggtcgcatggctctgactgccaaagatttatcgctctgagatggactcgcgtctgattagctggttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaagcaagtgacggtacctgaggaataagccacggctaactacgtgccagcagccgcggtaatacgtaggtggcgagcgttatccggatttactgggtgtaaagggcgtgtaggcgggactgcaagtcagatgtgaaaaccatgggctcaacctgtggcctgcatttgaaactgtagttcttgagtactggagaggcagacggaattcctagtgtagcggtgaaatgcgtagatattaggaggaacaccagtggcgaaggcggtctgctggacagcaactgacgctgaggcgcgaaagcgtggggagcaaacaggattagataccctggtagtccacgctgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccggtgaccggtgtagagatacaccttcttcttcggaagcgccggtgacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaagaccgcgaggtggagcaaacccctaaaagccatcccagttcggattgcaggctgcaactcgcctgtatgaagttggaatcgctagtaatcgcggatcagcatgccgcggtgaatacgttcccgggccttgtacacaccgcccgtcacaccatgagagtcgggaacacccgaagtccgtagcct

SEQ ID NO: 208 - DNA, 1370 bp, unidentified
  misc_feature 1..1370: note = I41
  source 1..1370: mol_type = other DNA
gtgctcatgacggagttttcggacaacggattgggttacttagtggcggacgggtgagtaacgcgtgaggaacctgcctcggagtggggaataacacaccgaaaggtgtgctaataccgcataatgcagttgggtcgcatgactctgactgccaaagatttatcgctctgagatggcctcgcgtctgattagctagttggcggggtaacggcccaccaaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaaacaaatgacggtacctgacgaataagccacggctaactacgtgccagcagccgcggtaatacgtaggtggcaagcgttatccggatttactgggtgtaaagggcgtgtaggcgggactgcaagtcaggtgtgaaaaccaggggctcaacctctggcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggactgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggccacacacgtactacaatggtggttaacagaggggagcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccg

SEQ ID NO: 209 - DNA, 1341 bp, unidentified
  misc_feature 1..1341: note = I42
  misc_feature 120..121: note = n is a, c, g, or t
  source 1..1341: mol_type = other DNA
ttgcaccttcaagttagtggcggacgggtgagtaacgcgtgagcaacctgcctcaaagagggggataacgtctggaaacggacgctaataccgcatgacgtattcgataggcatctattnnataccaaaggagcaatccgctttgagatgggctcgcgtctgattagctggttggtggggtaaaggcctaccaaggcgacgatcagtagccggactgagaggttgaacggccacattgggactgagacacggcccagactcctacgggaggcagcagtgggggatattgcacaatgggggaaaccctgatgcagcaacgccgcgtgaaggaagacggttttcggattgtaaacttctgttcttagtgacgataatgacggtagctaaggagaaagctccggctaactacgtgccagcagccgcggtaatacgtagggagcgagcgttgtccggaattactgggtgtaaagggagcgtaggcgggagatcaagtcagatgtgaaaactatgggctcaacccataacctgcatttgaaactggttttcttgagtgaagtagaggcaggcggaattccgagtgtagcggtgaaatgcgtagatattcggaggaacaccagtggcgaaggcggcctgctgggcttttactgacgctgaggctcgaaagcatggggagcaaacaggattagataccctggtagtccatgccgtaaacgatgattactaggtgtggggtggctgacccattccgtgccggagttaacacaataagtaatccacctggggagtacggccgcaaggttgaaactcaaaggaattgacgggggcccgcacaagcagtggagtatgtggtttaattcgaagcaacgcgaagaaccttaccaggtcttgacatccgactaacgaagtagagatacattaggtgcccttcggggaaagtcgagacaggtggtgcatggttgtcgtcagctcgtgtcgtgagatgttgggttaagtcccgcaacgagcgcaacccttgtcattagttgctacgcaagagcactctaatgagactgccgttgacaaaacggaggaaggtggggacgacgtcaaatcatcatgccccttatgacctgggctacacacgtactacaatggccgttaacagagggaagcaatactgtgaagtggagcaaacccctaaaaacggtcccagttcagattgcaggctgcaacccgcctgcatgaagtcggaattgctagtaatcgcggatcagcatgccgcggtgaatacgttcccgggccttgtacacaccgcccgtcacaccatgggagccggtaatacccgaagtcggtagtctaacc

SEQ ID NO: 210 - DNA, 1391 bp, unidentified
  misc_feature 1..1391: note = K01
  source 1..1391: mol_type = other DNA
gcaagtcgagcgaagcgctgttttcagaatcttcggaggaagaggacagcgactgagcggcggacgggtgagtaacgcgtgggcaacctgcctcatacagggggataacagttagaaatgactgctaataccgcataagcgcacgggaccgcatggtctagtgtgaaaaactccggtggtatgagatggacccgcgtctgattaggtagttggtggggtaaaggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgagtaagaagcaccggctaaatacgtgccagcagccgcggtaatacgtatggtgcaagcgttatccggatttactgggtgtaaagggagcgtagacggataggcaagtctggagtgaaaacccagggctcaaccctgggactgctttggaaactgcagatctggagtgccggagaggtaagcggaattcctagtgtagcggtgaaatgcgtagatattaggaggaacaccagtggcgaaggcggcttactggacggtgactgacgttgaggctcgaaagcgtggggagcaaacaggattagataccctggtagtccacgccgtaaacgatgactactaggtgtcggtgtgcaaagcacatcggtgccgcagcaaacgcaataagtagtccacctggggagtacgttcgcaagaatgaaactcaaaggaattgacggggacccgcacaagcggtggagcatgtggtttaattcgaagcaacgcgaagaaccttacctggtcttgacatccggatgacgggcgagtaatgtcgccgtcccttcggggcatccgagacaggtggtgcatggttgtcgtcagctcgtgtcgtgagatgttgggttaagtcccgcaacgagcgcaacccttatcttcagtagccagcatataaggtgggcactctggagagactgccagggagaacctggaggaaggtggggatgacgtcaaatcatcatgccccttatggccagggctacacacgtgctacaatggcgtaaacaaagggaagcgagagggtgacctgaagcgaatcccaaaaataacgtctcagttcggattgtagtctgcaactcgactacatgaagctggaatcgctagtaatcgcggatcagcatgccgcggtgaatacgttcccgggtcttgtacacaccgcccgtcacaccatgggagtcagtaacgcccgaagccagtgacccaaccttgaggagg

SEQ ID NO: 211 - DNA, 1383 bp, unidentified
  misc_feature 1..1383: note = K02
  source 1..1383: mol_type = other DNA
tcgaggggcagcattttagtttgcttgcaaactgaagatggcgaccggcgcacgggtgagtaacacgtatccaacctgccgataactccggaatagcctttcgaaagaaagattaataccggatagcatacgaatatcgcatgatatttttattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatgggcgagagcctgaaccagccaagtagcgtgaaggatgaaggctctatgggtcgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgttactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaacagaatatattggaaacagtatagccgtaaggctgttgtgaaggtgctgcatggttgtcgtcagctcgtgccgtgaggtgtcggcttaagtgccataacgagcgcaacccttatctttagttactaacaggttatgctgaggactctagagagactgccgtcgtaagatgtgaggaaggtggggatgacgtcaaatcagcacggcccttacgtccggggctacacacgtgttacaatggggggtacagaaggcggctacctggtgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaa

SEQ ID NO: 212 - DNA, 1396 bp, unidentified
  misc_feature 1..1396: note = K03
  source 1..1396: mol_type = other DNA
tgcagtcgagcgaagcgctaagacaggatttcttcggattgaagtctttgtgactgagcggcggacgggtgagtaacgcgtgggtaacctgcctcatacagggggataacagttagaaatgactgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgttgttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccgtcccgtaacgggggcttcccttcggggcagaggagacaggtggtgcatggttgtcgtcagctcgtgtcgtgagatgttgggttaagtcccgcaacgagcgcaacccttatccttagtagccagcacatgatggtgggcactctagggagactgccggggataacccggaggaaggcggggacgacgtcaaatcatcatgccccttatgatttgggctacacacgtgctacaatggcgtaaacaaagggaagcgagacagcgatgttgagcgaatcccaaaaataacgtcccagttcggactgcagtctgcaactcgactgcacgaagctggaatcgctagtaatcgcggatcagaatgccgcggtgaatacgttcccgggtcttgtacacaccgcccgtcacaccatgggagtcagtaacgcccgaagtcagtgacctaaccgaaaggaaggag

SEQ ID NO: 213 - DNA, 1395 bp, unidentified
  misc_feature 1..1395: note = K04
  source 1..1395: mol_type = other DNA
cagtcgagcgaagcgtcttagaatgatctcttcggattgagtcttatatgactgagcggcggacgggtgagtaacgcgtgggtaacctgcctcatacagggggataacagttagaaatgactgctaataccgcataagcgcacagggctgcatggcctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccttctgacgtgcccgtaacggggcattcccttcggggcagaggagacaggtggtgcatggttgtcgtcagctcgtgtcgtgagatgttgggttaagtcccgcaacgagcgcaacccctatccttagtagccagcacatcatggtgggcactctagggagactgccggggataacccggaggaaggcggggacgacgtcaaatcatcatgccccttatgatttgggctacacacgtgctacaatggcgtaaacaaagggaagcgagacagcgatgtttagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaacctcacggagggagc

SEQ ID NO: 214 - DNA, 1411 bp, unidentified
  misc_feature 1..1411: note = K05
  source 1..1411: mol_type = other DNA
gcagtcgaacgtgaaagtccttcgggacgagtaaagtggcgcacgggtgagtaacgcgtggataatctacccttaagatggggataacggctggaaacggtcgctaataccgaatacgctcccgattttatcattggggggaaagatggcctctgcttgcaagctatcgcttaaggatgagtccgcgtcccattagctagttggcggggtaacggcccaccaaggcgacgatgggtagccggtctgagaggatgaccggccacactggaactggaacacggtccagactcctacgggaggcagcagtggggaatattgcgcaatgggcgaaagcctgacgcagcgacgccgcgtgagggatgaaggttctcggatcgtaaacctctgtcaggggggaagaaaccccctcgtgtgaataatgcgagggcttgacggtacccccaaaggaagcaccggctaactccgtgccagcagccgcggtaatacggagggtgcaagcgttaatcggaatcactgggcgtaaagcgcacgtaggcggcttggtaagtcaggggtgaaatcccacagcccaactgtggaactgcctttgatactgccaggcttgagtaccggagagggtggcggaattccaggtgtaggagtgaaatccgtagatatctggaggaacaccggtggcgaaggcggccacctggacggtaactgacgctgaggtgcgaaagcgtgggtagcaaacaggattagataccctggtagtccacgctgtaaacgatgggtgctgggtgctgggatgtatgtctcggtgccgtagctaacgcgataagcaccccgcctggggagtacggtcgcaaggctgaaactcaaagaaattgacgggggcccgcacaagcggtggagtatgtggtttaattcgatgcaacgcgaagaaccttacccaggcttgacatctagggaacccttcggaaatgaaggggtgcccttcggggagccctaagacaggtgctgcatggctgtcgtcagctcgtgccgtgaggtgttgggttaagtcccgcaacgagcgcaacccctatcttcagttgccagcaggtaaggctgggcactctggagagaccgccccggtcaacggggaggaaggtggggacgacgtcaagtcatcatggcccttacgcctggggctacacacgtactacaatggcgcgcacaaagggtagcgagaccgcgaggtggagccaatcccaaaaaacgcgtcccagtccggattggagtctgcaactcgactccatgaagtcggaatcgctagtaattcgagatcagcatgctcgggtgaatgcgttcccgggccttgtacacaccgcccgtcacaccacgaaagtcggttttacccgaagccggtgagctaactcgcaagagaagc

SEQ ID NO: 215 - DNA, 1383 bp, unidentified
  misc_feature 1..1383: note = K06
  source 1..1383: mol_type = other DNA
gaggggcatcgggattgaagcttgcttcaattgccggcgaccggcgcacgggtgcgtaacgcgtatgtaacctacctataacaggggcataacactgagaaattggtactaattccccataatattcggagaggcatctctccgggttgaaaactccggtggttatagatggacatgcgttgtattagctagttggtgaggtaacggctcaccaaggcaacgatacatagggggactgagaggttaaccccccacactggtactgagacacggaccagactcctacgggaggcagcagtgaggaatattggtcaatggacgcaagtctgaaccagccatgccgcgtgcaggaagacggctctatgagttgtaaactgcttttgtacgagggtaaactcacctacgtgtaggtgactgaaagtatcgtacgaataaggatcggctaactccgtgccagcagccgcggtaatacggaggattcaagcgttatccggatttattgggtttaaagggtgcgtaggcggtttgataagttagaggtgaaatcccggggcttaactccggaactgcctctaatactgttagactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgttgtcggcgatacacagtcggtgactaagcgaaagcgataagttatccacctgggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggtcgctaccccgtgaggggatgccaatctcgaaagcctatctcagttcggattggaggctgaaacccgcctccatgaagttggattcgctagtaatcgcgcatcagccatggcgcggtgaatacgttcccgggccttgtacacaccgcccgtcaagccatggaagctgggggtgcctgaagttcgtgaccgcaagg

SEQ ID NO: 216 - DNA, 1393 bp, unidentified
  misc_feature 1..1393: note = K07
  source 1..1393: mol_type = other DNA
cagtcgaacggagttatgcagaggaagttttcggatggaatcggcgtaacttagtggcggacgggtgagtaacgcgtgggaaacctgccctgtaccgggggataacacttagaaataggtgctaataccgcataagcgcacagcttcacatgaggcagtgtgaaaaactccggtggtacaggatggtcccgcgtctgattagccagttggcagggtaacggcctaccaaagcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatgacaagccagatgtgaaaacccagggctcaaccctgggactgcatttggaactgccaggctggagtgcaggagaggtaagcggaattcctagtgtagcggtgaaatgcgtagatattaggaggaacaccagtggcgaaggcggcttactggactgtaactgacgttgaggctcgaaagcgtggggagcaaacaggattagataccctggtagtccacgcggtaaacgatgattgctaggtgtaggtgggtatggacccatcggtgccgcagctaacgcaataagcaatccacctgggggagtacgttcgcaagaatgaaactcaaaggaattgacggggacccgcacaagcggtggagcatgtggtttaattcgaagcaacgcgaagaaccttaccaagtcttgacatcccaatgacgtgtccgtaacggggcattctcttcggagcattggagacaggtggtgcatggttgtcgtcagctcgtgtcgtgagatgttgggttaagtcccgcaacgagcgcaacccttatccttagtagccagcaggtagagctgggcactctagggagactgccggggataacccggaggaaggcggggatgacgtcaaatcatcatgccccttatgatttgggctacacacgtgctacaatggcgtaaacaaagggaagcgagacagtgatgttgagcaaatcccagaaataacgtctcagttcggattgtagtctgcaactcgactacatgaagctggaatcgctagtaatcgcgaatcagcatgtcgcggtgaatacgttcccgggtcttgtacacaccgcccgtcacaccatgggagttggaaatgcccgaagcctgtgacctaaccgcaagggagga

SEQ ID NO: 217 - DNA, 1402 bp, unidentified
  misc_feature 1..1402: note = K08
  misc_feature 947..949: note = n is a, c, g, or t
  misc_feature 953..955: note = n is a, c, g, or t
  misc_feature 967..970: note = n is a, c, g, or t
  source 1..1402: mol_type = other DNA
caagtcgaacgaagtttcgaggaagcttgcttccaaagagacttagtggcgaacgggtgagtaacacgtaggtaacctgcccatgtgtccgggataactgctggaaacggtagctaaaaccggataggtatacagagcgcatgctcagtatattaaagcgcccatcaaggcgtgaacatggatggacctgcggcgcattagctagttggtgaggtaacggcccaccaaggcgatgatgcgtagccggcctgagagggtaaacggccacattgggactgagacacggcccaaactcctacgggaggcagcagtagggaattttcgtcaatgggggaaaccctgaacgagcaatgccgcgtgagtgaagaaggtcttcggatcgtaaagctctgttgtaagtgaagaacggctcatagaggaaatgctatgggagtgacggtagcttaccagaaagccacggctaactacgtgccagcagccgcggtaatacgtaggtggcaagcgttatccggaatcattgggcgtaaagggtgcgtaggtggcgtactaagtctgtagtaaaaggcaatggctcaaccattgtaagctatggaaactggtatgctggagtgcagaagagggcgatggaattccatgtgtagcggtaaaatgcgtagatatatggaggaacaccagtggcgaaggcggtcgcctggtctgtaactgacactgaggcacgaaagcgtggggagcaaataggattagataccctagtagtccacgccgtaaacgatgagaactaagtgttggaggaattcagtgctgcagttaacgcaataagttctccgcctggggagtatgcacgcaagtgtgaaactcaaaggaattgacgggggcccgcacaagcggtggagtatgtggtttaattcgaagcaacgcgaagaaccttaccaggccttgacatggannnaaannncctagagatagnnnnataattatggatcacacaggtggtgcatggttgtcgtcagctcgtgtcgtgagatgttgggttaagtcccgcaacgagcgcaacccttgtcgcatgttaccagcatcaagttggggactcatgcgagactgccggtgacaaaccggaggaaggtggggatgacgtcaaatcatcatgccccttatggcctgggctacacacgtactacaatggcgaccacaaagagcagcgacacagtgatgtgaagcgaatctcataaaggtcgtctcagttcggattgaagtctgcaactcgacttcatgaagtcggaatcgctagtaatcgcagatcagcatgctgcggtgaatacgttctcgggccttgtacacaccgcccgtcaaaccatgggagtcagtaatacccgaagccggtggcataaccgtaaggagtgag

SEQ ID NO: 218 - DNA, 1386 bp, unidentified
  misc_feature 1..1386: note = K09
  source 1..1386: mol_type = other DNA
agtcgaggggcatcaggaagaaagcttgctttctttgctggcgaccggcgcacgggtgagtaacacgtatccaacctgccctttactcggggatagcctttcgaaagaaagattaatacccgatggcataatgattccgcatggtttcattattaaaggattccggtaaaggatggggatgcgttccattaggttgttggtgaggtaacggctcaccaagccttcgatggataggggttctgagaggaaggtcccccacattggaactgagacacggtccaaactcctacgggaggcagcagtgaggaatattggtcaatgggcgctagcctgaaccagccaagtagcgtgaaggatgaaggctctatgggtcgtaaacttcttttatataagaataaagtgcagtatgtatactgttttgtatgtattatatgaataaggatcggctaactccgtgccagcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcagtggaatgatgtggaaacatgtcagtgagcaatcaccgctgtgaaggtgctgcatggttgtcgtcagctcgtgccgtgaggtgtcggcttaagtgccataacgagcgcaacccttatcttcagttactaacaggtcatgctgaggactctggagagactgccgtcgtaagatgtgaggaaggtggggatgacgtcaaatcagcacggcccttacgtccggggctacacacgtgttacaatggggggtacagaaggcagctagcgggtgaccgtatgctaatcccaaaatcctctctcagttcggatcgaagtctgcaacccgacttcgtgaagctggattcgctagtaatcgcgcatcagccacggcgcggtgaatacgttcccgggccttgtacacaccgcccgtcaagccatgggagccgggggtacctgaagtacgtaaccgcaag

SEQ ID NO: 219 - DNA, 1366 bp, unidentified
  misc_feature 1..1366: note = K10
  source 1..1366: mol_type = other DNA
agtcgaacgatgaaaccgccctcgggcggacatgaagtggcgaacgggtgagtaacacgtgaccaacctgccccttgctccgggacaaccttgggaaaccgaggctaataccggatactcctcgcccccctcctggggggcccgggaaagcccagacggcaagggatggggtcgcggcccattaggtagtaggcggggtaacggcccacctagcccgcgatgggtagccgggttgagagaccgaccggccacattgggactgagatacggcccagactcctacgggaggcagcagtggggaattttgcgcaatgggggaaaccctgacgcagcaacgccgcgtgcgggacgacggccttcgggttgtaaaccgctttcagcagggaagaaattcgacggtacctgcagaagaagctccggctaactacgtgccagcagccgcggtaatacgtagggagcgagcgttatccggattcattgggcgtaaagagcgcgtaggcggcctctcaagcgggatctctaatccgagggctcaacccccggccggatcccgaactgggaggctcgagttcggtagaggcaggcggaattcccggtgtagcggtggaatgcgcagatatcgggaagaacaccgatggcgaaggcagcctgctgggccgcaactgacgctgaggcgcgaaagctaggggagcgaacaggattagataccctggtagtcctagccgtaaacgatggatactaggtgtggggggctccgccctccgtgccgcagccaacgcattaagtatcccgcctggggagtacggccgcaaggctaaaactcaaaggaattgacgggggcccgcacaagcagcggagcatgtggcttaattcgaagcaacgcgaagaaccttaccagggcttgacatggacgtgaagccggggaaacccggtggccgagaggagcgtccgcaggtggtgcatggctgtcgtcagctcgtgtcgtgagatgttgggttaagtcccgcaacgagcgcaacccctgccccatgttgccagcattaggttggggactcatgggggactgccggcgtcaagccggaggaaggtggggacgacgtcaagtcatcatgccctttatgccctgggctgcacacgtgctacaatggccggtacaacgggctgcgagaccgcgaggtcgagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcg

SEQ ID NO: 220 - DNA, 1395 bp, unidentified
  misc_feature 1..1395: note = K11
  source 1..1395: mol_type = other DNA
cagtcgaacgaagtgaagatagcttgctattggaacttagtggcgaacgggtgagtaacacgtagataacctgcctgtatgaccgggataacagttggaaacgactgctaataccggataggcagagaggaggcatctcttctctgttaaagttgggatacaacgcaaacagatggatctgcggtgcattagctagttggtgaggtaacggcccaccaaggcgatgatgcatagccggcctgagagggcgaacggccacattgggactgagacacggcccaaactcctacgggaggcagcagtagggaattttcggcaatgggggaaaccctgaccgagcaatgccgcgtgagtgaagacggccttcgggttgtaaagctctgttgtaagggaagaacggcatagagagggaatgctctatgagtgacggtaccttaccagaaagccacggctaactacgtgccagcagccgcggtaatacgtaggtggcaagcgttatccggaattattgggcgtaaagggtgcgtaggcggcgagataagtctgaggtaaaagcccgtggctcaaccacggtaagccttggaaactgtctggctggagtgcaggagaggacaatggaattccatgtgtagcggtaaaatgcgtagatatatggaggaacaccagtggcgaaggcggttgtctggcctgtaactgacgctgaagcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggccttgacatggtatcaaaggccctagagatagggagatagttatgatacacacaggtggtgcatggttgtcgtcagctcgtgtcgtgagatgttgggttaagtcccgcaacgagcgcaacccttgtttctagttaccaacagtaagatggggactctagagagactgccggtgacaaaccggaggaaggtggggatgacgtcaaatcatcatgccccttatggcctgggctacacacgtactacaatggcgtctacaaagagcagcgagcaggtgactgtaagcgaatctcataaaggacgtctcagttcggattgaagtctgcaactcgacttcatgaagtcggaatcgctagtaatcgcggatcagcatgccgcggtgaatacgttctcgggccttgtacacaccgcccgtcaaaccatgggagttgataatacccgaagccggtggcctaaccatttatggagggag

SEQ ID NO: 221 - DNA, 1383 bp, unidentified
  misc_feature 1..1383: note = K12
  source 1..1383: mol_type = other DNA
cagtcgaacgcgagcacttgtgctcgagtggcgaacgggtgagtaatacataagtaacctgccctagacagggggataactattggaaacgatagctaagaccgcataggtacggacactgcatggtgaccgtattaaaagtgcctcaaagcactggtagaggatggacttatggcgcattagctggttggcggggtaacggcccaccaaggcgacgatgcgtagccgacctgagagggtgaccggccacactgggactgagacacggcccagactcctacgggaggcagcagtagggaattttcggcaatgggggaaaccctgaccgagcaacgccgcgtgaaggaagaaggttttcggattgtaaacttctgttataaaggaagaacggcggctacaggaaatggtagccgagtgacggtactttattagaaagccacggctaactacgtgccagcagccgcggtaatacgtaggtggcaagcgttatccggaattattgggcgtaaagagggagcaggcggcagcaagggtctgtggtgaaagcctgaagcttaacttcagtaagccatagaaaccaggcagctagagtgcaggagaggatcgtggaattccatgtgtagcggtgaaatgcgtagatatatggaggaacaccagtggcgaaggcgacgatctggcctgcaactgacgctcagtcccgaaagcgtggggagcaaataggattagataccctagtagtccacgccgtaaacgatgagtactaagtgttggatgtcaaagttcagtgctgcagttaacgcaataagtactccgcctgagtagtacgttcgcaagaatgaaactcaaaggaattgacgggggcccgcacaagcggtggagcatgtggtttaattcgaagcaacgcgaagaaccttaccaggtcttgacatactcataaaggctccagagatggagagatagctatatgagatacaggtggtgcatggttgtcgtcagctcgtgtcgtgagatgttgggttaagtcccgcaacgagcgcaacccttatcgttagttaccatcattaagttggggactctagcgagactgccagtgacaagctggaggaaggcggggatgacgtcaaatcatcatgccccttatgacctgggctacacacgtgctacaatggatggtgcagagggaagcgaagccgcgaggtgaagcaaaacccataaaaccattctcagttcggattgtagtctgcaactcgactacatgaagttggaatcgctagtaatcgcgaatcagcatgtcgcggtgaatacgttctcgggccttgtacacaccgcccgtcacaccacgagagttgataacacccgaagccggtggcctaaccgcaagga

SEQ ID NO: 222 - DNA, 1423 bp, unidentified
  misc_feature 1..1423: note = K13
  misc_feature 4: note = n is a, c, g, or t
  source 1..1423: mol_type = other DNA
tgcnagtcgaacgcttctttcctcccgagtgcttgcactcaattggaaagaggagtggcggacgggtgagtaacacgtgggtaacctacccatcagagggggataacacttggaaacaggtgctaataccgcataacagtttatgccgcatggcataagagtgaaaggcgctttcgggtgtcgctgatggatggacccgcggtgcattagctagttggtgaggtaacggctcaccaaggccacgatgcatagccgacctgagagggtgatcggccacactgggactgagacacggcccagactcctacgggaggcagcagtagggaatcttcggcaatggacgaaagtctgaccgagcaacgccgcgtgagtgaagaaggttttcggatcgtaaaactctgttgttagagaagaacaaggacgttagtaactgaacgtcccctgacggtatctaaccagaaagccacggctaactacgtgccagcagccgcggtaatacgtaggtggcaagcgttgtccggatttattgggcgtaaagcgagcgcaggcggtttcttaagtctgatgtgaaagcccccggctcaaccggggagggtcattggaaactgggagacttgagtgcagaagaggagagtggaattccatgtgtagcggtgaaatgcgtagatatatggaggaacaccagtggcgaaggcggctctctggtctgtaactgacgctgaggctcgaaagcgtggggagcaaacaggattagataccctggtagtccacgccgtaaacgatgagtgctaagtgttggagggtttccgcccttcagtgctgcagcaaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcctttgaccactctagagatagagctttcccttcggggacaaagtgacaggtggtgcatggttgtcgtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgggaagtacaacgagtcgctagaccgcgaggtcatgcaaatctcttaaagcttctctcagttcggattgcaggctgcaactcgcctgcatgaagccggaatcgctagtaatcgcggatcagcacgccgcggtgaatacgttcccgggccttgtacacaccgcccgtcacaccacgagagtttgtaacacccgaagtcggtgaggtaacctttttggagcc

SEQ ID NO: 223 - DNA, 1381 bp, unidentified
  misc_feature 1..1381: note = K14
  source 1..1381: mol_type = other DNA
aagtcgaggggcatcatgacctagcaataggttgatggcgaccggcgcacgggtgagtaacacgtatccaacctgccgattattccgggatagcctttcgaaagaaagattaatactggatagcataacgagaaggcatcttcttgttattaaagaatttcgataatcgatggggatgcgttccattagtttgttggcggggtaacggcccaccaagacatcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtgcagtatgtatactgttttgtatgtaccatacgaataaggatcggctaactccgtgccagcagccgcggtaatacggaggatccgagcgttatccggatttattgggtttaaagggagcgtaggcggattattaagtcagttgtgaaagtttgcggctcaaccgtaaaattgcagttgatactggtagtcttgagtgcagcagaggtaggcggaattcgtggtgtagcggtgaaatgcttagatatcacgaagaactccgattgcgaaggcagcttactggactgtaactgacgctgatgctcgaaagtgtgggtatcaaacaggattagataccctggtagtccacacagtaaacgatgaatactcgctgtttgcgatatacagcaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgacggatttggaaacagatcttccttcgggcagttgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacacagcgatgtgatgctaatcccaaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcaag

SEQ ID NO: 224 - DNA, 1391 bp, unidentified
  misc_feature 1..1391: note = K15
  source 1..1391: mol_type = other DNA
cagtcgaacgaagcgatttaacggaagttttcggatggaagttggattgactgagtggcggacgggtgagtaacgcgtgggtaacctgccttgtactgggggacaacagttagaaatgactgctaataccgcataagcgcacagtatcgcatgatacagtgtgaaaaactccggtggtacaagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggtaaagcaagtctgaagtgaaagcccgcggctcaactgcgggactgctttggaaactgtttaactggagtgtcggagaggtaagtggaattcctagtgtagcggtgaaatgcgtagatattaggaggaacaccagtggcgaaggcgacttactggacgataactgacgttgaggctcgaaagcgtggggagcaaacaggattagataccctggtagtccacgccgtaaacgatgaatactaggtgttggggagcaaagctcttcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaggtcttgacatcgatccgacgggggagtaacgtccccttcccttcggggcggagaagacaggtggtgcatggttgtcgtcagctcgtgtcgtgagatgttgggttaagtcccgcaacgagcgcaacccttattctaagtagccagcggttcggccgggaactcttgggagactgccagggataacctggaggaaggtggggatgacgtcaaatcatcatgccccttatgatctgggctacacacgtgctacaatggcgtaaacaaagagaagcaagaccgcgaggtggagcaaatctcaaaaataacgtctcagttcggactgcaggctgcaactcgcctgcacgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggaggga

SEQ ID NO: 225 - DNA, 1402 bp, unidentified
  misc_feature 1..1402: note = K16
  source 1..1402: mol_type = other DNA
agtcgaacgaagcaatactgtgtgaagagattagcttgctaagatcagaactttgtattgactgagtggcggacgggtgagtaacgcgtgggcaacctgccttacacagggggataacagctagaaatggctgctaataccgcataagacctcagtaccgcatggtagaggggtaaaaactccggtggtgtaagatgggcccgcgtctgattaggtagttggtagggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaagatgacggtacctgactaagaagccccggctaactacgtgccagcagccgcggtaatacgtagggggcaagcgttatccggatttactgggtgtaaagggagcgtagacggcatggcaagtctgaagtgaaagcccggggctcaaccccgggactgctttggaaactgtcaggctagagtgtcggagaggcaagtggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgattactaggtgtcgggaagcaaagcttttcggtgccgcagccaacgcaataagtaatccacctggggagtacgttcgcaagaatgaaactcaaaggaattgacggggacccgcacaagcggtggagcatgtggtttaattcgaagcaacgcgaagaaccttacctgatcttgacatcccggtgacaaagtatgtaatgtactctttcttcggaacaccggtgacaggtggtgcatggttgtcgtcagctcgtgtcgtgagatgttgggttaagtcccgcaacgagcgcaacccttatctttagtagccagcatttgaggtgggcactctagagagactgccagggataacctggaggaaggtggggatgacgtcaaatcatcatgccccttatgaccagggctacacacgtgctacaatggcgtaaacaaagggaagcgaccctgtgaaggcaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagccggtgacctaaccgaaaggaagga

SEQ ID NO: 226 - DNA, 1393 bp, unidentified
  misc_feature 1..1393: note = K17
  misc_feature 942..945: note = n is a, c, g, or t
  misc_feature 953..956: note = n is a, c, g, or t
  misc_feature 1067..1069: note = n is a, c, g, or t
  source 1..1393: mol_type = other DNA
gcaagtcgagcgaagcggtttcgatgaagttttcggatggatttgaaattgacttagcggcggacgggtgagtaacgcgtgggtaacctgccttacactgggggataacagttagaaatgactgctaataccgcataagcgcacagggccgcatggtctggtgtgaaaaactccggtggtgtaagatggacccgcgtctgattaggtagttggtggggtaacggcccaccaagccgacgatcagtagccgacctgagagggtgaccggccacattgggactgagacacggcccaaactcctacgggaggcagcagtggggaatattggacaatgggcgaaagcctgatccagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggttaagcaagtctgaagtgaaagcccggggctcaaccccggtactgctttggaaactgtttgacttgagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatactaggtgtcgggggacaaagtccttcggtgccgccgctaacgcaataagtattccacctggggagtacgttcgcaagaatgaaactcaaaggaattgacggggacccgcacaagcggtggagcatgtggtttaattcgaagcaacgcgaagaaccttaccaagtcttgacatcccattgaaaannnnttaaccgnnnnccctcttcggagcaatggagacaggtggtgcatggttgtcgtcagctcgtgtcgtgagatgttgggttaagtcccgcaacgagcgcaacccttatccttagtagccagcannnaatggtgggcactctggggagactgccagggataacctggaggaaggtggggatgacgtcaaatcatcatgccccttatgatttgggctacacacgtgctacaatggcgtaaacaaagggaagcaaaggagcgatctggagcaaaccccaaaaataacgtctcagttcggattgcaggctgcaactcgcctgcatgaagctggaatcgctagtaatcgcgaatcagaatgtcgcggtgaatacgttcccgggtcttgtacacaccgcccgtcacaccatgggagttggtaacgcccgaagtcagtgacccaaccgcaaggaggg

SEQ ID NO: 227 - DNA, 1389 bp, unidentified
  misc_feature 1..1389: note = K18
  source 1..1389: mol_type = other DNA
gtcgaggggcagcatggtcttagcttgctaaggctgatggcgaccggcgcacgggtgagtaacacgtatccaacctgccgtctactcttggccagccttctgaaaggaagattaatccaggatggcatcatgagttcacatgtccgcatgattaaaggtattttccggtagacgatggggatgcgttccattagatagtaggcggggtaacggcccacctagtcaacgatggataggggttctgagaggaaggtcccccacattggaactgagacacggtccaaactcctacgggaggcagcagtgaggaatattggtcaatgggcgatggcctgaaccagccaagtagcgtgaaggatgactgccctatgggttgtaaacttcttttataaaggaataaagtcgggtatgcatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatgtcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatacggcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcactcgaatgatccggaaacggttcagctagcaatagcgagtgtgaaggtgctgcatggttgtcgtcagctcgtgccgtgaggtgtcggcttaagtgccataacgagcgcaacccttgttgtcagttactaacaggtgatgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccctaaaacccctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgagg

SEQ ID NO: 228 - DNA, 1393 bp, unidentified
  misc_feature 1..1393: note = K19
  misc_feature 1205..1206: note = n is a, c, g, or t
  source 1..1393: mol_type = other DNA
gcagtcgaacgaagcaattaagatgaagttttcggatggaatcttgattgactgagtggcggacgggtgagtaacgcgtggataacctgcctcacactgggggataacagttagaaatgactgctaataccgcataagcgcacagtgccgcatggcagtgtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgccgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatccccctgacgggccggtaacgcggcctttccttcgggacaggggagacaggtggtgcatggttgtcgtcagctcgtgtcgtgagatgttgggttaagtcccgcaacgagcgcaacccttatccttagtagccagcacgtgaaggtgggcactctagggagactgccagggataacctggaggaaggtggggatgacgtcaaatcatcatgccccttatgatttgggctacacacgtgctacaatggcgtaaacaaagggaagcgagacagtgatgnngagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaaccgaaaggaggga

SEQ ID NO: 229 - DNA, 1394 bp, unidentified
  misc_feature 1..1394: note = K20
  source 1..1394: mol_type = other DNA
tgcaagtcgaacggggtgctcatgacggaggattcgtccaacggattgagttacctagtggcggacgggtgagtaacgcgtgaggaacctgccttggagaggggaataacactccgaaaggagtgctaataccgcatgatgcagttgggtcgcatggctctgactgccaaagatttatcgctctgagatggcctcgcgtctgattagctagtaggcggggtaacggcccacctaggcgacgatcagtagccggactgagaggttgaccggccacattgggactgagacacggcccagactcctacgggaggcagcagtggggaatattgggcaatgggcgcaagcctgacccagcaacgccgcgtgaaggaagaaggctttcgggttgtaaacttcttttgtcggggacgaaacaaatgacggtacccgacgaataagccacggctaactacgtgccagcagccgcggtaatacgtaggtggcaagcgttatccggatttactgggtgtaaagggcgtgtaggcgggattgcaagtcagatgtgaaaactgggggctcaacctccagcctgcatttgaaactgtagttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaggtgtggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcccactaacgaagcagagatgcattaggtgcccttcggggaaagtggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggttaacagagggaggcaataccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagcctaaccgcaaggaggg

SEQ ID NO: 230 - DNA, 1385 bp, unidentified
  misc_feature 1..1385: note = K21
  source 1..1385: mol_type = other DNA
agtcgaggggcagcattttagtttgcttgcaaactaaagatggcgaccggcgcacgggtgagtaacacgtatccaacctgccgataactcggggatagcctttcgaaagaaagattaatatccgatggtatattaaaaccgcatggttttactattaaagaatttcggttatcgatggggatgcgttccattagtttgttggcggggtaacggcccaccaagactacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatatgggaataaagtattccacgtgtgggattttgtatgtaccatatgaataaggatcggctaactccgtgccagcagccgcggtaatacggaggatccgagcgttatccggatttattgggtttaaagggagcgtaggtggattgttaagtcagttgtgaaagtttgcggctcaaccgtaaaattgcagttgaaactggcagtcttgagtacagtagaggtgggcggaattcgtggtgtagcggtgaaatgcttagatatcacgaagaactccgattgcgaaggcagctcactagactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaataatctggaaacaggttagccgcaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggcgacaggatgctaatcccaaaaacctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaa

SEQ ID NO: 231 - DNA, 1385 bp, unidentified
  misc_feature 1..1385: note = K22
  source 1..1385: mol_type = other DNA
agtcgaggggcagcatttcagtttgcttgcaaactggagatggcgaccggcgcacgggtgagtaacacgtatccaacctgccgataactcggggatagcctttcgaaagaaagattaatacccgatggtataattagaccgcatggtcttgttattaaagaatttcggttatcgatggggatgcgttccattaggcagttggtgaggtaacggctcaccaaaccttcgatggataggggttctgagaggaaggtcccccacattggaactgagacacggtccaaactcctacgggaggcagcagtgaggaatattggtcaatgggcgcaggcctgaaccagccaagtagcgtgaaggatgactgccctatgggttgtaaacttcttttatatgggaataaagttttccacgtgtggaattttgtatgtaccatatgaataaggatcggctaactccgtgccagcagccgcggtaatacggaggatccgagcgttatccggatttattgggtttaaagggagcgtaggtggacagttaagtcagttgtgaaagtttgcggctcaaccgtaaaattgcagttgatactggctgtcttgagtacagtagaggtgggcggaattcgtggtgtagcggtgaaatgcttagatatcacgaagaactccgattgcgaaggcagctcactggactgcaactgacactgatgctcgaaagtgtgggtatcaaacaggattagataccctggtagtccacacagtaaacgatgaatactcgctgtttgcgatatacagtaagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcatttgaatatattggaaacagtatagccgtaaggcaaatgtgaaggtgctgcatggttgtcgtcagctcgtgccgtgaggtgtcggcttaagtgccataacgagcgcaacccttatctttagttactaacaggtcatgctgaggactctagagagactgccgtcgtaagatgtgaggaaggtggggatgacgtcaaatcagcacggcccttacgtccggggctacacacgtgttacaatggggggtacagaaggcagctacctggtgacaggatgctaatcccaaaagcctctctcagttcggatcgaagtctgcaacccgacttcgtgaagctggattcgctagtaatcgcgcatcagccatggcgcggtgaatacgttcccgggccttgtacacaccgcccgtcaagccatgaaagccgggggtacctgaagtacgtaaccgcaa

SEQ ID NO: 232 - DNA, 1387 bp, unidentified
  misc_feature 1..1387: note = K23
  source 1..1387: mol_type = other DNA
agtcgaggggcagcatgatttgtagcaatacagattgatggcgaccggcgcacgggtgagtaacgcgtatgcaacttacctatcagagggggatagcccggcgaaagtcggattaataccccataaaacaggggtcccgcatgggaatatttgttaaagattcatcgctgatagataggcatgcgttccattaggcagttggcggggtaacggcccaccaaaccgacgatggataggggttctgagaggaaggtcccccacattggtactgagacacggaccaaactcctacgggaggcagcagtgaggaatattggtcaatggccgagaggctgaaccagccaagtcgcgtgaaggaagaaggatctatggtttgtaaacttcttttataggggaataaagtggaggacgtgtccttttttgtatgtaccctatgaataagcatcggctaactccgtgccagcagccgcggtaatacggaggatgcgagcgttatccggatttattgggtttaaagggtgcgtaggtggtgatttaagtcagcggtgaaagtttgtggctcaaccataaaattgccgttgaaactgggttacttgagtgtgtttgaggtaggcggaatgcgtggtgtagcggtgaaatgcatagatatcacgcagaactccgattgcgaaggcagcttactaaaccataactgacactgaagcacgaaagcgtggggatcaaacaggattagataccctggtagtccacgcagtaaacgatgattactaggagtttgcgatacaatgtaagctctacagcgaaagcgttaagtaatccacctggggagtacgccggcaacggtgaaactcaaaggaattgacgggggcccgcacagcggaggaacatgtggtttaattcgatgatacgcgaggaaccttacccgggtttgaacgtagtctgaccggagtggaaacactctttctagcaatagcagattacgaggtgctgcatggttgtcgtcagctcgtgccgtgaggtgtcggcttaagtgccataacgagcgcaacccttatcactagttactaacaggtgaagctgaggactctggtgagactgccagcgtaagctgtgaggaaggtggggatgacgtcaaatcagcacggcccttacatccggggcgacacacgtgttacaatggcatggacaaagggcagctacctggtgacaggatgctaatctccaaaccatgtctcagttcggatcggagtctgcaactcgactccgtgaagctggattcgctagtaatcgcgcatcagccatggcgcggtgaatacgttcccgggccttgtacacaccgcccgtcaagccatgggagccgggggtacctgaagtccgtaaccgcaag

SEQ ID NO: 233 - DNA, 1384 bp, unidentified
  misc_feature 1..1384: note = K24
  source 1..1384: mol_type = other DNA
gtcgaggggcagcatggtcttagcttgctaaggccgatggcgaccggcgcacgggtgagtaacacgtatccaacctgccgtctactcttggacagccttctgaaaggaagattaatacaagatggcatcatgagtccgcatgttcacatgattaaaggtattccggtagacgatggggatgcgttccattagatagtaggcggggtaacggcccacctagtcttcgatggataggggttctgagaggaaggtcccccacattggaactgagacacggtccaaactcctacgggaggcagcagtgaggaatattggtcaatgggcgagagcctgaaccagccaagtagcgtgaaggatgactgccctatgggttgtaaacttcttttataaaggaataaagtcgggtatggatacccgtttgcatgtactttatgaataaggatcggctaactccgtgccagcagccgcggtaatacggaggatccgagcgttatccggatttattgggtttaaagggagcgtagatggatgtttaagtcagttgtgaaagtttgcggctcaaccgtaaaattgcagttgatactggatatcttgagtgcagttgaggcaggcggaattcgtggtgtagcggtgaaatgcttagatatcacgaagaactccgattgcgaaggcagcctgctaagctgcaactgacattgaggctcgaaagtgtgggtatcaaacaggattagataccctggtagtccacacggtaaacgatgaatactcgctgtttgcgatatactgcaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcagatgaattacggtgaaagccgtaagccgcaaggcatctgtgaaggtgctgcatggttgtcgtcagctcgtgccgtgaggtgtcggcttaagtgccataacgagcgcaacccttgttgtcagttactaacaggttccgctgaggactctgacaagactgccatcgtaagatgtgaggaaggtggggatgacgtcaaatcagcacggcccttacgtccggggctacacacgtgttacaatggggggtacagagggccgctaccacgcgagtggatgccaatccccaaaacctctctcagttcggactggagtctgcaacccgactccacgaagctggattcgctagtaatcgcgcatcagccacggcgcggtgaatacgttcccgggccttgtacacaccgcccgtcaagccatgggagccgggggtacctgaagtgcgtaaccgcgag

SEQ ID NO: 234 - DNA, 1396 bp, unidentified
  misc_feature 1..1396: note = K25
  misc_feature 943..944: note = n is a, c, g, or t
  misc_feature 1072: note = n is a, c, g, or t
  misc_feature 1290: note = n is a, c, g, or t
  source 1..1396: mol_type = other DNA
gcagtcgaacgaagcgatctgggatgaagttttcggatggattcctggttgactgagtggcggacgggtgagtaacgcgtggataacctgcctcacactgggggataacagttagaaatggctgctaataccgcataagcgcacagtaccgcatggtacggtgtgaaaaacccaggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatctcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgacgcaagtctggagtgaaagcccggggcccaaccccgggactgctttggaaactgtgctgctggagtgcaggagaggtaagtggaattcctagtgtagcggtgaaatgcgtagatattaggaggaacaccagtggcgaaggcggcttactggactgtaactgacgttgaggctcgaaagcgtggggagcaaacaggattagataccctggtagtccacgccgtaaacgatgaatgctaggtgtcggggggcaaagcccttcggtgccgccgctaacgcaataagcattccacctggggagtacgttcgcaagaatgaaactcaaaggaattgacggggacccgcacaagcggtggagcatgtggtttaattcgaagcaacgcgaagaaccttaccaagtcttgacatccccctgaccggnncgtaacggtgcccttccttcgggacaggggagacaggtggtgcatggttgtcgtcagctcgtgtcgtgagatgttgggttaagtcccgcaacgagcgcaacccttatccttagtagccagcacgtganggtgggcactctagggagactgccagggataacctggaggaaggtggggatgacgtcaaatcatcatgccccttatgatttgggctacacacgtgctacaatggcgtaaacaaagggaggcgaccctgcgaaggcaagcaaatcccaaaaataacgtcccagttcggactgtagtctgcaacccgactacacgaagctggaatcgctagtaatcgcgnatcagaatgccgcggtgaatacgttcccgggtcttgtacacaccgcccgtcacaccatgggagtcagcaacgcccgaagtcagtgacccaaccttaacaggaggga

SEQ ID NO: 235 - DNA, 1382 bp, unidentified
  misc_feature 1..1382: note = K26
  source 1..1382: mol_type = other DNA
gtcgaggggcatcaggaagaaagcttgctttctttgctggcgaccggcgcacgggtgagtaacacgtatccaacctgccgatgactcggggatagcctttcgaaagaaagattaatacccgatggtatatctgaaaggcatctttcagctattaaagaatttcggtcattgatggggatgcgttccattaggttgttggcggggtaacggcccaccaagccgtcgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttaggcacgtgtgcctttttgtatgtaccgtatgaataaggatcggctaactccgtgccagcagccgcggtaatacggaggatccgagcgttatccggatttattgggtttaaagggagcgtaggcggatgcttaagtcagttgtgaaagtttgcggctcaaccgtaaaattgcagttgatactgggtgtcttgagtacagtagaggcaggcggaattcgtggtgtagcggtgaaatgcttagatatcacgaagaactccgattgcgaaggcagcttgctggactgtaactgacgctgatgctcgaaagtgtgggtatcaaacaggattagataccctggtagtccacacagtaaacgatgaatactcgctgtttgcgatatacagtaagcggccaagcgaaagcgttaagtattccacctggggagtacgccggcaacggtgaaactcaaaggaattgacgggggcccgcacaagcggaggaacatgtggtttaattcgatgatacgcgaggaaccttacccgggcttaaattgcaaatgaatgttctggaaacagatcagccgcaaggcatttgtgaaggtgctgcatggttgtcgtcagctcgtgccgtgaggtgtcggcttaagtgccataacgagcgcaacccttatcgatagttaccatcaggttatgctggggactctgtcgagactgccgtcgtaagatgtgaggaaggtggggatgacgtcaaatcagcacggcccttacgtccggggctacacacgtgttacaatggggggtacagaaggcagctacacggtgacgtgatgctaatccctaaaacctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtgcgtaaccgcga

SEQ ID NO: 236 - DNA, 1370 bp, unidentified
  misc_feature 1..1370: note = K27
  misc_feature 916..917: note = n is a, c, g, or t
  source 1..1370: mol_type = other DNA
tgcaagtcgaacggttaaggcgccttcgggcgcgaatagagtggcgaacgggtgagtaacacgtgaccaacctgcccccctccccgggataacgcgaggaaacccgcgctaataccggatactccgcccctcccgcatgggaggggcgggaaagccccgacggagggggatggggtcgcggcccattaggtagacggcggggcaacggcccaccgtgcctgcgatgggtagccgggttgagagaccgaccggccacattgggactgagatacggcccagactcctacgggaggcagcagtggggaattttgcgcaatggggggaaccctgacgcagcaacgccgcgtgcgggacgaaggccttcgggttgtaaaccgctttcagcagggaagaagttgacggtacctgcagaagaagccccggctaactacgtgccagcagccgcggtaatacgtagggggcgagcgttatccggattcattgggcgtaaagcgcgcgtaggcggcccgtcaagcggaacctctaacccgagggctcaacccccggtcgggttccgaactggcaggctcgagtttggtagaggaagatggaattcccggtgtagcggtggaatgcgcagatatcgggaagaacaccgatggcgaaggcagtcttctgggccatcaactgacgctgaggcgcgaaagctgggggagcgaacaggattagataccctggtagtcccagccgtaaacgatgggcgctaggtgtggggggatcatccctccgtgccgcagccaacgcattaagcgccccgcctggggagtacggccgcaaggctaaaactcaaaggaattgacgggggcccgcacaagcagcggagcatgtggcttaattcgaagcaacgcgaagaaccttaccagggcttgacatgctnntgaagccggggaaacccggtggccgagaggagccagcgcaggtggtgcatggctgtcgtcagctcgtgtcgtgagatgttgggttaagtcccgcaacgagcgcaacccctgccatatgttgccagcattcagttggggactcatatgggactgccggcgtcaagccggaggaaggtggggacgacgtcaagtcatcatgccctttatgccctgggctgcacacgtgctacaatggccggtacaacgggccgcgacctggcgacaggaagcgaatccctcaaagccggccccagttcggatcggaggctgcaacccgcctccgtgaagtcggagttgctagtaatcgcggatcagcatgccgcggtgaatacgttcccgggccttgtacacaccgcccgtcacaccacccgagtcgtctgcacccgaagccgccggccgaacccgcaaggggcg

SEQ ID NO: 237 - DNA, 1394 bp, unidentified
  misc_feature 1..1394: note = K28
  misc_feature 909..911: note = n is a, c, g, or t
  source 1..1394: mol_type = other DNA
caagtcgaacgaagcacttacttccaaatcttcggaagaggaggtatttgactgagtggcggacgggtgagtaacgcgtggggaacctgccccgtaccgggggataacagtcagaaatgactgctaataccgcataagcgcacgaaggcgcatgcttttgtgtgaaaaactccggtggtacgggatggtcccgcgtctgattagccagttggcggggtaacggcccaccaaagcgacgatcagtagccggcctgagagggtggacggccacattgggactgagacacggcccagactcctacgggaggcagcagtggggaatattgcacaatgggggaaaccctgatgcagcgacgccgcgtgagcgaagaagtatttcggtatgtaaagctctgtcagcagggaagaaaatgacggtacctgaccaagaagcaccggctaaatacgtgccagcagccgcggtaatacgtatggtgcaagcgttatccggatttactgggtgtaaagggagcgtagacggaggggcaagtctgaagtgaaagcccggggcccaaccccgggactgctttggaaactgtccgtctggagtgccggagaggtaagcggaattcccagtgtagcggtgaaatgcgtagatattgggaggaacaccagtggcgaaggcggcttactggacggtcactgacgttgaggctcgaaagcgtggggagcaaacaggattagataccctggtagtccacgccgtaaacgatgactactaggtgtcgggtggcagagccattcggtgccgcagccaacgcagtaagtagtccacctggggagtacgttcgcaagaatgaaactcaaaggaattgacggggacccgcacaagcggtggagcatgtggtttaattcgaagcaacgcgaagnnncttacctggccttgacatccccctgaccggcgcgtaatggtgcctttccttcgggacaggggagacaggtggtgcatggttgtcgtcagctcgtgtcgtgagatgttgggttaagtcccgcaacgagcgcaacccttatcttcagtagccagcattcaggatgggcactctggagagactgccagggacaacctggaggaaggtggggatgacgtcaaatcatcatgccccttatggccagggctacacacgtgctacaatggcgtaaacagagggaagcgagcccgcgagggggagcaaatcccaaaaataacgtcccagttcggactgcaggctgcaacccgcctgcacgaagctggaatcgctagtaatcgcgaatcagcatgtcgcggtgaatacgttcccgggtcttgtacacaccgcccgtcacaccatgggagtcggtaacgcccgaagtcagtgacccaacctccgggagggagc

SEQ ID NO: 238 - DNA, 1387 bp, unidentified
  misc_feature 1..1387: note = K29
  misc_feature 895..896: note = n is a, c, g, or t
  misc_feature 910..912: note = n is a, c, g, or t
  misc_feature 942..946: note = n is a, c, g, or t
  misc_feature 953..954: note = n is a, c, g, or t
  source 1..1387: mol_type = other DNA
agtcgaacgaagcatttaggattgaagttttcggatggatttcctttatgactgagtggcggacgggtgagtaacgcgtggggaacctgccctatacagggggataacagctggaaacggctgctaataccgcataagcgcacagaatcgcatgattcagtgtgaaaagccctggcagtataggatggtcccgcgtctgattagctggttggtgaggtaacggctcaccaaggcgacgatcagtagccggcttgagagagtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaacagacggtacctgactaagaagccccggctaactacgtgccagcagccgcggtaatacgtagggggcaagcgttatccggaattactgggtgtaaagggtgcgtaggtggcatggtaagtcagaagtgaaagcccggggcttaaccccgggactgcttttgaaactgtcatgctggagtgcaggagaggtaagcggaattcctagtgtagcggtgaaatgcgtagatattaggaggaacaccagtggcgaaggcggcttactggactgtcactgacactgatgcacgaaagcgtggggagcaaacaggattagataccctggtagtccacgccgtaaacgatgaatactaggtgtcggggccgtagaggcttcggtgccgcagcaaacgcagtaagtattccacctggggagtacgttcgcaagaatgaaactcaaaggaattgacggggacccgcacaagcggtggagcatgtggtttaattnnaagcaacgcgaagnnncttacctggtcttgacatcccaatgaccgnnnnntaaccgnntttttctttcgagacattggagacaggtggtgcatggttgtcgtcagctcgtgtcgtgagatgttgggttaagtcccgcaacgagcgcaacccctatctttagtagccagcattagaggtgggcactctagagagactgccagggataacctggaggaaggtggggaggacgtcaaatcatcatgccccttatggccagggctacacacgtgctacaatggcgtaaacaaagggaagcgaagtcgtgaggcgaagcaaatcccagaaataacgtctcagttcggattgtagtctgcaactcgactacatgaagctggaatcgctagtaatcgtgaatcagaatgtcacggtgaatacgttcccgggtcttgtacacaccgcccgtcacaccatgggagtcagtaacgcccgaagtcagtgacccaaccgcaag

SEQ ID NO: 239 - DNA, 1392 bp, unidentified
  misc_feature 1..1392: note = K30
  source 1..1392: mol_type = other DNA
gcaagtcgagcgaagcaccttgacggatttcttcggattgaagccttggtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaagccgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttccttcgggacagaggagacaggtggtgcatggttgtcgtcagctcgtgtcgtgagatgttgggttaagtcccgcaacgagcgcaacccctatctttagtagccagcattttggatgggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggaggg

SEQ ID NO: 240 - DNA, 1394 bp, unidentified
  misc_feature 1..1394: note = K31
  misc_feature 1073..1074: note = n is a, c, g, or t
  source 1..1394: mol_type = other DNA
cagtcgagcgaagcgctttgtgcggatttcttcggattgaagcaactgtgactgagcggcggacgggtgagtaacgcgtgggtaacctgcctcatacagggggataacagttggaaacggctgctaataccgcataagcgcacagtaccgcatggtacggtgtgaaaaactccggtggtatgagatggacccgcgtctgattaggtagttggtggggtaacggcctaccaaggcgacgatcagtagccgacctgagagggtgaccggccacattgggactgagacacggcccaaactcctacgggaggcagcagtggggaatattgcacaatgggggaaaccctgatgcagcgacgccgcgtgagcgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcatggcaagccagatgtgaaagcccggggctcaaccccgggactgcatttggaactgtcaggctagagtgtcggagaggaaagcggaattcctagtgtagcggtgaaatgcgtagatattaggaggaacaccagtggcgaaggcggctttctggacgatgactgacgttgaggctcgaaagcgtggggagcaaacaggattagataccctggtagtccacgccgtaaacgatgaatactaggtgtcgggtggcaaagccattcggtgccgcagcaaacgcaataagtattccacctgggggagtacgttcgcaagaatgaaactcaaaggaattgacggggacccgcacaagcggtggagcatgtggtttaattcgaagcaacgcgaagaaccttacctggtcttgacatccctctgaccgctctttaatcggagctttctttcgggacagaggagacaggtggtgcatggttgtcgtcagctcgtgtcgtgagatgttgggttaagtcccgcaacgagcgcaacccctatctttagtagccagcatttaagnngggcactctagagagactgccagggataacctggaggaaggtggggatgacgtcaaatcatcatgccccttatgaccagggctacacacgtgctacaatggcgtaaacaaagggaagcgagcccgcgagggggagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggag

SEQ ID NO: 241 - DNA, 1383 bp, unidentified
  misc_feature 1..1383: note = K32
  source 1..1383: mol_type = other DNA
aagtcgaggggcagcacggtgtagcaatacactggtggcgaccggcgcacgggtgcgtaacgcgtatgcaacctacccataacagggggataacactgagaaattggtactaataccccataacatcaggaccggcatcggttctggttgaaaactccggtggttatggatgggcatgcgttgtattagctggttggtgaggtaacggctcaccaaggcaacgatacatagggggactgagaggttaaccccccacattggtactgagacacggaccaaactcctacgggaggcagcagtgaggaatattggtcaatggacgcaagtctgaaccagccatgccgcgtgcaggaagacggctctatgagttgtaaactgcttttgtacgagggtaaacgctcttacgtgtaagagcctgaaagtatcgtacgaataaggatcggctaactccgtgccagcagccgcggtaatacggaggatccaagcgttatccggatttattgggtttaaagggtgcgtaggcggtttgataagttagaggtgaaataccggtgcttaacaccggaactgcctctaatactgttgaactagagagtagttgcggtaggcggaatgtatggtgtagcggtgaaatgcttagagatcatacagaacaccgattgcgaaggcagcttaccaaactatatctgacgttgaggcacgaaagcgtggggagcaaacaggattagataccctggtagtccacgcagtaaacgatgataactcgctgtcggcgatacacagtcggcggctaagcgaaagcgataagttatccacctggggagtacgttcgcaagaatgaaactcaaaggaattgacgggggcccgcacaagcggaggaacatgtggtttaattcgatgatacgcgaggaaccttacccgggcttgaaagttactgacgattctggaaacaggatttcccttcggggcaggaaactaggtgctgcatggttgtcgtcagctcgtgccgtgaggtgtcgggttaagtcccataacgagcgcaacccctaccgttagttgccatcaggtcaagctgggcactctggcgggactgccggtgtaagccgagaggaaggtggggatgacgtcaaatcagcacggcccttacgtccggggctacacacgtgttacaatggtaggtacagagggcagctacccagtgatgggatgcgaatctcgaaagcctatctcagttcggatcggaggctgaaacccgcctccgtgaagttggattcgctagtaatcgcgcatcagccatggcgcggtgaatacgttcccgggccttgtacacaccgcccgtcaagccatggaagctgggggtgcctgaagttcgtgaccgcaagg

SEQ ID NO: 242 - DNA, 1378 bp, unidentified
  misc_feature 1..1378: note = K33
  source 1..1378: mol_type = other DNA
gaggggcagcatcattaaagcttgctttgatggatggcgaccggcgcacgggtgagtaacacgtatccaacctgccgacaacactgggatagcctttcgaaagaaagattaataccggatggcatagttttcccgcatgggataattattaaagaatttcggttgtcgatggggatgcgttccattaggcagttggcggggtaacggcccaccaaaccaacgatggataggggttctgagaggaaggtcccccacattggaactgagacacggtccaaactcctacgggaggcagcagtgaggaatattggtcaatggacgagagtctgaaccagccaagtagcgtgaaggatgactgccctatgggttgtaaacttcttttatacgggaataaagttagccacgtgtggctttttgtatgtaccgtatgaataaggatcggctaactccgtgccagcagccgcggtaatacggaggatccgagcgttatccggatttattgggtttaaagggagcgtaggcgggttgttaagtcagttgtgaaagtttgcggctcaaccgtaaaattgcagttgatactggcgaccttgagtgcaacagaggtaggcggaattcgtggtgtagcggtgaaatgcttagatatcacgaagaactccgattgcgaaggcagcttactggattgtaactgacgctgatgctcgaaagtgtgggtatcaaacaggattagataccctggtagtccacacagtaaacgatgaatactcgctgttggcgatatacggtcagcggccaagcgaaagcattaagtattccacctggggagtacgccggcaacggtgaaactcaaaggaattgacgggggcccgcacaagcggaggaacatgtggtttaattcgatgatacgcgaggaaccttacccgggcttaaattgcaactgactgaaccggaaacggttctttcttcggacagttgtgaaggtgctgcatggttgtcgtcagctcgtgccgtgaggtgtcggcttaagtgccataacgagcgcaacccttatcgatagttactagcaggtcatgctgaggactctattgagactgccgtcgtaagatgtgaggaaggtggggatgacgtcaaatcagcacggcccttacgtccggggctacacacgtgttacaatggggggtacagaaggcagctacacggcgacgtggtgctaatcccgaaagcctctctcagttcggattggagtctgcaacccgactccatgaagctggattcgctagtaatcgcgcatcagccacggcgcggtgaatacgttcccgggccttgtacacaccgcccgtcaagccatgaaagccgggggtacctgaagtacgtaaccgcg

SEQ ID NO: 243 - DNA, 1394 bp, unidentified
  misc_feature 1..1394: note = K34
  source 1..1394: mol_type = other DNA
cagtcgagcgaagcacttaagtggatctcttcggattgaaacttattgtgactgagcggcggacgggtgagtaacgcgtgggtaacctgcctcatacagggggataacagttagaaatggctgctaataccgcataagcgcacaggaccgcatggtctggtgtgaaaaactccggtggtatgagatggacccgcgtctgattagctagttggaggggtaacggcccaccaaggcgacgatcagtagccggcctgagagggtgaacggccacattgggactgagacacggcccagactcctacgggaggcagcagtggggaatattgcacaatgggggaaaccctgatgcagcgacgccgcgtgaaggaagaagtatctcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggaagagcaagtctgatgtgaaaggctggggcttaaccccaggactgcattggaaactgtttttctagagtgccggagaggtaagcggaattcctagtgtagcggtgaaatgcgtagatattaggaggaacaccagtggcgaaggcggcttactggacggtaactgacgttgaggctcgaaagcgtggggagcaaacaggattagataccctggtagtccacgccgtaaacgatgaatactaggtgtcgggtggcaaagccattcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatccctctgaccggcccgtaacggggccttcccttcggggcagaggagacaggtggtgcatggttgtcgtcagctcgtgtcgtgagatgttgggttaagtcccgcaacgagcgcaacccctatccttagtagccagcaggtgaagctgggcactctagggagactgccggggataacccggaggaaggcggggacgacgtcaaatcatcatgccccttatgatttgggctacacacgtgctacaatggcgtaaacaaagggaagcgagacagcgatgttgagcaaatcccaaaaataacgtcccagttcggactgcagtctgcaactcgactgcacgaagctggaatcgctagtaatcgcgaatcagaatgtcgcggtgaatacgttcccgggtcttgtacacaccgcccgtcacaccatgggagtcagtaacgcccgaagtcagtgacccaaccttacaggaggga

SEQ ID NO: 244 - DNA, 1392 bp, unidentified
  misc_feature 1..1392: note = K35
  misc_feature 901: note = n is a, c, g, or t
  source 1..1392: mol_type = other DNA
cagtcgaacggagctcagttttggaaactttcttcgggagtggaattctcgacttagtggcggacgggtgagtaacgcgtgagcaatctgcctttaagagggggataacagtcggaaacggctgctaataccgcataaagcattgaattcgcatgttttcgatgccaaaggagcaatccgcttttagatgagctcgcgtctgattagctagttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatcagggacgaaacaaatgacggtacctgaagaataagctccggctaactacgtgccagcagccgcggtaatacgtagggagcaagcgttatccggatttactgggtgtaaagggcgcgcaggcgggccggcaagttggaagtgaaatctatgggcttaacccataaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattngaagcaacgcgaagaaccttaccaggtcttgacatcccgatgaccgccttagagataaggctttttttcggaacatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaaaaccgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccttcttggag

SEQ ID NO: 245 - DNA, 1394 bp, unidentified
  misc_feature 1..1394: note = K36
  misc_feature 1068..1069: note = n is a, c, g, or t
  source 1..1394: mol_type = other DNA
agtcgaacgaagcaattaaaatgaagttttcggatggatttttgattgactgagtggcggacgggtgagtaacgcgtggataacctgcctcacactgggggataacagttagaaatgactgctaataccgcataagcgcacagtaccgcatggtacggtgtgaaaaactccggtggtgtgagatggatccgcgtctgattagccagttggcggggtaacggcccaccaaagcgacgatcagtagccgacctgagagggtgaccggccacattgggactgagacacggcccaaactcctacgggaggcagcagtggggaatattgcacaatgggcgaaagcctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcgaagcaagtctgaagtgaaaacccagggctcaaccctgggactgctttggaaactgttttgctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatgctaggtgttggggggcaaagcccttcggtgccgtcgcaaacgcagtaagcattccacctggggagtacgttcgcaagaatgaaactcaaaggaattgacggggacccgcacaagcggtggagcatgtggtttaattcgaagcaacgcgaagaaccttaccaagtcttgacatcctcttgaccggcgtgtaacggcgccttcccttcggggcaagagagacaggtggtgcatggttgtcgtcagctcgtgtcgtgagatgttgggttaagtcccgcaacgagcgcaacccttatccttagtagccagcaggtanngctgggcactctagggagactgccagggataacctggaggaaggtggggatgacgtcaaatcatcatgccccttatgatttgggctacacacgtgctacaatggcgtaaacaaagggaagcaagacagtgatgtggagcaaatcccaaaaataacgtcccagttcggactgtagtctgcaacccgactacacgaagctggaatcgctagtaatcgcgaatcagaatgtcgcggtgaatacgttcccgggtcttgtacacaccgcccgtcacaccatgggagtcagcaacgcccgaagtcagtgacccaactcgcaagagagggag

SEQ ID NO: 246 - DNA, 1371 bp, unidentified
  misc_feature 1..1371: note = K37
  source 1..1371: mol_type = other DNA
cagtcgaacgagccgaggggagcttgctccccagagctagtggcggacgggtgagtaacacgtgagcaacctgcctttcagagggggataacgtttggaaacgaacgctaataccgcataacataccgggaccgcatgattctggtatcaaaggagcaatccgctgaaagatgggctcgcgtccgattagctagttggcggggtaacggcccaccaaggcgacgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtctttggggacgataatgacggtacccaaggaggaagctccggctaactacgtgccagcagccgcggtaatacgtagggagcgagcgttgtccggaattactgggtgtaaagggagcgtaggcggggtctcaagtcgaatgttaaatctaccggctcaactggtagctgcgttcgaaactggggctcttgagtgaagtagaggcaggcggaattcctagtgtagcggtgaaatgcgtagatattaggaggaacaccagtggcgaaggcggcctgctgggcttttactgacgctgaggctcgaaagcgtggggagcaaacaggattagataccctggtagtccacgccgtaaacgatgattactaggtgtggggggactgaccccttccgtgccggagttaacacaataagtaatccacctggggagtacgaccgcaaggttgaaactcaaaggaattgacgggggcccgcacaagcagtggattatgtggtttaattcgaagcaacgcgaagaaccttaccaggtcttgacatcgagtgacggctctagagatagagctttccttcgggacacaaagacaggtggtgcatggttgtcgtcagctcgtgtcgtgagatgttgggttaagtcccgcaacgagcgcaacccttattattagttgctacattcagttgagcactctaatgagactgccgttgacaaaacggaggaaggtggggatgacgtcaaatcatcatgccccttatgacctgggctacacacgtaatacaatggcgatcaacagagggaagcaagaccgcgaggtggagcaaacccctaaaagtcgtctcagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagtcagtagcctaaccgcaaggagg

SEQ ID NO: 247 - DNA, 1396 bp, unidentified
  misc_feature 1..1396: note = K38
  source 1..1396: mol_type = other DNA
tgcagtcgaacgctttgtaaaggagcttgcttctttacgaggagtggcgaacgggtgagtaatacataagcaatctgcccatcggcctgggataacagttggaaacgactgctaataccggataggttagtttctggcatcagggactaattaaagttgggatacaacacggatggatgagcttatggcgtattagctagtaggtgaggtaacggcccacctaggcgatgatacgtagccgacctgagagggtgaccggccacattgggactgagacacggcccaaactcctacgggaggcagcagtagggaattttcggcaatgggcgaaagcctgaccgagcaacgccgcgtgagtgaagaaggccttcgggttgtaaagctctgttgtgaaggaagaacggctcatagagggaatgctatgggagtgacggtactttaccagaaagccacggctaactacgtgccagcagccgcggtaatacgtaggtggcgagcgttatccggaattattgggcgtaaagggtgcgcaggcggtttgaaaagtttaaggtgaaagcgtggggcttaaccccatacagccttagaaactgtcagactagagtacaggagagggcaatggaattccatgtgtagcggtaaaatgcgtagatatatggaggaacaccagtggcgaaggcggttgcctggcctgtaactgacgctcatgcacgaaagcgtggggagcaaataggattagataccctagtagtccacgccgtaaacgatgagaactaagtgttggggaaactcagtgctgcagttaacgcaataagttctccgcctggggagtatgcacgcaagtgtgaaactcaaaggaattgacgggggcccgcacaagcggtggagtatgtggtttaattcgacgcaacgcgaagaaccttaccaggtcttgacataccaggcaaagctatagagatatagtggaggatatcctggatacaggtggtgcatggttgtcgtcagctcgtgtcgtgagatgttgggttaagtcccgcaacgagcgcaacccttgtctttagttactaacattaagttgaggactctagagagactgccggtgacaaaccggaggaaggtggggatgacgtcaaatcatcatgccccttatgacctgggctacacacgtactacaatggcggatacaacgagaagcaagacagcaatgtggagcaaacctcagaaagtccgtctcagttcggattgaagtctgcaacccgacttcatgaagccggaatcgctagtaatcgcggatcagcatgccgcggtgaatacgttctcgggccttgtacacaccgcccgtcaaaccatgagagttggcaatacccgaagccggtggcctaaccctgcaaaggga

SEQ ID NO: 248 - DNA, 1399 bp, unidentified
  misc_feature 1..1399: note = K39
  source 1..1399: mol_type = other DNA
cagtcgaacggtaacaggaagcagcttgctgctttgctgacgagtggcggacgggtgagtaatgtctgggaaactgcctgatggagggggataactactggaaacggtagctaataccgcataacgtcgcaagaccaaagagggggaccttagggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacggaagttttcagagatgagaatgtgccttcgggaaccgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggag

SEQ ID NO: 249 - DNA, 1384 bp, unidentified
  misc_feature 1..1384: note = K40
  source 1..1384: mol_type = other DNA
agtcgaacggggttattttggaaatctcttcggagatggaattcttaacctagtggcggacgggtgagtaacgcgtgagcaatctgcctttaggagggggataacagtcggaaacggctgctaataccgcataatacgtttgggaggcatctcttgaacgtcaaagattttatcgcctttagatgagctcgcgtctgattagctggttggcggggtaacggcccaccaaggcgacgatcagtagccggactgagaggttgaacggccacattgggactgagacacggcccagactcctacgggaggcagcagtggggaatattgcgcaatgggggaaaccctgacgcagcaacgccgcgtgattgaagaaggccttcgggttgtaaagatctttaatcagggacgaaaaatgacggtacctgaagaataagctccggctaactacgtgccagcagccgcggtaatacgtagggagcaagcgttatccggatttactgggtgtaaagggcgcgcaggcgggccggcaagttgggagtgaaatcccggggcttaaccccggaactgctttcaaaactgctggtcttgagtgatggagaggcaggcggaattccgtgtgtagcggtgaaatgcgtagatatacggaggaacaccagtggcgaaggcggcctgctggacattaactgacgctgaggcgcgaaagcgtggggagcaaacaggattagataccctggtagtccacgccgtaaacgatggatactaggtgtgggaggtattgaccccttccgtgccgcagttaacacaataagtatcccacctggggagtacggccgcaaggttgaaactcaaaggaattgacgggggcccgcacaagcagtggagtatgtggtttaattcgaagcaacgcgaagaaccttaccaggtcttgacatcccgatgaccggcgtagagatacgccctctcttcggagcatcggtgacaggtggtgcatggttgtcgtcagctcgtgtcgtgagatgttgggttaagtcccgcaacgagcgcaacccttacggttagttgatacgcaagatcactctagccggactgccgttgacaaaacggaggaaggtggggacgacgtcaaatcatcatgccccttatgacctgggctacacacgtactacaatggcagtcatacagagggaagcaataccgcgaggtggagcaaatccctaaaagctgtcccagttcagattgcaggctgcaacccgcctgcatgaagtcggaattgctagtaatcgcggatcagcatgccgcggtgaatacgttcccgggccttgtacacaccgcccgtcacaccatgagagccgtcaatacccgaagtccgtagcctaaccgcaagggg

SEQ ID NO: 250 - DNA, 1392 bp, unidentified
  misc_feature 1..1392: note = K41
  source 1..1392: mol_type = other DNA
agtcgaacgaagcactttatttgatttccttcgggactgattattttgtgactgagtggcggacgggtgagtaacgcgtgggtaacctgccttgtacagggggataacagttggaaacggctgctaataccgcataagcgcacggcatcgcatgatgcagtgtgaaaaactccggtggtataagatggacccgcgttggattagctagttggtgaggtaacggcccaccaaggcgacgatccatagccgacctgagagggtgaccggccacattgggactgagacacggcccaaactcctacgggaggcagcagtggggaatattgcacaatgggcgaaagcctgatgcagcgacgccgcgtgagcgaagaagtatttcggtatgtaaagctctatcagcagggaagataatgacggtacctgactaagaagcaccggctaaatacgtgccagcagccgcggtaatacgtatggtgcaagcgttatccggatttactgggtgtaaagggagcgcaggcggtgcggcaagtctgatgtgaaagcccggggctcaaccccggtactgcattggaaactgtcgtactagagtgtcggaggggtaagcggaattcctagtgtagcggtgaaatgcgtagatattaggaggaacaccagtggcgaaggcggcttactggacgataactgacgctgaggctcgaaagcgtggggagcaaacaggattagataccctggtagtccacgccgtaaacgatgaatactaggtgttgggaagcattgcttctcggtgccgtcgcaaacgcagtaagtattccacctggggagtacgttcgcaagaatgaaactcaaaggaattgacggggacccgcacaagcggtggagcatgtggtttaattcgaagcaacgcgaagaaccttaccaagtcttgacatccttctgaccggtacttaaccgtaccttctcttcggagcaggagtgacaggtggtgcatggttgtcgtcagctcgtgtcgtgagatgttgggttaagtcccgcaacgagcgcaacccttatctttagtagccagcggttcggccgggcactctagagagactgccagggataacctggaggaaggcggggatgacgtcaaatcatcatgccccttatgacttgggctacacacgtgctacaatggcgtaaacaaagggaagcaaagctgtgaagccgagcaaatctcaaaaataacgtctcagttcggactgtagtctgcaacccgactacacgaagctggaatcgctagtaatcgcagatcagaatgctgcggtgaatacgttcccgggtcttgtacacaccgcccgtcacaccatgggagttgggaatgcccgaagccagtgacctaaccgaaaggaagga

SEQ ID NO: 251 - DNA, 1397 bp, unidentified
  misc_feature 1..1397: note = K42
  source 1..1397: mol_type = other DNA
cagtcgaacgaagcatatagagacgagtatttcggtatgagtaactatatgactgagtggcggacgggtgagtaacgcgtggataacctgcctcatacagggggataacagttagaaatgactgctaataccgcataagcgcacagtgctgcatggcacagtgtgaaaagctccggcggtatgagatggatccgcgtttgattagctagttggtggggtaaaggcctaccaaggcgacgatcaatagccgacctgagagggtgaccggccacattgggactgagacacggcccaaactcctacgggaggcagcagtggggaatattgcacaatgggggaaaccctgatgcagcgacgccgcgtgaaggaagaagtatttcggtatgtaaacttctatcagcagggaagaaaatgacggtacctgactaagaagccccggctaattacgtgccagcagccgcggtaatacgtaaggggcaagcgttatccggatttactgggtgtaaagggagcgtagacggcagtgcaagtctgatgtgaaagcccggggctcaaccccgggactgcattggaaactgtgcagctagagtgtcggagaggtaagtggaattcctagtgtagcggtgaaatgcgtagatattaggaggaacaccagtggcgaaggcggcttactggacgataactgacgttgaggctcgaaagcgtggggagcaaacaggattagataccctggtagtccacgccgtaaacgatgaatactaggtgtcgggcgccaaaggcgttcggtgccgcagcaaacgcaataagtattccacctggggagtacgttcgcaagaatgaaactcaaaggaattgacggggacccgcacaagcggtggagcatgtggtttaattcgaagcaacgcgaagaaccttaccaagtcttgacatctgcctgaccggtccgtaacaggacccttccttcgggacaggcaagacaggtggtgcatggttgtcgtcagctcgtgtcgtgagatgttgggttaagtcccgcaacgagcgcaacccttatccttagtagccagcaggtagagctgggcactctagggagactgccagggacaacctggaggaaggtggggatgacgtcaaatcatcatgccccttatgatttgggctacacacgtgctacaatggcgtaaacaaagggaagcgaaggggtgacctgaagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcggatatgcccgaagccggtgaccgaacccgaaagggaaggag

SEQ ID NO: 252 - DNA, 1388 bp, unidentified
  misc_feature 1..1388: note = K43
  source 1..1388: mol_type = other DNA
agtcgaacggagcacccttgactgaggtttcggccaaatgataggaatgcttagtggcggactggtgagtaacgcgtgaggaacctgccttccagagggggacaacagttggaaacgactgctaataccgcatgacgcatgaccggggcatcccgggcatgtcaaagattttatcgctggaagatggcctcgcgtctgattagctagatggtggggtaacggcccaccatggcgacgatcagtagccggactgagaggttgaccggccacattgggactgagatacggcccagactcctacgggaggcagcagtggggaatattgggcaatggacgcaagtctgacccagcaacgccgcgtgaaggaagaaggctttcgggttgtaaacttcttttgtcagggaagagtagaagacggtacctgacgaataagccacggctaactacgtgccagcagccgcggtaatacgtaggtggcaagcgttgtccggatttactgggtgtaaagggcgtgcagccgggccggcaagtcagatgtgaaatctggaggcttaacctccaaactgcatttgaaactgtaggtcttgagtaccggagaggttatcggaattccttgtgtagcggtgaaatgcgtagatataaggaagaacaccagtggcgaaggcggataactggacggcaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgaagcaacgcgaagaaccttaccagggcttgacatcctactaacgaagtagagatacatcaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttatgtcctgggctacacacgtaatacaatggcggtcaacagagggaggcaaagccgcgaggcagagcaaacccccaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagg

SEQ ID NO: 253 - DNA, 1393 bp, unidentified
  misc_feature 1..1393: note = K44
  source 1..1393: mol_type = other DNA
tgcagtcgaacggagtgccttagaaagaggattcgtccaattgataaggttacttagtggcggacgggtgagtaacgcgtgaggaacctgcctcggagtggggaataacagaccgaaaggcctgctaataccgcatgatgcagttggaccgcatggtcctgactgccaaagatttatcgctctgagatggcctcgcgtctgattagcttgttggcggggtaatggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctcagggacgaacaaatgacggtacctgaggaataagccacggctaactacgtgccagcagccgcggtaatacgtaggtggcaagcgttatccggatttactgggtgtaaagggcgtgtaggcgggaaggcaagtcagatgtgaaaactatgggctcaacccatagcctgcatttgaaactgtttttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgctgtaaacgatggatactaggtgtgggggggtctgaccccctccgtgccgcagttaacacaataagtatcccacctggggagtacgatcgcaaggttgaaactcaaaggaattgacgggggcccgcacaagcggtggagtatgtggtttaattcgaagcaacgcgaagaaccttaccagggcttgacatcctactaacgaagcagagatgcattaggtgcccttcggggaaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggcggggacgacgtcaaatcatcatgccccttatgtcctgggctacacacgtactacaatggtggtaaacagagggaagcaagaccgcgaggtggagcaaatccctaaaagccatcccagttcggattgcaggctgaaacccgcctgtatgaagttggaatcgctagtaatcgcggatcagcatgccgcggtgaatacgttcccgggccttgtacacaccgcccgtcacaccatgagagtcgggaacacccgaagtccgtagtctaaccgcaaggggga

SEQ ID NO: 254 - DNA, 1409 bp, unidentified
  misc_feature 1..1409: note = K45
  source 1..1409: mol_type = other DNA
caagtagaacgctgactactttagcttgctagagtagaaggagttgcgaacgggtgagtaacgcgtaggtaacctgcctactagcgggggataactattggaaacgatagctaataccgcataacagtgtttaacacatgttagatgcttgaaagatgcaattgcatcactagtagatggacctgcgttgtattagctagttggtggggtaacggcccaccaaggcgacgatacatagccgacctgagagggtgatcggccacactgggactgagacacggcccagactcctacgggaggcagcagtagggaatcttcggcaatgggggcaaccctgaccgagcaacgccgcgtgagtgaagaaggttttcggatcgtaaagctctgttgtaagagaagaacgtgtgtgagagtggaaagttcacacagtgacggtaacttaccagaaagggacggctaactacgtgccagcagccgcggtaatacgtaggtcccgagcgttgtccggatttattgggcgtaaagcgagcgcaggcggtttaataagtctgaagttaaaggcagtggcttaaccattgttcgctttggaaactgttaaacttgagtgcagaaggggagagtggaattccatgtgtagcggtgaaatgcgtagatatatggaggaacaccggtggcgaaagcggctctctggtctgtaactgacgctgaggctcgaaagcgtggggagcaaacaggattagataccctggtagtccacgccgtaaacgatgagtgctaggtgttaggccctttccggggcttagtgccgcagctaacgcattaagcactccgcctggggagtacgaccgcaaggttgaaactcaaaggaattgacgggggcccgcacaagcggtggagcatgtggtttaattcgaagcaacgcgaagaaccttaccaggtcttgacatcccgatgctatttctagagatagaaagtttcttcggaacatcggtgacaggtggtgcatggttgtcgtcagctcgtgtcgtgagatgttgggttaagtcccgcaacgagcgcaacccctattgttagttgccatcatttagttgggcactctagcgagactgccggtaataaaccggaggaaggtggggatgacgtcaaatcatcatgccccttatgacctgggctacacacgtgctacaatggttggtacaacgagtcgcaagtcggtgacggcaagcaaatctcttaaagccaatctcagttcggattgtaggctgcaactcgcctacatgaagtcggaatcgctagtaatcgcggatcagcacgccgcggtgaatacgttcccgggccttgtacacaccgcccgtcacaccacgagagtttgtaacacccgaagtcggtgaggtaaccatttaggagcc

SEQ ID NO: 255 - DNA, 1395 bp, unidentified
  misc_feature 1..1395: note = K46
  source 1..1395: mol_type = other DNA
caagtcgaacggcagcgcggggagcttgctccctggcggcgagtggcgcacgggtgagtaatacatcggaacgtgtcttctagtgggggataactgcccgaaagggcagctaataccgcatgagacctgagggtgaaagcgggggatcgcaagacctcgcgctggaagagcggccgatgtccgattagctagttggtgaggtaaaggctcaccaaggcgacgatcggtagctggtctgagaggacgaccagccacactgggactgagacacggcccagactcctacgggaggcagcagtggggaattttggacaatgggggcaaccctgatccagccatgccgcgtgcaggatgaaggtcttcggattgtaaactgcttttgtcagggacgaaaagggatgcgataacaccgcattccgctgacggtacctgaagaataagcaccggctaactacgtgccagcagccgcggtaatacgtagggtgcaagcgttaatcggaattactgggcgtaaagcgtgcgcaggcggttctgtaagatagatgtgaaatccccgggctcaacctgggaattgcatatatgactgcaggacttgagtttgtcagaggagggtggaattccacgtgtagcagtgaaatgcgtagatatgtggaagaacaccgatggcgaaggcagccctctgggacatgactgacgctcatgcacgaaagcgtggggagcaaacaggattagataccctggtagtccacgccctaaacgatgtctactagttgttggggacgatagtccttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctagccttgacatgccaggaaggcctgagagatcaggccgtgcccgcaagggaatctggacacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatggctagggcctcacacgtcatacaatggtcggaacagagggaagcgaagccgcgaggtgaagccaatcccagaaaaccgatcgtagtccggattgcagtctgcaactcgactgcatgaagtcggaatcgctagtaatcgcggatcagcatgccgcggtgaatacgttcccgggtcttgtacacaccgcccgtcacaccatgggagtggggttcaccagaagacgtttgcccaaccgaaaggaagg

(19) United States
(12) Patent Application Publication
(10) Pub. No.: US 2026/0240924 A1
(43) Pub. Date: Aug. 20, 2026

(54) ANTIBACTERIAL COMPOSITION COMPRISING GLUCONIC ACID-CONSUMING BACTERIA AS ACTIVE INGREDIENT

(71) Applicants: KEIO UNIVERSITY, Tokyo (JP); JSR CORPORATION, Tokyo (JP)

(72) Inventors: Kenya Honda, Shinjuku-ku, Tokyo (JP); Munehiro Furuichi, Shinjuku-ku, Tokyo (JP); Naomi Hasegawa, Shinjuku-ku, Tokyo (JP); Satoshi Sasajima, Shinjuku-ku, Tokyo (JP); Koji Atarashi, Shinjuku-ku, Tokyo (JP); Takashi Ohya, Shinjuku-ku, Tokyo (JP); Yoshimasa Aoto, Shinjuku-ku, Tokyo (JP)

(73) Assignees: KEIO UNIVERSITY, Tokyo (JP); JSR CORPORATION, Tokyo (JP)

(21) Appl. No.: 19/162,586

(22) PCT Filed: Mar. 4, 2024
(86) PCT No.: PCT/JP2024/008014

§ 371 Date: Sep. 5, 2025

Related U.S. Application Data
(60) Provisional application No. 63/599,806, filed on Nov. 16, 2023.

(30) Foreign Application Priority Data
Mar. 7, 2023 (JP) 2023-034667

(51) Int. Cl. A61K 35/745; A61K 35/741; A61K 35/742; A61P 31/04; C12Q 1/689

(52) U.S. Cl. CPC A61K 35/745; A61K 35/741; A61K 35/742; A61P 31/04; C12Q 1/689; C12Q 2600/158

(57) ABSTRACT

An antibacterial composition comprising gluconate-consuming first bacteria as an active ingredient, the composition being against gluconate-consuming second bacteria such as proinflammatory bacteria and drug-resistant bacteria. The first bacteria can comprise, in the same gene cluster, at least one gene selected from the group consisting of a gene encoding gluconate kinase and a gene encoding gluconate dehydratase, and a gene encoding a gluconate transporter.

### RELATED APPLICATIONS

This application is a national stage filing under 35 U.S.C. § 371 of internation application number PCT/JP2024/008014 filed Mar. 4, 2024, which claims priority from Japanese application No. 2023-034667 filed Mar. 7, 2023, and U.S. provisional application No. 63/599,806. The entire contents of each of these referenced applications are incorporated by reference herein.

### FIELD

### Reference to an Electronic Sequence Listing

The contents of the electronic sequence listing (P250701US00 Sequence Listing: 515, 664 bytes; and Dates of Creation Sep. 3, 2025) is herein incorporated by reference in its entirety.

The present invention relates to an antibacterial composition comprising gluconate-utilizing bacteria as an active agent. More specifically, the present invention relates to an antibacterial composition comprising first bacteria as an active agent, in which the first bacteria capable of utilizing gluconate and the composition exhibits antibacterial activity against second bacteria capable of utilizing gluconate. The present invention also relates to a method for screening bacteria having antibacterial activity against gluconate-utilizing bacteria.

### BACKGROUND

Various resident bacteria exist in mucous membranes such as the intestinal tract and oral cavity, forming a flora as a whole. The resident bacterial flora plays a very important role in the host's physiology and health maintenance. An imbalance in the composition of the resident bacterial flora is called dysbiosis, and it is gradually becoming clear that it causes various diseases. Elucidation of the mucosal resident bacterial flora is highly likely to lead to the development of new disease control and treatment strategies for various diseases; however, the detailed mechanisms have not been fully elucidated due to its complexity.

Regarding such mucosal resident bacteria, the present inventors have succeeded in isolating, culturing, and identifying bacteria that colonize the intestine (intestinal tract) and induce Th1 cells, thereby being involved in the onset of the disease such as Crohn's disease, from bacteria contained in the saliva (namely oral bacteria) of patients with Crohn's disease, etc. (WO 2018/084172). More specifically, the present inventors have found that oral administration of saliva derived from a patient with Crohn's disease to germ-free mice resulted in a marked increase in IFN-γ-producing CD4-positive T cells (Th1 cells) in the large intestine. The present inventors have succeeded in isolating and culturing the strain Kp2H7, which is thought to belong to Klebsiella pneumoniae, from the intestines of mice in which this increase in Th1 cells was observed. The present inventors have also indicated that the bacteria derived from the saliva of Crohn's disease patients colonize the intestinal tract and induce the proliferation or activation of Th1 cells, thereby being involved in the development of enteritis.

Furthermore, the present inventors assumed the existence of bacteria that suppress the intestinal colonization of Th1 cell-inducing bacteria in human intestinal bacteria, and attempted to identify them. As a result, the present inventors have succeeded in isolating and culturing 37 intestinal bacterial strains from fecal samples derived from a healthy individual (subject number: #F), and determining the sequence of 16S rDNA of each bacterial strain. Also, the present inventors have further shown that administration of these bacterial strains suppresses intestinal colonization of Th1 cell-inducing bacteria (WO 2019/017389).

It has also been found that the bacteria that suppress the intestinal colonization of the aforementioned Th1 cell-inducing bacteria (37 intestinal bacterial strains derived from healthy individual #F, 42 intestinal bacterial strains derived from healthy individual #I, 47 intestinal bacterial strains derived from healthy individual #K, etc.) can also suppress the intestinal colonization of multidrug-resistant bacteria and proinflammatory bacteria.

Furthermore, regarding such intestinal bacterial colonization suppressive ability, the present inventors have also succeeded in selecting 31 intestinal bacterial strains (F31mix) excluding duplicate bacteria from the 37 intestinal bacterial strains derived from healthy individual #F, and further selecting 18 strains (F18mix) capable of exhibiting bacterial colonization suppressive ability comparable to these (WO 2020/179868).

However, it has not been clarified that by what mechanism such intestinal bacterial strains can suppress intestinal colonization of proinflammatory bacteria and drug-resistant bacteria such as Kp2H7 strain.

### SUMMARY

### Technical Problem

The present invention has been made in view of the aforementioned problems of the related art. An object thereof is to clarify the mechanism by which intestinal colonization of proinflammatory bacteria, drug-resistant bacteria, etc. can be suppressed, thereby providing an antibacterial composition against such bacteria.

### Solution to Problem

In order to achieve the above object, the present inventors first prepared a mutant library (Kp2H7_tp) of Kp2H7 strain using a transposon. This library was administered to germ-free mice, followed by administration of F18mix or 13 bacterial strains in F31 mix that are not present in F18mix (F31-18mix); the present inventors then analyzed which gene was mutated in Kp2H7 strain contained in the feces, and the proportion of the mutant strain present, over time. As a result, the strain with a mutation in gntR became dominant in the F31-18mix administration group and the group administered only with Kp2H7_tp, while a gradual decrease in the gntR mutant strain was observed in the group administered with F18mix.

gntR is a gene related to gluconate metabolism. As shown in FIG. 5A, Klebsiella, Escherichia coli, etc. have the Entner-Doudoroff pathway (ED pathway) in addition to the common glycolytic pathway (EMP pathway) as a metabolic pathway for carbohydrate, and gluconate directly enters this ED pathway and is metabolized to pyruvate in three steps. gntR is known to act suppressively on gntU, gntK, edd, and eda, which are genes that metabolize gluconate in the ED pathway.

However, if there is a mutation in gntR, as shown in FIG. 5B, the suppression of gluconate metabolism is eliminated, so that the ED pathway works more actively. In a gluconate-rich environment, gntR mutant strains are expected to have a survival advantage. In other words, the present inventors presumed that the mouse's intestinal environment is gluconate-rich when only Kp2H7_tp was administered, or when F31-18mix is additionally administered.

On the other hand, in the presence of F18mix, the dominance of the gntR mutant strain is lost, indicating that gluconate in the environment is scarce and Klebsiella cannot utilize gluconate, which is an important carbon source. In other words, the present inventors considered this to be the result of a decrease in intestinal gluconate due to the utilization of gluconate by F18mix.

Therefore, based on the above presumption, the present inventors have conducted intensive studies and found that both Kp2H7 strain etc. and the 10 bacterial species in F31mix (including the 8 bacterial species in F18mix) are gluconate-utilizing bacteria, and by competing in this gluconate utilization, the 10 bacterial species etc. suppress the intestinal colonization of Kp2H7 strain etc. Similarly, it has been clarified that 9 bacterial species in the 42 intestinal bacterial derived from the healthy strains individual #I and 13 bacterial species in the 47 intestinal bacterial strains derived from the healthy individual #K are gluconate-utilizing bacteria.

Furthermore, as a result of analyzing the genes of such gluconate-utilizing bacteria, it has been found that in many of these bacteria, the gene encoding gluconate kinase or the gene encoding gluconate dehydratase and the gene encoding gluconate transporter are included in the same gene cluster, leading to the completion of the present invention.

That is, the present invention provides the following aspects.
[1] An antibacterial composition comprising at least one gluconate-utilizing first bacterial strain as an active agent, in which the composition exhibits antibacterial activity against a gluconate-utilizing second bacterial strain.
[2] The composition according to [1], in which the first bacterial strain comprises a gene encoding a gluconate transporter and at least one gene selected from the group consisting of a gene encoding gluconate kinase and a gene encoding gluconate dehydratase, in the same gene cluster.
[3] The composition according to [1], in which the first bacterial strain comprises a gene encoding gluconate dehydratase and a gene encoding a gluconate transporter in the same cluster.
[4] The composition according to any one of [1] to [3], in which the composition is ingested by a subject in combination with a food containing gluconate at a low concentration.
[5] The composition according to [4], in which the composition is ingested simultaneously with the food or separately from the food.
[6] The composition according to any one of [1] to [5], in which the second bacterial strain is pathogenic.
[7] The composition according to [6], in which the composition is a pharmaceutical composition.
[8] A method for screening a bacterial strain having antibacterial activity against a gluconate-utilizing bacterial strain, comprising: detecting gluconate utilization ability of a strain; and test bacterial determining that the test bacterial strain has antibacterial activity when the gluconate utilization ability is detected.
[9] A method for screening a bacterial strain having antibacterial activity against a gluconate-utilizing bacterial strain, comprising: detecting a gene encoding gluconate transporter and at least one gene selected from the group consisting of a gene encoding gluconate kinase and a gene encoding gluconate dehydratase, of a test bacterial strain; identifying the gene cluster corresponding to each detected gene; and determining that the test bacterial strain has antibacterial activity when the detected genes are located in the same gene cluster.
[10] A method for screening a bacterial strain having antibacterial activity against a gluconate-utilizing bacterial strain, comprising: detecting a gene encoding gluconate dehydratase and a gene encoding a gluconate transporter, of a test bacterial strain; identifying the gene cluster corresponding to each detected gene; and determining that the test bacterial stain has the antibacterial activity when the detected genes are located in the same gene cluster.
[11] A bacterial strain comprising a gene encoding a gluconate transporter and at least one gene selected from the group consisting of a gene encoding gluconate kinase and a gene encoding gluconate dehydratase, in the same gene cluster.
[12] A bacterial strain comprising a gene encoding gluconate dehydratase and a gene encoding a gluconate transporter in the same gene cluster.
[13] The composition according to [1], wherein the first bacterial strain is selected a group consisting of: a bacterial strain having a nucleotide sequence having at least 99% identity with the nucleotide sequence of SEQ ID NO: 17, a bacterial strain having a nucleotide sequence having at least 99% identity with the nucleotide sequence of SEQ ID NO: 18, a bacterial strain having a nucleotide sequence having at least 99% identity with the nucleotide sequence of SEQ ID NO: 20, a bacterial strain having a nucleotide sequence having at least 99% identity with the nucleotide sequence of SEQ ID NO: 21, a bacterial strain having a nucleotide sequence having at least 99% identity with the nucleotide sequence of SEQ ID NO: 22, a bacterial strain having a nucleotide sequence having at least 99% identity with the nucleotide sequence of SEQ ID NO: 23, a bacterial strain having a nucleotide sequence having at least 99% identity with the nucleotide sequence of SEQ ID NO: 32, and a bacterial strain having a nucleotide sequence having at least 99% identity with the nucleotide sequence of SEQ ID NO: 37.
[14] The composition according to [13], wherein the composition comprises eight gluconate-utilizing first bacterial strains.
[15] The composition according to [14], wherein the gluconate-utilizing first bacterial strains are: a bacterial strain having a nucleotide sequence having at least 99% identity with the nucleotide sequence of SEQ ID NO: 17, a bacterial strain having a nucleotide sequence having at least 99% identity with the nucleotide sequence of SEQ ID NO: 18, a bacterial strain having a nucleotide sequence having at least 99% identity with the nucleotide sequence of SEQ ID NO: 20, a bacterial strain having a nucleotide sequence having at least 99% identity with the nucleotide sequence of SEQ ID NO: 21, a bacterial strain having a nucleotide sequence having at least 99% identity with the nucleotide sequence of SEQ ID NO: 22, a bacterial strain having a nucleotide sequence having at least 99% identity with the nucleotide sequence of SEQ ID NO: 23, a bacterial strain having a nucleotide sequence having at least 99% identity with the nucleotide sequence of SEQ ID NO: 32, and a bacterial strain having a nucleotide sequence having at least 99% identity with the nucleotide sequence of SEQ ID NO: 37.
[16] The composition according to [6], wherein the second bacterial strain is Enterobacteriaceae.

### Advantageous Effects of Invention

According to the present invention, by using gluconate-utilizing bacteria (first bacteria) such as the 32 species (10 species derived from the healthy individual #F, 9 species derived from the healthy individual #I, and 13 species derived from the healthy individual #K) to compete with the gluconate utilization of proinflammatory bacteria or drug-resistant bacteria, etc. (second bacteria), it is possible to suppress the intestinal colonization of the second bacteria. Consequently, it becomes possible to prevent or treat diseases caused by the second bacteria. Furthermore, according to the present invention, it is also possible to screen bacteria having antibacterial activity against the second bacteria using gluconate utilization ability as an index.

### BRIEF DESCRIPTION OF THE DRAWINGS

FIG. 1 is a graph showing the time-dependent change in CFU of the bacterial amount of Kp2H7 strain in feces when Klebsiella (Kp2H7 strain) is administered to germ-free mice and, one week later, 37 intestinal bacterial strains (F37mix) or 18 strains (F18mix) isolated from a fecal sample of healthy individual #F are administered.

FIG. 2 is a diagram showing an overview of the preparation of a mutant library of Kp2H7 strain using a transposon.

FIG. 3 is a graph showing the time-dependent change in CFU of the bacterial amount of Kp2H7 strain in feces when the Kp2H7 strain mutant library (Kp2H7_tp) is administered to germ-free mice and, one week later, F18mix or 13 bacterial strains in F31mix that are not present in F18 mix (F31-18mix) are administered.

FIG. 4 is a graph showing the analysis of genes into which mutations were introduced in Kp-2H7 in feces collected on days 0, 4, 10, and 28 in the test process shown in FIG. 3 (Tn-seq) and the proportion thereof. In the figure, genes with a mutation introduction rate of 1% or less at all collection time points are shown in black. White bar graphs indicate the gntR gene.

FIG. 5A is a diagram showing an overview of the normal glycolytic pathway (EMP pathway) and Entner-Doudoroff (ED) pathway possessed by Klebsiella, etc.

FIG. 5B is a diagram showing an overview of changes in the progression of the EMP pathway and the ED pathway when there is a mutation in the gntR gene.

FIG. 6 is a graph showing the results of measuring the gluconate concentration in feces when F18mix or F31-18mix is administered to germ-free mice (GF).

FIG. 7 is a graph showing the time-dependent change in gluconate concentration in feces when Kp2H7 strain is administered to germ-free mice and, one week later, F18mix is administered.

FIG. 8 is a graph showing the results of a growth test of the wild-type strain (Kp_wt), gntK-deficient strain (Kp_ΔgntK), and gntR-deficient strain (Kp_ΔgntR) of Kp-2H7 using a minimum medium containing only glucose or gluconate as a carbon source.

FIG. 9 is a graph showing the amount of Klebsiella bacteria (CFU) in feces and the ratio in bacterial amount of mutant strain to wild-type strain in feces (Competition index (ΔgntR/WT)) when the wild-type strain and gntR-deficient strain of Kp2H7 are mixed in equal amounts and administered to germ-free mice, and F18mix or F31-18mix is administered 2 days later.

FIG. 10 is a graph showing the amount of Klebsiella bacteria (CFU) in feces and the ratio in bacterial amount of mutant strain to wild-type strain in feces (Competition index (ΔgntK/WT)) when the wild-type strain and gntK-deficient strain of Kp2H7 are mixed in equal amounts and administered to germ-free mice, and F18mix or F31-18mix is administered 2 days later.

FIG. 11 is a graph showing the amount of Klebsiella bacteria in feces one week after feeding germ-free mice colonized with Kp2H7 and F18mix with feed containing gluconate (GA) at concentrations of 0%, 2.5%, or 10%.

FIG. 12 is a graph showing the results of analyzing the expression of the gntK gene in Kp2H7 strain when Kp2H7 strain is administered to germ-free mice and F18mix or F31-18mix is administered 2 days later.

FIG. 13 is a graph showing the results of measuring the gluconate consumption concentration of 31 intestinal bacterial strains isolated from a fecal sample of healthy individual #F using LC-MS. In this graph, independent tests were performed three times, and the average value of the measured concentrations obtained is shown.

FIG. 14 is a graph showing the results of measuring the gluconate consumption concentration of pathogenic microorganisms using LC-MS.

FIG. 15 is a graph showing the time-dependent change in CFU of the bacterial amount of Kp2H7 strain in feces when Kp2H7 strain is administered to germ-free mice and, one week later, F18mix, F8mix, or F10mix is administered. F8mix indicates the 8 bacterial strains that were found to have ability to utilize gluconate in F18mix as shown in FIG. 13. F10mix indicates the 10 bacterial strains in F18 mix that are not in F8mix.

FIG. 16A is a graph showing the time-dependent change in CFU of the bacterial amount of Kp2H7 strain in feces when Kp2H7 strain is administered to germ-free mice, F18mix is administered one week later, and from the 21st day, feed containing gluconate (GA) at concentrations of 08, 2.5%, or 10% is given.

FIG. 16B is a graph showing the time-dependent change in CFU of the bacterial amount of Kp2H7 strain in feces on Day 28 in the test shown in FIG. 16A.

FIG. 16C is a graph showing the time-dependent change in CFU of the bacterial amounts (relative amounts) of Kp2H7 strain and F18mix in feces in the test shown in FIG. 16A.

FIG. 16D is a graph showing the time-dependent change in CFU of the bacterial amounts (absolute amounts) of Kp2H7 strain and F18mix in feces in the test shown in FIG. 16A.

FIG. 17A is a graph showing the results of RNA-seq analysis of bacteria in feces after administering either Kp-2H7 alone or Kp-2H7 and F18mix to germ-free mice and collecting fecal samples 2 days later. In the figure, “Kp+F18-mix>Kp only” shows the pathway composed of genes whose expression was increased in the Kp-2H7 and F18mix administration group compared to the Kp-2H7 alone administration group. “Kp+F18-mix<Kp only” shows the pathway composed of genes whose expression was decreased in the Kp-2H7 and F18mix administration group compared to the Kp-2H7 alone administration group.

FIG. 17B is a heatmap showing the expression profile of genes involved in carbohydrates metabolism in bacteria in feces after administering either Kp-2H7 alone or Kp-2H7 and F18mix to germ-free mice and collecting fecal samples 2 days later.

FIG. 18A is a graph showing the results of analyzing the gluconate concentration in the feces of germ-free mice raised on either gluconate-containing nutrient-rich feed or gluconate-free feed. In the figure, “GF on CL-2” shows the results of the gluconate-containing nutrient-rich feed intake group, and “GF on AlN93G” shows the results of the gluconate-free feed intake group.

FIG. 18B is a graph showing the results of administering Kp-2H7 to germ-free mice raised on gluconate-containing nutrient-rich feed (CL-2), changing to gluconate-free feed (AlN93G) on day 21, and analyzing the Kp-2H7 concentration in feces over time.

FIG. 19A is a diagram showing the results of measuring the gluconate concentration in culture supernatants after culturing each bacterial strain isolated from healthy individuals #F, #K, and #I in medium containing 300 UM gluconate, and an overview of gene clusters related to the classical pathway (gluconate kinase) in each bacterial strain.

FIG. 19B is a diagram showing the results of measuring the gluconate concentration in culture supernatants after culturing each bacterial strain isolated from healthy individuals #F, #K, and #I in medium containing 300 UM gluconate, and an overview of gene clusters related to the alternative pathway (gluconate dehydratase) in each bacterial strain.

FIG. 19C is a diagram showing the results of measuring the gluconate concentration in culture supernatants after culturing each bacterial strain isolated from healthy individuals #F, #K, and #I in medium containing 300 UM gluconate, and an overview of gene clusters related to the gluconate transporter in each bacterial strain.

FIG. 19D is a diagram showing the results of measuring the gluconate concentration in culture supernatants after culturing each bacterial strain isolated from healthy individuals #F, #K, and #I, in which no possession of genes involved in gluconate metabolism was observed, in medium containing 300 UM gluconate.

FIG. 19E is a diagram showing an overview of the gluconate metabolic pathway (classical pathway or alternative pathway) involving gluconate kinase or gluconate dehydratase.

FIG. 20 is a graph showing the results of administering 18 intestinal bacterial strains (F18mix) or 13 strains (F13mix) isolated from a fecal sample of healthy individual #F to germ-free mice, or administering nothing, administering Kp2H7 strain two weeks later, and analyzing the time-dependent change in the fecal concentration of the bacterial strain (4 mice in each administration group).

### DETAILED DESCRIPTION

The present inventors have previously found that 37 strains of intestinal bacterial strains isolated and cultured from a fecal sample derived from healthy individual #F (37 bacterial species shown in Table 1 below) can suppress intestinal colonization of proinflammatory bacteria such as Kp2H7 strain. The present inventors have also succeeded in selecting 31 intestinal bacterial strains (F31mix) excluding duplicate bacteria from the 37 intestinal bacterial strains, and further selecting 18 strains (F18mix) capable of exhibiting intestinal colonization suppressive ability comparable to these.

TABLE 1

| | | Depositary Information | | | | | |
|---|---|---|---|---|---|---|---|
| | SEQ | Identi- | Accession | Date of | | | Ability |
| | ID | fiation | Number | Original | F31 | F18 | to utilize |
| No. | NO: | Reference | (Nite BP-) | Deposit | mix | mix | gluconate |
| f01 | 1 | f1_42H6 | 3147 | Mar. 2, 2020 | ● | ● | − |
| f02 | 2 | — | — | — | ● | — | − |
| f03 | 3 | — | — | — | ● | — | − |
| f04 | 4 | — | — | — | ● | — | − |
| f05 | 5 | — | — | — | ● | — | − |
| f06 | 6 | — | — | — | — | — | n.d. |
| f07 | 7 | — | — | — | ● | — | − |
| f08 | 8 | — | — | — | — | — | n.d. |
| f09 | 9 | — | — | — | ● | — | − |
| f10 | 10 | f10_43J3 | 3800 | Jan. 12, 2023 | ● | — | + |
| f11 | 11 | — | — | — | ● | — | − |
| f12 | 12 | f12_42H4 | 3148 | Mar. 2, 2020 | ● | ● | − |
| f13 | 13 | — | — | — | ● | — | − |
| f14 | 14 | — | — | — | ● | — | − |
| f15 | 15 | — | — | — | — | — | n.d. |
| f16 | 16 | — | — | — | ● | — | − |
| f17 | 17 | f17_42I7 | 3149 | Mar. 2, 2020 | ● | ● | + |
| f18 | 18 | f18_42I2 | 3150 | Mar. 2, 2020 | ● | ● | + |
| f19 | 19 | f19_43G2 | 3151 | Mar. 2, 2020 | ● | ● | − |
| f20 | 20 | f20_43G1 | 3152 | Mar. 2, 2020 | ● | ● | + |
| f21 | 21 | f21 42A8 | 3153 | Mar. 2, 2020 | ● | ● | + |
| f22 | 22 | f22 43C3 | 3154 | Mar. 2, 2020 | ● | ● | + |
| f23 | 23 | f23_42K4 | 3155 | Mar. 2, 2020 | ● | ● | + |
| f24 | 24 | f24_42I4 | 3156 | Mar. 2, 2020 | ● | ● | − |
| f25 | 25 | — | — | — | — | — | n.d. |
| f26 | 26 | f26_42K2 | 3157 | Mar. 2, 2020 | ● | ● | − |
| f27 | 27 | — | — | — | — | — | n.d. |
| f28 | 28 | f28 43A3 | 3158 | Mar. 2, 2020 | ● | ● | − |
| f29 | 29 | f29_43J8 | 3801 | Jan. 12, 2023 | ● | — | + |
| f30 | 30 | f30_43A5 | 3159 | Mar. 2, 2020 | ● | ● | − |
| f31 | 31 | f31_43J5 | 3160 | Mar. 2, 2020 | ● | ● | − |
| f32 | 32 | f32_42A7 | 3161 | Mar. 2, 2020 | ● | ● | + |
| f33 | 33 | f33 43N2 | 3162 | Mar. 2, 2020 | ● | ● | − |
| f34 | 34 | — | — | — | ● | — | − |
| f35 | 35 | f35_42L8 | 3163 | Mar. 2, 2020 | ● | ● | − |
| f36 | 36 | — | — | — | — | — | n.d. |
| f37 | 37 | f37_42G1 | 3164 | Mar. 2, 2020 | ● | ● | + |

The 37 bacterial strains listed in Table 1 above correspond to f01 to f37 described in WO 2019/017389 and WO 2020/179868. The sequences of their 16S rDNA are set forth in SEQ ID NOs: 1 to 37, respectively. “F31mix” and “F18mix” are bacteria selected in WO 2020/179868 as described above, and the bacteria belonging to these are each indicated by a black circle in Table 1 above. Furthermore, for 37 bacterial strains that have been deposited as microorganisms, Table 1 above shows their identification references, accession numbers, and the dates of original deposits. All of these deposited bacterial strains are deposited at the National Institute of Technology and Evaluation (NITE), Patent Microorganisms Depositary (Room 122, 2-5-8 Kazusa-Kamatari, Kisarazu, Chiba 292-0818, Japan).

The present inventors have identified that both Kp2H7 strain etc. and the 10 bacterial strains in F31mix (including the 8 bacterial strains in F18mix) are gluconate-utilizing bacteria, and by competing in this gluconate utilization, the 10 bacterial strains etc. suppress the intestinal colonization of Kp2H7 strain etc. The 10 bacterial strains are indicated by + (plus) in Table 1. On the other hand, − (minus) indicates that the bacteria do not utilize gluconate. Also, “n.d.” indicates bacteria whose gluconate utilization ability has not been evaluated.

The present inventors have also found that 42 species of intestinal bacterial strains isolated and cultured from a fecal sample derived from healthy individual #I (42 bacterial species excluding I14 in Table 2 below) can suppress intestinal colonization of proinflammatory bacteria such as Kp2H7 strain. I01 to I13 and I15 to I43 in Table 2 below correspond to I01 to I13 and I14 to I42 described in WO 2019/017389 and WO 2020/179868, respectively. The sequences of their 16S rDNA are set forth in SEQ ID NOs: 168 to 209, respectively.

TABLE 2

| | | SEQ ID | | NCBI | % | Ability to utilize |
|---|---|---|---|---|---|---|
| No. | Species | NO: | subject id | TAX ID | identity | gluconate |
| I01 | Bifidobacterium faecale | 168 | NR_133982.1 | 1454229 | 98.06 | + |
| I02 | Bifidobacterium pseudocatenulatum | 169 | NR_037117.1 | 28026 | 99.63 | + |
| I03 | Bifidobacterium bifidum | 170 | NR_044771.1 | 1681 | 100.00 | − |
| I04 | Bifidobacterium longum | 171 | NR_145535.1 | 1931217 | 99.70 | − |
| I05 | Collinsella aerofaciens | 172 | NR_113316.1 | 74426 | 99.92 | − |
| I06 | Collinsella aerofaciens | 173 | NR_113316.1 | 74426 | 99.72 | − |
| I07 | Bifidobacterium longum | 174 | NR_145535.1 | 1931217 | 99.70 | − |
| I08 | Bacteroides stercoris | 175 | NR_112943.1 | 449673 | 99.18 | − |
| I09 | Bacteroides massiliensis | 176 | NR_042745.1 | 204516 | 99.70 | − |
| I10 | Bacteroides vulgatus | 177 | NR_074515.1 | 435590 | 100.00 | − |
| I11 | Bacteroides dorei | 178 | NR_041351.1 | 357276 | 100.00 | − |
| I12 | Parabacteroides merdae | 179 | NR_041343.1 | 46503 | 99.85 | − |
| I13 | Parabacteroides distasonis | 180 | NR_041342.1 | 823 | 99.03 | − |
| I14 | — | — | — | — | — | n.d. |
| I15 | Alistipes putredinis | 181 | NR_113152.1 | 28117 | 100.00 | − |
| I16 | Bacteroides uniformis | 182 | NR_040866.1 | 820 | 97.75 | − |
| I17 | Bacteroides koreensis | 183 | NR_159117.1 | 1912896 | 99.26 | − |
| I18 | Alistipes shahii | 184 | NR_113153.1 | 328814 | 99.63 | − |
| I19 | Odoribacter splanchnicus | 185 | NR_113075.1 | 28118 | 99.77 | − |
| I20 | Faecalibacterium prausnitzii | 186 | NR_028961.1 | 853 | 97.24 | + |
| I21 | Faecalibacterium prausnitzii | 187 | NR_028961.1 | 853 | 97.24 | + |
| I22 | Blautia wexlerae | 188 | NR_044054.1 | 1121115 | 98.38 | + |
| I23 | Ruminococcus lactaris | 189 | NR_027579.1 | 471875 | 97.24 | + |
| I24 | Ruminococcus albus | 190 | NR_113032.1 | 1264 | 94.68 | − |
| I25 | Faecalibacterium prausnitzii | 191 | NR_028961.1 | 853 | 96.86 | − |
| I26 | Dorea longicatena | 192 | NR_028883.1 | 88431 | 99.47 | − |
| I27 | Dorea formicigenerans | 193 | NR_044645.2 | 39486 | 97.94 | − |
| I28 | Anaerostipes hadrus | 194 | NR_117138.2 | 649756 | 99.85 | − |
| I29 | Intestinibacter bartlettii | 195 | NR_027573.1 | 261299 | 99.55 | − |
| I30 | Flavonifractor plautii | 196 | NR_029356.1 | 292800 | 99.71 | − |
| I31 | Pseudoflavonifractor phocaeensis | 197 | NR_147370.1 | 1870988 | 97.86 | − |
| I32 | [Clostridium] spiroforme | 198 | NR_114393.1 | 29348 | 93.09 | − |
| I33 | Megasphaera elsdenii | 199 | NR_102980.1 | 1064535 | 99.35 | + |
| I34 | Dialister succinatiphilus | 200 | NR_041666.1 | 742743 | 97.02 | − |
| I35 | Acidaminococcus intestini | 201 | NR_041894.1 | 187327 | 99.93 | − |
| I36 | Allisonella histaminiformans | 202 | NR_028862.1 | 209880 | 99.50 | n.d. |
| I37 | Megasphaera massiliensis | 203 | NR_133027.1 | 1232428 | 99.00 | + |
| I38 | Sutterella wadsworthensis | 204 | NR_104851.1 | 40545 | 99.78 | − |
| I39 | Clostridium baratii | 205 | NR_029229.1 | 1561 | 99.85 | − |
| I40 | Anaeromassilibacillus senegalensis | 206 | NR_144727.1 | 1673717 | 97.27 | − |
| I41 | Colidextribacter massiliensis | 207 | NR_147375.1 | 1870986 | 95.00 | − |
| I42 | Flavonifractor plautii | 208 | NR_029356.1 | 292800 | 97.96 | − |
| I43 | [Clostridium] leptum | 209 | NR_114789.1 | 1535 | 94.55 | − |

Furthermore, it has been found that 47 strains of intestinal bacterial strains isolated and cultured from a fecal sample derived from healthy individual #K (47 bacterial species shown in Table 3 below) can suppress intestinal colonization of proinflammatory bacteria such as Kp2H7 strain. K01 to K44 and K46 to K47 in Table 3 below correspond to K01 to K44 and K45 to K46 described in WO 2019/017389 and WO 2020/179868, respectively. The sequences of their 16s rDNA are set forth in SEQ ID NOs: 210 to 255, respectively.

TABLE 3

| | | SEQ ID | | NCBI | % | Ability to utilize |
|---|---|---|---|---|---|---|
| No. | Species | NO: | subject id | TAX ID | identity | gluconate |
| K01 | Drancourtella massiliensis | 210 | NR_144722.1 | 1632013 | 99.57 | − |
| K02 | Bacteroides ovatus | 211 | NR_112940.1 | 28116 | 99.86 | − |
| K03 | Blautia coccoides | 212 | NR_104700.1 | 1532 | 99.86 | + |
| K04 | Blautia hominis | 213 | NR_163638.1 | 2025493 | 99.43 | + |
| K05 | Desulfovibrio vulgaris | 214 | NR_074446.1 | 882 | 91.58 | − |
| K06 | Alistipes onderdonkii | 215 | NR_043318.1 | 328813 | 99.86 | − |
| K07 | Eisenbergiella massiliensis | 216 | NR_144731.1 | 1720294 | 99.50 | − |
| K08 | [Clostridium] innocuum | 217 | NR_029164.1 | 1522 | 97.93 | + |
| K09 | Bacteroides fragilis | 218 | NR_112936.1 | 817 | 99.71 | − |
| K10 | Eggerthella lenta | 219 | NR_074377.1 | 84112 | 100.00 | − |
| K11 | Dielma fastidiosa | 220 | NR_125593.1 | 1034346 | 99.71 | − |
| K12 | Erysipelatoclostridium ramosum | 221 | NR_113243.1 | 1547 | 100.00 | − |
| K13 | Enterococcus faecalis | 222 | NR_115765.1 | 1351 | 99.93 | + |
| K14 | Bacteroides intestinalis | 223 | NR_041307.1 | 329854 | 99.349 | − |
| K15 | [Clostridium] symbiosum | 224 | NR_118730.1 | 1512 | 98.345 | − |
| K16 | [Clostridium] hylemonae | 225 | NR_024719.1 | 89153 | 99.50 | − |
| K17 | Hungatella effluvii | 226 | NR_133762.1 | 1096246 | 98.49 | + |
| K18 | Bacteroides dorei | 227 | NR_041351.1 | 357276 | 99.93 | − |
| K19 | [Clostridium] clostridioforme | 228 | NR_044715.2 | 1531 | 98.99 | + |
| K20 | Flavonifractor plautii | 229 | NR_029356.1 | 292800 | 100.00 | − |
| K21 | Bacteroides xylanisolvens | 230 | NR_112947.1 | 657309 | 99.50 | − |
| K22 | Bacteroides thetaiotaomicron | 231 | NR_112944.1 | 818 | 99.93 | − |
| K23 | Parabacteroides merdae | 232 | NR_041343.1 | 46503 | 99.78 | − |
| K24 | Bacteroides vulgatus | 233 | NR_074515.1 | 435590 | 100.00 | − |
| K25 | [Clostridium] aldenense | 234 | NR_043680.1 | 358742 | 99.41 | − |
| K26 | Bacteroides uniformis | 235 | NR_112945.1 | 820 | 97.39 | − |
| K27 | Gordonibacter urolithinfaciens | 236 | NR_148261.1 | 1335613 | 99.56 | − |
| K28 | Coprococcus comes | 237 | NR_044048.1 | 470146 | 92.77 | − |
| K29 | Anaerostipes caccae | 238 | NR_028915.1 | 105841 | 98.78 | − |
| K30 | [Ruminococcus] gnavus | 239 | NR_036800.1 | 411470 | 99.78 | + |
| K31 | [Ruminococcus] gnavus | 240 | NR_036800.1 | 411470 | 98.71 | + |
| K32 | Alistipes shahii | 241 | NR_113153.1 | 328814 | 100.00 | − |
| K33 | Bacteroides stercoris | 242 | NR_112943.1 | 449673 | 98.77 | − |
| K34 | Blautia hominis | 243 | NR_163634.1 | 2025493 | 98.71 | + |
| K35 | Butyricicoccus faecihominis | 244 | NR_152060.1 | 1712515 | 97.89 | − |
| K36 | [Clostridium] bolteae | 245 | NR_025567.1 | 208479 | 99.50 | + |
| K37 | Phocea massiliensis | 246 | NR_144748.1 | 1841867 | 99.93 | − |
| K38 | Holdemania massiliensis | 247 | NR_125628.1 | 1211819 | 99.71 | − |
| K39 | Escherichia coli | 248 | NR_114042.1 | 562 | 99.79 | + |
| K40 | Agathobaculum desmolans | 249 | NR_044644.2 | 39484 | 96.68 | + |
| K41 | [Eubacterium] rectale | 250 | NR_074634.1 | 515619 | 100.00 | − |
| K42 | Lactonifactor longoviformis | 251 | NR_043551.1 | 341220 | 100.00 | + |
| K43 | Oscillibacter ruminantium | 252 | NR_118156.1 | 1007096 | 95.90 | − |
| K44 | Pseudoflavonifractor phocaeensis | 253 | NR_147370.1 | 1870988 | 97.42 | − |
| K45 | | | | | | n.d. |
| K46 | Streptococcus pasteurianus | 254 | NR_043660.1 | 197614 | 100.00 | − |
| K47 | Sutterella wadsworthensis | 255 | NR_117778.1 | 40545 | 99.93 | − |

The bacteria that have been identified to utilize gluconate are indicated by + (plus) in Tables 2 and 3. On the other hand, − (minus) indicates that the bacteria do not consume gluconate. Also, “n.d.” indicates bacteria whose gluconate utilization ability has not been evaluated.

### Antibacterial Composition

Therefore, the present invention relates to a method etc. that use gluconate-utilizing bacteria (first bacteria) such as the 32 strains (10 species derived from the healthy individual #F, 9 strains derived from the healthy individual #I, and 13 strains derived from the healthy individual #K) described above to compete with gluconate utilization/consumption of proinflammatory bacteria or drug-resistant bacteria etc. (second bacteria), thereby suppressing intestinal colonization of these bacteria. That is, the present invention provides an antibacterial composition including one and more gluconate-utilizing first bacterium/bacteria as an active ingredient, the composition being against one and more gluconate-utilizing second bacterium/bacteria.

In the present invention, “antibacterial” means suppression of the activity of bacteria, more specifically, suppression of proliferation, growth, or colonization of bacteria, or killing of bacteria, and includes, for example, suppression of bacterial colonization in the intestine and elimination of bacteria from the intestine.

In the present invention, “gluconate” means a carboxylic acid produced by oxidizing the carbon at position 1 of glucose, also referred to as (2R, 3S, 4R, 5R)-2, 3, 4, 5, 6-pentahydroxyhexanoic acid. Further, the gluconate according to the present invention includes not only the carboxylic acid but also gluconolactone (glucono-8-lactone) in equilibrium in an aqueous solution. “Consumption of gluconate” and “utilization of gluconate” mean that at least the bacteria take up gluconate from the surrounding environment, and may further include conversion (catabolism or anabolism, metabolism or assimilation, etc.) of the taken-up gluconate into other compounds. The surrounding environment is not particularly limited, and examples thereof include the host's intestine and a culture medium.

In the present invention, gluconate-utilizing bacteria mean bacteria that have gluconate consumption ability/gluconate utilization ability. Here, whether or not bacteria have gluconate utilization ability can be evaluated, for example, as shown in Examples described later, by culturing the bacteria in a medium containing gluconate and determining whether the amount of gluconate remaining in the medium is significantly reduced compared to before culture. More specifically, as shown in Examples described later, if the gluconate concentration remaining in the culture supernatant after culturing a bacterial culture in the stationary phase in a 300 μM gluconate-containing medium at a volume ratio of 100:1 at 37° C. under anaerobic conditions for 48 hours is 100 μM or less (preferably 70 μM or less, more preferably 50 μM or less, still more preferably 30 μM or less, more preferably 25 μM or less, still more preferably 20 μM or less, more preferably 15 UM or less), the bacteria can be evaluated to have gluconate utilization (i.e. consumption) ability.

Furthermore, as shown in Examples described later, bacteria can also be evaluated to have gluconate utilization/consumption ability if the bacteria are ingested by an animal, the amount of gluconate in the feces of the animal is detected, and it is significantly reduced compared to the amount when the bacteria are not ingested. The animal is not particularly limited, and examples thereof include humans, pigs, cattle, horses, sheep, goats, chickens, wild ducks, ostriches, domestic ducks, dogs, cats, rabbits, hamsters, mice, rats, and monkeys.

### Gluconate-Utilizing First Bacteria

In the present invention, “gluconate-utilizing first bacterial strain”, “gluconate-utilizing first bacterium” and “gluconate-utilizing first bacteria” are not particularly limited as long as they are bacteria that have gluconate utilization/consumption ability and can colonize the intestine; however, they are intestinal bacteria whose residual gluconate concentration in the method is preferably 30 μM or less, more preferably 25 μM or less, still more preferably 20 μM or less, more preferably 15 μM or less, still more preferably 10 μM or less, more preferably 5 μM or less, still more preferably 2 μM or less.

More specifically, examples thereof include at least one bacterium (preferably 2 or more (for example, 3 or 4), more preferably 5 or more (for example, 6 or 7), still more preferably 8 or more (for example, 9 or 10) bacteria, more preferably 11 or more (for example, 12, 13, or 14) bacteria, still more preferably 15 or more (for example, 16 or 17), more preferably 18 or more (for example, 19 or 20) bacteria, still more preferably 21 or more (for example, 22, 23, or 24) bacteria, more preferably 25 or more (for example, 26 or 27), and still more preferably 28 or more (for example, 29, 30, 31, or 32) bacteria) selected from the group consisting of bacteria having DNA showing at least 90% identity to the sequence set forth in SEQ ID NO: 10, bacteria having DNA showing at least 90% identity to the sequence set forth in SEQ ID NO: 17, bacteria having DNA showing at least 90% identity to the sequence set forth in SEQ ID NO: 18, bacteria having DNA showing at least 90% identity to the sequence set forth in SEQ ID NO: 20, bacteria having DNA showing at least 90% identity to the sequence set forth in SEQ ID NO: 21, bacteria having DNA showing at least 90% identity to the sequence set forth in SEQ ID NO: 22, bacteria having DNA showing at least 90% identity to the sequence set forth in SEQ ID NO: 23, bacteria having DNA showing at least 90% identity to the sequence set forth in SEQ ID NO: 29, bacteria having DNA showing at least 90% identity to the sequence set forth in SEQ ID NO: 32, bacteria having DNA showing at least 90% identity to the sequence set forth in SEQ ID NO: 37, bacteria having DNA showing at least 90% identity to the sequence set forth in SEQ ID NO: 168, bacteria having DNA showing at least 90% identity to the sequence set forth in SEQ ID NO: 169, bacteria having DNA showing at least 90% identity to the sequence set forth in SEQ ID NO: 186, bacteria having DNA showing at least 90% identity to the sequence set forth in SEQ ID NO: 187, bacteria having DNA showing at least 90% identity to the sequence set forth in SEQ ID NO: 188, bacteria having DNA showing at least 90% identity to the sequence set forth in SEQ ID NO: 189, bacteria having DNA showing at least 90% identity to the sequence set forth in SEQ ID NO: 191, bacteria having DNA showing at least 90% identity to the sequence set forth in SEQ ID NO: 199, bacteria having DNA showing at least 90% identity to the sequence set forth in SEQ ID NO: 203, bacteria having DNA showing at least 90% identity to the sequence set forth in SEQ ID NO: 212, bacteria having DNA showing at least 90% identity to the sequence set forth in SEQ ID NO: 213, bacteria having DNA showing at least 90% identity to the sequence set forth in SEQ ID NO: 217, bacteria having DNA showing at least 90% identity to the sequence set forth in SEQ ID NO: 222, bacteria having DNA showing at least 90% identity to the sequence set forth in SEQ ID NO: 226, bacteria having DNA showing at least 90% identity to the sequence set forth in SEQ ID NO: 228, bacteria having DNA showing at least 90% identity to the sequence set forth in SEQ ID NO: 239, bacteria having DNA showing at least 90% identity to the sequence set forth in SEQ ID NO: 240, bacteria having DNA showing at least 90% identity to the sequence set forth in SEQ ID NO: 243, bacteria having DNA showing at least 90% identity to the sequence set forth in SEQ ID NO: 245, bacteria having DNA showing at least 90% identity to the sequence set forth in SEQ ID NO: 248, bacteria having DNA showing at least 90% identity to the sequence set forth in SEQ ID NO: 249, and bacteria having DNA showing at least 90% identity to the sequence set forth in SEQ ID NO: 251.

Furthermore, from the viewpoint of higher gluconate utilization/consumption ability among the 32 bacteria, examples thereof include at least one bacterium (preferably 2 or more (for example, 3 or 4), more preferably 5 or more (for example, 6 or 7), still more preferably 8 or more (for example, 9 or 10) bacteria, more preferably 11 or more (for example, 12, 13, or 14) bacteria, still more preferably 15 or more (for example, 16 or 17), more preferably 18 or more (for example, 19 or 20) bacteria, and still more preferably 21 or more (for example, 22, 23, or 24) bacteria) selected from the group consisting of bacteria having DNA showing at least 90% identity to the sequence set forth in SEQ ID NO: 10, bacteria having DNA showing at least 90% identity to the sequence set forth in SEQ ID NO: 17, bacteria having DNA showing at least 90% identity to the sequence set forth in SEQ ID NO: 18, bacteria having DNA showing at least 90% identity to the sequence set forth in SEQ ID NO: 21, bacteria having DNA showing at least 90% identity to the sequence set forth in SEQ ID NO: 22, bacteria having DNA showing at least 90% identity to the sequence set forth in SEQ ID NO: 29, bacteria having DNA showing at least 90% identity to the sequence set forth in SEQ ID NO: 32, bacteria having DNA showing at least 90% identity to the sequence set forth in SEQ ID NO: 37, bacteria having DNA showing at least 90% identity to the sequence set forth in SEQ ID NO: 168, bacteria having DNA showing at least 90% identity to the sequence set forth in SEQ ID NO: 169, bacteria having DNA showing at least 90% identity to the sequence set forth in SEQ ID NO: 188, bacteria having DNA showing at least 90% identity to the sequence set forth in SEQ ID NO: 189, bacteria having DNA showing at least 90% identity to the sequence set forth in SEQ ID NO: 199, bacteria having DNA showing at least 90% identity to the sequence set forth in SEQ ID NO: 203, bacteria having DNA showing at least 90% identity to the sequence set forth in SEQ ID NO: 212, bacteria having DNA showing at least 90% identity to the sequence set forth in SEQ ID NO: 213, bacteria having DNA showing at least 90% identity to the sequence set forth in SEQ ID NO: 226, bacteria having DNA showing at least 90% identity to the sequence set forth in SEQ ID NO: 228, bacteria having DNA showing at least 90% identity to the sequence set forth in SEQ ID NO: 239, bacteria having DNA showing at least 90% identity to the sequence set forth in SEQ ID NO: 240, bacteria having DNA showing at least 90% identity to the sequence set forth in SEQ ID NO: 243, bacteria having DNA showing at least 90% identity to the sequence set forth in SEQ ID NO: 245, bacteria having DNA showing at least 90% identity to the sequence set forth in SEQ ID NO: 248, and bacteria having DNA showing at least 90% identity to the sequence set forth in SEQ ID NO: 251.

Furthermore, the first bacteria may be, for example, at least one bacterium (preferably 2 or more (for example, 3 or 4), more preferably 5 or more (for example, 6 or 7), particularly preferably 8 bacteria) selected from the group consisting of bacteria having DNA showing at least 90% identity to the sequence set forth in SEQ ID NO: 17, bacteria having DNA showing at least 90% identity to the sequence set forth in SEQ ID NO: 18, bacteria having DNA showing at least 90% identity to the sequence set forth in SEQ ID NO: 20, bacteria having DNA showing at least 90% identity to the sequence set forth in SEQ ID NO: 21, bacteria having DNA showing at least 90% identity to the sequence set forth in SEQ ID NO: 22, bacteria having DNA showing at least 90% identity to the sequence set forth in SEQ ID NO: 23, bacteria having DNA showing at least 90% identity to the sequence set forth in SEQ ID NO: 32, and bacteria having DNA showing at least 90% identity to the sequence set forth in SEQ ID NO: 37.

Furthermore, in the present invention, the first bacteria are preferably at least one bacterium, more preferably 2 or more bacteria, still more preferably 3 or more bacteria, and particularly preferably 4 bacteria, selected from the group consisting of bacteria having DNA showing at least 90% identity to the sequence set forth in SEQ ID NO: 18, bacteria having DNA showing at least 90% identity to the sequence set forth in SEQ ID NO: 21, bacteria having DNA showing at least 90% identity to the sequence set forth in SEQ ID NO: 22, and bacteria having DNA showing at least 90% identity to the sequence set forth in SEQ ID NO: 37, from the viewpoint of higher gluconate consumption ability (specifically, the gluconate concentration in the culture supernatant after 48 hours in Example 3 described later is 10 μM or less).

Furthermore, as shown in Examples described later, bacteria having, in the same gene cluster, a gene encoding a gluconate transporter and at least one gene selected from the group consisting of a gene encoding gluconate kinase and a gene encoding gluconate dehydratase may have gluconate utilization/consumption ability.

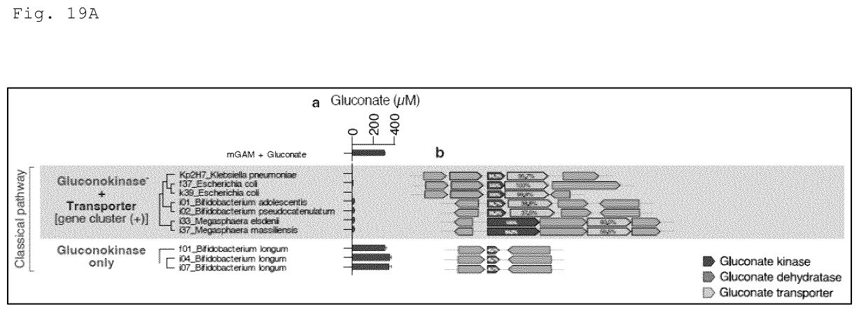

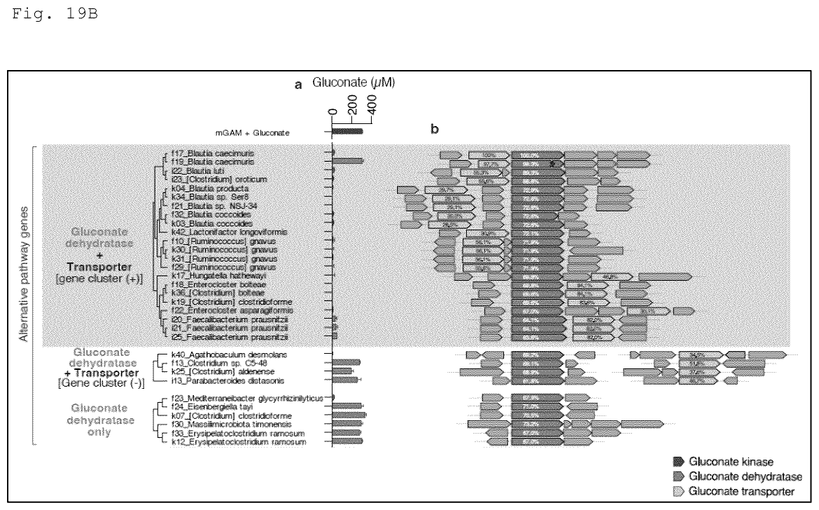

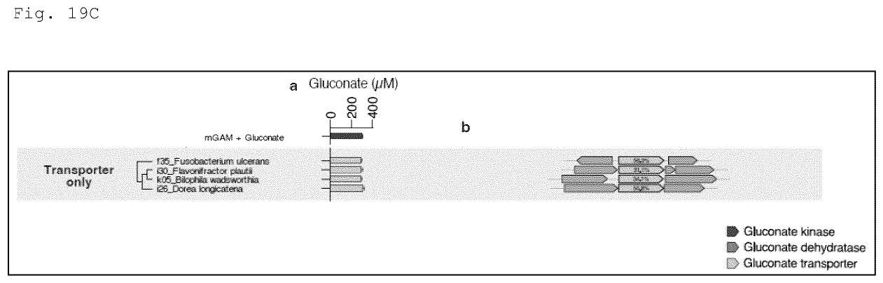

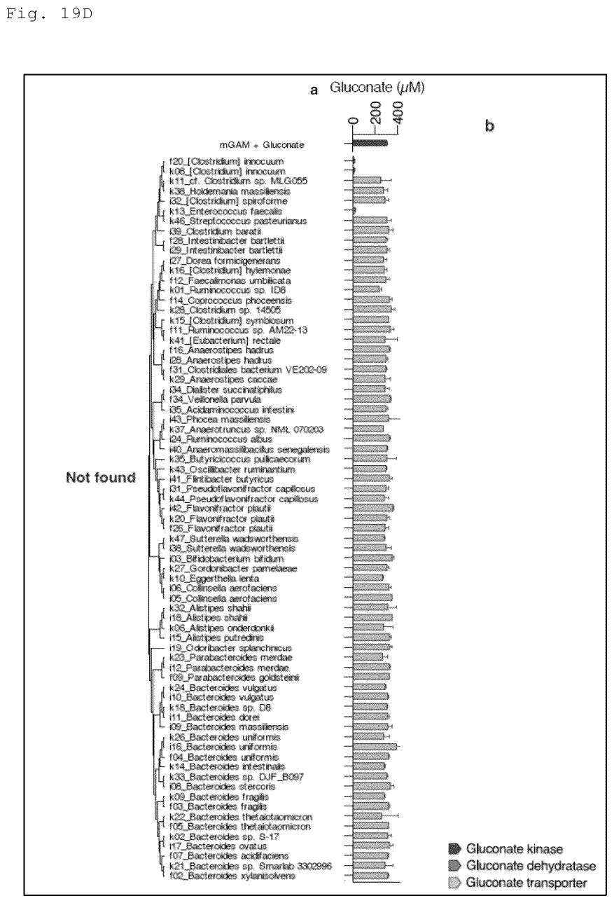

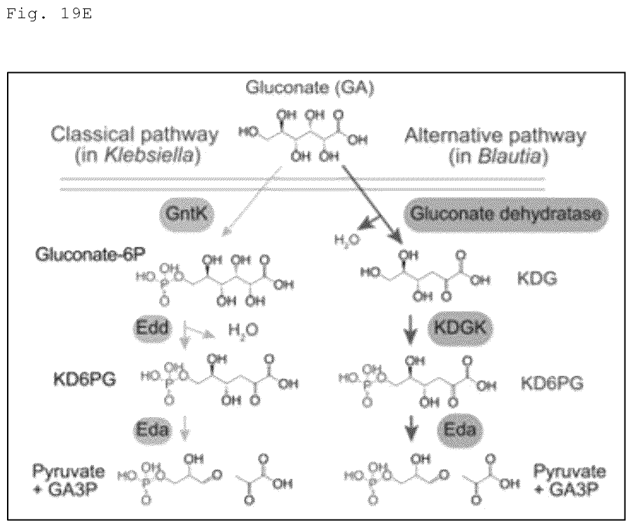

In the present invention, “gluconate kinase (gluconokinase; gntK)” means a phosphotransferase (EC 2.7.1.12) that catalyzes the following reaction (see FIG. 19E):

ATP+gluconate⇔ADP+6-phosphogluconate.

Examples of such gluconate kinase include proteins having an amino acid sequence showing homology to at least one amino acid sequence of SEQ ID NOs: 76 to 85 (see Tables 9 to 12 presented later).

In the present invention, “gluconate dehydratase (gad)” means a dehydrogenase (EC 4.2.1.39) that catalyzes the following reaction (see FIG. 21):

Gluconate⇔2-keto-3-deoxygluconate (KDG)+H2O.

Examples of such gluconate dehydratase include proteins having an amino acid sequence showing homology to at least one amino acid sequence of SEQ ID NOs: 86 to 117 (see Tables 9 to 11 presented later).

In the present invention, “gluconate transporter” means a protein that transports sugar acid molecules such as gluconate or sugar-keto acids.

Examples of such gluconate transporters include proteins having an amino acid sequence showing homology to at least one amino acid sequence of SEQ ID NOs: 118 to 167 (see Tables 9 to 12 presented later).

In the present invention, “showing homology” means showing homology of 60% or more coverage and 60% or more percent identity to the amino acid sequence shown in each of the above SEQ ID NOs. Here, coverage (query cover, qcovhsp) represents the ratio of the alignment length to the total length of the query amino acid sequence, and percent identity (id, pident) represents the percentage of amino acid identity to the alignment length. The 60% or more here is preferably 70% or more, more preferably 80% or more, still more preferably 85% or more, more preferably 90% or more (91% or more, 92% or more, 93% or more, 94% or more), and still more preferably 95% or more (96% or more, 97% or more, 98% or more, 99% or more, 100%), independently of each other for coverage and percent identity.

In the present invention, a “gene cluster” means a group of genes that are encoded on the same chromosome and are encoded in close proximity within a range of several thousand to several tens of thousands of base pairs on the genomic sequence. Here, the range “within several thousand to several tens of thousands of base pairs” is specifically within 10,000 base pairs (10000 bp), within 9,000 base pairs (9000 bp), or within 8,000 base pairs (8000 bp). Moreover, the gene cluster according to the present invention may also take the form of an operon.

Examples of bacteria having, in the same gene cluster, a gene encoding gluconate kinase and a gene encoding a gluconate transporter in the present invention include bacteria having DNA showing at least 90% identity to the sequence set forth in SEQ ID NO: 37, bacteria having DNA showing at least 90% identity to the sequence set forth in SEQ ID NO: 248, bacteria having DNA showing at least 90% identity to the sequence set forth in SEQ ID NO: 168, bacteria having DNA showing at least 90% identity to the sequence set forth in SEQ ID NO: 169, bacteria having DNA showing at least 90% identity to the sequence set forth in SEQ ID NO: 199, and bacteria having DNA showing at least 90% identity to the sequence set forth in SEQ ID NO: 203 (see “Gluconate kinase+ Transporter [gene cluster (+)]” in FIG. 19A).

Furthermore, examples of bacteria having, in the same gene cluster, a gene encoding gluconate kinase and a gene encoding a gluconate transporter include 403 bacterial species shown in twelve Tables, such as Tables 18, 20, 22, 24, 26, 28, 30, 32, 34, 36, 38 and 40 below, and among these, preferable examples are 63 bacterial species in which the item “gut_microbes” is “1” in Tables 18, 20, 22, 24, 26, 28, 30, 32, 34, 36, 38 and 40.

Examples of bacteria having, in the same gene cluster, a gene encoding gluconate dehydratase and a gene encoding a gluconate transporter in the present: invention include bacteria having DNA showing at least 90% identity to the sequence set forth in SEQ ID NO: 17, bacteria having DNA showing at least 90% identity to the sequence set forth in SEQ ID NO: 19, bacteria having DNA showing at least 90% identity to the sequence set forth in SEQ ID NO: 188, bacteria having DNA showing at least 90% identity to the sequence set forth in SEQ ID NO: 189, bacteria having DNA showing at least 90% identity to the sequence set forth in SEQ ID NO: 213, bacteria having DNA showing at least 90% identity to the sequence set forth in SEQ ID NO: 243, bacteria having DNA showing at least 90% identity to the sequence set forth in SEQ ID NO: 21, bacteria having DNA showing at least 90% identity to the sequence set forth in SEQ ID NO: 32, bacteria having DNA showing at least 90% identity to the sequence set forth in SEQ ID NO: 212, bacteria having DNA showing at least 90% identity to the sequence set forth in SEQ ID NO: 251, bacteria having DNA showing at least 90% identity to the sequence set forth in SEQ ID NO: 10, bacteria having DNA showing at least 90% identity to the sequence set forth in SEQ ID NO: 239, bacteria having DNA showing at least 90% identity to the sequence set forth in SEQ ID NO: 240, bacteria having DNA showing at least 90% identity to the sequence set forth in SEQ ID NO: 29, bacteria having DNA showing at least 90% identity to the sequence set forth in SEQ ID NO: 226, bacteria having DNA showing at least 90% identity to the sequence set forth in SEQ ID NO: 18, bacteria having DNA showing at least 90% identity to the sequence set forth in SEQ ID NO: 245, bacteria having DNA showing at least 90% identity to the sequence set forth in SEQ ID NO: 228, bacteria having DNA showing at least 90% identity to the sequence set forth in SEQ ID NO: 186, bacteria having DNA showing at least 90% identity to the sequence set forth in SEQ ID NO: 187, and bacteria having DNA showing at least 90% identity to the sequence set forth in SEQ ID NO: 191 (see “Gluconate dehydratase+Transporter [gene cluster (+)]” in FIG. 19B).

Furthermore, examples of bacteria having, in the same gene cluster, a gene encoding gluconate dehydratase and a gene encoding a gluconate transporter include 70 bacterial species shown in Tables 14 and 16 below, and among these, preferable examples are 10 bacterial species in which the item “gut_microbes” is “1” in Tables 14 and 16.

### Non-Gluconate-Utilizing Bacteria

In the present invention, the use of the first bacteria described above suppresses the intestinal colonization of the second bacteria described later. Furthermore, as shown in Examples described later, this suppression can be enhanced by use in combination with specific bacteria that do not utilize/consume gluconate. Therefore, in the present invention, in addition to the above-described first bacteria, non-gluconate-utilizing bacteria (for example, bacteria having an effect of enhancing the gluconate utilization ability of the first bacteria, or bacteria having an effect of maintaining the proliferation or colonization of the first bacteria) may be used in combination.

“Non-gluconate-utilizing bacteria” mean bacteria that do not have gluconate utilization/consumption ability, and for example, as shown in Examples described later, when the bacteria are cultured in a medium containing gluconate, if the amount of gluconate remaining in the medium is not significantly reduced compared to before culture, the bacteria can be evaluated as not having gluconate utilization/consumption ability. More specifically, examples thereof include intestinal bacteria whose residual gluconate concentration in the above method is 250 μM or more (preferably 270 μM or more, more preferably 280 μM or more, still more preferably 290 μM or more).

Examples of the non-gluconate-consuming bacteria used in combination with the first bacteria include at least one bacterium (preferably 2 or more (for example, 3 or 4), more preferably 5 or more (for example, 6, 7, 8, or 9), still more preferably 10 or more (for example, 11, 12, 13, or 14), more preferably 15 or more (for example, 16, 17, 18, or 19), and still more preferably 20 or more (for example, 21) bacteria) selected from the group consisting of bacteria having DNA showing at least 90% identity to the sequence set forth in SEQ ID NO: 1, bacteria having DNA showing at least 90% identity to the sequence set forth in SEQ ID NO: 2, bacteria having DNA showing at least 90% identity to the sequence set forth in SEQ ID NO: 3, bacteria having DNA showing at least 90% identity to the sequence set forth in SEQ ID NO: 4, bacteria having DNA showing at least 90% identity to the sequence set forth in SEQ ID NO: 5, bacteria having DNA showing at least 90% identity to the sequence set forth in SEQ ID NO: 7, bacteria having DNA showing at least 90% identity to the sequence set forth in SEQ ID NO: 9, bacteria having DNA showing at least 90% identity to the sequence set forth in SEQ ID NO: 11, bacteria having DNA showing at least 90% identity to the sequence set forth in SEQ ID NO: 12, bacteria having DNA showing at least 90% identity to the sequence set forth in SEQ ID NO: 13, bacteria having DNA showing at least 90% identity to the sequence set forth in SEQ ID NO: 14, bacteria having DNA showing at least 90% identity to the sequence set forth in SEQ ID NO: 16, bacteria having DNA showing at least 90% identity to the sequence set forth in SEQ ID NO: 19, bacteria having DNA showing at least 90% identity to the sequence set forth in SEQ ID NO: 24, bacteria having DNA showing at least 90% identity to the sequence set forth in SEQ ID NO: 26, bacteria having DNA showing at least 90% identity to the sequence set forth in SEQ ID NO: 28, bacteria having DNA showing at least 90% identity to the sequence set forth in SEQ ID NO: 30, bacteria having DNA showing at least 90% identity to the sequence set forth in SEQ ID NO: 31, bacteria having DNA showing at least 90% identity to the sequence set forth in SEQ ID NO: 33, bacteria having DNA showing at least 90% identity to the sequence set forth in SEQ ID NO: 34, and bacteria having DNA showing at least 90% identity to the sequence set forth in SEQ ID NO: 35.

Furthermore, examples of the non-gluconate-utilizing/consuming bacteria include at least one bacterium (preferably 2 or more (for example, 3 or 4), more preferably 5 or more (for example, 6, 7, 8, or 9), and particularly preferably 10 bacteria) selected from the group consisting of bacteria having DNA showing at least 90% identity to the sequence set forth in SEQ ID NO: 1, bacteria having DNA showing at least 90% identity to the sequence set forth in SEQ ID NO: 12, bacteria having DNA showing at least 90% identity to the sequence set forth in SEQ ID NO: 19, bacteria having DNA showing at least 90% identity to the sequence set forth in SEQ ID NO: 24, bacteria having DNA showing at least 90% identity to the sequence set forth in SEQ ID NO: 26, bacteria having DNA showing at least 90% identity to the sequence set forth in SEQ ID NO: 28, bacteria having DNA showing at least 90% identity to the sequence set forth in SEQ ID NO: 30, bacteria having DNA showing at least 90% identity to the sequence set forth in SEQ ID NO: 31, bacteria having DNA showing at least 90% identity to the sequence set forth in SEQ ID NO: 33, and bacteria having DNA showing at least 90% identity to the sequence set forth in SEQ ID NO: 35.

Furthermore, from the viewpoint that the non-gluconate-utilizing/consuming bacteria tend to have a higher activity of suppressing the second bacteria, they are preferably at least one bacterium, more preferably 2 or more bacteria, still more preferably 3 or more bacteria, and particularly preferably 4 bacteria, selected from the group consisting of bacteria having DNA showing at least 90% identity to the sequence set forth in SEQ ID NO: 1, bacteria having DNA showing at least 90% identity to the sequence set forth in SEQ ID NO: 19, bacteria having DNA showing at least 90% identity to the sequence set forth in SEQ ID NO: 24, and bacteria having DNA showing at least 90% identity to the sequence set forth in SEQ ID NO: 35. Furthermore, it is desirable that the first bacteria used in combination be at least one bacterium selected from the group consisting of bacteria having DNA showing at least 90% identity to the sequence set forth in SEQ ID NO: 18, bacteria having DNA showing at least 90% identity to the sequence set forth in SEQ ID NO: 21, bacteria having DNA showing at least 90% identity to the sequence set forth in SEQ ID NO: 22, and bacteria having DNA showing at least 90% identity to the sequence set forth in SEQ ID NO: 37, as described above.

As described above, the bacteria according to the present invention (the first bacteria described above, and further the non-gluconate-utilizing bacteria) may be a single strain of bacteria or a mixture of bacterial strains composed of multiple strains of bacteria. Furthermore, the bacteria according to the present invention include not only bacteria that retain 16S rDNA having a specific sequence (for example, bacteria specified by accession number: NITE BP-03147, which retain 16S rDNA having the sequence set forth in SEQ ID NO: 1) but also bacteria having DNA showing at least 90% identity to the specific sequence.

In the present invention, “at least 90% identity” means that the identity to each sequence is 90% or more (for example, 91% or more, 92% or more, 93% or more, 94% or more), preferably 95% or more (for example, 96% or more, 97% or more, 98% or more), and still more preferably 99% or more (particularly preferably 100%). The sequence identity can be determined using, for example, a program of BLAST (Basic Local Alignment Search) (Altschul et al. J. Mol. Biol., 215:403-410, 1990). This program is based on the algorithm BLAST by Karlin and Altschul (Proc. Natl. Acad. Sci. USA, 87:2264-2268, 1990, Proc. Natl. Acad. Sci. USA, 90:5873-5877, 1993). When analyzing the identity between sequences by BLAST, it can be determined using, for example, BLAST of the National Center for Biotechnology Information (NCBI) (for example, using default parameters, that is, initial setting parameters).

The bacteria according to the present invention may also be bacteria bred by mutation treatment, genetic recombination, selection of natural mutant strains, or the like. Such a “mutant strain” means a strain that has been mutated to a specific bacterium (bacterial strain) by a method well known to those skilled in the art within a range that does not change its properties, a strain bred by selection of a natural mutant strain, or the like, or a strain that a person skilled in the art can confirm to be equivalent thereto. Further, the bacteria according to the present invention are not limited to the strains themselves that have been deposited or registered with a prescribed institution as described above (hereinafter also referred to as “deposited strains”), and also include substantially equivalent strains (also referred to as “derived strains” or “induced strains”). Examples of the deposited strains of the present invention include the bacteria specified by NITE BP-03147 described above. A “strain substantially equivalent to a deposited strain” means a strain that at least belongs to the same species as the deposited strain. A strain substantially equivalent to a deposited strain may be, for example, a derived strain whose parent strain is the deposited strain. Examples of derived strains include strains bred from a deposited strain and strains naturally occurring from a deposited strain.

### Gluconate-Utilizing Second Bacteria

In the present invention, “gluconate-utilizing second bacterial strain”, “gluconate-utilizing second bacterium”, and “gluconate-utilizing second bacteria” are not particularly limited as long as they are bacteria that have gluconate utilization/consumption ability and can colonize the intestine; however, from the viewpoint that their colonization in the intestine is suppressed by competing with the first bacteria in gluconate utilization, it is preferable that the gluconate utilization/consumption ability of the second bacteria is lower than that of the first bacteria. More specifically, the second bacteria are intestinal bacteria whose residual gluconate concentration in the above method is preferably 70 μM or less, more preferably 50 μM or less, and still more preferably 30 μM or less.

Examples of such second bacteria include bacteria belonging to the family Enterobacteriaceae, more specifically, bacteria belonging to Klebsiella, bacteria belonging to Escherichia, bacteria belonging to the genus Proteus, bacteria belonging to the genus Salmonella, and bacteria belonging to the genus Pseudomonas. Examples of bacteria other than those belonging to the family Enterobacteriaceae include bacteria belonging to the genus Staphylococcus, bacteria belonging to the genus Bacillus, bacteria belonging to the genus Peptostreptococcus, bacteria belonging to the genus Megasphaera, and bacteria belonging to the genus Enterococcus.

Examples of bacteria belonging to Klebsiella include Klebsiella pneumoniae (Kp2H7 strain, 34E1 strain, BAA-1705 strain, 700603 strain, 40B3 strain, etc.) and Klebsiella aerogenes (Ka11E12 strain, etc.). For details of these bacteria, refer to WO 2018/084172 and Table 4 below.

TABLE 4

| Bacterial Strain Name | Information | Registration Number |
|---|---|---|
| Kp2H7 | https://www.ncbi.nlm.nih.gov/biosample/?term=SAMD00083910 | BioSample: SAMD00083910 |
| 34E1 | https://www.ncbi.nlm.nih.gov/biosample/?term=SAMD00083911 | BioSample: SAMD00083911 |
| BAA-1705 | https://www.atcc.org/Products/All/BAA-1705.aspx | NCBI Taxonomy ID: 1276652 |
| 700603 | https://www.atcc.org/Products/All/700603.aspx | NCBI Taxonomy ID: 1276653 |
| 4083 | https://www.ncbi.nlm.nih.gov/biosample/?term=SAMD00083913 | BioSample: SAMD00083913 |
| Ka11E12 | https://www.ncbi.nlm.nih.gov/biosample/?term=SAMD00083912 | BioSample: SAMD00083912 |

Examples of bacteria belonging to Escherichia include Escherichia coli, such as adherent invasive Escherichia coli (AIEC) (E. coli LF82, etc.) and extended-spectrum β-lactamase (ESBL)-producing Escherichia coli (E. coli ATCC BAA2777, etc.). Examples of bacteria belonging to the genus Proteus include Proteus mirabilis (P. mirabilis JCM1669T, etc.) and Proteus vulgaris (P. vulgaris JCM20013, etc.). Examples of bacteria belonging to the genus Salmonella include Salmonella enteritidis (S. enterica subsp. enterica SL1344, etc.). Examples of bacteria belonging to the genus Pseudomonas include Pseudomonas aeruginosa (P. aeruginosa ATCC10145, etc.). Examples of bacteria belonging to the genus Staphylococcus include Staphylococcus aureus (S. aureus JCM16555, etc.), Staphylococcus mutans, and Staphylococcus epidermidis. Examples of bacteria belonging to the genus Bacillus include Bacillus cereus (B. cereus JCM2152, etc.). Examples of bacteria belonging to the genus Peptostreptococcus include Peptostreptococcus prevotii. Examples of bacteria belonging to the genus Megasphaera include Megasphaera elsdenii. Examples of bacteria belonging to the genus Enterococcus include Enterococcus faecalis and Enterococcus faecium.

Such second bacteria may be bacteria that cause diseases by colonizing the intestine. Examples of diseases caused by the second bacteria include sepsis, peritonitis, meningitis, enteritis, gastroenteritis (infectious gastroenteritis, etc.), respiratory infections (pneumonia, etc.), urinary tract infections, surgical site infections, soft tissue infections, medical device-related infections (medical device-related bloodstream infections, etc.), inflammatory bowel diseases (chronic inflammatory bowel diseases such as Crohn's disease and ulcerative colitis, etc.), type 1 diabetes, rheumatoid arthritis, experimental autoimmune encephalomyelitis (EAE), multiple sclerosis, and autoimmune diseases such as systemic lupus erythematosus, chronic inflammatory diseases, opportunistic infections caused by methicillin-resistant Staphylococcus aureus (MRSA), food poisoning (cereus food poisoning, etc.), and toxic shock syndrome (TSS).

### Aspects of the Composition

The composition of the present invention is an antibacterial composition including at least one gluconate-utilizing first bacterial strain as an active agent/or ingredient, the composition exhibits antibacterial activity against gluconate-utilizing second bacteria. The bacteria according to the present invention contained in such a composition (the first bacteria described above, and further the non-gluconate-utilizing bacteria) may be not only live bacteria but also cultures thereof. The culture may be one including the bacteria (a medium (culture solution, solid medium, etc.) comprising the proliferated bacteria), and the form thereof is not particularly limited and may be either liquid or solid, and as long as it can be restored and resume growth in the host's intestine after administration, it may be in a dry form (for example, a culture-dried product). Such a dried product can be prepared by drying a suspension obtained by dispersing bacterial cells in a solvent (water or the like). A drying protectant described later may be appropriately added to the suspension and dried. The drying method is not particularly limited, and examples thereof include freeze drying, spray drying, and heat drying, but freeze drying is preferable.

The composition of the present invention may be in the form of a pharmaceutical composition (pharmaceutical product, quasi-drug, etc.), food composition (food and/or beverage, animal feed, etc.), or reagent used in cell experiments, model animal experiments, etc.

When the composition of the present invention is used as a pharmaceutical composition, it can be formulated by a known pharmaceutical method. For example, capsules, tablets, pills, liquids, powders, granules, fine granules, film-coated preparations, pellets, troches, sublingual tablets, chewable tablets, buccal tablets, pastes, syrups, suspensions, elixirs, emulsions, liniments, ointments, plasters, cataplasms, transdermal preparations, lotions, inhalants, aerosols, injections, suppositories, etc. can be used orally or parenterally, but oral administration is desirable from the viewpoint of non-invasive and easy ingestion.

In these formulations, pharmacologically or food and/or beverage acceptable carriers can be appropriately combined; specifically, these include physiological saline, sterile water, media (modified GAM broth (manufactured by Nissui Pharmaceutical Co., Ltd.), reinforced clostridial medium, BHI medium, BL medium, LB medium, EG medium, etc.), drying protectants (sugars or sugar alcohols such as sucrose, lactose, trehalose, dextran, erythritol, arabitol, xylitol, sorbitol, and mannitol; amino acids such as arginine and histidine; and polyols such as propylene glycol, glycerol, poly(ethylene glycol), and poly(propylene glycol)), vegetable oils, solvents, excipients, bases, emulsifiers, suspending agents, surfactants, stabilizers, flavoring agents, fragrances, vehicles, preservatives, binders, diluents, isotonic agents, soothing agents, bulking agents, disintegrating agents, buffering agents (phosphates, citrates, acetates, etc.), coating agents, lubricants, coloring agents, sweeteners, viscous agents, flavor modifiers, solubilizers, or other additives and the like.

In view of the fact that the bacteria according to the present invention are intestinal bacteria, particularly in preparations intended for oral administration, the bacteria according to the present invention may be combined with a composition that enables efficient delivery of the bacteria to the intestine. The composition that enables such delivery to the intestine is not particularly limited, and a known composition can be appropriately adopted, and examples thereof include pH-sensitive compositions, compositions that suppress release until the intestine (cellulose-based polymers, acrylic acid polymers and copolymers, vinyl acid polymers and copolymers, etc.), bio adhesive compositions that specifically adhere to the intestinal mucosa (for example, polymers described in US Patent No. 6, 368, 586), protease inhibitor-containing compositions, and compositions that are specifically decomposed by intestinal enzymes.

In the present invention, when the second bacteria are pathogenic or disease-causing bacteria, the composition of the present invention can be used for treating or preventing the disease. “Treatment” includes not only complete recovery from the disease but also alleviating or ameliorating the symptoms of the disease, suppressing the progression thereof, and suppressing recurrence thereof. “Prevention” includes suppressing or delaying the onset of the disease, or suppressing recurrence thereof.

When the composition of the present invention is used as a food composition, it can be, for example, a health food, functional food, food for specified health use, food with nutrient function claims, food with function claims, dietary supplement, food for patients, or animal diets or feed. Although functional foods are usually classified into four types: probiotics, biogenics, prebiotics, and synbiotics based on their mechanism of action, the present invention may take the form of probiotics.

The composition of the present invention can also be ingested as various foods and/or beverages. Specific examples of foods and/or beverages include liquid foods such as functional beverages, drink preparations, jelly-like beverages, soft drinks, milk beverages, tea beverages, alcoholic beverages, and soups; fermented foods and fermented beverages such as yogurt and drinking yogurt; products containing oils and fats such as edible oil, dressing, mayonnaise, and margarine; carbohydrate-containing foods such as cooked rice, noodles, and bread; livestock processed foods such as ham and sausage; marine processed foods such as kamaboko, dried fish, and salted fish guts; vegetable processed foods such as pickles; semi-solid foods such as jelly; fermented foods such as miso; various confectioneries such as Western confectioneries, Japanese confectioneries, candies, gums, gummies, frozen desserts, and ice confections; retort products such as curry, thick starchy sauce, and Chinese soups; instant foods such as instant soups and instant miso soup, and microwave-ready foods. Furthermore, powdered, granulated, tableted, encapsulated, liquid, paste, or jelly-like health foods and/or beverages are also included. The production of foods and/or beverages in the present invention can be carried out by production techniques known in the art.

The food and/or beverage composition proposed in the present invention can also be provided and sold as a food and/or beverage with health uses. The act of “indication” includes all acts that make the consumer aware of the uses, and may be an expression that allows direct recognition of the uses, or an expression that evokes or analogizes the uses, attached to the composition itself, or an expression attached to a container, packaging material, or package insert containing the composition. The “indication” may also be a display or advertisement stating that the composition of the present invention is effective, as information related to the composition of the present invention, in the forms of leaflets, pamphlets, pops, catalogs, posters, books, storage media such as DVDs, electronic bulletin boards, and advertisements such as those on the Internet.

The composition of the present invention may comprise one or more agents/ingredients effective in antibacterial activity against the second bacteria (for example, ingredients effective in the growth of the bacteria according to the present invention in the intestine). It may also be combined with known ingredients, drugs, or foods for treating or preventing the above-mentioned diseases. Furthermore, it may be combined with other ingredients, drugs, or foods that exhibit functions than other the above-mentioned antibacterial, therapeutic, and prophylactic functions.

In particular, as shown in Examples described later, by ingesting food containing a low concentration of gluconate to create a situation in which the amount of gluconate in the intestine is low, it is possible to exert stronger antibacterial activity against the second bacteria. Therefore, the composition of the present invention may also take the form of a composition that is ingested in combination with a food containing a low concentration of gluconate.

Here, “low concentration” means, for example, that the concentration of gluconate in the food is preferably 2.5% by weight or less, more preferably 2% by weight or less, still more preferably 1.5% by weight or less, more preferably 18 by weight or less, still more preferably 0.5% by weight or less, and particularly preferably 0% by weight.

Examples of such foods containing a low concentration of gluconate include bananas, blueberries, strawberries, grapefruit, carrots, eggplants, potatoes, pumpkins, gluten-free bread (rice flour bread, bran bread, soybean flour bread, etc.), rice, oats, fermented foods such as hard cheese, tofu, and sugar.

The phrase “ingested in combination” in the present invention includes ingesting the bacteria according to the present invention and the food containing a low concentration of gluconate at the same time or at different times, through the same route or different routes, respectively. In the case of ingestion at different times, it is preferable to ingest the bacteria according to the present invention after ingesting the food from the viewpoint that the influence of gastric acid is reduced, and the administered bacteria are more easily colonized in the intestine. In this case, it is usually sufficient to ingest the bacteria according to the present invention within 1 hour after ingesting the food, and it is preferable to ingest the bacteria 20 to 30 minutes after ingesting the food. The number of times of ingesting the bacteria according to the present invention and the food may be the same or different. Therefore, the composition of the present invention may be in a form in which the bacteria according to the present invention and the food are contained in one composition, or in a form in which the bacteria according to the present invention and the food are contained in separate compositions.

### Method for Suppressing Bacteria in the Intestine

The present invention also provides a method for suppressing gluconate-utilizing second bacteria in the intestine of a subject, including the step of administering the bacteria according to the present invention (the first bacteria described above, and further the non-gluconate-utilizing bacteria described above) or a composition including the bacteria (the composition of the present invention described above) to the subject, or allowing the subject to ingest the same.

Here, “suppression” means suppression of the activity of bacteria, more specifically, suppression of proliferation, growth, or colonization of bacteria, or killing of bacteria, similar to the antibacterial activity described above, and includes, for example, suppression of bacterial colonization in the intestine and elimination of bacteria from the intestine.

The “subject” in the present invention is an animal including a human. Animals other than humans are not particularly limited, and various livestock, poultry, pets, laboratory animals, etc. can be targeted. Specific examples include, but are not limited to, pigs, cattle, horses, sheep, goats, chickens, wild ducks, ostriches, domestic ducks, dogs, cats, rabbits, hamsters, mice, rats, and monkeys. Further, examples of the subject in the present invention include a person whose intestine is colonized with second bacteria, a person who may have second bacteria colonized in the intestine, a person who is at risk of having second bacteria colonized in the intestine, a person suffering from the above-mentioned diseases, a person who may suffer from the above-mentioned diseases, and a person who is at risk of suffering from the above-mentioned diseases.

Further, the present technology may be used for therapeutic purposes or non-therapeutic purpose uses. “Non-therapeutic use” is a concept that does not include medical practice, that is, treatment of the human body. Examples thereof include health promotion.

When the bacteria according to the present invention or the above-described composition of the present invention is administered or ingested, the dosage or intake amount is appropriately selected depending on the age, weight, and health condition of the subject, the type of composition (pharmaceutical product, food and/or beverage, etc.), the form of the active ingredient (for example, live bacteria or culture), and the like.

The content or amount used of the bacteria (live bacteria) according to the present invention is not particularly limited, but is 1×10⁹ to 1×10¹², more preferably 1×10⁹ to 1×10¹¹, in the composition. The unit is CFU/g or CFU/mL, or cells/g or cells/mL. CFU stands for Colony forming unit.

The daily dose of the microorganisms (live bacteria) of the present invention is not particularly limited, but is preferably 1×10⁹ to 1×10¹³ per day, and may be 1×10⁹ to 1×10¹², or even 1×10⁹ to 1×10¹¹. The unit is CFU/day or cells/day.

When the active ingredient is a culture, the amount of the culture can be, for example, an amount corresponding to the amount of bacterial cells.

The dosage or intake amount of the bacteria according to the present invention or the above-described composition of the present invention is as described above; however, the bacteria or the composition may be administered or ingested once or multiple times (for example, twice or three times) per day. The period of administration or ingestion can be discontinued depending on the condition of the subject, or it may be administered or ingested continuously without discontinuation. As for “continuous”, daily continuation or continuation at intervals is possible, but daily continuation of administration or ingestion of the bacteria according to the present invention or the above-described composition of the present invention is preferable from the viewpoint of effects.

### Method for Screening Bacteria Having Antibacterial Activity

The present invention provides a method for screening bacteria having antibacterial activity against gluconate-utilizing bacteria, including the steps of: detecting gluconate utilization ability of a test bacterial strain; and determining that the test bacterial strain has the antibacterial activity when the gluconate utilization ability is detected in the step.

The present invention also provides a method for screening bacteria having antibacterial activity against gluconate-utilizing bacteria, including the steps of: detecting a gene encoding a gluconate transporter and at least one gene selected from the group consisting of a gene encoding gluconate kinase and a gene encoding gluconate dehydratase, of a test bacterial strain; identifying the gene cluster corresponding to each detected gene; and determining that the test bacterial strain has the antibacterial activity when the detected genes are located in the same gene cluster.

The “gluconate-utilizing bacteria” in the present screening method are not particularly limited, but include the second bacteria described above. The “test bacterial strain” to be subjected to the present screening method are not particularly limited either, and examples thereof include bacteria present in the intestine of an animal. Examples of such animals include humans and non-human animals (mice, rats, monkeys, pigs, cattle, horses, sheep, goats, chickens, wild ducks, ostriches, domestic ducks, dogs, cats, rabbits, hamsters, etc.). The test bacteria may be isolated bacteria, but include samples containing the bacteria (for example, fecal samples of the animals or cultures thereof).

“Detecting gluconate utilization ability” and “detecting gluconate utilization by a test bacterium” can be performed, for example, as described above, by culturing the bacteria in a medium containing gluconate and detecting the amount of gluconate remaining in the medium. If the amount of gluconate is significantly reduced compared to that before culture, the bacteria are determined to have gluconate utilization/consumption ability. More specifically, when the gluconate concentration remaining in the culture supernatant after culturing bacteria for 48 hours by the method shown in Examples described later is 100 μM or less (preferably 70 μM or less, more preferably 50 μM or less, still more preferably 30 μM or less, more preferably 20 μM or less, still more preferably 10 μM or less, more preferably 3 μM or less), the bacteria can also be determined to have gluconate utilization/consumption ability.

Furthermore, as shown in Examples described later, bacteria can also be determined to have gluconate utilization (or consumption) ability, when the bacteria are ingested by an animal, the amount of gluconate in the animal's feces is detected, and is significantly reduced compared to the amount when the bacteria are not ingested. The animal is not particularly limited, and examples thereof include humans, pigs, cattle, horses, sheep, goats, chickens, wild ducks, ostriches, domestic ducks, dogs, cats, rabbits, hamsters, mice, rats, and monkeys.

The method for detecting the amount of gluconate is not particularly limited, and examples thereof include a detection method using a liquid chromatograph mass spectrometer (LC-MS/MS), as shown in Examples described later.

“Gluconate kinase”, “gluconate dehydratase”, “gluconate transporter”, and “gene cluster” in the present screening method are as described above. Identification of whether at least two genes according to the present invention are located in the same gene cluster may be performed, for example, by using the next-generation sequencing (NGS) method as shown in Examples described later. More specifically, the “next-generation sequencing method” includes a sequencing-by-synthesis method (for example, sequencing by Solexa Genome Analyzer, HiSeq or MiSeq manufactured by Illumina, Inc.), a pyrosequencing method (for example, sequencing by sequencers GSLX or FLX manufactured by Roche Diagnostics (454) (so-called 454 sequencing)), a ligase reaction sequencing method (for example, sequencing by SoLiD or 5500×1 manufactured by Life Technologies), and an ion semiconductor sequencing method (for example, Ion Torrent technology, Thermo Fisher Scientific Inc.).

### EXAMPLES

Hereinafter, the present invention will be described more specifically based on Examples, but the present invention is not limited to the following Examples. The Examples were conducted using the materials and methods described below.

### Mouse Experiment 1

For Example 2 described later, the following mouse experiments were conducted. Germ-free (GF) C57BL/6N mice, 4 to 8 weeks of age, were purchased from CLEA Japan, Inc. or Sankyo Labo Service Corporation, Inc. They were maintained in a vinyl breeding isolator (sterile isolator) (manufactured by ICM Co., Ltd.; ICM-1B), and mice 8 to 14 weeks of age were used in the following experiments.

Klebsiella pneumoniae 2H7 strain (hereinafter also referred to as Kp2H7 strain), which was administered to germ-free mice in the Examples of the present application, is described in WO 2018/084172, WO 2019/017389, and WO 2020/179868, each of which is hereby incorporated by reference. Isolated bacteria (bacteria belonging to F31-18mix or F18mix) administered to germ-free mice are referenced in WO 2019/017389, WO 2020/179868, and is Table 5, each of which hereby incorporated by reference. F31-18mix refers to the 13 bacterial strains in F31mix that are not present in F18mix.

TABLE 5

| | | SEQ ID | | NCBI | % | F31 | F18 |
|---|---|---|---|---|---|---|---|
| No. | Species | NO: | subject id | TAX ID | identity | mix | mix |
| f01 | Bifidobacterium longum | 1 | NR_145535.1 | 1931217 | 99.60 | ● | ● |
| f02 | Bacteroides xylanisolvens | 2 | NR_112947.1 | 657309 | 99.59 | ● | — |
| f03 | Bacteroides fragilis | 3 | NR_074784.2 | 817 | 98.95 | ● | — |
| f04 | Bacteroides uniformis | 4 | NR_112945.1 | 820 | 99.93 | ● | — |
| f05 | Bacteroides thetaiotaomicron | 5 | NR_074277.1 | 818 | 99.80 | ● | — |
| f06 | Bacteroides uniformis | 6 | NR_112945.1 | 820 | 99.93 | — | — |
| f07 | Bacteroides acidifaciens | 7 | NR_112931.1 | 85831 | 97.18 | ● | — |
| f08 | Bacteroides fragilis | 8 | NR_074784.2 | 817 | 98.95 | — | — |
| f09 | Parabacteroides goldsteinii | 9 | NR_113076.1 | 927665 | 98.98 | ● | — |
| f10 | [Ruminococcus] gnavus | 10 | NR_036800.1 | 411470 | 99.80 | ● | — |
| f11 | Blautia luti | 11 | NR_114315.1 | 649762 | 97.57 | ● | — |
| f12 | Faecalimonas umbilicata | 12 | NR_156907.1 | 1912855 | 99.87 | ● | ● |
| f13 | [Clostridium] saccharolyticum | 13 | NR_102852.1 | 84030 | 94.34 | ● | — |
| f14 | Tyzzerella nexilis | 14 | NR_029248.1 | 500632 | 98.49 | ● | — |
| f15 | [Ruminococcus] gnavus | 15 | NR_036800.1 | 411470 | 99.80 | — | — |
| f16 | Anaerostipes hadrus | 16 | NR_117138.2 | 649756 | 99.74 | ● | — |
| f17 | Blautia glucerasea | 17 | NR_113231.1 | 536633 | 96.91 | ● | ● |
| f18 | [Clostridium] bolteae | 18 | NR_025567.1 | 208479 | 99.60 | ● | ● |
| f19 | Blautia caecimuris | 19 | NR_144607.1 | 1796615 | 99.13 | ● | ● |
| f20 | [Clostridium] innocuum | 20 | NR_029164.1 | 1522 | 98.71 | ● | ● |
| f21 | Blautia marasmi | 21 | NR_147395.1 | 1917868 | 98.20 | ● | ● |
| f22 | [Clostridium] lavalense | 22 | NR_044289.1 | 460384 | 98.97 | ● | ● |
| f23 | [Clostridium] glycyrrhizinilyticum | 23 | NR_112553.1 | 342942 | 99.13 | ● | ● |
| f24 | Eisenbergiella massiliensis | 24 | NR_144731.1 | 1720294 | 97.73 | ● | ● |
| f25 | [Clostridium] glycyrrhizinilyticum | 25 | NR_112553.1 | 342942 | 99.13 | — | — |
| f26 | Flavonifractor plautii | 26 | NR_029356.1 | 292800 | 100.00 | ● | ● |
| f27 | Blautia luti | 27 | NR_114315.1 | 649762 | 97.57 | — | — |
| f28 | Intestinibacter bartlettii | 28 | NR_027573.1 | 261299 | 99.93 | ● | ● |
| f29 | [Ruminococcus] gnavus | 29 | NR_036800.1 | 411470 | 98.85 | ● | — |
| f30 | Massilimicrobiota timonensis | 30 | NR_144738.1 | 1776392 | 100.00 | ● | ● |
| f31 | Anaerostipes caccae | 31 | NR_028915.1 | 105841 | 97.32 | ● | ● |
| f32 | Blautia coccoides | 32 | NR_104700.1 | 1532 | 99.93 | ● | ● |
| f33 | Erysipelatoclostridium ramosum | 33 | NR_029247.1 | 1547 | 99.41 | ● | ● |
| f34 | Veillonella parvula | 34 | NR_074980.1 | 479436 | 99.16 | ● | — |
| f35 | Fusobacterium varium | 35 | NR_113384.1 | 856 | 99.73 | ● | ● |
| f36 | Fusobacterium varium | 36 | NR_113384.1 | 856 | 99.73 | — | — |
| f37 | Escherichia fergusonii | 37 | NR_074902.1 | 585054 | 99.87 | ● | ● |

A bacterial solution containing Kp2H7 strain was placed in LB liquid medium and incubated overnight at 37° C., and the OD of the bacterial solution was adjusted to 1.2 (equivalent to 1×10⁹ CFU/mL). Then, aliquots of the bacterial solution were administered into the stomachs of individual mice using an oral gavage needle at a dose of 200 μL per mouse (equivalent to 2×10⁸ CFU). In experiments using gene-deficient strains, a bacterial solution containing a wild-type strain and a bacterial solution containing a gene-deficient strain were prepared in the same manner as described above, and the two solutions were mixed in equal volumes. 200 μL of the bacterial solution was administered into the stomachs of individual mice (namely, at a dose of 200 μL/mouse, equivalent to 1×10⁸ CFU for each bacterial strain) using an oral gavage needle.

Isolated bacterial strains were individually cultured in mGAM liquid medium, EG medium, or CM0149 medium at 37° C. in an anaerobic chamber for 24 to 48 hours. The resulting culture solutions, each containing an isolated strain, were appropriately mixed in equal volumes. Furthermore, these mixed solutions were concentrated fivefold to prepare F18mix, F31-18mix, F8mix described later, and F10mix (F18-8mix) described later. Aliquots of the bacterial solution were administered into the stomachs of individual mice using an oral gavage needle at a dose of 200 μL per mouse (the total quantity of each bacterial strain per mouse is equivalent to 1×10⁹ CFU).

Fecal samples from mice were dissolved in a PBS containing glycerol (final concentration 20%) and EDTA (final concentration 10 mM) at a ratio of 50 mg of feces per mL of PBS. The resulting fecal lysate was diluted to an appropriate concentration, plated onto DHL medium containing 50 mg/L ampicillin and 50 mg/L spectinomycin, and incubated overnight at 37° C. Then the number of colonies was counted, and CFU per 1 g of feces was calculated.

CL-2 (CLEA Japan) was used as a normal diet for mice. In experiments in which the amount of gluconate intake was changed midway, a diet consisting of AlN93G (Oriental Yeast Co., Ltd.) was used from Day 21 onward. The diet consisting of AlN93G was prepared by supplementing gluconate to achieve concentration of 0% by weight (without gluconate), 2.5% by weight (80 μmol/g), or 10% by weight (320 μmol/g), and the diet was fed to the mice. The gluconate content of the normal diet (CL-2) was 5 μmol/g. In all experiments, mice were fed ad libitum.

### Amount of Bacterial DNA in Feces

After administering Kp2H7 and F18mix to germ-free mice, the diet with adjusted gluconate concentration was fed, and the change in the abundance of each bacterial strain in the mice was examined. Specifically, feces from each mouse were collected once a week, and on Day 21 and Day 25 after the diet was changed, and bacterial genomes were extracted from them.

For extraction of bacterial genomes, 5 times the weight of PBS containing EDTA and glycerol (final concentration of EDTA: 10 mM, final concentration of glycerol: 20% by volume) was added to 10 mg of mouse feces, and the mixture was vigorously shaken and stirred to crush and suspend the feces. To 100 μL of the obtained sample solution, 800 μL of 10 mM Tris/10 mM EDTA buffer (pH 8.0, hereinafter referred to as “TE10”) was added, in which the TE10 was obtained by dissolving 15 mg of lysozyme (manufactured by Sigma-Aldrich, Lysozyme from chicken egg white; L4919) and 5 μL of RNase (manufactured by Thermo Fisher Scientific, PureLink RNase A (20 mg/mL); 12091-021), and the mixture was shaken at 37° C. for 1 hour. Subsequently, 2,000 U of Achromopeptidase (registered trademark) (Wako; 015-09951) was added, and the mixture was shaken at 37° C. for 30 minutes to lyse the cells. A volume of 50 μL of 20% SDS TE10 solution together with 50 μL of TE10 solution, which had proteinase K (Roche, Proteinase K, recombinant, PCR Grade; 03115852001) dissolved to a final concentration of 20 mg/ml, was added, and the mixture was shaken at 55° C. for 60 minutes. DNA was extracted from 400 μL of the mixed solution using Maxwell (registered trademark) RSC Cultured Cells DNA Kit (Promega). Using the obtained DNA as a template, qPCR using primers specific to each bacterial strains was performed to quantify the amount of each bacterial strain. The primers specific to each bacterial species are indicated in Table 6.

TABLE 6

| | | | SEQ ID | | | SEQ ID |
|---|---|---|---|---|---|---|
| | Primer Name | Fw | NO: | Primer Name | Rv | NO: |
| f01 | f01_42H6_Fw1 | AGCTTTCGCGGTATGGGATG | 38 | f01_42H6_Rv1 | GCCGTATCTCAGTCCCAATG | 39 |
| f12 | f12_42H4_Fw1 | ACACGTGATCGCATGATCGAG | 40 | f12 42H4_Rv1 | CCAACTAGCTAATCAGACGC | 41 |
| f17 | f17_4217_Fw3 | ATGGAAGCTTCGGTGGAAATAG | 42 | f17_4217_Rv3 | CACACTGAATCATGCAGTCC | 43 |
| f18 | f18_4212_Fw5 | GCGAAGCAAGTCTGAAGTGA | 44 | f18_4212_Rv5 | CTCCGACACTCTAGCAAAAC | 45 |
| f19 | f19_43G2_Fw2 | CGGTTGAAGTCGTTATAATCC | 46 | f19_43G2_Rv2 | CCATGCAGTCCCGTGCGCTTA | 47 |
| f20 | f20 43G1_Fw2 | CTTGCTTCCAAAGAGACTTAG | 48 | f20 43G1_Rv2 | GCAGTTATCCCGGACACATG | 49 |
| f21 | f21_42A8_Fw2 | AAGTGGATCTCTTCGGATTG | 50 | f21_42A8_Rv6 | TTATGCGGTATTAGCAGCCA | 51 |
| f22 | f22_43C3_Fw2 | GATTCTGAGATGACTGAGTG | 52 | f22_43C3_Rv2 | GCAGTCATTTCTAACTGTTG | 53 |
| f24 | f24_4214_Fw1 | TCGGTATAACTTAGTGGCGG | 54 | f24_4214_Rv1 | ACAACCATGCGGTTCCGTGC | 55 |
| f25 | f25_42J1_Fw2 | TACCGGATTTCTTCGGGATG | 56 | f25_42J1_Rv2 | GATCATGCAATCCTGTGGTC | 57 |
| f26 | f26_42K2_Fw1 | TTTATCGCTCTGAGATGGCC | 58 | f26_42K2_Rv1 | TCAACCTCTCAGTCCGGCTA | 59 |
| f28 | f28_43A3_Fw1 | GTACACACGGATAACATACC | 60 | f28_43A3_Rv1 | ACCAACTAGCTAATCAGACG | 61 |
| f30 | f30_43A5_Fw1 | TATCCCACGGGATAGCACAG | 62 | f30_43A5_Rv1 | TCTCAGGCCGGCTACGCATC | 63 |
| f31 | f31_43J5_Fw1 | TTCCTTAATGACTGAGTGGC | 64 | f31_43J5_Rv2 | CACACCGAATCATGCGATTC | 65 |
| f32 | f32_42A7_Fw1 | TGAAGTCTTTGTGACTGAGC | 66 | f32_42A7_Rv2 | ATGCGGTATTAGCAGTCA | 67 |
| f33 | f33_43N2_Fw1 | GTGCCTCAAAGCACTGGTAG | 68 | f33_43N2_Rv1 | TCTCAGGTCGGCTACGCATC | 69 |
| f35 | f35_42L8_Fw2 | ACTTGATCCTTCGGGTGAAG | 70 | f35_42L8_Rv2 | ATGTTGTCCCAGTCTGTAAG | 71 |
| f37 | f37_42G1_Fw3 | GCTGCTTTGCTGACGAGTGG | 72 | f37_42G1_Rv4 | TTGGTCTTGCGACGTTATGC | 73 |
| Kp2H7 | Sca4_298_F | AGCACTAGCGGCTGTGGTAT | 74 | Sca4_298_R | ACTTACTCGGGCCCTTGATT | 75 |

### Preparation of Kp2H7 Gene-Deficient Strain

The Kp2H7 gene-deficient strain was prepared using Quick and Easy E. coli Gene Deletion Kit (Gene Bridges, Heidelberg) according to the protocol of the kit.

First, the pRED/ET plasmid was introduced into Kp2H7 strain by electroporation, and the gene-introduced bacterial strain was selected by culturing at 30° C. using LB agar medium containing 30 mg/L tetracycline. After taking the selected pRED/ET-introduced bacterial strain into LB liquid medium and culturing it overnight at 30° C., 1 mL thereof was placed in 40 mL of LB liquid medium and cultured at 30° C. until the OD reached 0.2. After that, L-arabinose was added to 0.3%, the temperature was changed to 37° C., and the cells were further cultured for 1 hour; then the bacterial solution was collected to prepare competent cells. A linear DNA with the target recombination sequences added to both ends of the FRT-PGK-gb2-neo-FRT cassette was prepared by PCR according to the protocol of the kit. Then, the obtained DNA was introduced into the competent cells by electroporation, and the gene-deficient strain was selected by culturing in LB agar medium containing 90 mg/L kanamycin.

### Measurement of Gluconate Concentration 1

For Examples 3 and 4, the gluconate concentration was measured as described below. To examine the gluconate utilization ability of the bacterial strains, 10 μL of bacterial solution was added to 1 mL of modified GAM broth containing 300 gluconate (manufactured by Nissui Pharmaceutical Co., Ltd.), and the mixture was statically cultured at 37° C. under anaerobic conditions (in an anaerobic chamber) for 48 hours. The obtained culture solution was centrifuged, and the gluconate concentration in the supernatant was measured using LC-MS/MS. The bacterial solution used was one in the stationary phase that was obtained by taking bacterial strains from a single colony, put them into a medium and culturing them overnight at 37° C. under anaerobic conditions. A strain in which the gluconate concentration was significantly reduced (reduced to 100 μM or less) was determined as a strain capable of utilizing gluconate.

Further, regarding Example 2 described later, to measure the gluconate concentration in feces, mouse feces were dissolved in 20 mg/mL MQ, centrifuged, and the gluconate concentration in the supernatant was measured using LC-MS/MS. Liquid chromatography was performed using ExionLC AD, and mass spectrometry was performed using SCIEX TQ6500+. The measurement conditions are as follows.

TABLE 7
[LC Conditions]
Column: Intrada Organic Acid, 150 × 2 mm
Mobile Phase: A: acetonitrile/water/formic acid = 10/90/0.1,
B: acetonitrile/100 mM ammonium formate = 10/90

| Time (min) | Flow (mL/min) | A.conc | B.conc |
|---|---|---|---|
| | 0.2 | 100 | 0 |
| 3 | 0.2 | 100 | 0 |
| 10 | 0.2 | 0 | 100 |
| 13 | 0.2 | 0 | 100 |
| 13.1 | 0.2 | 100 | 0 |
| 18 | 0.2 | 100 | 0 |

Column Temperature: 40°
Injection Volume: 2 μL

TABLE 8
[MS Conditions]
Curtain Gas (CUR): 30 psi
Collision Gas (CAD): 9
IonSpray Voltage (IS): −4500 V
Temperature (TEM): 400°
Ion Source Gas 1 (GS1): 50 psi
Ion Source Gas 2 (GS2): 80 psi

| Q1 Mass | Q3 Mass | Dwell Time | ID | DP (volts) | EP (volts) | CE (volts) | CXP (volts) |
|---|---|---|---|---|---|---|---|
| 194.936 | 74.9 | 20 | gluconate-H2 | −40 | −10 | −24 | −11 |

### Transposon Sequencing (Tn-Seq)

To 80 μL of electrocompetent cells of Kp2H7 strain, 0.5 μL of EZ-Tn5TM <KAN-2> (Lucigen Corp, USA) was added, and electroporation was performed. Electroporation was performed using ELEPO21 (Nepa Gene Co. Ltd., Japan) under the conditions of poring pulse (voltage: 1800 V, pulse length: 5.0 msec, pulse interval: 50 msec, number of pulses: 1, and polarity: +). After electroporation, the cells were placed in 1 mL of LB liquid medium, incubated at 37° C. for 3 hours, and then cultured overnight on LB agar medium containing 90 mg/L kanamycin. After counting the number of colonies, all colonies (approximately 800,000 colonies) were collected and stored as 20% glycerol stock at −80° C.

The prepared glycerol stock of the transposon-inserted Kp strain was administered to germ-free C57BL/6 mice at a dose of 200 μL per mouse. Seven days later, the mice were divided into three groups: a group administered with isolated bacteria mix of F18mix, a group administered with isolated bacteria mix of F31-18mix, and a group administered with no bacterial mix. With the day of administration of isolated bacteria as Day 0, feces were collected from mice on Days 0, 4, 10, and 28, suspended in 50 mg/mL PBS, and then cultured on LB agar medium containing 90 mg/L kanamycin. After culturing overnight at 37° C., all colonies were collected, and DNA was extracted.

The extracted genomic DNA was fragmented to a length of about 300 bases, and a poly(C) base was added to the 3′-end of the DNA fragment. The sequence near the transposon was amplified using a biotinylated primer containing a sequence complementary to the transposon sequence and a primer containing a poly(G) base. The target fragment was purified with streptavidin beads and then amplified. The sequence adjacent to the transposon was obtained by Illumina HiSeq 2500 sequencing.

The first 24 bp of the obtained read sequences were trimmed to remove the PCR primer-complementary sequence and mosaic end sequence. Furthermore, trimming of Illumina sequencing adapters and filtering based on sequencing quality were performed using Trimmomatic-v0.39 and FASTX-Toolkit-v0.0.13. Trimmomatic-v0.39 is referenced in A. M. Bolger, M. Lohse, and B. Usadel, “Trimmomatic: a flexible trimmer for Illumina sequence data,” Bioinformatics, vol. 30, no. 15, pp. 2114-2120, August 2014, doi: 10.1093/BIOINFORMATICS/BTU170. Trimmomatic-v0.39 was used with options: ILLUMINACLIP: 2:30:10 LEADING: 3 TRAILING: 20 SLIDINGWINDOW: 4:15 MINLEN:5. FASTX-Toolkit-v0.0. 13 is provided on, http://hannonlab.cshl.edu/fastx_toolkit/. FASTX-Toolkit-v0.0.13 was used with options: -q 20 -p 80. The quality-controlled read sequences thus obtained were mapped to the genomic sequence of Kp2H7 strain using bowtie2-v2.4.2. Bowtie 2 is referenced in B. Langmead and S. L. Salzberg, “Fast gapped-read alignment with Bowtie 2,” Nature Methods 2012 9:4, vol. 9, no. 4, pp. 357-359, March 2012, doi: 10.1038/nmeth. 1923. The number of mapped reads was counted for each gene using samtools-v1.11 and featureCounts-v1.5.2 and normalized by TPM (Transcripts Per Million). SAMtools is referenced in H. Li et al., “The Sequence Alignment/Map format and SAMtools,” Bioinformatics, vol. 25, no. 16, pp. 2078-2079, 2009, doi: 10.1093/bioinformatics/btp352. FeatureCounts is referenced in Y. Liao, G. K. Smyth, and W. Shi, “featureCounts: an efficient general purpose program for assigning sequence reads to genomic features,” Bioinformatics, vol. 30, no. 7, pp. 923-930, April 2014, doi: 10.1093/BIOINFORMATICS/BTT656. By assuming that the read sequence corresponds to the mutation position of the transposon-inserted Kp strain and that each Kp mutant strain has one transposon mutation in the genome thereof, TPM was regarded as the mutation rate of each gene in the specimen, and comparative analysis was performed.

### Transcriptome Analysis of Kp-2H7 in Feces

Total RNA was isolated from fecal samples using NucleoSpin RNA (MACHEREY-NAGEL). Libraries for RNA sequencing were prepared using TruSeq Stranded mRNA Library Prep (Illumina Inc.) and sequenced in 150-bp paired-end mode using HiSeq X (Illumina Inc.). To analyze the transcriptome profile of Kp-2H7 with or without F18-mix (same as F18mix in WO 2020/179868), the genome sequence of Kp-2H7 and the genome sequence of F18-mix were concatenated to create a reference genome (hereinafter referred to as a concatenated reference genome). The sequenced paired-end reads were trimmed and filtered using Trimmomatic version 0.39 with “ILLUMINACLIP: 2: 30:10 LEADING: 3 TRAILING: 20 SLIDINGWINDOW: 4:15 MINLEN: 30” options and FASTX-Toolkit version 0.0.13 with “-q20 -p80” options. FASTX-Toolkit version 0.0.13 is provided on, http://hannonlab.cshl.edu/fastx_toolkit/index.html). Unpaired reads were excluded from further analysis.

The remaining reads were mapped to the mouse reference genome (mm10) and Phix reference genomes using minimap2 version 2.17-r941 with “-N1 -a” options. Reads that did not map to either the mouse genome or the Phix genome were extracted and used for subsequent analysis. These reads were mapped to the concatenated reference genome using bowtie2 version 2.3.4.1. The reads mapped to each Kp-2H7 gene were counted. Differential expression analysis between the treatments with or without F18-mix was performed using DESeq2 version 1.28.1. Genes with a False Discovery Rate of 5% or less by Benjamini Hochberg-correction were identified as differentially expressed genes. The heatmap was obtained from variance stabilizing transformation (VST) values obtained from the output of DESeq2.

### Measurement of Gluconate Concentration 2

For Example 10 described later, the gluconate concentration was measured as shown below. To evaluate bacterial gluconate utilization in vitro, isolated strains were cultured in mGAM or RCM medium containing 300 UM gluconate at 37° C. under anaerobic conditions for 48 hours. The supernatant of each culture medium was collected, and the gluconate concentration was measured using ExionLC AD and the SCIEX Triple Quad 6500+LC-MS/MS system (both manufactured by SCIEX). To evaluate carbon concentration in feces, each fecal sample was suspended in water (50 mg/mL), and carbon concentration in the culture supernatant was measured by LC-MS/MS. The measurement conditions for gluconate were as follows: chromatographic separation was performed using an Intrada Organic Acid column 150×2 mm (manufactured by Imtakt); the column temperature was 40° C.; and the injection volume was 2 μL. Each mobile phase consisting of phase A (acetonitrile/water/formic acid=10/90/0.1 (volume ratio)) and phase B (acetonitrile/100 mM ammonium formate=10/90 (volume ratio)) was used under the following gradient conditions: 0-3 min, A 100% B 0%; 3-10 min, A 100%, B 0%; 10-13 min, A 0%, B 100%; 13-13.1 min, A 0%, B 100%; and 13.1-18 min, A 100%, B 0%. The flow rate was 0.2 mL/min.

Detailed MS conditions are as follows: curtain gas, 30 psi; collision gas, 9 psi; ion spray voltage, −4500 V; temperature, 400° C.; ion source gas 1, 50 psi; ion source gas 2, 80 psi.

### Search for Gluconate Metabolism-Related Genes

Illumina MiSeq and PacBio Sequel platforms were used for bacterial whole genome sequencing. Illumina MiSeq sequencing was performed using a library with a target insert size of 550 bp, prepared using the TruSeq DNA PCR-free library prep kit (manufactured by Illumina). All Illumina MiSeq sequencing reads obtained were trimmed and filtered using FASTX-toolkit (version 0.0.13). Sequencing using PacBio Sequel sequencing was performed using a library prepared using the SMRTbell template prep kit 1.0. Genome assembly was performed using the hybrid assembler Unicycler using both types of sequencing data. Genome classification was performed using classify wf of GTDB-tk version 2.3.0 and the GTDB database R214. NCBI-genome-download version 0.3.3 (DOI: 10.5281/zenodo. 8192432) and the NCBI taxonomy database unknownlineage.dmp (downloaded on 2023 Sep. 14) were used to search for the NCBI taxonomy of the FastANI reference genome related to each strain's genome. Genes were predicted using Prokka version 1.14.0 with the “--kingdom Bacteria --rnammer” option and rnammer version 1.2. Homology searches for predicted genes were performed using diamond version 2.0.15 with the “blastp --evalue 0.00001 --id 30 --query-cover 60 --ultra-sensitive” options, with the KEGG (downloaded on 19/04/2022), COG (downloaded on 19/05/2021), and UniRef90 (downloaded on 24/05/2022; https://www.uniprot.org/help/uniref) databases.

### Mouse Experiment 2

For Example 12 described later, the following mouse experiments were conducted. Germ-free (GF) C57BL/6N mice, 4 to 8 weeks of age, were purchased from CLEA Japan, Inc. or Sankyo Labo Service Corporation, Inc. They were maintained in a vinyl breeding isolator (sterile isolator, ICM-1B), and mice 8 to 14 weeks of age were used in the following experiments. CL-2 (CLEA Japan) was used as a diet for mice.

Isolated bacterial strains were cultured in mGAM liquid medium, EG medium, or CM0149 medium at 37° C. in an anaerobic chamber for 24 to 48 hours. The resulting culture solutions, each containing an isolated strain were appropriately mixed in equal volumes. Furthermore, these mixed solutions were concentrated fivefold to prepare F18mix and F13mix (F31-18mix). Aliquots of the bacterial solution were administered into the stomachs of the mouse using an oral gavage needle at a dose of 200 μL per mouse (the total quantity of each bacterial strain per mouse is equivalent to 1×10⁹ CFU).

A bacterial solution containing Kp2H7 strain was placed in LB liquid medium and cultured overnight at 37° C., and the OD of the bacterial solution was adjusted to 1.2 (equivalent to 1×10⁹ CFU/mL), and then diluted 100000-fold. Then, aliquots of the bacterial solution were administered into the stomachs of individual mice using an oral gavage needle at a dose of 200 μL per mouse (equivalent to 2×10⁸ CFU).

Fecal samples from mice were dissolved in a PBS containing glycerol (final concentration 20%) and EDTA (final concentration 10 mM) at a ratio of 50 mg of feces/mL of PBS. The resulting fecal lysate was diluted to an appropriate concentration, plated onto DHL medium containing 50 mg/L ampicillin and 50 mg/L spectinomycin, and incubated overnight at 37° C. Then the number of colonies was counted, and CFU per 1 g of feces was calculated.

The results obtained using the above materials and methods are shown below.

### Example 1

### Analysis of the Mechanism of Bacterial Suppression in the Intestine by F18Mix Using Transposon-Inserted Kp2H7 Strain

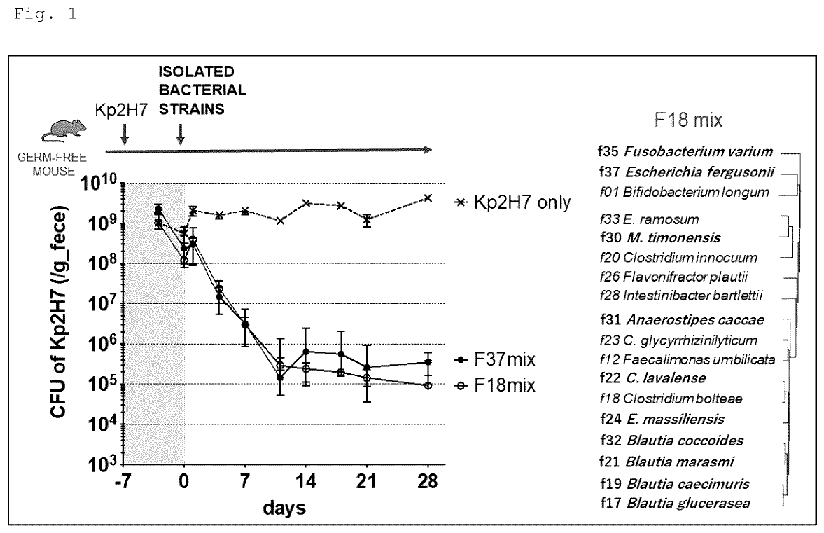

The present inventors have previously identified Kp2H7 strain, which is thought to belong to Klebsiella pneumoniae, as a bacterium that colonizes the intestinal tract and induces proliferation or activation of Th1 cells, thereby being involved in the onset of enteritis etc. (WO 2018/084172). The present inventors also assumed the existence of bacteria that suppress intestinal colonization of such Th1 cell-inducing bacteria in human intestinal bacteria, and attempted to identify them, resulting in successful isolation and culture of 37, 47, and 42 intestinal bacterial strains, respectively, from fecal samples derived from healthy individuals (subject numbers: #F, #K, and #I). Furthermore, it has been identified that administration of these bacterial strains suppresses intestinal colonization of Kp2H7 strain (WO 2019/017389). Regarding the ability to suppress intestinal bacterial colonization, the present inventors have successfully selected 31 intestinal bacterial strains (F31mix) excluding duplicate bacteria from the 37 intestinal bacterial derived from the healthy strains individual #F (F37mix). Further, as shown in FIG. 1, the present inventors have also successfully selected 18 strains (F18mix) capable of exhibiting the ability to suppress bacterial colonization comparable to F37mix (WO 2020/179868). However, it has not been found that by what mechanism such intestinal bacteria can suppress intestinal colonization of proinflammatory bacteria such as Kp2H7 strain.

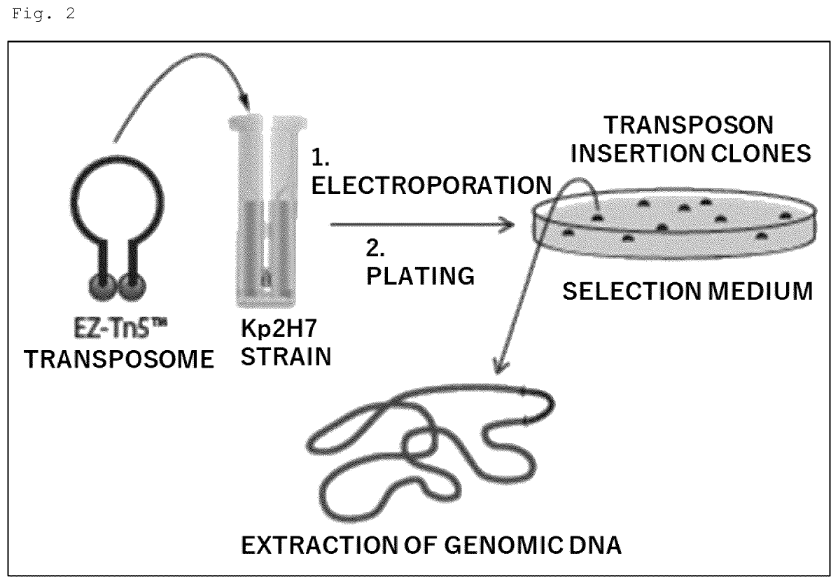

Therefore, in order to explore such mechanisms, first, a transposon, which is a complex of a transposon and a transposase, was introduced into Kp2H7 strain by electroporation. The transposon contained a kanamycin resistance gene, and when the introduced transposon was integrated into the chromosome of Kp2H7 strain, it became a kanamycin-resistant strain, so that a transposon-inserted mutant strain was selected in a kanamycin-containing medium. About 800,000 of these strains were collected and mixed to create a mutant strain library (FIG. 2). Since the transposon is inserted randomly, each colony that has grown on the selection medium is presumed to have a mutation at a different site. Since the genome size of Kp2H7 is about 5.5 Mbp, it can be estimated by simple calculation that there is one mutation per 7 bp.

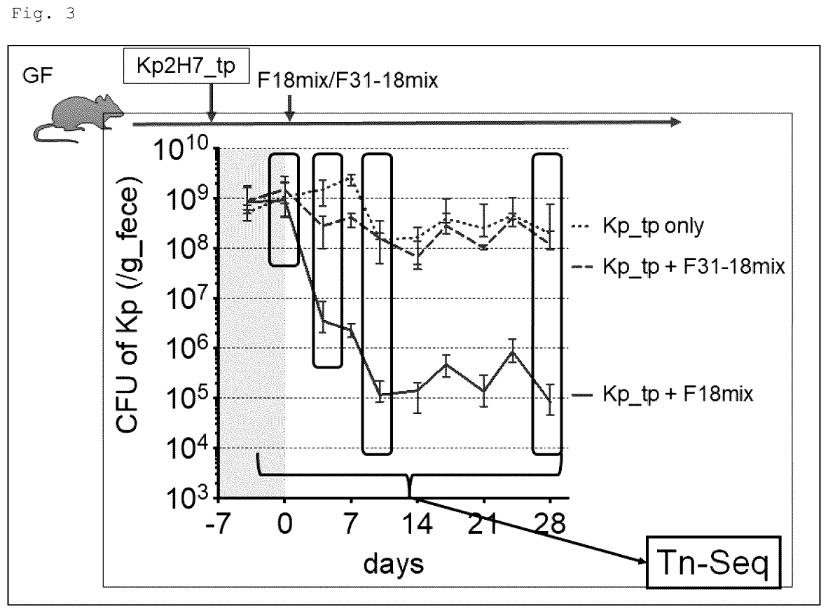

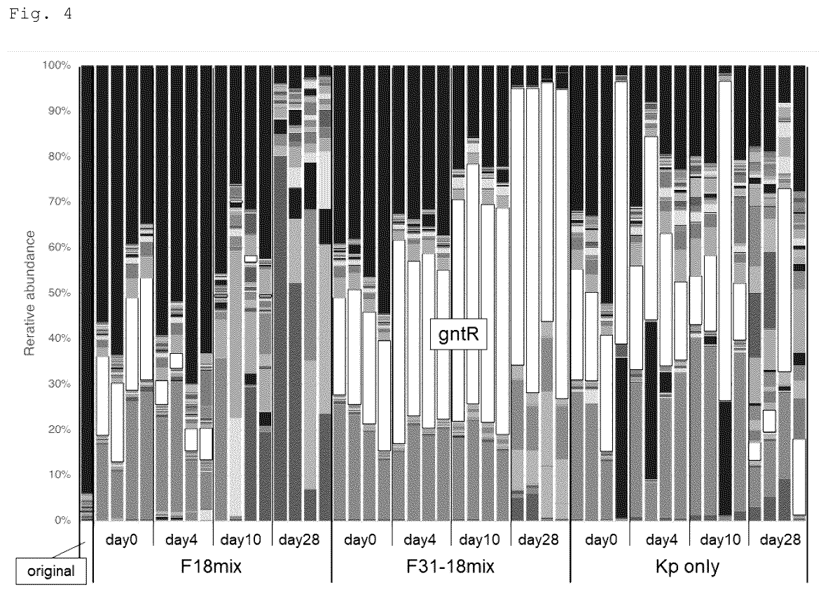

Next, as shown in FIG. 3, the mutant strain library (Kp2H7_tp) was administered to germ-free mice, followed by administration of F18mix or F31-18mix. After that, feces were collected at Days 0, 4, 10, and 28, and analysis was performed as to which gene was mutated in Kp2H7 strain contained in the feces and the proportion of the mutant strain present (Tn-seq); the proportion of mutated genes was shown for each mouse at each time point (FIG. 4). As a result, the strain with a mutation in gntR became dominant in the F31-18mix administration group and the group administered only with Kp2H7_tp (Kp only), while a gradual decrease in the gntR mutant strain was observed in the group administered with F18mix.

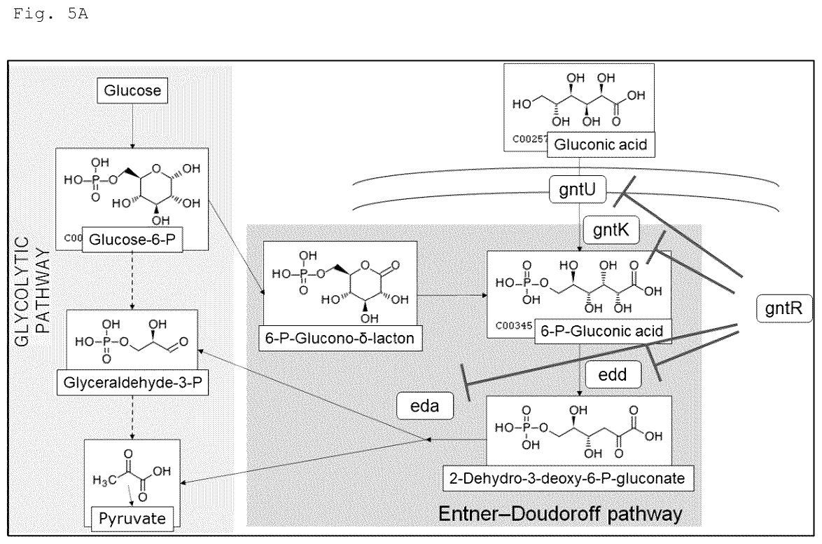

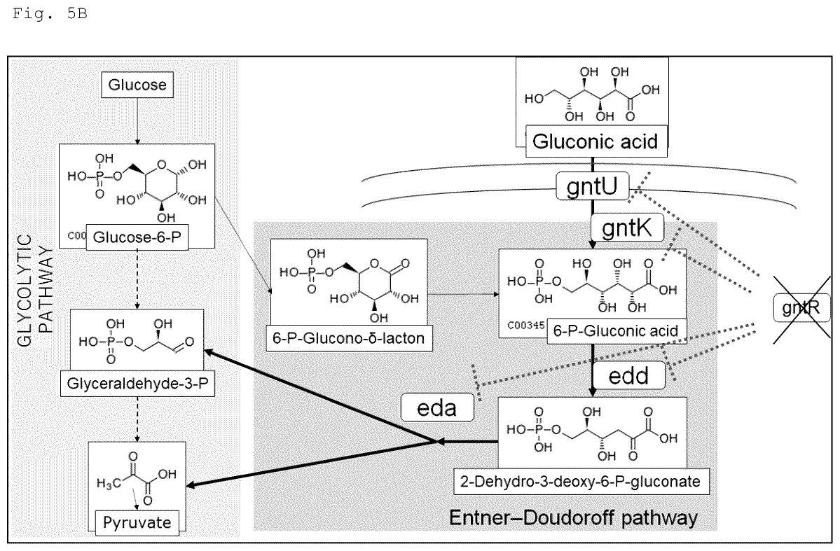

gntR is a gene related to gluconate metabolism. As shown in FIG. 5A, Klebsiella, Escherichia coli, etc. have the Entner-Doudoroff pathway (ED pathway) in addition to the common glycolytic pathway (EMP pathway) as a metabolic pathway for carbohydrate, and gluconate directly enters this ED pathway and is metabolized to pyruvate in three steps. gntR is known to act suppressively on gntU, gntK, edd, and eda, which are genes that metabolize gluconate in the ED pathway.

However, if there is a mutation in gntR, as shown in

FIG. 5B, the suppression of gluconate metabolism is eliminated, so that the ED pathway works more actively. In a gluconate-rich environment, gntR mutant strains are expected to have a survival advantage. In other words, it can be presumed that the mouse's intestinal environment is gluconate-rich when only Kp2H7_tp was administered, or when F31-18mix is additionally administered.

On the other hand, in the presence of F18mix, the dominance of the gntR mutant strain is lost, indicating that gluconate in the environment is scarce and Klebsiella cannot utilize gluconate, which is an important carbon source. In other words, this is considered to be the result of a decrease in intestinal gluconate due to the utilization/consumption of gluconate by F18mix.

### Example 2

### Analysis of the Ability of F18Mix and Kp2H7 Strain to Utilize Gluconate

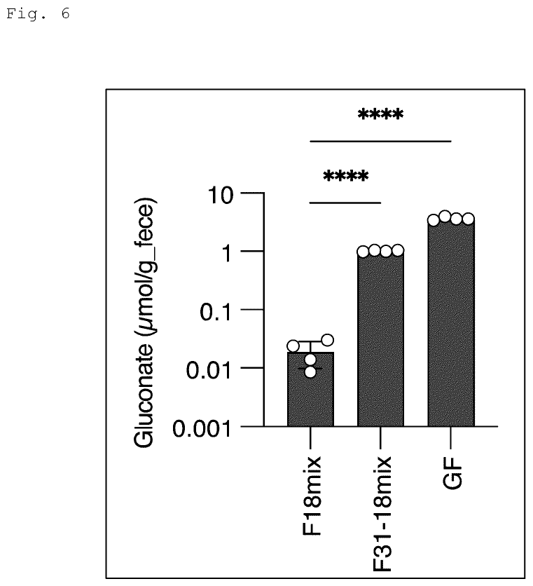

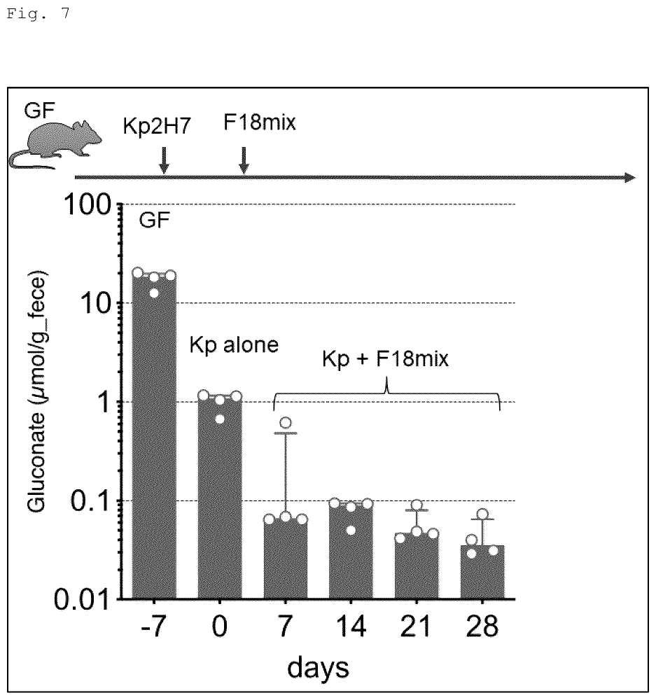

Based on the above presumption, the gluconate concentration in feces was actually measured. As shown in FIG. 6, when F18mix was administered to germ-free mice, the gluconate concentration in feces was significantly decreased compared to that in mice administered with F31-18mix or in germ-free mice. Furthermore, when Kp2H7 strain was administered to germ-free mice and then F18mix was additionally administered, the gluconate concentration in feces decreased, as shown in FIG. 7.

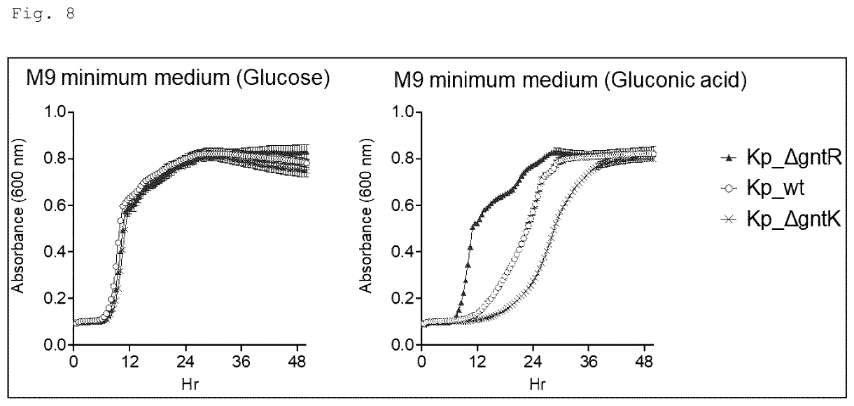

Furthermore, gene deletion strains for gntK and gntR were generated. These deletion strains were cultured in a minimum medium containing only glucose or only gluconate as a carbon source, and growth curves were drawn. As a result, as shown in FIG. 8, all deletion strains grew similarly in glucose-containing medium, but in gluconate-containing medium, the gntK-deficient strain (Kp ΔgntK) grew slowly and the gntR-deficient strain (Kp_ΔgntR) grew rapidly.

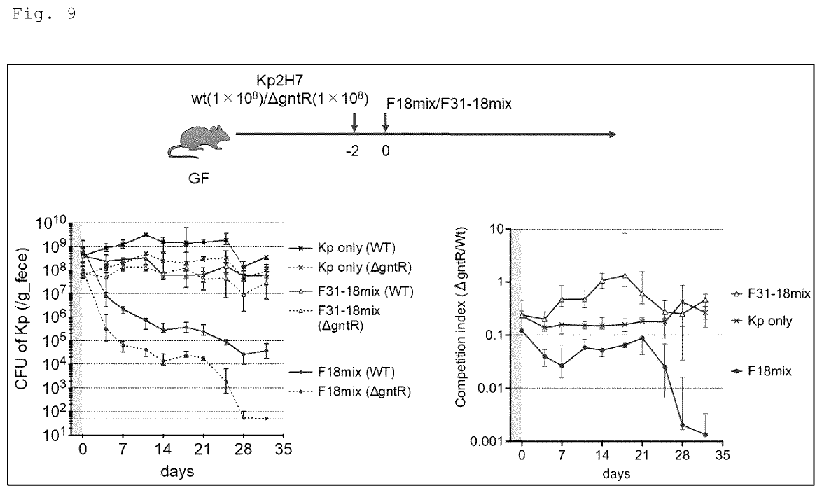

Furthermore, germ-free mice were administered a mixture of Kp2H7 strain (wild-type strain) and the gntR-deficient strain (ΔgntR) in which the bacterial amounts of the each strain were equal. Two days later, F18mix or F31-18mix was additionally administered, and the bacterial amount of Kp2H7 strain in the feces of these mice was measured. As a result, as shown in the graph on the left side of FIG. 9, the bacterial amount of the gntR-deficient strain decreased earlier than that of the wild-type strain by administration of F18mix. On the other hand, in the F31-18mix administration group and the group administered only with Kp2H7 strain (Kp only), the bacterial amount of Kp2H7 strain in feces did not change significantly. Furthermore, the ratio in bacterial amount of gntR-deficient strain to that of the wild-type strain in feces (Competition index (ΔgntR/WT)) was analyzed. As a result, as shown in the graph on the right side of FIG. 9, the decrease in the relative amount of, particularly, gntR-deficient strain was significant after Day 21 in the F18mix administration group. This suggests that gluconate was depleted in the intestines of these mice.

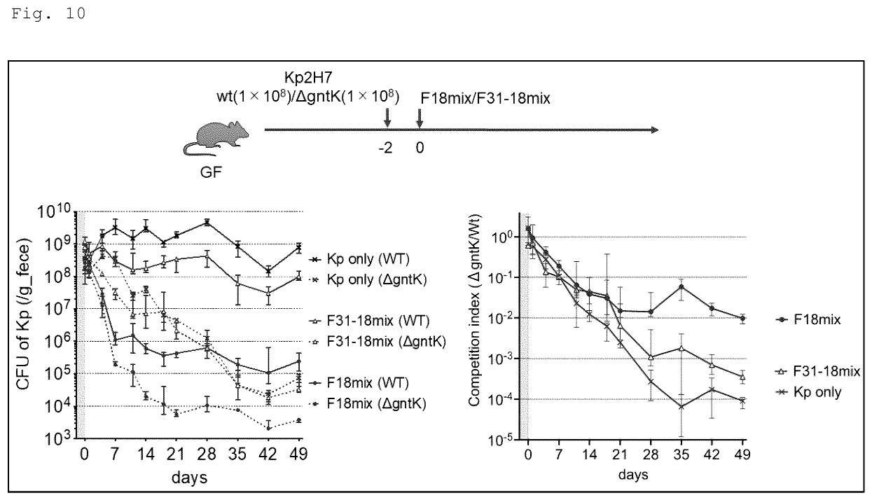

Similar to the above, germ-free mice were administered a mixture of Kp2H7 strain (wild-type strain) and the gntK-deficient strain (ΔgntK) in which the bacterial amounts of the each strain were equal. Two days later, F18mix or F31-18mix was additionally administered, and the bacterial amount of Kp2H7 strain in the feces of these mice was measured. As a result, as shown in the graph on the left side of FIG. 10, the bacterial amount of the gntK-deficient strain was significantly decreased in the F31-18mix administration group and the group administered only with Kp2H7 strain (Kp only). As shown by the competition index (as shown in the graph on the right side of FIG. 10), the decrease in the gntK-deficient strain stopped after Day 21 in the intestines of the F18mix administration group, suggesting that gntK-deficient Klebsiella strain did not utilize gluconate as a nutrient source.

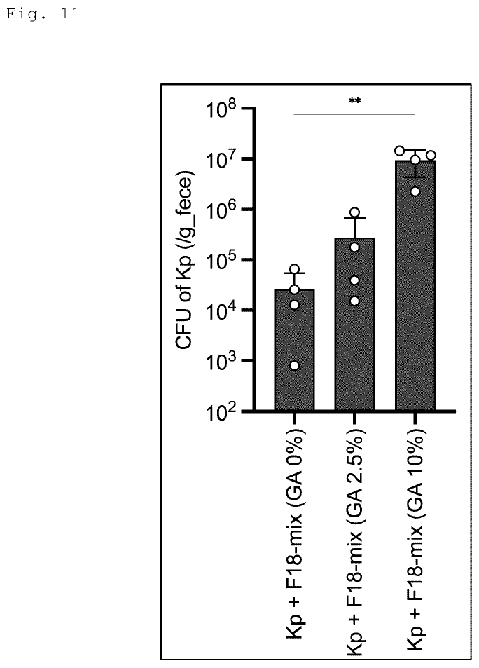

Next, as described above, mice colonized with Kp2H7 strain and F18mix in the intestine were prepared. These mice were fed CL-2 (gluconate content: 5 μmol/g) until Day 20 after the administration of F18mix, and from Day 21 onward, they were fed a diet containing either 0%, 2.5% (80 μmol/g), or 10% (320 μmol/g) gluconate. As a result, as shown in FIG. 11, the bacterial amount of Kp2H7 strain in feces was significantly increased in the group fed a diet containing 10% gluconate, one week after the diet was switched.

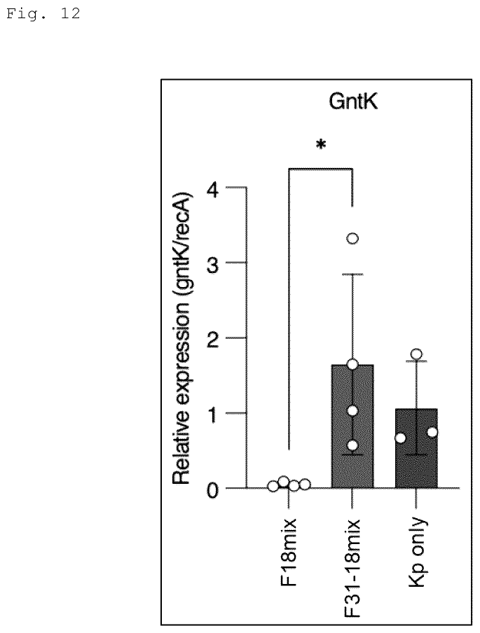

Furthermore, F18mix or F31-18mix was administered to mice colonized with only Kp2H7 strain, and the expression of the gntK gene in Kp2H7 strain was analyzed 2 days later. As a result, as shown in FIG. 12, the expression of this gene was low in the F18mix administration group.

From the above, it was found that F18mix utilizes/consumes gluconate in the intestine and suppresses the proliferation of Klebsiella.

### Example 3

### Identification of Gluconate-Utilizing Bacteria in F18mix

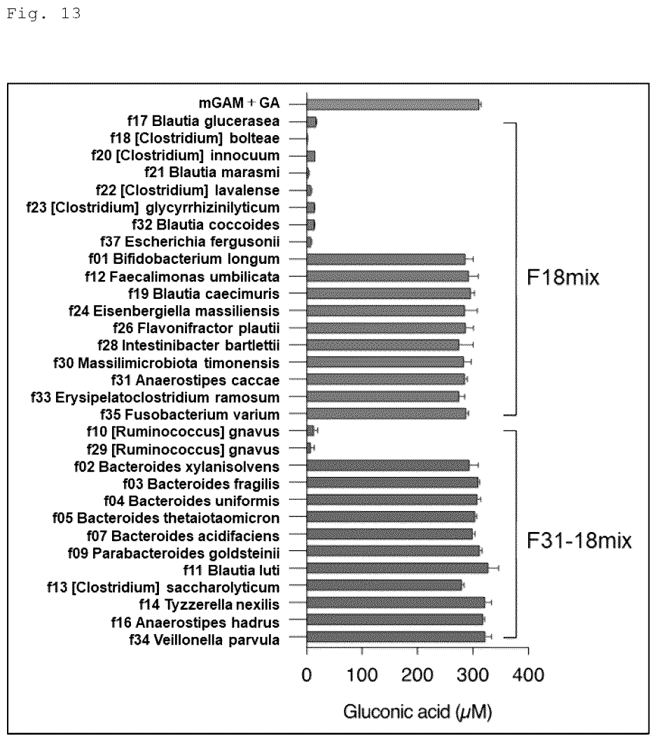

Next, in order to investigate which bacteria in F18mix can utilize/consume gluconate, various isolated bacterial strains were cultured in modified GAM broth containing 300 μM gluconate. Then 48 hours later, the culture supernatant was collected to measure the gluconate concentration. A bacterial strain whose culture supernatant shows a decrease in gluconate concentration was identified as capable of utilizing gluconate. As a result, as shown in FIG. 13, it was found that 8 strains in F18mix were bacterial strains capable of utilizing gluconate. Furthermore, it was found that the 13 bacterial strains of F31-18mix also contained 2 bacterial strains capable of utilizing gluconate. Specifically, 8 bacterial strains-f17 (18.4 μM), f18 (1.7 UM), f20 (16.5 μM), f21 (4.2 μM), f22 (9.0 UM), f23 (15.4 UM), f32 (15.0 μM), and f37 (9.2 μM), and 2 bacterial strains-f10 (14.2 μM) and f29 (8.2 μM) have the ability to consume gluconate. For the notation (No.) of these bacterial strains, refer to Tables 1 and 3. The values in parentheses indicate the residual gluconate concentrations (average value of three measurements) in the culture supernatants of each bacterial strain after 48 hours of culture. The residual gluconate concentrations (average value of three measurements) in culture supernatants of the 21 bacterial strains other than these 10 bacterial strains was over 270 UM.

### Example 4

### Analysis of the Ability of Pathogenic Microorganisms to Utilize Gluconate

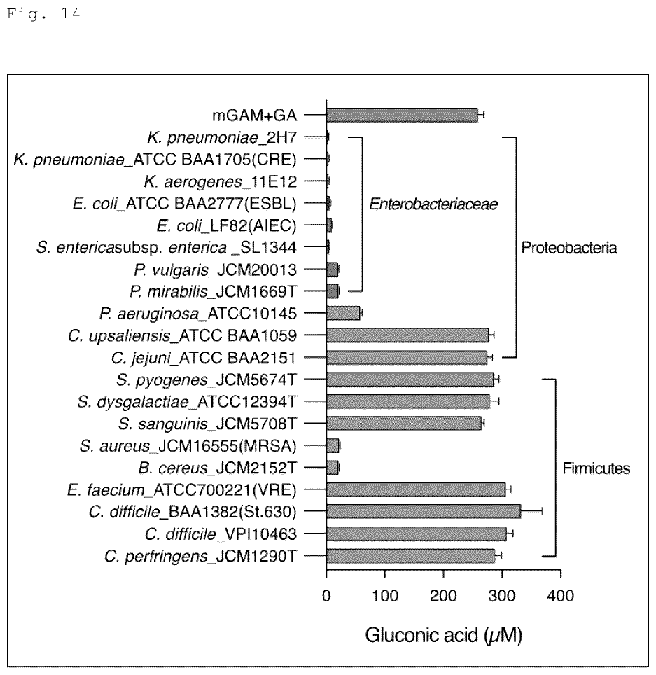

Next, it was investigated whether pathogenic microorganisms other than Klebsiella could utilize/consume gluconate. As a result, as shown in FIG. 14, bacteria belonging to the family Enterobacteriaceae, including Klebsiella (as well as Escherichia coli, Salmonella enteritidis, Proteus vulgaris, Proteus mirabilis, Pseudomonas aeruginosa), consumed gluconate. Furthermore, it was found that Staphylococcus aureus and Bacillus cereus could also consume gluconate.

On the other hand, Campylobacter (Campylobacter upsaliensis, Campylobacter jejuni), Streptococcus (Streptococcus pyogenes, Streptococcus dysgalactiae, Streptococcus sanguinis), Enterococcus faecium, and Clostridium (Clostridium difficile, Clostridium perfringens) could not consume gluconate.

### Example 5

### Combined Effect of Gluconate-Utilizing Bacteria and Non-Utilizing Bacteria in F18Mix

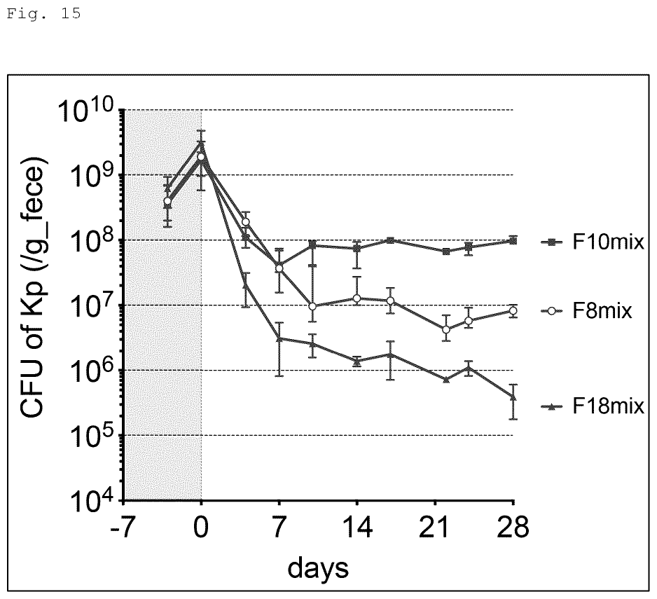

After colonizing germ-free mice with Kp2H7 strain, F18mix, F8mix, or F10mix was administered. The F8mix consisted of eight strains that utilize gluconate, selected from the 18-strains of F18mix. The F10 mix consisted of ten strains that do not utilize gluconate, selected from F18mix (F18-8mix). As a result, as shown in FIG. 15, the bacterial amount of Kp2H7 strain in feces decreased in the order of F18mix administration group, F8mix administration group, and F10mix administration group.

As described above, when F18mix was divided into strains utilizing gluconate (F8mix) and strains not utilizing gluconate (F10mix), F8mix suppressed Kp2H7 strain stronger than F10mix. This is probably because F8mix competed with Kp2H7 strain for gluconate consumption in the intestine, resulting in stronger suppression of the growth of Klebsiella etc. compared with F10mix.

On the other hand, when comparing F18mix and F8mix, the decrease in the bacterial amount of Kp2H7 strain was greater with F18mix. This suggests that, rather than using only gluconate-utilizing strains, additionally using other specific strains may lead to interactions or the like, resulting in a stronger Klebsiella-suppressive effect.

### Example 6

### Analysis of the Suppressive Ability of F18Mix on Bacteria in the Intestine Depending on the Concentration of Gluconate

The present inventors focused on the fact that most of the gluconate in the intestine is derived from diet. Therefore, the relationship between diet and the amount of Klebsiella bacteria in mice administered with F18mix was examined.

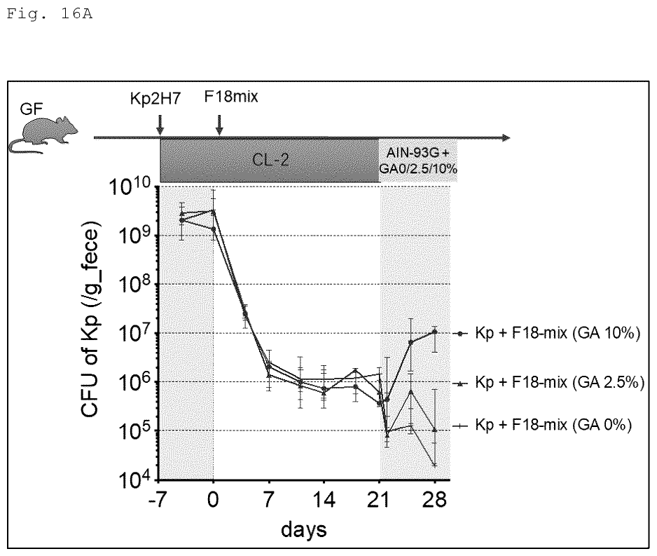

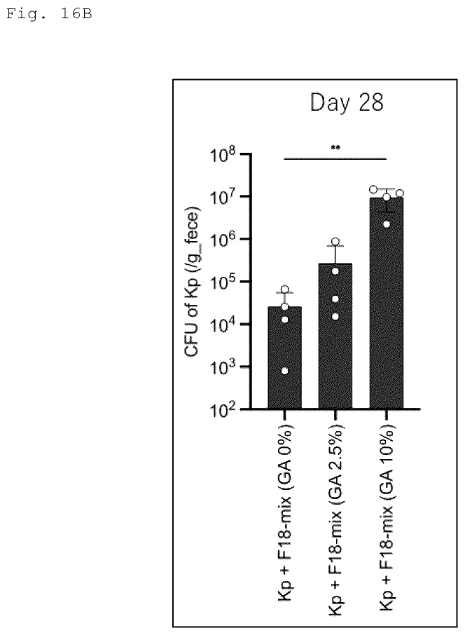

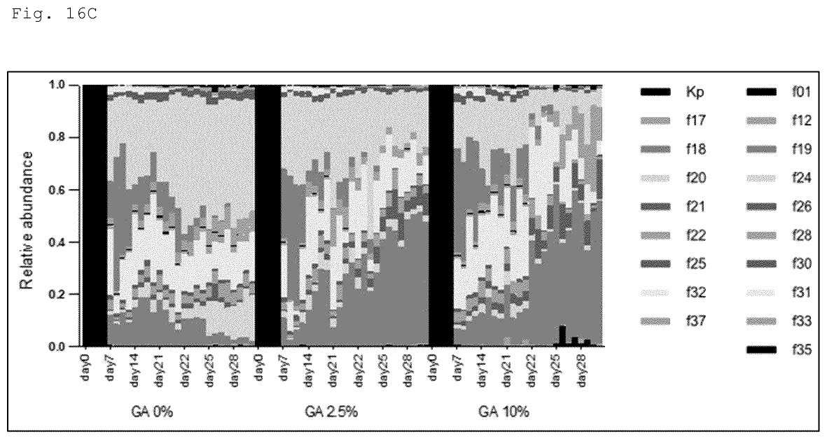

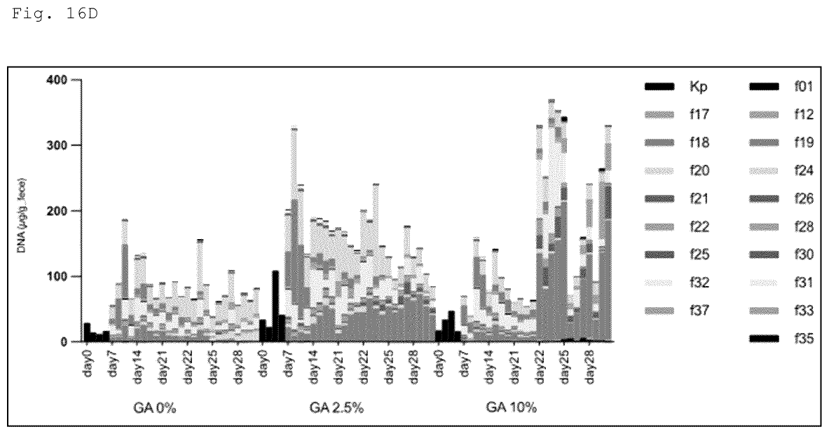

After colonizing germ-free mice with Kp2H7 strain, F18mix was administered. The diet was CL-2 until Day 21. After that, AlN-93G with various gluconate contents was given to the mice as the diet. As a result, as shown in FIGS. 16A and 16B, a decrease in the bacterial amount of Kp2H7 strain was observed when the diets with a gluconate content of 0% or 2.5% by weight was used. On the other hand, when the diet with a gluconate content of 10% by weight was used, an increase in the bacterial amount of Kp2H7 strain was observed.

As described above, when the gluconate content was 2.5% by weight or less, a decrease in the bacterial amount of Kp2H7 strain was observed. This suggests that, in addition to the competition between Klebsiella and F18mix for gluconate, the creating a low-gluconate environment may itself be effective in suppressing Klebsiella. On the other hand, when the gluconate content was high, an increase in the bacterial amount of Klebsiella was observed even with competition with F18mix. However, regarding this increase, the rate of increase was suppressed by administration of F18mix.

### Example 7

### Analysis of the Growth of F18Mix in the Intestine

The same test as described above was performed, and feces from each mouse were collected. Bacterial genomes were extracted from the obtained feces, and the amount of each bacterium was quantified. The amount of bacteria was confirmed in both relative amount and absolute amount. As a result, as shown in FIGS. 16C and 16D, a rapid decrease in the bacterial amount of Kp2H7 strain was observed in the period from the day of administration of F18mix to Day 21. Also, as a result until Day 21, there was a tendency for the amount of non-gluconate-utilizing bacteria f19 and f24 to increase significantly. This suggests the possibility that even bacteria other than gluconate-utilizing bacteria may have some Klebsiella suppressive effect.

Next, from Day 21 onward, when the diet with various gluconate contents was given, there was a tendency for the amount of gluconate-utilizing bacteria f18, f21, f22, f32, and f37 in particular to increase significantly when the diet with a gluconate content of 10% was used, compared to when mouse diet with a gluconate content of 0% was used. This suggests the possibility that these 5 species of bacterial strains especially utilize gluconate, compared to other gluconate-utilizing bacteria.

### Example 8

### Gene Expression Analysis in the Intestine

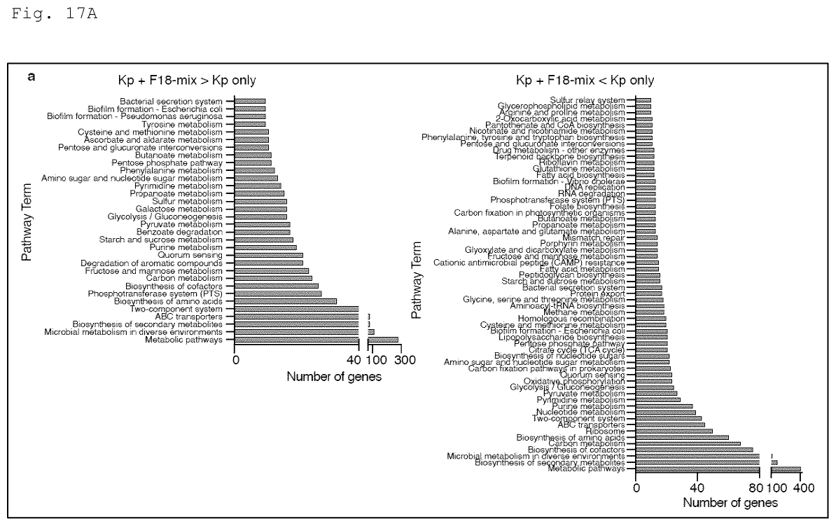

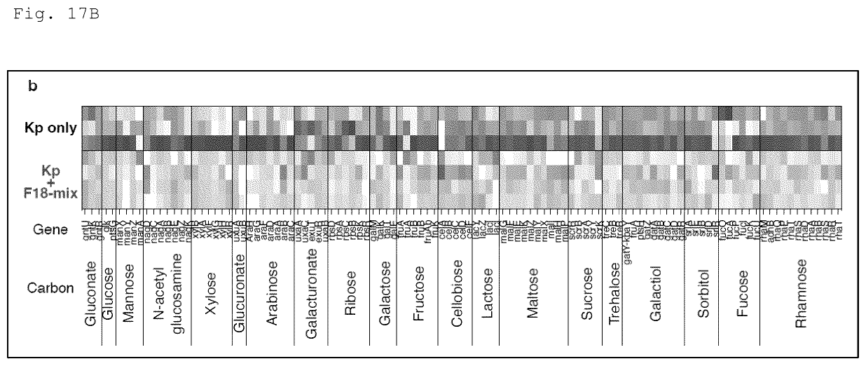

Either Kp-2H7 alone or Kp-2H7+F18-mix was administered to germ-free mice. Fecal samples were collected from mice 2 days after administration of F18-mix, and RNA-seq analysis of bacteria in feces was performed. KEGG pathway analysis was performed on genes with a significant difference (Benjamini-Hochberg corrected p-value <0.001) between groups in terms of the expression level of RNA derived from Klebsiella. Pathways consisting of 10 or more genes whose expression was increased or decreased in the Kp-2H7+F18-mix group compared to the Kp-2H7 alone group are shown in FIG. 17A. The expression profiles of genes involved in carbohydrate metabolism are shown in FIG. 17B.

As shown in FIG. 17A, significant changes were observed, especially in pathways related to carbohydrate metabolism and amino acid metabolism. Furthermore, it is considered that from the results shown in FIG. 17B, regarding genes related to carbohydrate metabolism, genes related to gluconate metabolism and glucose metabolism were highly expressed in the Kp-2H7 alone group. On the other hand, the main carbon source is considered to have shifted to other carbon sources, such as fructose, lactose, and sorbitol, due to coexistence with F18mix.

### Example 9

### Analysis of Gluconate Concentration in the Intestine

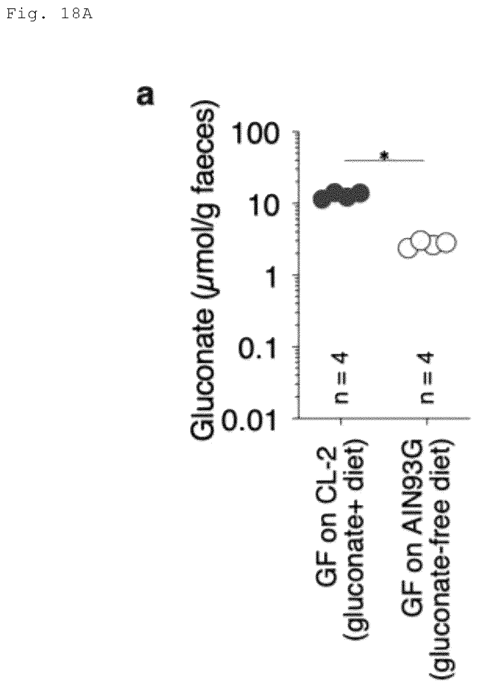

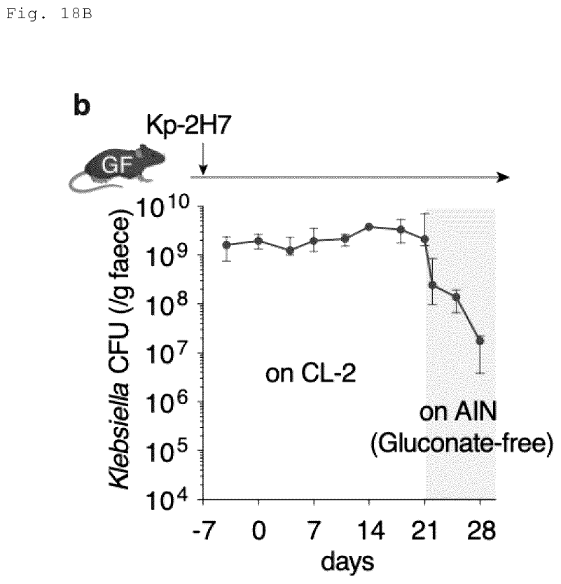

Germ-free mice were raised on either CL-2 (manufactured by CLEA Japan, Inc.) as nutrient-rich diet containing gluconate or AlN93G (manufactured by Oriental Yeast Co., Ltd.) as gluconate-free diet. The gluconate concentration in the feces of these germ-free mice was measured by LC-MS. As shown in FIG. 18A, the amount of gluconate in feces varies depending on the diet, but gluconate is detected even if it is not contained in the diet, so that the gluconate in feces is considered to be supplied from the diet and the host.

Furthermore, Kp-2H7 was administered to germ-free mice raised on CL-2, and the diet was changed from CL-2 to AlN93G on Day 21. Fecal CFU of Kp-2H7 is shown as median±IQR. As a result, as shown in FIG. 18B, the bacterial amount of Kp-2H7 in feces decreased after the change to AlN93G. Therefore, it is considered that the change in the concentration of gluconate derived from diet affects the change in the bacterial amount of Kp-2H7.

### Example 10

### Gene Cluster Analysis in Gluconate-Utilizing Bacteria

Bacterial strains isolated from healthy individuals #F, #K, and #I (WO 2020/179868) were cultured in mGAM medium containing 300 UM gluconate at 37° C. for 48 hours (n=3). Then, the gluconate concentration in the culture supernatant was measured by LC-MS/MS. If the gluconate concentration in the medium decreased, the bacteria were determined to be capable of gluconate utilization. Among the genes considered to be involved in gluconate metabolism from the nucleotide sequence of the genome of all bacterial strains, those capable of forming gene clusters are shown in FIGS. 19A to 19D.

As a result, it was found that the strains capable of utilizing gluconate have gluconate kinase and gluconate transporter, or gluconate dehydratase and gluconate transporter as gene clusters, and also have KDGK or Eda genes necessary for subsequent metabolism as neighboring or separate gene clusters, as indicated in FIGS. 19A to 19D.

The asterisk in FIG. 19B indicates a frameshift mutation in gluconate dehydratase of f19 Blautia caecimuris strain. This enzyme is non-functional due to the frameshift mutation, so that gluconate is not considered to have decreased.

### Example 11

### Operon Search

Genome sequence, protein sequence, and gene annotation information were obtained for representative sequences at the species level of prokaryotes registered in the NCBI Assembly (https://www.ncbi.nlm.nih.gov/assembly/) as of Sep. 20, 2023 (14 reference species; 2 sequences only for Escherichia coli, 18, 326 representative species).

Gluconate dehydratase (gad), gluconate kinase (gntK), and transporter protein sequences identified in FIGS. 19A to 19D were collected to create a gluconate metabolism-related gene database (DB).

TABLE 9

| | Gluconate Kinase | | Gluconate Dehydrogenase | | Gluconate Transporter | |
|---|---|---|---|---|---|---|
| | | SEQ | | SEQ | | SEQ |
| No. | Enzyme Name | ID: | Enzyme Name | ID: | Enzyme Name | ID: |
| F01 | f01_CNLHCNON_00027_Glu- | 76 | — | — | — | — |
| | conokinase | | | | | |
| F10 | — | — | f10_FONBLEPD_00269_Dihydroxy- | 86 | f10_FONBLEPD_00271_Low- | 118 |
| | | | acid_dehydratase | | affinity_gluconate_transporter | |
| F13 | — | — | f13_FHKCONON_03198_Dihydroxy- | 87 | f13_FHKCONON_00253_Low- | 119 |
| | | | acid_dehydratase | | affinity_gluconate_transporter | |
| F17 | — | — | f17_EAOGLLOI_00767_Dihydroxy- | 88 | f17_EAOGLLOI_00766_Low- | 120 |
| | | | acid_dehydratase | | affinity_gluconate_transporter | |
| | | | | | f17_EAOGLLOI_00912_High- | 121 |
| | | | | | affinity_gluconate_transporter | |
| F18 | — | — | f18_PGAODDDK_05264_Dihydroxy- | 89 | f18_PGAODDDK_02227_High- | 122 |
| | | | acid_dehydratase | | affinity_gluconate_transporter | |
| | | | | | f18_PGAODDDK_05263_Low- | 123 |
| | | | | | affinity_gluconate_transporter | |
| F19 | — | — | f19_DHAAOBAE_00692_Dihydroxy- | 90 | f19_DHAAOBAE_00574_Gnt- | 124 |
| | | | acid_dehydratase | | II_system_L-idonate_transporter | |
| | | | | | f19_DHAAOBAE_00693_Low- | 125 |
| | | | | | affinity_gluconate_transporter | |
| F21 | — | — | f21_EKJEFAGC_00541_Dihydroxy- | 91 | f21_EKJEFAGC_00543_In- | 126 |
| | | | acid_dehydratase | | ner_membrane_permease_YgbN | |
| | | | | | f21_EKJEFAGC_05068_Low- | 127 |
| | | | | | affinity_gluconate_transporter | |
| F22 | — | — | f22_PKNBPMIK_04122_Dihydroxy- | 92 | f22_PKNBPMIK_01433_High- | 128 |
| | | | acid_dehydratase | | affinity_gluconate_transporter | |
| | | | | | f22_PKNBPMIK_04125_High- | 129 |
| | | | | | affinity_gluconate_transporter | |
| | | | | | f22_PKNBPMIK_04206_In- | 130 |
| | | | | | ner_membrane_permease_YgbN | |
| F23 | — | — | f23_EBMMFAOD_00508_Dihydroxy- | 93 | — | — |
| | | | acid_dehydratase | | | |
| F24 | — | — | f24_DLFENNED_05018_Dihydroxy- | 94 | — | — |
| | | | acid_dehydratase | | | |
| F29 | — | — | f29_ALOIBIFK_03476_Dihydroxy- | 95 | f29_ALOIBIFK_03478_Low- | 131 |
| | | | acid_dehydratase | | affinity_gluconate_transporter | |
| F30 | — | — | f30_DFNANFDB_01672_Dihydroxy- | 96 | — | — |
| | | | acid_dehydratase | | | |
| F32 | — | — | f32_GFFMIMOM_00656_Dihydroxy- | 97 | f32_GFFMIMOM_00655_High- | 132 |
| | | | acid_dehydratase | | affinity_gluconate_transporter | |
| F33 | — | — | f33_IDFBFOFA_01313_Dihydroxy- | 98 | — | — |
| | | | acid_dehydratase | | | |
| F35 | — | — | — | — | f35_EMDMBBGG_02505_High- | 133 |
| | | | | | affinity_gluconate_transporter | |
| F37 | f37_MKMCEHOJ_02531_Thermo- | 77 | — | — | f37_MKMCEHOJ_02505_High- | 134 |
| | resistant_gluconokinase | | | | affinity_gluconate_transporter | |
| | | | | | f37_MKMCEHOJ_02530_Low- | 135 |
| | | | | | affinity_gluconate_transporter | |
| | | | | | f37_MKMCEHOJ_03564_High- | 136 |
| | | | | | affinity_gluconate_transporter | |

TABLE 10

| | Gluconate Kinase | | Gluconate Dehydrogenase | | Gluconate Transporter | |
|---|---|---|---|---|---|---|
| | | SEQ | | SEQ | | SEQ |
| No. | Enzyme Name | ID: | Enzyme Name | ID: | Enzyme Name | ID: |
| I01 | i01_CAKGJPAO_00191_Glu- | 78 | — | — | i01_CAKGJPAO_00190_High- | 137 |
| | conokinase | | | | affinity_gluconate_transporter | |
| I02 | i02_JFDANPEK_00221_Glu- | 79 | — | — | i02_JFDANPEK_00220_Gnt- | 138 |
| | conokinase | | | | II_system_L-idonate_transporter | |
| I04 | i04_ALIAHKGE_00021_Glu- | 80 | — | — | — | — |
| | conokinase | | | | | |
| I07 | i07_H8OOHHLN_00021_Glu- | 81 | — | — | — | — |
| | conokinase | | | | | |
| I13 | — | — | i13_AFPGDEAI_01673_Dihydroxy- | 99 | i13_AFPGDEAI_02973_Gnt- | 139 |
| | | | acid dehydratase | | II_system_L-idonate_transporter | |
| I20 | — | — | i20_GFDADFBJ_02480_Dihydroxy- | 100 | i20_GFDADFBJ_02479_Low- | 140 |
| | | | acid_dehydratase | | affinity_gluconate_transporter | |
| I21 | — | — | i21_BEAJJIIG_02823_Dihydroxy- | 101 | i21_BEAJJIG_02822_Low- | 141 |
| | | | acid_dehydratase | | affinity_gluconate_transporter | |
| I22 | — | — | i22_MMKBGNBN_03241_Dihydroxy- | 102 | i22_MMKBGNBN_03243_Low- | 142 |
| | | | acid_dehydratase | | affinity_gluconate_transporter | |
| I23 | — | — | i23_IEINNFOL_02285_Dihydroxy- | 103 | i23_IEINNFOL_02286_Gnt- | 143 |
| | | | acid_dehydratase | | II_system_L-idonate_Lransporter | |
| I25 | — | — | i25_HOMMCNHA_02663_Dihydroxy- | 104 | i25_HOMMCNHA_02662_Low- | 144 |
| | | | acid_dehydratase | | affinity_gluconate_transporter | |
| I26 | — | — | — | — | i26_CBJLMLAF_00843_Low- | 145 |
| | | | | | affinity_gluconate_transporter | |
| I30 | — | — | — | — | i30_ILBOFAMM_02808_High- | 146 |
| | | | | | affinity_gluconate_transporter | |
| I33 | i33_ABMIDDIK_01308_Xylu- | 82 | — | — | i33_ABMIDDIX_01308_High- | 147 |
| | lose_kinase | | | | affinity_gluconate_transporter | |
| 137 | i37_IIDCKEOG_02051_Xylu- | 83 | — | — | i37_IIDCKEOG_02049_High- | 148 |
| | lose_kinase | | | | affinity_gluconate_transporter | |

TABLE 11

| | Gluconate Kinase | | Gluconete Dehydrogenase | | Gluconate Transporter | |
|---|---|---|---|---|---|---|
| | | SEQ | | SEQ | | SEQ |
| No. | Enzyme Name | ID: | Enzyme Name | ID: | Enzyme Name | ID: |
| K03 | — | — | k03_EAPGLPFA_04135_Dihydroxy- | 105 | k03_EAPGLPFA_04133_High- | 149 |
| | | | acid_dehydratase | | affinity_gluconate_transporter | |
| K04 | — | — | k04_BNLHKKFH_05311_Dihydroxy- | 106 | k04_BNLHKKFH_03808_Low- | 150 |
| | | | acid_dehydratase | | affinity_gluconate_transporter | |
| | | | | | k04_BNLHKKFH_05313_High- | 151 |
| | | | | | affinity_gluconate_transporter | |
| K05 | — | — | — | — | k05_GIKLEFGL_00727_High- | 152 |
| | | | | | affinity_gluconate_transporter | |
| K07 | — | — | k07_NGPHAEGG_00086_Dihydroxy- | 107 | — | — |
| | | | acid_dehydratase | | | |
| K12 | — | — | k12_LLKJONED_01597_Dihydroxy- | 108 | — | — |
| | | | acid_dehydratase | | | |
| K17 | — | — | k17_FCCFCLNI_05334_Dihydroxy- | 109 | k17_FCCFCLNI_01939_High- | 153 |
| | | | acid_dehydratase | | affinity_gluconate_transporter | |
| | | | | | k17_FCCFCLNI_05332_In- | 154 |
| | | | | | ner_membrane_permease_YgbN | |
| K19 | — | — | k19_HGDCKBFN_00437_Dihydroxy- | 110 | k19_HGDCKBFN_00438_Low- | 155 |
| | | | acid_dehydratase | | affinity_gluconate_transporter | |
| K25 | — | — | k25_DPGHCJGG_05294_Dihydroxy- | 111 | k25_DPGHCJGG_02370_Gnt- | 155 |
| | | | acid_dehydratase | | II_system_L-idonate_transporter | |
| K30 | — | — | k30_GGDNGPNC_02218_Dihydroxy- | 112 | k30_GGDNGPNC_02220_Low- | 157 |
| | | | acid_dehydratase | | affinity_gluconate_transporter | |
| K31 | — | — | k31_JDEDMANH_02262_Dihydroxy- | 113 | k31_JDEDMANH_02264_Low- | 158 |
| | | | acid_dehydratase | | affinity_gluconate_transporter | |
| K34 | — | — | k34_HOEMBMAP_06207_Dihydroxy- | 114 | k34_HOEMBMAP_05033_Low- | 159 |
| | | | acid_dehydratase | | affinity_gluconate_transporter | |
| | | | | | k34_HOEMBMAP_06209_In- | 160 |
| | | | | | ner_membrane_permease_YgbN | |
| L36 | — | — | k36_FDCNGLDG_04719_Dihydroxy- | 115 | k36_FDCNGLDG_02453_High- | 161 |
| | | | acid_dehydratase | | affinity_gluconate_transporter | |
| | | | | | k36_FDCNGLDG_04718_Low- | 162 |
| | | | | | affinity_gluconate_transporter | |
| K39 | k39_IHLFMFDM_00307_Thermo- | 84 | — | — | k39_IHLFMFDM_00308_Low- | 163 |
| | resistant_gluconokinase | | | | affiwnity_gluconate_transporter | |
| | | | | | k39_IHLFMFDM_00337_High- | 164 |
| | | | | | affinity_gluconate_transporter | |
| K40 | — | — | k40_AFILLPBB_01494_Dihydroxy- | 116 | k40_AFILLPBB_00534_In- | 165 |
| | | | acid_dehydratase | | ner_membrane_permease_YgbN | |
| K42 | — | — | k42_MFPDOCHA_03257_Dihydroxy- | 117 | k42_MFPDOCHA_03253_In- | 166 |
| | | | acid_dehydratase | | ner_membrane_permease_YgbN | |

TABLE 12

| | Gluconate Kinase | | Gluconate Dehydrogenase | | Gluconate Transporter | |
|---|---|---|---|---|---|---|
| | | SEQ | Enzyme | SEQ | | SEQ |
| No. | Enzyme Name | ID: | Name | ID: | Enzyme Name | ID: |
| Kp-2H7 | Kp2H7_NOCLEBFK_02257_ | 85 | — | — | Kp2H7_NOCLEBFK_02256_ | 167 |
| | Thermoresistant_gluconokinase | | | | Low-affinity_gluconate_transporter | |

Using DIAMOND version 2.0.15, for this gluconate metabolism-related gene DB, the protein sequence of each bacterial species obtained from NCBI was used as a query for homology searches under the following conditions: “diamond blastp --evlaue 0.00001 --id 60 --query-cover 60 --max-target-seqs 1 --more-sensitive”. DIAMOND is referenced in B. Buchfink et al., 2021, Nature Methods, https://www.nature.com/articles/s41592-021-01101-x.

Criteria were set such that protein sequences with homology of 60% or more coverage and 60% or more percent identity were considered hits. Here, coverage (query cover, qcovhsp) represents the ratio of the alignment length to the total length of the query amino acid sequence, and percent identity (id, pident) represents the percentage of amino acid identity to the alignment length.

Based on the results of this homology search and gene annotation information, bacteria having a gene encoding the enzyme (gad or gntK) and a gene encoding transporter in close proximity were searched for under the following conditions:

The enzyme and transporter are encoded on the same DNA;

The distance between the enzyme and transporter is 10,000 bp or less; and

There are two or fewer genes encoded between the enzyme and transporter.

As bacteria having a gene encoding enzyme and a gene encoding transporter genes in close proximity to each other that satisfy all three of these conditions, 70 species (gad) and 403 species (gnt) were identified (70 species (gad) are shown in Tables 13 to 16, and 403 species (gnt) are shown in Tables 17 to 40). Tables 13 and 14, Tables 15 and 16, Tables 17 and 18, Tables 19 and 20, Tables 21 and 22, Tables 23 and 24, Tables 25 and 26, Tables 27 and 28, Tables 29 and 30, Tables 31 and 32, Tables 33 and 34, Tables 35 and 36, Tables 37 and 38, and Tables 39 and 40 correspond to each other, and the former (odd number) of each table pair shows information on bacteria and gluconate dehydratase (gad) or gluconate kinase (gntK), and the latter of each table pair shows information on gluconate transporters corresponding to the bacteria listed in the former table and “gut_microbes” described later.

TABLE 13

| | | | | gad_ | gad_ | gad_ |
|---|---|---|---|---|---|---|
| accession | organism.taxId | species | gad_sseqid | qcovhsp | pident | evalue |
| GCF_000018685.1 | 357809 | Lachnoclostridium | gad_k17_FCCFCLNI_05334_Dihydroxy- | 100 | 82.7 | 0 |
| | | phytofermentans | acid_dehydratase | | | |
| GCF_000146185.1 | 515620 | Lachnospira eligens | gad_i22_MMKBGNBN_03241_Dihydroxy- | 99.7 | 91.6 | 0 |
| | | | acid_dehydratase | | | |
| GCF_000154385.1 | 411485 | Faecalibacterium | gad_i20_GFDADFBJ_02480_Dihydroxy- | 100 | 91.8 | 0 |
| | | prausnitzii | acid_dehydratase | | | |
| GCF_000218855.1 | 991791 | Clostridium | gad_i13_AFPGDEAI_01673_Dihydroxy- | 99.8 | 74.4 | 8.35e−315 |
| | | acetobutylicum | acid_dehydratase | | | |
| GCF_000246895.1 | 545243 | Clostridium arbusti | gad_i13_AFPGDEAI_01673_Dihydroxy- | 99.8 | 73.2 | 9.22e−312 |
| | | | acid_dehydratase | | | |
| GCF_000371485.1 | 999409 | Enterocloster | gad_k19_HGDCKBFN_00437_Dihydroxy- | 100 | 99.8 | 0 |
| | | clostridioformis | acid_dehydratase | | | |
| GCF_000686725.1 | 1121293 | Clostridium akagii | gad_i13_AFPGDEAI_01673_Dihydroxy- | 99.8 | 73.9 | 1.31e−311 |
| | | | acid_dehydratase | | | |
| GCF_000821305.1 | 1499683 | Clostridium | gad_k17_FCCFCLNI_05334_Dihydroxy- | 100 | 77.8 | 0 |
| | | culturomicium | acid_dehydratase | | | |
| GCF_001487105.1 | 1720300 | Mediterraneibacter | gad_f17_EAOGLLOI_00767_Dihydroxy- | 99.5 | 81.4 | 0 |
| | | massiliensis | acid_dehydratase | | | |
| GCF_001593985.1 | 1121305 | Clostridium colicanis | gad_i13_AFPGDEAI_01673_Dihydroxy- | 99.8 | 74.1 | 3.94017e−320 |
| | | | acid_dehydratase | | | |
| GCF_001689125.2 | 1796616 | Blautia | gad_k04_BNLHKKFH_05311_Dihydroxy- | 100 | 98.8 | 0 |
| | | pseudococcoides | acid_dehydratase | | | |
| GCF_001856695.1 | 1501 | Clostridium | gad_i13_AFPGDEAI_01673_Dihydroxy- | 99.8 | 72.2 | 0 |
| | | pasteurianum | acid_dehydratase | | | |
| GCF_002080475.1 | 1497 | Clostridium | gad_k17_FCCFCLNI_05334_Dihydroxy- | 100 | 78.5 | 0 |
| | | formicaceticum | acid_dehydratase | | | |
| GCF_002160355.1 | 501571 | Butyricicoccus | gad_k40_AFILLPBB_01494_Dihydroxy- | 99.7 | 85.8 | 0 |
| | | pullicaecorum | acid_dehydratase | | | |
| GCF_002234575.2 | 208479 | Enterocloster bolteae | gad_f18_PGAODDDK_05264_Dihydroxy- | 100 | 99.7 | 0 |
| | | | acid_dehydratase | | | |
| GCF_002250835.2 | 2020949 | Romboutsia | gad_k17_FCCFCLNI_05334_Dihydroxy- | 99.6 | 77.9 | 0 |
| | | weinsteinii | acid_dehydratase | | | |
| GCF_002270465.1 | 2025493 | Blautia hominis | gad_k04_BNLHKKFH_05311_Dihydroxy- | 100 | 100 | 0 |
| | | | acid_dehydratase | | | |
| GCF_002995795.1 | 1121340 | Clostridium | gad_i13_AFPGDEAI_01673_Dihydroxy- | 99.8 | 74.1 | 3.94017e−320 |
| | | thermopalmarium | acid_dehydratase | | | |
| GCF_003149245.1 | 1544 | Faecalicatena | gad_i22_MMKBGNBN_03241_Dihydroxy- | 100 | 88.5 | 0 |
| | | orotica | acid_dehydratase | | | |
| GCF_003201875.1 | 1096246 | Hungatella effluvii | gad_k17_FCCFCLNI_05334_Dihydroxy- | 100 | 99.1 | 0 |
| | | | acid_dehydratase | | | |
| GCF_003363435.1 | 1812858 | Anaerosacchariphilus | gad_f10_FONBLEPD_00269_Dihydroxy- | 100 | 81.1 | 0 |
| | | polymeriproducens | acid_dehydratase | | | |
| GCF_003885045.1 | 2044587 | Schaedlerella | gad_f17_EAOGLLOI_00767_Dihydroxy- | 99.8 | 90.7 | 0 |
| | | arabinosiphila | acid_dehydratase | | | |
| GCF_004006535.1 | 2487351 | Anaerosphaera | gad_f18_PGAODDDK_05264_Dihydroxy- | 100 | 82.7 | 0 |
| | | multitolerans | acid_dehydratase | | | |
| GCF_004011155.1 | 2769420 | Clostridium | gad_i13_AFPGDEAI_01673_Dihydroxy- | 99.8 | 70.2 | 6.76E−300 |
| | | prolinivorans | acid_dehydratase | | | |
| GCF_004339095.1 | 680378 | Natranaerovirga | gad_k17_FCCFCLNI_05334_Dihydroxy- | 99.6 | 75.2 | 0 |
| | | hydrolytica | acid_dehydratase | | | |
| GCF_009721605.1 | 154046 | Hungatella | gad_k17_FCCFCLNI_05334_Dihydroxy- | 100 | 95.5 | 0 |
| | | hathewayi | acid_dehydratase | | | |
| GCF_009831375.1 | 411470 | [Ruminococcus] | gad_f10_FONBLEPD_00269_Dihydroxy- | 100 | 100 | 0 |
| | | gnavus | acid_dehydratase | | | |
| GCF_009917485.1 | 2320879 | Parablautia muri | gad_i22_MMKBGNBN_03241_Dihydroxy- | 99.5 | 88.1 | 0 |
| | | | acid_dehydratase | | | |
| GCF_010179735.1 | 2662126 | Anaerotalea | gad_k17_FCCFCLNI_05334_Dihydroxy- | 100 | 79.9 | 0 |
| | | alkaliphila | acid_dehydratase | | | |
| GCF_010669205.1 | 1121114 | Blautia producta | gad_f32_GFFMIMOM_00656_Dihydroxy- | 100 | 100 | 0 |
| | | | acid_dehydratase | | | |
| GCF_013300845.1 | 536633 | Blautia glucerasea | gad_i22_MMKBGNBN_03241_Dihydroxy- | 100 | 97.9 | 0 |
| | | | acid_dehydratase | | | |
| GCF_014287475.1 | 2763054 | Mediterraneibacter | gad_f17_EAOGLLOI_00767_Dihydroxy- | 99.5 | 81.4 | 0 |
| | | hominis | acid_dehydratase | | | |
| GCF_014287515.2 | 2763062 | Roseburia rectibacter | gad_i20_GFDADFBJ_02480_Dihydroxy- | 100 | 80 | 0 |
| | | | acid_dehydratase | | | |
| GCF_014287615.1 | 2763026 | Blautia celeris | gad_f21_EKJEFAGC_00541_Dihydroxy | 100 | 99.8 | 0 |
| | | | -acid_dehydratase | | | |

TABLE 14

| | | trans- | trans- | trans- | gut_mi- |
|---|---|---|---|---|---|
| accession | transporter_sseqid | porter_qcovhsp | porter_pident | porter_evalue | crobes |
| GCF_000018685.1 | transporter_k19_HGDCKBFN_00438_Low- | 96.6 | 69.4 | 2.75E−213 | 1 |
| | affinity_gluconate_transporter | | | | |
| GCF_000146185.1 | transporter_i22_MMKBGNBN_03243_Low- | 100 | 89.1 | 5.16E−275 | 0 |
| | affinity_gluconate_transporter | | | | |
| GCF_000154385.1 | transporter_i25_HOMMCNHA_02662_Low- | 100 | 98 | 5.65E−302 | 1 |
| | affinity_gluconate_transporter | | | | |
| GCF_000218855.1 | transporter_f37_MKMCEHOJ_02505_High- | 99.8 | 63.2 | 4.91E−184 | 0 |
| | affinity_gluconate_transporter | | | | |
| GCF_000246895.1 | transporter_f37_MKMCEHOJ_02505_High- | 100 | 62.6 | 1.09E−185 | 0 |
| | affinity_gluconate_transporter | | | | |
| GCF_000371485.1 | transporter_k19_HGDCKBFN_00438_Low- | 100 | 100 | 1.02E−305 | 0 |
| | affinity_gluconate_transporter | | | | |
| GCF_000686725.1 | transporter_f37_MKMCEHOJ_02505_High- | 100 | 62.3 | 3.46E−184 | 0 |
| | affinity_gluconate_transporter | | | | |
| GCF_000821305.1 | transporter_k19_HGDCKBFN_00438_Low- | 98.7 | 69.5 | 4.73E−206 | 0 |
| | affinity_gluconate_transporter | | | | |
| GCF_001487105.1 | transporter_f22_PKNBPMIK_04125_High- | 99.6 | 75.6 | 9.58E−247 | 0 |
| | affinity_gluconate_transporter | | | | |
| GCF_001593985.1 | transporter_k42_MFPDOCHA_03253_In- | 100 | 72 | 9.72E−226 | 0 |
| | ner_membrane_permease_YgbN | | | | |
| GCF_001689125.2 | transporter_k04_BNLHKKFH_05313_High- | 100 | 99.8 | 9.25E−300 | 0 |
| | affinity_gluconate_transporter | | | | |
| GCF_001856695.1 | transporter_f37_MKMCEHOJ_02505_High- | 100 | 62.3 | 4.34E−180 | 0 |
| | affinity_gluconate_transporter | | | | |
| GCF_002080475.1 | transporter_k19_HGDCKBFN_00438_Low- | 98.4 | 70.5 | 8.09E−214 | 0 |
| | affinity_gluconate_transporter | | | | |
| GCF_002160355.1 | transporter_k19_HGDCKBFN_00438_Low- | 98 | 69.6 | 3.54E−213 | 1 |
| | affinity_gluconate_transporter | | | | |
| GCF_002234575.2 | transporter_f18_PGAODDDK_05263_Low- | 100 | 99.3 | 3.27E−302 | 0 |
| | affinity_gluconate_transporter | | | | |
| GCF_002250835.2 | transporter_k34_HOEMBMAP_05033_Low- | 98.9 | 68.9 | 2.44E−210 | 0 |
| | affinity_gluconate_transporter | | | | |
| GCF_002270465.1 | transporter_k04_BNLHKKFH_05313_High- | 100 | 100 | 6.51E−300 | 0 |
| | affinity_gluconate_transporter | | | | |
| GCF_002995795.1 | transporter_k42_MFPDOCHA_03253_In- | 100 | 72 | 9.72E−226 | 0 |
| | ner_membrane_permease_YgbN | | | | |
| GCF_003149245.1 | transporter_i23_IEINNFOL_02286_Gnt- | 100 | 86.9 | 4.63E−269 | 1 |
| | II_system_L-idonate_transporter | | | | |
| GCF_003201875.1 | transporter_k17_FCCFCLNI_05332_In- | 100 | 100 | 5.74E−308 | 0 |
| | ner_membrane_permease_YgbN | | | | |
| GCF_003363435.1 | transporter_k19_HGDCKBFN_00438_Low- | 98.5 | 67.1 | 7.23E−206 | 0 |
| | affinity_gluconate_transporter | | | | |
| GCF_003885045.1 | transporter_f17_EAOGLLOI_00766_Low- | 100 | 88.1 | 2.26E−274 | 0 |
| | affinity_gluconate_transporter | | | | |
| GCF_004006535.1 | transporter_k19_HGDCKBFN_00438_Low- | 95.7 | 78.3 | 1.30E−241 | 0 |
| | affinity_gluconate_transporter | | | | |
| GCF_004011155.1 | transporter_f18_PGAODDDK_05263_Low- | 98.9 | 66.1 | 1.82E−196 | 0 |
| | affinity_gluconate_transporter | | | | |
| GCF_004339095.1 | transporter_k19_HGDCKBFN_00438_Low- | 99.8 | 65.8 | 1.79E−198 | 0 |
| | affinity_gluconate_transporter | | | | |
| GCF_009721605.1 | transporter_k17_FCCFCLNI_05332_In- | 100 | 98 | 3.03E−303 | 1 |
| | ner_membrane_permease_YgbN | | | | |
| GCF_009831375.1 | transporter_f10_FONBLEPD_00271_Low- | 100 | 100 | 2.15E−306 | 1 |
| | affinity_gluconate_transporter | | | | |
| GCF_009917485.1 | transporter_f17_EAOGLLOI_00766_Low- | 100 | 88.1 | 9.20E−274 | 0 |
| | affinity_gluconate_transporter | | | | |
| GCF_010179735.1 | transporter_k19_HGDCKBFN_00438_Low- | 94.4 | 72.1 | 5.61E−223 | 0 |
| | affinity_gluconate_transporter | | | | |
| GCF_010669205.1 | transporter_f32_GFFMIMOM_00658_High- | 100 | 100 | 5.50E−308 | 1 |
| | affinity_gluconate_transporter | | | | |
| GCF_013300845.1 | transporter_i22_MMKBGNBN_03243_Low- | 100 | 94 | 3.03E−290 | 0 |
| | affinity_gluconate_transporter | | | | |
| GCF_014287475.1 | transporter_f22_PKNBPMIK_04125_High- | 99.8 | 75.6 | 4.10E−248 | 0 |
| | affinity_gluconate_transporter | | | | |
| GCF_014287515.2 | transporter_f17_EAOGLLOI_00766_Low- | 98.4 | 82.7 | 1.35E−254 | 0 |
| | affinity_gluconate_transporter | | | | |
| GCF_014287615.1 | transporter_f21_EKJEFAGC_00543_In- | 100 | 100 | 2.09E−311 | 0 |
| | ner_membrane_permease_YgbN | | | | |

TABLE 15

| | | | | gad_ | gad_ | gad_ |
|---|---|---|---|---|---|---|
| accession | organism.taxId | species | gad_sseqid | qcovhsp | pident | evalue |
| GCF_014288005.1 | 2763050 | Hungatella hominis | gad_k17_FCCFCLNI_05334_Dihydroxy- | 100 | 99.3 | 0 |
| | | | acid_dehydratase | | | |
| GCF_014306135.1 | 2764329 | Paeniclostridium | gad_k17_FCCFCLNI_05334_Dihydroxy- | 99.6 | 76.8 | 0 |
| | | hominis | acid_dehydratase | | | |
| GCF_014385265.1 | 2763663 | Enterocloster | gad_f17_EAOGLLOI_00767_Dihydroxy- | 99.8 | 90 | 0 |
| | | hominis | acid_dehydratase | | | |
| GCF_016458825.1 | 2931922 | Miniphocaeibacter | gad_f18_PGAODDDK_05264_Dihydroxy- | 100 | 84.5 | 0 |
| | | halophilus | acid_dehydratase | | | |
| GCF_018332455.1 | 69208 | Clostridium | gad_i13_AFPGDEAI_01673_Dihydroxy- | 99.8 | 73 | 3.07e−317 |
| | | polyendosporum | acid_dehydratase | | | |
| GCF_018861735.1 | 205328 | Clostridium | gad_i13_AFPGDEAI_01673_Dihydroxy- | 99.8 | 75 | 0 |
| | | lacusfryxellense | acid_dehydratase | | | |
| GCF_019207025.1 | 2794346 | Clostridium | gad_i13_AFPGDEAI_01673_Dihydroxy- | 99.8 | 76.7 | 0 |
| | | thailandense | acid_dehydratase | | | |
| GCF_020554845.1 | 871665 | Blautia faecis | gad_i22_MMKBGNBN_03241_Dihydroxy- | 100 | 97.9 | 0 |
| | | | acid_dehydratase | | | |
| GCF_020687245.1 | 2881271 | Faecalibacterium | gad_f17_EAOGLLOI_00767_Dihydroxy- | 99.5 | 81 | 0 |
| | | longum | acid_dehydratase | | | |
| GCF_021043395.1 | 1662055 | Clostridium | gad_i13_AFPGDEAI_01673_Dihydroxy- | 99.8 | 75 | 9.24002e−319 |
| | | guangxiense | acid_dehydratase | | | |
| GCF_024160185.1 | 2949333 | Ohessyouella | gad_f30_DFNANFDB_01672_Dihydroxy- | 100 | 81.3 | 0 |
| | | blattaphilus | acid_dehydratase | | | |
| GCF_024160205.1 | 2949332 | Aequitasia | gad_f30_DFNANFDB_01672_Dihydroxy- | 100 | 82.5 | 0 |
| | | blattaphilus | acid_dehydratase | | | |
| GCF_024622975.1 | 1796615 | Blautia caecimuris | gad_f17_EAOGLLOI_00767_Dihydroxy- | 100 | 99.5 | 0 |
| | | | acid_dehydratase | | | |
| GCF_025148285.1 | 478749 | Marvinbryantia | gad_i22_MMKBGNBN_03241_Dihydroxy- | 100 | 88.6 | 0 |
| | | formatexigens | acid_dehydratase | | | |
| GCF_025567005.1 | 2981772 | Muricoprocola aceti | gad_i20_GFDADFBJ_02480_Dihydroxy- | 100 | 85 | 0 |
| | | | acid_dehydratase | | | |
| GCF_025567095.1 | 2981768 | Anthro- | gad_i22_MMKBGNBN_03241_Dihydroxy- | 100 | 92.2 | 0 |
| | | pogastromicrobium | acid_dehydratase | | | |
| | | aceti | | | | |
| GCF_025567175.1 | 2981790\| | Faecalicatena | gad_f17_EAOGLLOI_00767_Dihydroxy- | 99.5 | 81.4 | 0 |
| | | acetigenes | acid_dehydratase | | | |
| GCF_025567195.1 | 2981724 | Alitiscatomonas aceti | gad_f17_EAOGLLOI_00767_Dihydroxy- | 99.8 | 89.8 | 0 |
| | | | acid_dehydratase | | | |
| GCF_030585465.1 | 238834 | Clostridium | gad_i13_AFPGDEAI_01673_Dihydroxy- | 99.8 | 75.7 | 0 |
| | | estertheticum | acid_dehydratase | | | |
| GCF_900074625.1 | 1507512 | Romboutsia hominis | gad_k17_FCCFCLNI_05334_Dihydroxy- | 99.6 | 76.3 | 0 |
| | | | acid_dehydratase | | | |
| GCF_900096945.1 | 1619234 | Anaerobium | gad_k17_FCCFCLNI_05334_Dihydroxy- | 99.8 | 82.3 | 0 |
| | | acetethylicum | acid_dehydratase | | | |
| GCF_900128885.1 | 1120975 | Alkalibacter | gad_k17_FCCFCLNI_05334_Dihydroxy- | 100 | 77.5 | 0 |
| | | saccharofermentans | acid_dehydratase | | | |
| GCF_900129955.1 | 1121326 | Clostridium magnum | gad_i13_AFPGDEAI_01673_Dihydroxy- | 99.8 | 75.1 | 5.62e−320 |
| | | | acid_dehydratase | | | |
| GCF_900176305.1 | 1121291 | Clostridium acidisoli | gad_i13_AFPGDEAI_01673_Dihydroxy- | 99.8 | 74.1 | 2.78e−313 |
| | | | acid_dehydratase | | | |
| GCF_900205965.1 | 253257 | Lacrimispora | gad_k40_AFILLPBB_01494_Dihydroxy- | 100 | 82.5 | 0 |
| | | amygdalina | acid_dehydratase | | | |
| GCF_900232905.1 | 2041841 | Miniphocaeibacter | gad_k19_HGDCKBFN_00437_Dihydroxy- | 100 | 83.8 | 0 |
| | | massiliensis | acid_dehydratase | | | |
| GCF_900240225.1 | 2040292 | Dysgonomonas | gad_i13_AFPGDEAI_01673_Dihydroxy- | 99.8 | 76.6 | 0 |
| | | massiliensis | acid_dehydratase | | | |
| GCF_900248245.1 | 879566 | Acetatifactor muris | gad_i22_MMKBGNBN_03241_Dihydroxy- | 100 | 89.2 | 0 |
| | | | acid_dehydratase | | | |
| GCF_900258535.1 | 1917868 | Blautia marasmi | gad_k04_BNLHKKFH_05311_Dihydroxy- | 100 | 99.8 | 0 |
| | | | acid_dehydratase | | | |
| GCF_900461125.1 | 1532 | Blautia coccoides | gad_f32_GFFMIMOM_00656_Dihydroxy- | 100 | 100 | 0 |
| | | | acid_dehydratase | | | |
| GCF_900537995.1 | 536231 | Roseburia intestinalis | gad_i20_GFDADFBJ_02480_Dihydroxy- | 100 | 79.7 | 0 |
| | | | acid_dehydratase | | | |
| GCF_900626095.1 | 2040295 | Clostridium rectalis | gad_i13_AFPGDEAI_01673_Dihydroxy- | 99.5 | 72.2 | 0.E+00 |
| | | | acid_dehydratase | | | |
| GCF_943192995.1 | 2941327 | Flintibacter muris | gad_f17_EAOGLLOI_00767_Dihydroxy- | 99.3 | 82.2 | 0 |
| | | | acid_dehydratase | | | |
| GCF_943193015.1 | 2941517 | Hominisplanchenecus | gad_f17_EAOGLLOI_00767_Dihydroxy- | 98.8 | 84 | 0 |
| | | murintestinalis | acid_dehydratase | | | |
| GCF_943193195.1 | 2941333 | Mediterraneibacter | gad_f13_FHKCONON_03198_Dihydroxy- | 99.8 | 90.9 | 0 |
| | | agrestimuris | acid_dehydratase | | | |
| GCF_949738545.1 | 2320100 | Parablautia | gad_i22_MMKBGNBN_03241_Dihydroxy- | 99.7 | 88 | 0 |
| | | intestinalis | acid_dehydratase | | | |

TABLE 16

| accession | transporter_sseqid | transporter_qcovhsp | transporter_pident | transporter_evalue | gut_microbes |
|---|---|---|---|---|---|
| GCF_014288005.1 | transporter_k17_FCCFCLNI_05332_Inner_membrane_permease_YgbN | 100 | 99.6 | 1.64E−307 | 0 |
| GCF_014306135.1 | transporter_f21_EKJEFAGC_00543_Inner_membrane_permease_YgbN | 100 | 72.3 | 3.27E−227 | 0 |
| GCF_014385265.1 | transporter_f17_EAOGLLOI_00766_Low-affinity_gluconate_transporter | 100 | 88.2 | 4.42E−276 | 0 |
| GCF_016458825.1 | transporter_k19_HGDCKBFN_00438_Low-affinity_gluconate_transporter | 95.7 | 77.7 | 7.66E−239 | 0 |
| GCF_018332455.1 | transporter_f37_MKMCEHOJ_02505_High-affinity_gluconate_transporter | 100 | 63.9 | 1.54E−192 | 0 |
| GCF_018861735.1 | transporter_k42_MFPDOCHA_03253_Inner_membrane_permease_YgbN | 100 | 67.1 | 1.21E−209 | 0 |
| GCF_019207025.1 | transporter_f37_MKMCEHOJ_02505_High-affinity_gluconate_transporter | 99.3 | 67.1 | 1.50E−197 | 0 |
| GCF_020554845.1 | transporter_i22_MMKBGNBN_03243_Low-affinity_gluconate_transporter | 100 | 94 | 3.03E−290 | 0 |
| GCF_020687245.1 | transporter_f22_PKNBPMIK_04125_High-affinity_gluconate_transporter | 100 | 77 | 3.20E−252 | 0 |
| GCF_021043395.1 | transporter_f37_MKMCEHOJ_02505_High-affinity_gluconate_transporter | 99.3 | 63.7 | 3.76E−181 | 0 |
| GCF_024160185.1 | transporter_k42_MFPDOCHA_03253_Inner_membrane_permease_YgbN | 100 | 86.1 | 3.14E−272 | 0 |
| GCF_024160205.1 | transporter_k42_MFPDOCHA_03253_Inner_membrane_permease_YgbN | 99.8 | 86.5 | 3.66E−271 | 0 |
| GCF_024622975.1 | transporter_f19_DHAAOBAE_00693_Low-affinity_gluconate_transporter | 100 | 100 | 4.77E−234 | 0 |
| GCF_025148285.1 | transporter_f17_EAOGLLOI_00766_Low-affinity_gluconate_transporter | 98.9 | 87.7 | 2.18E−274 | 1 |
| GCF_025567005.1 | transporter_f17_EAOGLLOI_00766_Low-affinity_gluconate_transporter | 99.3 | 83.9 | 3.65E−259 | 0 |
| GCF_025567095.1 | transporter_i22_MMKBGNBN_03243_Low-affinity_gluconate_transporter | 100 | 82.3 | 5.83E−251 | 0 |
| GCF_025567175.1 | transporter_f22_PKNBPMIK_04125_High-affinity_gluconate_transporter | 99.6 | 75.6 | 9.58E−247 | 0 |
| GCF_025567195.1 | transporter_f17_EAOGLLOI_00766_Low-affinity_gluconate_transporter | 100 | 88.2 | 4.42E−276 | 0 |
| GCF_030585465.1 | transporter_f37_MKMCEHOJ_02505_High-affinity_gluconate_transporter | 99.8 | 63.5 | 6.80E−182 | 0 |
| GCF_900074625.1 | transporter_f21_EKJEFAGC_00543_Inner_membrane_permease_YgbN | 100 | 72.6 | 2.67E−226 | 0 |
| GCF_900096945.1 | transporter_k19_HGDCKBFN_00438_Low-affinity_gluconate_transporter | 96.8 | 71.8 | 9.34E−223 | 0 |
| GCF_900128885.1 | transporter_k19_HGDCKBFN_00438_Low-affinity_gluconate_transporter | 96.9 | 72.6 | 1.18E−221 | 0 |
| GCF_900129955.1 | transporter_f37_MKMCEHOJ_02505_High-affinity_gluconate_transporter | 100 | 65.5 | 7.44E−198 | 0 |
| GCF_900176305.1 | transporter_f37_MKMCEHOJ_02505_High-affinity_gluconate_transporter | 99.3 | 62.3 | 6.41E−187 | 0 |
| GCF_900205965.1 | transporter_k19_HGDCKBFN_00438_Low-affinity_gluconate_transporter | 98 | 70.9 | 4.51E−219 | 0 |
| GCF_900232905.1 | transporter_f17_EAOGLLOI_00766_Low-affinity_gluconate_transporter | 98.3 | 75.1 | 6.92E−235 | 0 |
| GCF_900240225.1 | transporter_i13_AFPGDEAI_02973_Gnt-II_system_L-idonate_transporter | 100 | 72.5 | 3.72E−213 | 0 |
| GCF_900248245.1 | transporter_f17_EAOGLLOI_00766_Low-affinity_gluconate_transporter | 99.3 | 87.1 | 3.57E−271 | 0 |
| GCF_900258535.1 | transporter_k04_BNLHKKFH_05313_High-affinity_gluconate_transporter | 100 | 99.8 | 1.87E−299 | 1 |
| GCF_900461125.1 | transporter_f32_GFFMIMOM_00658_High-affinity_gluconate_transporter | 100 | 97.9 | 9.25E−300 | 0 |
| GCF_900537995.1 | transporter_f17_EAOGLLOI_00766_Low-affinity_gluconate_transporter | 98.4 | 82.9 | 2.33E−255 | 1 |
| GCF_900626095.1 | transporter_k42_MFPDOCHA_03253_Inner_membrane_permease_YgbN | 100 | 69.5 | 6.35E−212 | 0 |
| GCF_943192995.1 | transporter_f21_EKJEFAGC_00543_Inner_membrane_permease_YgbN | 98.9 | 63.1 | 4.84E−185 | 0 |
| GCF_943193015.1 | transporter_f17_EAOGLLOI_00766_Low-affinity_gluconate_transporter | 99.8 | 85.8 | 2.14E−270 | 0 |
| GCF_943193195.1 | transporter_f17_EAOGLLOI_00766_Low-affinity_gluconate_transporter | 99.1 | 83.7 | 6.03E−258 | 0 |
| GCF_949738545.1 | transporter_f17_EAOGLLOI_00766_Low-affinity_gluconate_transporter | 100 | 86.9 | 1.25E−271 | 0 |

TABLE 17

| | organism. | | | gntK_ | gntK_ | gntK_ |
|---|---|---|---|---|---|---|
| accession | taxId | species | gntK_sseqid | qcovhsp | pident | evalue |
| GCF_000005845.2 | 511145 | Escherichia coli | gntK_f37_MKMCEHOJ_02531_Thermoresistant_gluconokinase | 100 | 99.4 | 1.45E−127 |
| GCF_000006925.2 | 198214 | Shigella flexneri | gntK_f37_MKMCEHOJ_02531_Thermoresistant_gluconokinase | 100 | 98.1 | 1.57E−114 |
| GCF_000006945.2 | 99287 | Salmonella | gntK_f37_MKMCEHOJ_02531_Thermoresistant_gluconokinase | 98.3 | 97.1 | 2.14E−125 |
| | | enterica | | | | |
| GCF_000008865.2 | 386585 | Escherichia coli | gntK_f37_MKMCEHOJ_02531_Thermoresistant_gluconokinase | 100 | 99.4 | 1.45E−127 |
| GCF_000018045.1 | 290338 | Citrobacter koseri | gntK_f37_MKMCEHOJ_02531_Thermoresistant_gluconokinase | 98.9 | 96 | 6.90E−124 |
| GCF_000026185.1 | 465817 | Erwinia | gntK_Kp2H7_NOCLEBFK_02257_Thermoresistant_gluconokinase | 94.5 | 78.5 | 3.67E−98 |
| | | tasmaniensis | | | | |
| GCF_000069965.1 | 529507 | Proteus mirabilis | gntK_f37_MKMCEHOJ_02531_Thermoresistant_gluconokinase | 93.5 | 70.3 | 1.94E−84 |
| GCF_000091565.1 | 665029 | Erwinia amylovora | gntK_Kp2H7_NOCLEBFK_02257_Thermoresistant_gluconokinase | 94.5 | 75.6 | 6.75E−94 |
| GCF_000165735.1 | 706434 | Megasphaera | gntK_i33_ABMIDDIK_01308_Xylulose_kinase | 99.2 | 83.4 | 0 |
| | | micronuciformis | | | | |
| GCF_000172135.1 | 473819 | Bifidobacterium | gntK_i01_CAKGJPAO_00191_Gluconokinase | 98.3 | 89.2 | 3.49E−114 |
| | | dentium | | | | |
| GCF_000196155.1 | 243265 | Photorhabdus | gntK_Kp2H7_NOCLEBFK_02257_Thermoresistant_gluconokinase | 96.1 | 70.9 | 1.42E−85 |
| | | laumondii | | | | |
| GCF_000196475.1 | 291112 | Photorhabdus | gntK_f37_MKMCEHOJ_02531_Thermoresistant_gluconokinase | 95.5 | 70.2 | 4.12E−87 |
| | | asymbiotica | | | | |
| GCF_000196615.1 | 634500 | Erwinia billingiae | gntK_f37_MKMCEHOJ_02531_Thermoresistant_gluconokinase | 97.2 | 76.2 | 6.53E−95 |
| GCF_000213975.1 | 888060 | Centipeda | gntK_i33_ABMIDDIK_01308_Xylulose_kinase | 98.3 | 67.8 | 4.41E−258 |
| | | periodontii | | | | |
| GCF_000214235.1 | 768492 | Serratia | gntK_f37_MKMCEHOJ_02531_Thermoresistant_gluconokinase | 94.4 | 75.1 | 1.11E−91 |
| | | plymuthica | | | | |
| GCF_000224675.1 | 885040 | Enterobacter soli | gntK_Kp2H7_NOCLEBFK_02257_Thermoresistant_gluconokinase | 98.9 | 94.2 | 3.66E−119 |
| GCF_000233595.1 | 1095774 | Pantoea ananatis | gntK_f37_MKMCEHOJ_02531_Thermoresistant_gluconokinase | 96.6 | 73.8 | 9.64E−95 |
| GCF_000240185.1 | 1125630 | Klebsiella | gntK_Kp2H7_NOCLEBFK_02257_Thermoresistant_gluconokinase | 100 | 100 | 1.34E−128 |
| | | pneumoniae | | | | |
| GCF_000261045.2 | 1154756 | Serratia inhibens | gntK_f37_MKMCEHOJ_02531_Thermoresistant_gluconokinase | 94.4 | 74 | 7.49E−90 |
| GCF_000262305.1 | 630626 | Shimwellia blattae | gntK_Kp2H7_NOCLEBFK_02257_Thermoresistant_gluconokinase | 98.9 | 83.8 | 4.91E−108 |
| GCF_000264765.2 | 667120 | Edwardsiella | gntK_f37_MKMCEHOJ_02531_Thermoresistant_gluconokinase | 97.7 | 72.7 | 1.05E−86 |
| | | anguillarum | | | | |
| GCF_000314855.2 | 1141662 | Providencia | gntK_f37_MKMCEHOJ_02531_Thermoresistant_gluconokinase | 92.5 | 69.4 | 8.40E−82 |
| | | burhodogranariea | | | | |
| GCF_000314895.2 | 1141660 | Providencia | gntK_Kp2H7_NOCLEBFK_02257_Thermoresistant_gluconokinase | 91.1 | 67.6 | 2.07E−83 |
| | | sneebia | | | | |
| GCF_000381065.1 | 1123511 | Propionispira | gntK_i33_ABMIDDIK_01308_Xylulose_kinase | 98.3 | 67.4 | 3.15E−265 |
| | | raffinosivorans | | | | |
| GCF_000412335.2 | 566551 | Cedecea davisae | gntK_Kp2H7_NOCLEBFK_02257_Thermoresistant_gluconokinase | 98.9 | 89.7 | 2.20E−116 |
| GCF_000421685.1 | 1121109 | Bifidobacterium | gntK_i02_JFDANPEK_00221_Gluconokinase | 93.8 | 77.1 | 3.48E−94 |
| | | minimum | | | | |
| GCF_000424065.1 | 1280706 | Selenomonas | gntK_i33_ABMIDDIK_01308_Xylulose_kinase | 98.1 | 74.3 | 3.49E−281 |
| | | ruminantium | | | | |
| GCF_000428145.1 | 529086 | Chitinilyticum | gntK_Kp2H7_NOCLEBFK_02257_Thermoresistant_gluconokinase | 91.5 | 69.6 | 1.20E−76 |
| | | litopenaei | | | | |
| GCF_000429785.1 | 1121274 | Chitinibacter | gntK_Kp2H7_NOCLEBFK_02257_Thermoresistant_gluconokinase | 93.6 | 68.5 | 7.51E−77 |
| | | tainanensis | | | | |
| GCF_000430805.1 | 1121275 | Chitinilyticum | gntK_f37_MKMCEHOJ_02531_Thermoresistant_gluconokinase | 93.8 | 72.1 | 6.37E−81 |
| | | aquatile | | | | |
| GCF_000439255.1 | 1197719 | Salmonella | gntK_f37_MKMCEHOJ_02531_Thermoresistant_gluconokinase | 98.3 | 96.6 | 6.15E−125 |
| | | bongori | | | | |
| GCF_000439375.1 | 480813 | Tatumella | gntK_f37_MKMCEHOJ_02531_Thermoresistant_gluconokinase | 98.3 | 71.1 | 6.65E−90 |
| | | saanichensis | | | | |
| GCF_000439895.1 | 1005995 | Tatumella ptyseos | gntK_Kp2H7_NOCLEBFK_02257_Thermoresistant_gluconokinase | 100 | 71.6 | 3.26E−88 |
| GCF_000455225.1 | 1232428 | Megasphaera | gntK_i37_IIDCKEOG_02051_Xylulose_kinase | 100 | 98.7 | 0 |
| | | massiliensis | | | | |
| GCF_000517265.1 | 1004151 | Photorhabdus | gntK_f37_MKMCEHOJ_02531_Thermoresistant_gluconokinase | 95.5 | 69 | 2.90E−87 |
| | | khanii | | | | |

TABLE 18

| accession | transporter_sseqid | transporter_qcovhsp | transporter_pident | transporter_evalue | gut_microbes |
|---|---|---|---|---|---|
| GCF_000005845.2 | transporter_f37_MKMCEHOJ_02530_Low-affinity_gluconate_transporter | 100 | 99.8 | 1.67E−312 | 1 |
| GCF_000006925.2 | transporter_f37_MKMCEHOJ_02530_Low-affinity_gluconate_transporter | 100 | 99.3 | 2.24E−300 | 0 |
| GCF_000006945.2 | transporter_f37_MKMCEHOJ_02530_Low-affinity_gluconate_transporter | 100 | 97.3 | 1.25E−307 | 1 |
| GCF_000008865.2 | transporter_f37_MKMCEHOJ_02530_Low-affinity_gluconate_transporter | 100 | 99.8 | 1.67E−312 | 1 |
| GCF_000018045.1 | transporter_f37_MKMCEHOJ_02530_Low-affinity_gluconate_transporter | 100 | 95.1 | 3.50E−298 | 1 |
| GCF_000026185.1 | transporter_f37_MKMCEHOJ_02530_Low-affinity_gluconate_transporter | 100 | 81.6 | 5.33E−263 | 0 |
| GCF_000069965.1 | transporter_f37_MKMCEHOJ_02530_Low-affinity_gluconate_transporter | 100 | 76.9 | 1.41E−246 | 1 |
| GCF_000091565.1 | transporter_f37_MKMCEHOJ_02530_Low-affinity_gluconate_transporter | 100 | 82.1 | 3.08E−262 | 0 |
| GCF_000165735.1 | transporter_i33_ABMIDDIK_01306_High-affinity_gluconate_transporter | 99.1 | 86.7 | 3.84E−257 | 1 |
| GCF_000172135.1 | transporter_i02_JFDANPEK_00220_Gnt-II_system_L-idonate_transporter | 100 | 94.1 | 1.79E−282 | 1 |
| GCF_000196155.1 | transporter_f37_MKMCEHOJ_02530_Low-affinity_gluconate_transporter | 100 | 78.2 | 1.34E−251 | 0 |
| GCF_000196475.1 | transporter_f37_MKMCEHOJ_02530_Low-affinity_gluconate_transporter | 99.3 | 77.6 | 2.10E−248 | 0 |
| GCF_000196615.1 | transporter_f37_MKMCEHOJ_02530_Low-affinity_gluconate_transporter | 100 | 82.1 | 3.76E−263 | 0 |
| GCF_000213975.1 | transporter_i33_ABMIDDIK_01306_High-affinity_gluconate_transporter | 100 | 77.9 | 1.11E−242 | 0 |
| GCF_000214235.1 | transporter_f37_MKMCEHOJ_02530_Low-affinity_gluconate_transporter | 99.8 | 72.1 | 7.68E−230 | 0 |
| GCF_000224675.1 | transporter_f37_MKMCEHOJ_02530_Low-affinity_gluconate_transporter | 100 | 95.5 | 2.58E−300 | 0 |
| GCF_000233595.1 | transporter_f37_MKMCEHOJ_02530_Low-affinity_gluconate_transporter | 100 | 80 | 1.39E−258 | 1 |
| GCF_000240185.1 | transporter_f37_MKMCEHOJ_02530_Low-affinity_gluconate_transporter | 100 | 95.7 | 2.70E−302 | 1 |
| GCF_000261045.2 | transporter_f37_MKMCEHOJ_02530_Low-affinity_gluconate_transporter | 99.8 | 72.5 | 2.83E−232 | 0 |
| GCF_000262305.1 | transporter_f37_MKMCEHOJ_02530_Low-affinity_gluconate_transporter | 100 | 89.9 | 4.42E−285 | 0 |
| GCF_000264765.2 | transporter_f37_MKMCEHOJ_02530_Low-affinity_gluconate_transporter | 100 | 78.4 | 2.70E−251 | 0 |
| GCF_000314855.2 | transporter_f37_MKMCEHOJ_02530_Low-affinity_gluconate_transporter | 97.8 | 74 | 6.14E−238 | 0 |
| GCF_000314895.2 | transporter_f37_MKMCEHOJ_02530_Low-affinity_gluconate_transporter | 98.2 | 74.1 | 1.24E−237 | 0 |
| GCF_000381065.1 | transporter_i33_ABMIDDIK_01306_High-affinity_gluconate_transporter | 100 | 74.7 | 1.73E−225 | 0 |
| GCF_000412335.2 | transporter_f37_MKMCEHOJ_02530_Low-affinity_gluconate_transporter | 100 | 92.4 | 1.97E−288 | 1 |
| GCF_000421685.1 | transporter_i01_CAKGJPAO_00190_High-affinity_gluconate_transporter | 95.1 | 67.3 | 1.25E−157 | 0 |
| GCF_000424065.1 | transporter_i33_ABMIDDIK_01306_High-affinity_gluconate_transporter | 99.3 | 82.3 | 4.33E−242 | 0 |
| GCF_000428145.1 | transporter_f37_MKMCEHOJ_02530_Low-affinity_gluconate_transporter | 100 | 79.3 | 9.43E−252 | 0 |
| GCF_000429785.1 | transporter_f37_MKMCEHOJ_02530_Low-affinity_gluconate_transporter | 100 | 76.9 | 8.99E−243 | 0 |
| GCF_000430805.1 | transporter_f37_MKMCEHOJ_02530_Low-affinity_gluconate_transporter | 100 | 79.3 | 5.71E−253 | 0 |
| GCF_000439255.1 | transporter_f37_MKMCEHOJ_02530_Low-affinity_gluconate_transporter | 100 | 96.4 | 2.42E−305 | 1 |
| GCF_000439375.1 | transporter_f37_MKMCEHOJ_02530_Low-affinity_gluconate_transporter | 100 | 76.9 | 7.27E−240 | 0 |
| GCF_000439895.1 | transporter_f37_MKMCEHOJ_02530_Low-affinity_gluconate_transporter | 100 | 76.2 | 4.64E−243 | 0 |
| GCF_000455225.1 | transporter_i37_IIDCKEOG_02049_High-affinity_gluconate_transporter | 100 | 99.5 | 2.59E−301 | 0 |
| GCF_000517265.1 | transporter_f37_MKMCEHOJ_02530_Low-affinity_gluconate_transporter | 100 | 77.3 | 8.98E−250 | 0 |

TABLE 19

| | organism. | | | gntK_ | gntK_ | gntK_ |
|---|---|---|---|---|---|---|
| accession | taxId | species | gntK_sseqid | qcovhsp | pident | evalue |
| GCF_000534275.1 | 208224 | Enterobacter | gntK_Kp2H7_NOCLEBFK_02257_ | 98.9 | 92.5 | 5.82E−116 |
| | | kobei | Thermoresistant_gluconokinase | | | |
| GCF_000590885.1 | 69222 | Erwinia | gntK_f37_MKMCEHOJ_02531_ | 97.7 | 76.3 | 2.65E−94 |
| | | mallotivora | Thermoresistant_gluconokinase | | | |
| GCF_000612035.1 | 2805098 | Photorhabdus | gntK_Kp2H7_NOCLEBFK_02257_ | 96.1 | 70.3 | 3.16E−82 |
| | | aegyptia | Thermoresistant_gluconokinase | | | |
| GCF_000620145.1 | 1121375 | Deefgea rivuli | gntK_f37_MKMCEHOJ_02531_ | 90.1 | 73.5 | 2.70E−81 |
| | | | Thermoresistant_gluconokinase | | | |
| GCF_000711895.1 | 1122960 | Photorhabdus | gntK_f37_MKMCEHOJ_02531_ | 95.5 | 69.6 | 1.37E−85 |
| | | australis | Thermoresistant_gluconokinase | | | |
| GCF_000722995.1 | 1393735 | Photorhabdus | gntK_f37_MKMCEHOJ_02531_ | 95.5 | 69.6 | 7.14E−88 |
| | | temperata | Thermoresistant_gluconokinase | | | |
| GCF_000734965.1 | 1005994 | Trabulsiella | gntK_Kp2H7_NOCLEBFK_02257_ | 99.4 | 93.1 | 4.45E−118 |
| | | guamensis | Thermoresistant_gluconokinase | | | |
| GCF_000735425.1 | 1005999 | Leminorella | gntK_f37_MKMCEHOJ_02531_ | 97.7 | 70.9 | 1.82E−87 |
| | | grimontii | Thermoresistant_gluconokinase | | | |
| GCF_000736675.1 | 1397852 | Xenorhabdus | gntK_Kp2H7_NOCLEBFK_02257_ | 96.6 | 69.4 | 1.65E−84 |
| | | bovienii | Thermoresistant_gluconokinase | | | |
| GCF_000737905.1 | 82992 | Buttiauxella | gntK_f37_MKMCEHOJ_02531_ | 98.3 | 92 | 2.48E−115 |
| | | noackiae | Thermoresistant_gluconokinase | | | |
| GCF_000738005.1 | 327277 | Bifidobacterium | gntK_i01_CAKGJPAO_00191_ | 91.3 | 73.2 | 3.03E−91 |
| | | crudilactis | Gluconokinase | | | |
| GCF_000741785.1 | 762211 | Bifidobacterium | gntK_i01_CAKGJPAO_00191_ | 94.9 | 77.7 | 2.71E−94 |
| | | stellenboschense | Gluconokinase | | | |
| GCF_000754995.1 | 585 | Proteus vulgaris | gntK_Kp2H7_NOCLEBFK_02257_ | 95.7 | 71.6 | 3.14E−88 |
| | | | Thermoresistant_gluconokinase | | | |
| GCF_000757425.2 | 642227 | Tatumella | gntK_f37_MKMCEHOJ_02531_ | 97.7 | 74.4 | 2.00E−91 |
| | | morbirosei | Thermoresistant_gluconokinase | | | |
| GCF_000757905.1 | 1247024 | Candidatus | gntK_Kp2H7_NOCLEBFK_02257_ | 92.7 | 67.5 | 3.01E−80 |
| | | Arsenophonus | Thermoresistant_gluconokinase | | | |
| | | nilaparvatae | | | | |
| GCF_000770305.1 | 796334 | Erwinia oleae | gntK_f37_MKMCEHOJ_02531_ | 95.5 | 76.6 | 4.75E−93 |
| | | | Thermoresistant_gluconokinase | | | |
| GCF_000771685.1 | 1437610 | Bifidobacterium | gntK_i01_CAKGJPAO_00191_ | 91.7 | 76.5 | 4.01E−93 |
| | | reuteri | Gluconokinase | | | |
| GCF_000773975.1 | 371042 | Erwinia | gntK_f37_MKMCEHOJ_02531_ | 100 | 77.4 | 8.33E−98 |
| | | typographi | Thermoresistant_gluconokinase | | | |
| GCF_000784735.1 | 1577887 | Dickeya undicola | gntK_f37_MKMCEHOJ_02531_ | 89 | 60.1 | 3.76E−67 |
| | | | Thermoresistant_gluconokinase | | | |
| GCF_000951135.1 | 1513468 | Rouxiella | gntK_Kp2H7_NOCLEBFK_02257_ | 86 | 62.7 | 5.98E−68 |
| | | chamberiensis | Thermoresistant_gluconokinase | | | |
| GCF_000968175.1 | 1354304 | Xenorhabdus | gntK_Kp2H7_NOCLEBFK_02257_ | 91.4 | 68.4 | 1.51E−82 |
| | | poinarii | Thermoresistant_gluconokinase | | | |
| GCF_000968195.1 | 351671 | Xenorhabdus | gntK_f37_MKMCEHOJ_02531_ | 92.4 | 70.8 | 6.06E−86 |
| | | doucetiae | Thermoresistant_gluconokinase | | | |
| GCF_000969395.1 | 470931 | Pantoea | gntK_f37_MKMCEHOJ_02531_ | 96.6 | 72.1 | 1.52E−91 |
| | | anthophila | Thermoresistant_gluconokinase | | | |
| GCF_000980985.1 | 82983 | Obesumbacterium | gntK_Kp2H7_NOCLEBFK_02257_ | 98.9 | 68.2 | 2.89E−87 |
| | | proteus | Thermoresistant_gluconokinase | | | |
| GCF_001006005.1 | 47917 | Serratia fonticola | gntK_f37_MKMCEHOJ_02531_ | 96.4 | 60.5 | 6.26E−72 |
| | | | Thermoresistant_gluconokinase | | | |
| GCF_001010285.1 | 230089 | Photorhabdus | gntK_f37_MKMCEHOJ_02531_ | 95.5 | 69.6 | 2.04E−87 |
| | | thracensis | Thermoresistant_gluconokinase | | | |
| GCF_001025195.1 | 566552 | Bifidobacterium | gntK_i02_JFDANPEK_00221_ | 82 | 99.3 | 7.40E−109 |
| | | catenulatum | Gluconokinase | | | |
| GCF_001037465.1 | 880157 | Xenorhabdus | gntK_f37_MKMCEHOJ_02531_ | 93 | 68.8 | 2.97E−84 |
| | | khoisanae | Thermoresistant_gluconokinase | | | |
| GCF_001042635.1 | 1150461 | Bifidobacterium | gntK_i01_CAKGJPAO_00191_ | 97.1 | 72.3 | 2.75E−88 |
| | | scardovii | Gluconokinase | | | |
| GCF_001050515.1 | 1161919 | Erwinia | gntK_Kp2H7_NOCLEBFK_02257_ | 94.5 | 77.3 | 5.80E−95 |
| | | piriflorinigrans | Thermoresistant_gluconokinase | | | |
| GCF_001083805.1 | 171439 | Photorhabdus | gntK_Kp2H7_NOCLEBFK_02257_ | 96.1 | 70.9 | 4.06E−85 |
| | | luminescens | Thermoresistant_gluconokinase | | | |
| GCF_001112925.1 | 685706 | Yersinia nurmii | gntK_f37_MKMCEHOJ_02531_ | 91.8 | 62.2 | 5.53E−71 |
| | | | Thermoresistant_gluconokinase | | | |
| GCF_001267535.1 | 1560201 | Erwinia iniecta | gntK_Kp2H7_NOCLEBFK_02257_ | 96.1 | 75.4 | 1.65E−93 |
| | | | Thermoresistant_gluconokinase | | | |
| GCF_001277175.1 | 1074000 | Cronobacter | gntK_Kp2H7_NOCLEBFK_02257_ | 99.4 | 82.8 | 7.79E−105 |
| | | universalis | Thermoresistant_gluconokinase | | | |
| GCF_001277195.1 | 1159613 | Cronobacter | gntK_Kp2H7_NOCLEBFK_02257_ | 99.4 | 83.3 | 3.86E−105 |
| | | muytjensii | Thermoresistant_gluconokinase | | | |

TABLE 20

| accession | transporter_sseqid | transporter_qcovhsp | transporter_pident | transporter_evalue | gut_microbes |
|---|---|---|---|---|---|
| GCF_000534275.1 | transporter_f37_MKMCEHOJ_02530_Low-affinity_gluconate_transporter | 100 | 96 | 3.14E−301 | 1 |
| GCF_000590885.1 | transporter_f37_MKMCEHOJ_02530_Low-affinity_gluconate_transporter | 100 | 82.7 | 4.81E−266 | 0 |
| GCF_000612035.1 | transporter_f37_MKMCEHOJ_02530_Low-affinity_gluconate_transporter | 100 | 77.8 | 3.14E−250 | 0 |
| GCF_000620145.1 | transporter_f37_MKMCEHOJ_02530_Low-affinity_gluconate_transporter | 100 | 77.1 | 4.69E−245 | 0 |
| GCF_000711895.1 | transporter_f37_MKMCEHOJ_02530_Low-affinity_gluconate_transporter | 99.3 | 77.1 | 4.04E−246 | 0 |
| GCF_000722995.1 | transporter_f37_MKMCEHOJ_02530_Low-affinity_gluconate_transporter | 100 | 76.6 | 2.10E−248 | 0 |
| GCF_000734965.1 | transporter_f37_MKMCEHOJ_02530_Low-affinity_gluconate_transporter | 100 | 94.2 | 2.47E−298 | 0 |
| GCF_000735425.1 | transporter_f37_MKMCEHOJ_02530_Low-affinity_gluconate_transporter | 100 | 76.6 | 6.65E−238 | 1 |
| GCF_000736675.1 | transporter_f37_MKMCEHOJ_02530_Low-affinity_gluconate_transporter | 100 | 78 | 6.91E−252 | 0 |
| GCF_000737905.1 | transporter_f37_MKMCEHOJ_02530_Low-affinity_gluconate_transporter | 100 | 92.2 | 1.25E−291 | 0 |
| GCF_000738005.1 | transporter_i01_CAKGJPAO_00190_High-affinity_gluconate_transporter | 99.8 | 73.2 | 1.57E−213 | 0 |
| GCF_000741785.1 | transporter_i01_CAKGJPAO_00190_High-affinity_gluconate_transporter | 100 | 80.3 | 5.64E−235 | 0 |
| GCF_000754995.1 | transporter_f37_MKMCEHOJ_02530_Low-affinity_gluconate_transporter | 100 | 76.4 | 2.57E−249 | 1 |
| GCF_000757425.2 | transporter_f37_MKMCEHOJ_02530_Low-affinity_gluconate_transporter | 100 | 78.3 | 5.13E−247 | 0 |
| GCF_000757905.1 | transporter_f37_MKMCEHOJ_02530_Low-affinity_gluconate_transporter | 96.9 | 69.9 | 4.61E−219 | 0 |
| GCF_000770305.1 | transporter_f37_MKMCEHOJ_02530_Low-affinity_gluconate_transporter | 100 | 82.3 | 5.08E−261 | 0 |
| GCF_000771685.1 | transporter_i02_JFDANPEK_00220_Gnt-II_system_L-idonate_transporter | 100 | 83.3 | 7.63E−247 | 0 |
| GCF_000773975.1 | transporter_f37_MKMCEHOJ_02530_Low-affinity_gluconate_transporter | 100 | 81.4 | 1.19E−259 | 0 |
| GCF_000534275.1 | transporter_f37_MKMCEHOJ_02530_Low-affinity_gluconate_transporter | 100 | 96 | 3.14E−301 | 1 |
| GCF_000951135.1 | transporter_f37_MKMCEHOJ_02505_High-affinity_gluconate_transporter | 100 | 83.8 | 6.84E−255 | 0 |
| GCF_000968175.1 | transporter_f37_MKMCEHOJ_02530_Low-affinity_gluconate_transporter | 100 | 77.8 | 1.27E−249 | 0 |
| GCF_000968195.1 | transporter_f37_MKMCEHOJ_02530_Low-affinity_gluconate_transporter | 100 | 76.6 | 7.00E−247 | 0 |
| GCF_000969395.1 | transporter_f37_MKMCEHOJ_02530_Low-affinity_gluconate_transporter | 100 | 80.9 | 1.97E−258 | 0 |
| GCF_000980985.1 | transporter_f37_MKMCEHOJ_02530_Low-affinity_gluconate_transporter | 99.8 | 73.6 | 9.89E−233 | 0 |
| GCF_001006005.1 | transporter_f37_MKMCEHOJ_02505_High-affinity_gluconate_transporter | 100 | 86.3 | 1.50E−265 | 0 |
| GCF_001010285.1 | transporter_f37_MKMCEHOJ_02530_Low-affinity_gluconate_transporter | 100 | 77.1 | 1.04E−248 | 0 |
| GCF_001025195.1 | transporter_i02_JFDANPEK_00220_Gnt-II_system_L-idonate_transporter | 100 | 95.9 | 3.25E−285 | 1 |
| GCF_001037465.1 | transporter_f37_MKMCEHOJ_02530_Low-affinity_gluconate_transporter | 100 | 78 | 1.34E−251 | 0 |
| GCF_001042635.1 | transporter_i01_CAKGJPAO_00190_High-affinity_gluconate_transporter | 100 | 80.1 | 1.87E−233 | 1 |
| GCF_001050515.1 | transporter_f37_MKMCEHOJ_02530_Low-affinity_gluconate_transporter | 100 | 81.4 | 5.08E−261 | 0 |
| GCF_001083805.1 | transporter_f37_MKMCEHOJ_02530_Low-affinity_gluconate_transporter | 99.3 | 78.3 | 4.46E−250 | 0 |
| GCF_001112925.1 | transporter_f37_MKMCEHOJ_02505_High-affinity_gluconate_transporter | 100 | 86.1 | 2.89E−263 | 0 |
| GCF_001267535.1 | transporter_f37_MKMCEHOJ_02530_Low-affinity_gluconate_transporter | 100 | 84.1 | 2.62E−270 | 0 |
| GCF_001277175.1 | transporter_f37_MKMCEHOJ_02530_Low-affinity_gluconate_transporter | 100 | 92.6 | 1.69E−289 | 0 |
| GCF_001277195.1 | transporter_f37_MKMCEHOJ_02530_Low-affinity_gluconate_transporter | 100 | 92.4 | 6.88E−289 | 0 |

TABLE 21

| | organism. | | | gntK_ | gntK_ | gntK_ |
|---|---|---|---|---|---|---|
| accession | taxId | species | gntK_sseqid | qcovhsp | pident | evalue |
| GCF_001277215.2 | 1159491 | Cronobacter | gntK_Kp2H7_NOCLEBFK_02257_Thermoresistant_gluconokinase | 99.4 | 82.8 | 7.79E−105 |
| | | malonaticus | | | | |
| GCF_001277235.1 | 1159554 | Cronobacter | gntK_Kp2H7_NOCLEBFK_02257_Thermoresistant_gluconokinase | 99.4 | 83.3 | 3.86E−105 |
| | | dublinensis | | | | |
| GCF_001277255.1 | 1073999 | Cronobacter | gntK_Kp2H7_NOCLEBFK_02257_Thermoresistant_gluconokinase | 99.4 | 82.2 | 1.57E−104 |
| | | condimenti | | | | |
| GCF_001280945.1 | 880156 | Photorhabdus | gntK_f37_MKMCEHOJ_02531_Thermoresistant_gluconokinase | 95.5 | 67.8 | 2.26E−84 |
| | | heterorhabditis | | | | |
| GCF_001417815.1 | 1329411 | Bifidobacterium | gntK_i01_CAKGJPAO_00191_Gluconokinase | 89.7 | 78.9 | 8.52E−96 |
| | | aesculapii | | | | |
| GCF_001461805.1 | 1334193 | [Enterobacter] | gntK_Kp2H7_NOCLEBFK_02257_Thermoresistant_gluconokinase | 98.3 | 90.8 | 3.13E−116 |
| | | lignolyticus | | | | |
| GCF_001484765.1 | 1384589 | Entomohabitans | gntK_f37_MKMCEHOJ_02531_Thermoresistant_gluconokinase | 100 | 85.5 | 4.19E−106 |
| | | teleogrylli | | | | |
| GCF_001485335.1 | 1560356 | Type-D symbiont | gntK_Kp2H7_NOCLEBFK_02257_Thermoresistant_gluconokinase | 100 | 76.4 | 1.48E−96 |
| | | of Plautia stali | | | | |
| GCF_001485355.1 | 1560357 | Type-E symbiont | gntK_Kp2H7_NOCLEBFK_02257_Thermoresistant_gluconokinase | 100 | 75.8 | 1.22E−95 |
| | | of Plautia stali | | | | |
| GCF_001518835.1 | 83655 | Leclercia | gntK_Kp2H7_NOCLEBFK_02257_Thermoresistant_gluconokinase | 98.9 | 93.6 | 8.61E−118 |
| | | adecarboxylata | | | | |
| GCF_001558935.2 | 35703 | Citrobacter | gntK_f37_MKMCEHOJ_02531_Thermoresistant_gluconokinase | 98.9 | 97.7 | 9.41E−124 |
| | | amalonaticus | | | | |
| GCF_001642805.2 | 2741499 | Serratia | gntK_Kp2H7_NOCLEBFK_02257_Thermoresistant_gluconokinase | 97.1 | 70.6 | 2.00E−84 |
| | | surfactantfaciens | | | | |
| GCF_001654835.1 | 1354253 | Buttiauxella | gntK_f37_MKMCEHOJ_02531_Thermoresistant_gluconokinase | 98.3 | 90.8 | 2.90E−114 |
| | | gaviniae | | | | |
| GCF_001654855.1 | 1354337 | Proteus myxofaciens | gntK_Kp2H7_NOCLEBFK_02257_Thermoresistant_gluconokinase | 93.5 | 71.5 | 4.04E−84 |
| GCF_001654925.1 | 1354251 | Buttiauxella | gntK_f37_MKMCEHOJ_02531_Thermoresistant_gluconokinase | 98.3 | 90.8 | 7.11E−115 |
| | | brennerae | | | | |
| GCF_001655675.1 | 1691903 | Mangrovibacter | gntK_Kp2H7_NOCLEBFK_02257_Thermoresistant_gluconokinase | 98.9 | 84.6 | 7.24E−106 |
| | | phragmitis | | | | |
| GCF_001656035.1 | 935293 | Yersinia | gntK_f37_MKMCEHOJ_02531_Thermoresistant_gluconokinase | 91.8 | 62.2 | 3.18E−70 |
| | | entomophaga | | | | |
| GCF_001658025.2 | 497725 | Kosakonia oryzae | gntK_f37_MKMCEHOJ_02531_Thermoresistant_gluconokinase | 98.9 | 94.8 | 3.83E−121 |
| GCF_001682915.1 | 73098 | Kluyvera georgiana | gntK_Kp2H7_NOCLEBFK_02257_Thermoresistant_gluconokinase | 98.3 | 92 | 2.29E−118 |
| GCF_001683395.1 | 1158459 | Kosakonia sacchari | gntK_Kp2H7_NOCLEBFK_02257_Thermoresistant_gluconokinase | 98.9 | 94.9 | 6.85E−120 |
| GCF_001684335.1 | 1851568 | Photorhabdus | gntK_Kp2H7_NOCLEBFK_02257_Thermoresistant_gluconokinase | 96.1 | 70.3 | 2.23E−82 |
| | | namnaonensis | | | | |
| GCF_001721185.1 | 351679 | Xenorhabdus | gntK_Kp2H7_NOCLEBFK_02257_Thermoresistant_gluconokinase | 93.5 | 71.7 | 1.33E−87 |
| | | hominickii | | | | |
| GCF_001729805.1 | 1812935 | Enterobacter | gntK_Kp2H7_NOCLEBFK_02257_Thermoresistant_gluconokinase | 98.9 | 93.1 | 4.10E−116 |
| | | roggenkampii | | | | |
| GCF_001750725.1 | 299767 | Enterobacter | gntK_Kp2H7_NOCLEBFK_02257_Thermoresistant_gluconokinase | 98.9 | 93.6 | 2.47E−117 |
| | | ludwigii | | | | |
| GCF_001908095.1 | 1873484 | Xenorhabdus | gntK_Kp2H7_NOCLEBFK_02257_Thermoresistant_gluconokinase | 92.4 | 70.2 | 1.20E−83 |
| | | thuongxuanensis | | | | |
| GCF_001984825.2 | 2494701 | Enterobacter | gntK_Kp2H7_NOCLEBFK_02257_Thermoresistant_gluconokinase | 98.9 | 92.5 | 5.82E−116 |
| | | chengduensis | | | | |
| GCF_002077695.1 | 1964541 | Pantoea latae | gntK_Kp2H7_NOCLEBFK_02257_Thermoresistant_gluconokinase | 97.2 | 74 | 4.72E−93 |
| GCF_002095315.1 | 472705 | Pantoea conspicua | gntK_Kp2H7_NOCLEBFK_02257_Thermoresistant_gluconokinase | 96.6 | 74.4 | 8.18E−94 |
| GCF_002095475.1 | 1076550 | Pantoea rwandensis | gntK_Kp2H7_NOCLEBFK_02257_Thermoresistant_gluconokinase | 100 | 77 | 2.11E−96 |
| GCF_002095485.1 | 1076551 | Pantoea wallisii | gntK_Kp2H7_NOCLEBFK_02257_Thermoresistant_gluconokinase | 100 | 75.3 | 1.22E−95 |
| GCF_002095535.1 | 55209 | Pantoea cypripedii | gntK_Kp2H7_NOCLEBFK_02257_Thermoresistant_gluconokinase | 100 | 74.2 | 2.01E−94 |
| GCF_002101395.1 | 1891675 | Pantoea alhagi | gntK_f37_MKMCEHOJ_02531_Thermoresistant_gluconokinase | 96.6 | 75 | 5.57E−94 |
| GCF_002127535.1 | 351656 | Xenorhabdus | gntK_Kp2H7_NOCLEBFK_02257_Thermoresistant_gluconokinase | 84.2 | 72.5 | 3.09E−86 |
| | | vietnamensis | | | | |
| GCF_002127545.1 | 40578 | Xenorhabdus | gntK_Kp2H7_NOCLEBFK_02257_Thermoresistant_gluconokinase | 91.9 | 71.9 | 7.95E−87 |
| | | beddingii | | | | |
| GCF_002163605.1 | 53336 | Tatumella citrea | gntK_f37_MKMCEHOJ_02531_Thermoresistant_gluconokinase | 97.7 | 74.4 | 9.90E−92 |

TABLE 22

| accession | transporter_sseqid | transporter_qcovhsp | transporter_pident | transporter_evalue | gut_microbes |
|---|---|---|---|---|---|
| GCF_001277215.2 | transporter_f37_MKMCEHOJ_02530_Low-affinity_gluconate_transporter | 100 | 92.8 | 8.38E−290 | 1 |
| GCF_001277235.1 | transporter_f37_MKMCEHOJ_02530_Low-affinity_gluconate_transporter | 100 | 92.2 | 1.39E−288 | 0 |
| GCF_001277255.1 | transporter_f37_MKMCEHOJ_02530_Low-affinity_gluconate_transporter | 100 | 92.4 | 4.84E−289 | 0 |
| GCF_001280945.1 | transporter_f37_MKMCEHOJ_02530_Low-affinity_gluconate_transporter | 99.3 | 73.1 | 2.83E−232 | 0 |
| GCF_001417815.1 | transporter_i02_JFDANPEK_00220_Gnt-II_system_L-idonate_transporter | 100 | 80.1 | 1.39E−235 | 0 |
| GCF_001461805.1 | transporter_f37_MKMCEHOJ_02530_Low-affinity_gluconate_transporter | 100 | 94.2 | 2.36E−296 | 0 |
| GCF_001484765.1 | transporter_f37_MKMCEHOJ_02530_Low-affinity_gluconate_transporter | 100 | 91.3 | 1.39E−288 | 0 |
| GCF_001485335.1 | transporter_f37_MKMCEHOJ_02530_Low-affinity_gluconate_transporter | 100 | 80.5 | 8.39E−260 | 0 |
| GCF_001485355.1 | transporter_f37_MKMCEHOJ_02530_Low-affinity_gluconate_transporter | 100 | 80.5 | 1.69E−259 | 0 |
| GCF_001518835.1 | transporter_f37_MKMCEHOJ_02530_Low-affinity_gluconate_transporter | 100 | 95.5 | 1.56E−301 | 1 |
| GCF_001558935.2 | transporter_f37_MKMCEHOJ_02530_Low-affinity_gluconate_transporter | 100 | 96.2 | 4.67E−303 | 0 |
| GCF_001642805.2 | transporter_f37_MKMCEHOJ_02505_High-affinity_gluconate_transporter | 100 | 86.8 | 1.01E−263 | 0 |
| GCF_001654835.1 | transporter_f37_MKMCEHOJ_02530_Low-affinity_gluconate_transporter | 100 | 92.6 | 9.18E−294 | 0 |
| GCF_001654855.1 | transporter_f37_MKMCEHOJ_02530_Low-affinity_gluconate_transporter | 100 | 75.5 | 2.57E−242 | 0 |
| GCF_001654925.1 | transporter_f37_MKMCEHOJ_02530_Low-affinity_gluconate_transporter | 100 | 92.2 | 1.52E−292 | 0 |
| GCF_001655675.1 | transporter_f37_MKMCEHOJ_02530_Low-affinity_gluconate_transporter | 100 | 92.2 | 1.02E−290 | 0 |
| GCF_001656035.1 | transporter_f37_MKMCEHOJ_02505_High-affinity_gluconate_transporter | 100 | 86.3 | 5.01E−264 | 0 |
| GCF_001658025.2 | transporter_f37_MKMCEHOJ_02530_Low-affinity_gluconate_transporter | 100 | 92.6 | 5.54E−295 | 0 |
| GCF_001682915.1 | transporter_f37_MKMCEHOJ_02530_Low-affinity_gluconate_transporter | 100 | 93.3 | 3.57E−291 | 0 |
| GCF_001683395.1 | transporter_f37_MKMCEHOJ_02530_Low-affinity_gluconate_transporter | 100 | 93.3 | 2.36E−296 | 0 |
| GCF_001684335.1 | transporter_f37_MKMCEHOJ_02530_Low-affinity_gluconate_transporter | 100 | 77.8 | 2.57E−249 | 0 |
| GCF_001721185.1 | transporter_f37_MKMCEHOJ_02530_Low-affinity_gluconate_transporter | 100 | 77.3 | 6.02E−248 | 0 |
| GCF_001729805.1 | transporter_f37_MKMCEHOJ_02530_Low-affinity_gluconate_transporter | 100 | 96 | 3.14E−301 | 0 |
| GCF_001750725.1 | transporter_f37_MKMCEHOJ_02530_Low-affinity_gluconate_transporter | 100 | 95.7 | 1.90E−302 | 0 |
| GCF_001908095.1 | transporter_f37_MKMCEHOJ_02530_Low-affinity_gluconate_transporter | 100 | 76.9 | 4.93E−247 | 0 |
| GCF_001984825.2 | transporter_f37_MKMCEHOJ_02530_Low-affinity_gluconate_transporter | 100 | 96 | 3.14E−301 | 0 |
| GCF_002077695.1 | transporter_f37_MKMCEHOJ_02530_Low-affinity_gluconate_transporter | 100 | 80.3 | 4.61E−257 | 0 |
| GCF_002095315.1 | transporter_f37_MKMCEHOJ_02530_Low-affinity_gluconate_transporter | 100 | 80.9 | 2.40E−259 | 1 |
| GCF_002095475.1 | transporter_f37_MKMCEHOJ_02530_Low-affinity_gluconate_transporter | 100 | 80.3 | 8.39E−260 | 0 |
| GCF_002095485.1 | transporter_f37_MKMCEHOJ_02530_Low-affinity_gluconate_transporter | 100 | 80.7 | 1.14E−257 | 0 |
| GCF_002095535.1 | transporter_f37_MKMCEHOJ_02530_Low-affinity_gluconate_transporter | 100 | 80.7 | 1.97E−258 | 0 |
| GCF_002101395.1 | transporter_f37_MKMCEHOJ_02530_Low-affinity_gluconate_transporter | 100 | 83.4 | 1.18E−266 | 0 |
| GCF_002127535.1 | transporter_f37_MKMCEHOJ_02530_Low-affinity_gluconate_transporter | 100 | 77.1 | 1.21E−247 | 0 |
| GCF_002127545.1 | transporter_f37_MKMCEHOJ_02530_Low-affinity_gluconate_transporter | 100 | 78.2 | 1.27E−249 | 0 |
| GCF_002163605.1 | transporter_f37_MKMCEHOJ_02530_Low-affinity_gluconate_transporter | 100 | 78 | 1.03E−246 | 0 |

TABLE 23

| | organism. | | | gntK_ | gntK_ | gntK_ |
|---|---|---|---|---|---|---|
| accession | taxId | species | gntK_sseqid | qcovhsp | pident | evalue |
| GCF_002206385.2 | 618 | Serratia odorifera | gntK_Kp2H7_NOCLEBFK_02257_Thermoresistant_gluconokinase | 95.5 | 72.6 | 5.38E−87 |
| GCF_002259705.1 | 1160091 | Alloscardovia | gntK_i02_JFDANPEK_00221_Gluconokinase | 91.8 | 78 | 4.85E−98 |
| | | macacae | | | | |
| GCF_002259745.1 | 1630166 | Bifidobacterium | gntK_i01_CAKGJPAO_00191_Gluconokinase | 92.8 | 76.8 | 9.86E−94 |
| | | myosotis | | | | |
| GCF_002259795.1 | 1653207 | Bifidobacterium | gntK_i01_CAKGJPAO_00191_Gluconokinase | 87.1 | 72.6 | 2.39E−90 |
| | | aquikefiri | | | | |
| GCF_002290485.1 | 621 | Shigella boydii | gntK_f37_MKMCEHOJ_02531_Thermoresistant_gluconokinase | 100 | 99.4 | 1.45E−127 |
| GCF_002291445.1 | 1109412 | Brenneria goodwinii | gntK_Kp2H7_NOCLEBFK_02257_Thermoresistant_gluconokinase | 92.7 | 71.7 | 2.46E−86 |
| GCF_002393445.1 | 158822 | Cedecea neteri | gntK_Kp2H7_NOCLEBFK_02257_Thermoresistant_gluconokinase | 98.9 | 90.9 | 5.40E−117 |
| GCF_002393505.1 | 126385 | Providencia | gntK_Kp2H7_NOCLEBFK_02257_Thermoresistant_gluconokinase | 96.1 | 69.9 | 7.69E−81 |
| | | alcalifaciens | | | | |
| GCF_002504285.1 | 636 | Edwardsiella tarda | gntK_Kp2H7_NOCLEBFK_02257_Thermoresistant_gluconokinase | 97.7 | 75.6 | 2.80E−89 |
| GCF_002607735.1 | 1973495 | Proteus alimentorum | gntK_Kp2H7_NOCLEBFK_02257_Thermoresistant_gluconokinase | 93.5 | 73.8 | 1.48E−86 |
| GCF_002632595.1 | 290112 | Xenorhabdus | gntK_Kp2H7_NOCLEBFK_02257_Thermoresistant_gluconokinase | 93.5 | 69.2 | 3.30E−83 |
| | | szentirmaii | | | | |
| GCF_002632615.1 | 351674 | Xenorhabdus | gntK_f37_MKMCEHOJ_02531_Thermoresistant_gluconokinase | 93 | 69.9 | 5.42E−87 |
| | | miraniensis | | | | |
| GCF_002632755.1 | 1034471 | Xenorhabdus | gntK_Kp2H7_NOCLEBFK_02257_Thermoresistant_gluconokinase | 92.4 | 71.3 | 7.28E−85 |
| | | ishibashii | | | | |
| GCF_002632825.1 | 351614 | Xenorhabdus | gntK_f37_MKMCEHOJ_02531_Thermoresistant_gluconokinase | 96.6 | 68.8 | 2.90E−87 |
| | | stockiae | | | | |
| GCF_002632875.1 | 351676 | Xenorhabdus | gntK_Kp2H7_NOCLEBFK_02257_Thermoresistant_gluconokinase | 93 | 69.2 | 7.28E−85 |
| | | kozodoii | | | | |
| GCF_002777965.1 | 1987580 | Proteus columbae | gntK_Kp2H7_NOCLEBFK_02257_Thermoresistant_gluconokinase | 93.5 | 73.3 | 3.65E−87 |
| GCF_002811195.1 | 1076549 | Pantoea rodasii | gntK_Kp2H7_NOCLEBFK_02257_Thermoresistant_gluconokinase | 100 | 76.4 | 2.99E−96 |
| GCF_002812485.1 | 1778540 | Dickeya | gntK_f37_MKMCEHOJ_02531_Thermoresistant_gluconokinase | 89 | 60.1 | 3.76E−67 |
| | | fangzhongdai | | | | |
| GCF_002813205.1 | 1435464 | Bifidobacterium | gntK_i01_CAKGJPAO_00191_Gluconokinase | 92.3 | 76 | 6.62E−92 |
| | | psychraerophilum | | | | |
| GCF_002843235.3 | 2027290 | Providencia | gntK_Kp2H7_NOCLEBFK_02257_Thermoresistant_gluconokinase | 96.6 | 71.7 | 3.32E−84 |
| | | huaxiensis | | | | |
| GCF_002858935.1 | 92488 | Pantoea endophytica | gntK_Kp2H7_NOCLEBFK_02257_Thermoresistant_gluconokinase | 100 | 74.7 | 3.48E−95 |
| GCF_002860365.1 | 361854 | Bifidobacterium | gntK_i01_CAKGJPAO_00191_Gluconokinase | 94.9 | 77.7 | 2.71E−94 |
| | | parmae | | | | |
| GCF_002861485.1 | 419015 | Alloscardovia | gntK_i02_JFDANPEK_00221_Gluconokinase | 97.1 | 78.3 | 1.17E−95 |
| | | omnicolens | | | | |
| GCF_002861965.1 | 2702 | Gardnerella | gntK_i02_JFDANPEK_00221_Gluconokinase | 94.9 | 77.1 | 1.37E−95 |
| | | vaginalis | | | | |
| GCF_002871945.2 | 595468 | Gleimia hominis | gntK_i01_CAKGJPAO_00191_Gluconokinase | 95.9 | 65.6 | 1.30E−79 |
| GCF_002872015.2 | 190146 | Actinotignum urinale | gntK_i01_CAKGJPAO_00191_Gluconokinase | 93.3 | 65.7 | 6.23E−80 |
| GCF_002895925.1 | 1458355 | Mixta theicola | gntK_Kp2H7_NOCLEBFK_02257_Thermoresistant_gluconokinase | 96.1 | 76.6 | 1.65E−93 |
| GCF_002915575.1 | 2022662 | Superficieibacter | gntK_f37_MKMCEHOJ_02531_Thermoresistant_gluconokinase | 98.3 | 96 | 6.90E−122 |
| | | electus | | | | |
| GCF_002951615.1 | 1805933 | Rahnella sikkimica | gntK_Kp2H7_NOCLEBFK_02257_Thermoresistant_gluconokinase | 89.5 | 62.1 | 5.31E−67 |
| GCF_002952315.1 | 79967 | Erwinia pyrifoliae | gntK_Kp2H7_NOCLEBFK_02257_Thermoresistant_gluconokinase | 94.5 | 75.6 | 2.36E−94 |
| GCF_002953195.1 | 665914 | Mixta gaviniae | gntK_Kp2H7_NOCLEBFK_02257_Thermoresistant_gluconokinase | 96.1 | 76.6 | 4.06E−94 |
| GCF_002968995.1 | 2918802 | Photorhabdus | gntK_Kp2H7_NOCLEBFK_02257_Thermoresistant_gluconokinase | 96.1 | 70.3 | 4.72E−84 |
| | | hindustanensis | | | | |
| GCF_003006415.1 | 907 | Megasphaera | gntK_i33_ABMIDDIK_01308_Xylulose_kinase | 100 | 99.6 | 0 |
| | | elsdenii | | | | |
| GCF_003019925.1 | 61647 | Pluralibacter | gntK_f37_MKMCEHOJ_02531_Thermoresistant_gluconokinase | 98.9 | 89.1 | 2.38E−115 |
| | | gergoviae | | | | |
| GCF_003030905.1 | 1680 | Bifidobacterium | gntK_i01_CAKGJPAO_00191_Gluconokinase | 100 | 98.9 | 2.48E−125 |
| | | adolescentis | | | | |

TABLE 24

| accession | transporter_sseqid | transporter_qcovhsp | transporter_pident | transporter_evalue | gut_microbes |
|---|---|---|---|---|---|
| GCF_002206385.2 | transporter_f37_MKMCEHOJ_02530_Low-affinity_gluconate_transporter | 100 | 80.4 | 1.04E−255 | 0 |
| GCF_002259705.1 | transporter_i02_JFDANPEK_00220_Gnt-II_system_L-idonate_transporter | 99.1 | 67.7 | 1.35E−197 | 0 |
| GCF_002259745.1 | transporter_i02_JFDANPEK_00220_Gnt-II_system_L-idonate_transporter | 100 | 83.3 | 7.63E−247 | 0 |
| GCF_002259795.1 | transporter_i01_CAKGJPAO_00190_High-affinity_gluconate_transporter | 99.5 | 72.5 | 3.03E−218 | 0 |
| GCF_002290485.1 | transporter_f37_MKMCEHOJ_02530_Low-affinity_gluconate_transporter | 100 | 99.8 | 1.67E−312 | 0 |
| GCF_002291445.1 | transporter_f37_MKMCEHOJ_02530_Low-affinity_gluconate_transporter | 99.3 | 73.5 | 4.71E−227 | 0 |
| GCF_002393445.1 | transporter_f37_MKMCEHOJ_02530_Low-affinity_gluconate_transporter | 100 | 93 | 2.06E−290 | 0 |
| GCF_002393505.1 | transporter_f37_MKMCEHOJ_02530_Low-affinity_gluconate_transporter | 98.5 | 74.2 | 2.63E−239 | 1 |
| GCF_002504285.1 | transporter_f37_MKMCEHOJ_02530_Low-affinity_gluconate_transporter | 100 | 78.9 | 4.03E−253 | 1 |
| GCF_002607735.1 | transporter_f37_MKMCEHOJ_02530_Low-affinity_gluconate_transporter | 100 | 77.1 | 2.21E−250 | 0 |
| GCF_002632595.1 | transporter_f37_MKMCEHOJ_02530_Low-affinity_gluconate_transporter | 100 | 75.1 | 9.44E−238 | 0 |
| GCF_002632615.1 | transporter_f37_MKMCEHOJ_02530_Low-affinity_gluconate_transporter | 100 | 77.3 | 3.65E−249 | 0 |
| GCF_002632755.1 | transporter_f37_MKMCEHOJ_02530_Low-affinity_gluconate_transporter | 100 | 77.5 | 8.55E−248 | 0 |
| GCF_002632825.1 | transporter_f37_MKMCEHOJ_02530_Low-affinity_gluconate_transporter | 100 | 78.4 | 6.33E−250 | 0 |
| GCF_002632875.1 | transporter_f37_MKMCEHOJ_02530_Low-affinity_gluconate_transporter | 100 | 76.9 | 1.21E−247 | 0 |
| GCF_002777965.1 | transporter_f37_MKMCEHOJ_02530_Low-affinity_gluconate_transporter | 100 | 77.5 | 2.21E−250 | 0 |
| GCF_002811195.1 | transporter_f37_MKMCEHOJ_02530_Low-affinity_gluconate_transporter | 100 | 80.7 | 7.22E−261 | 0 |
| GCF_002812485.1 | transporter_f37_MKMCEHOJ_02505_High-affinity_gluconate_transporter | 100 | 82.6 | 1.60E−253 | 0 |
| GCF_002813205.1 | transporter_i01_CAKGJPAO_00190_High-affinity_gluconate_transporter | 99.8 | 73.7 | 6.71E−215 | 0 |
| GCF_002843235.3 | transporter_f37_MKMCEHOJ_02530_Low-affinity_gluconate_transporter | 98.7 | 74.2 | 1.19E−237 | 0 |
| GCF_002858935.1 | transporter_f37_MKMCEHOJ_02530_Low-affinity_gluconate_transporter | 100 | 80.9 | 8.39E−260 | 0 |
| GCF_002860365.1 | transporter_i01_CAKGJPAO_00190_High-affinity_gluconate_transporter | 100 | 80.3 | 5.64E−235 | 0 |
| GCF_002861485.1 | transporter_i01_CAKGJPAO_00190_High-affinity_gluconate_transporter | 98 | 68.9 | 2.87E−198 | 1 |
| GCF_002861965.1 | transporter_i01_CAKGJPAO_00190_High-affinity_gluconate_transporter | 98.4 | 68.6 | 2.87E−198 | 1 |
| GCF_002871945.2 | transporter_i01_CAKGJPAO_00190_High-affinity_gluconate_transporter | 99.8 | 74.2 | 4.79E−222 | 0 |
| GCF_002872015.2 | transporter_i01_CAKGJPAO_00190_High-affinity_gluconate_transporter | 99.3 | 70 | 4.54E−201 | 0 |
| GCF_002895925.1 | transporter_f37_MKMCEHOJ_02530_Low-affinity_gluconate_transporter | 100 | 82.7 | 3.94E−265 | 0 |
| GCF_002915575.1 | transporter_f37_MKMCEHOJ_02530_Low-affinity_gluconate_transporter | 100 | 95.5 | 1.56E−301 | 0 |
| GCF_002951615.1 | transporter_f37_MKMCEHOJ_02505_High-affinity_gluconate_transporter | 100 | 84.5 | 1.30E−259 | 0 |
| GCF_002952315.1 | transporter_f37_MKMCEHOJ_02530_Low-affinity_gluconate_transporter | 100 | 81.8 | 1.53E−262 | 0 |
| GCF_002953195.1 | transporter_f37_MKMCEHOJ_02530_Low-affinity_gluconate_transporter | 100 | 83.4 | 5.59E−265 | 0 |
| GCF_002968995.1 | transporter_f37_MKMCEHOJ_02530_Low-affinity_gluconate_transporter | 100 | 77.5 | 1.56E−250 | 0 |
| GCF_003006415.1 | transporter_i33_ABMIDDIK_01306_High-affinity_gluconate_transporter | 100 | 100 | 8.10E−305 | 1 |
| GCF_003019925.1 | transporter_f37_MKMCEHOJ_02530_Low-affinity_gluconate_transporter | 100 | 92.2 | 3.05E−290 | 1 |
| GCF_003030905.1 | transporter_i01_CAKGJPAO_00190_High-affinity_gluconate_transporter | 100 | 100 | 7.36E−298 | 1 |

TABLE 25

| | organism. | | | gntK_ | gntK_ | gntK_ |
|---|---|---|---|---|---|---|
| accession | taxId | species | gntK_sseqid | qcovhsp | pident | evalue |
| GCF_003074995.2 | 67780 | Edwardsiella | gntK_f37_MKMCEHOJ_02531_Thermoresistant_gluconokinase | 97.7 | 72.1 | 1.28E−87 |
| | | ictaluri | | | | |
| GCF_003096015.2 | 2172103 | Limnobaculum | gntK_f37_MKMCEHOJ_02531_Thermoresistant_gluconokinase | 93.1 | 61.1 | 4.54E−71 |
| | | parvum | | | | |
| GCF_003144405.1 | 2050966 | Proteus cibi | gntK_Kp2H7_NOCLEBFK_02257_Thermoresistant_gluconokinase | 93.5 | 73.8 | 2.10E−86 |
| GCF_003148935.1 | 574096 | Pantoea allii | gntK_f37_MKMCEHOJ_02531_Thermoresistant_gluconokinase | 96.6 | 76.2 | 1.23E−97 |
| GCF_003182475.1 | 451513 | Mangrovibacter | gntK_Kp2H7_NOCLEBFK_02257_Thermoresistant_gluconokinase | 98.9 | 85.1 | 1.25E−106 |
| | | plantisponsor | | | | |
| GCF_003204135.1 | 587 | Providencia rettgeri | gntK_Kp2H7_NOCLEBFK_02257_Thermoresistant_gluconokinase | 96.6 | 71.1 | 9.51E−84 |
| GCF_003207695.1 | 357240 | Franconibacter | gntK_Kp2H7_NOCLEBFK_02257_Thermoresistant_gluconokinase | 99.4 | 85 | 2.15E−104 |
| | | helveticus | | | | |
| GCF_003261575.2 | 2153354 | Klebsiella | gntK_Kp2H7_NOCLEBFK_02257_Thermoresistant_gluconokinase | 100 | 95.5 | 1.68E−122 |
| | | huaxiensis | | | | |
| GCF_003287735.1 | 2029681 | Photorhabdus bodei | gntK_Kp2H7_NOCLEBFK_02257_Thermoresistant_gluconokinase | 96.1 | 71.5 | 2.46E−86 |
| GCF_003363015.1 | 1487935 | Enterobacillus | gntK_Kp2H7_NOCLEBFK_02257_Thermoresistant_gluconokinase | 98.9 | 65.9 | 2.14E−82 |
| | | tribolii | | | | |
| GCF_003367905.1 | 2144175 | Megasphaera | gntK_i33_ABMIDDIK_01308_Xylulose_kinase | 99.8 | 88.3 | 0 |
| | | stantonii | | | | |
| GCF_003386665.1 | 351673 | Xenorhabdus | gntK_f37_MKMCEHOJ_02531_Thermoresistant_gluconokinase | 96.1 | 68.4 | 1.13E−86 |
| | | cabanillasii | | | | |
| GCF_003397585.1 | 2792977 | Gardnerella piotii | gntK_i02_JFDANPEK_00221_Gluconokinase | 94.9 | 77.1 | 4.79E−96 |
| GCF_003429605.1 | 67828 | Citrobacter gillenii | gntK_Kp2H7_NOCLEBFK_02257_Thermoresistant_gluconokinase | 99.4 | 93.1 | 6.58E−120 |
| GCF_003516125.1 | 28141 | Cronobacter | gntK_Kp2H7_NOCLEBFK_02257_Thermoresistant_gluconokinase | 99.4 | 82.2 | 3.17E−104 |
| | | sakazakii | | | | |
| GCF_003516165.1 | 615 | Serratia marcescens | gntK_Kp2H7_NOCLEBFK_02257_Thermoresistant_gluconokinase | 97.1 | 70.6 | 8.13E−84 |
| GCF_003594915.1 | 2497875 | Enterobacter | gntK_Kp2H7_NOCLEBFK_02257_Thermoresistant_gluconokinase | 98.9 | 92.5 | 5.82E−116 |
| | | chuandaensis | | | | |
| GCF_003594935.2 | 2494702 | Enterobacter | gntK_Kp2H7_NOCLEBFK_02257_Thermoresistant_gluconokinase | 98.9 | 94.2 | 8.98E−120 |
| | | huaxiensis | | | | |
| GCF_003601925.1 | 82991 | Buttiauxella izardii | gntK_f37_MKMCEHOJ_02531_Thermoresistant_gluconokinase | 98.3 | 90.2 | 3.38E−113 |
| GCF_003602095.1 | 1510574 | Rahnella | gntK_Kp2H7_NOCLEBFK_02257_Thermoresistant_gluconokinase | 94.2 | 60.2 | 2.28E−68 |
| | | woolbedingensis | | | | |
| GCF_003610465.1 | 290111 | Xenorhabdus | gntK_Kp2H7_NOCLEBFK_02257_Thermoresistant_gluconokinase | 92.4 | 71.3 | 8.89E−86 |
| | | ehlersii | | | | |
| GCF_003610915.1 | 574964 | Rahnella variigena | gntK_Kp2H7_NOCLEBFK_02257_Thermoresistant_gluconokinase | 89.5 | 62.1 | 3.74E−67 |
| GCF_003612015.1 | 2364647 | Pantoea piersonii | gntK_Kp2H7_NOCLEBFK_02257_Thermoresistant_gluconokinase | 97.2 | 74 | 4.72E−93 |
| GCF_003614975.1 | 1510573 | Rahnella bruchi | gntK_f37_MKMCEHOJ_02531_Thermoresistant_gluconokinase | 94.2 | 60.2 | 4.44E−68 |
| GCF_003634235.1 | 158877 | Yokenella | gntK_Kp2H7_NOCLEBFK_02257_Thermoresistant_gluconokinase | 98.9 | 94.8 | 9.35E−120 |
| | | regensburgei | | | | |
| GCF_003710245.1 | 221276 | [Curtobacterium] | gntK_Kp2H7_NOCLEBFK_02257_Thermoresistant_gluconokinase | 96.1 | 74.3 | 1.35E−92 |
| | | plantarum | | | | |
| GCF_003795375.1 | 1914243 | Citrobacter | gntK_Kp2H7_NOCLEBFK_02257_Thermoresistant_gluconokinase | 99.4 | 92.6 | 8.97E−118 |
| | | europaeus | | | | |
| GCF_003812345.1 | 546 | Citrobacter freundii | gntK_Kp2H7_NOCLEBFK_02257_Thermoresistant_gluconokinase | 99.4 | 92.6 | 8.97E−118 |
| GCF_003812925.1 | 571 | Klebsiella oxytoca | gntK_Kp2H7_NOCLEBFK_02257_Thermoresistant_gluconokinase | 100 | 96.6 | 7.14E−124 |
| GCF_003846135.1 | 69224 | Erwinia psidii | gntK_f37_MKMCEHOJ_02531_Thermoresistant_gluconokinase | 94.9 | 79.2 | 1.32E−94 |
| GCF_003935895.2 | 1851514 | Scandinavium | gntK_Kp2H7_NOCLEBFK_02257_Thermoresistant_gluconokinase | 99.4 | 93.1 | 1.62E−120 |
| | | goeteborgense | | | | |
| GCF_003938205.1 | 67824 | Citrobacter farmeri | gntK_f37_MKMCEHOJ_02531_Thermoresistant_gluconokinase | 98.9 | 97.7 | 9.41E−124 |
| GCF_003951095.1 | 2306975 | Bifidobacterium | gntK_i01_CAKGJPAO_00191_Gluconokinase | 93.4 | 76 | 2.15E−93 |
| | | goeldii | | | | |
| GCF_003952345.1 | 2496266 | Iodobacter ciconiae | gntK_Kp2H7_NOCLEBFK_02257_Thermoresistant_gluconokinase | 91.1 | 70.7 | 2.32E−82 |
| GCF_003952945.1 | 2306974 | Bifidobacterium | gntK_i01_CAKGJPAO_00191_Gluconokinase | 100 | 71.3 | 2.34E−87 |
| | | samirii | | | | |

TABLE 26

| accession | transporter_sseqid | transporter_qcovhsp | transporter_pident | transporter_evalue | gut_microbes |
|---|---|---|---|---|---|
| GCF_003074995.2 | transporter_f37_MKMCEHOJ_02530_Low-affinity_gluconate_transporter | 100 | 78 | 3.14E−250 | 0 |
| GCF_003096015.2 | transporter_f37_MKMCEHOJ_02505_High-affinity_gluconate_transporter | 100 | 78.6 | 7.87E−237 | 0 |
| GCF_003144405.1 | transporter_f37_MKMCEHOJ_02530_Low-affinity_gluconate_transporter | 100 | 77.1 | 1.56E−250 | 0 |
| GCF_003148935.1 | transporter_f37_MKMCEHOJ_02530_Low-affinity_gluconate_transporter | 100 | 80.7 | 1.45E−260 | 0 |
| GCF_003182475.1 | transporter_f37_MKMCEHOJ_02530_Low-affinity_gluconate_transporter | 100 | 91.9 | 2.06E−290 | 0 |
| GCF_003204135.1 | transporter_f37_MKMCEHOJ_02530_Low-affinity_gluconate_transporter | 98.7 | 73.9 | 9.74E−237 | 1 |
| GCF_003207695.1 | transporter_f37_MKMCEHOJ_02530_Low-affinity_gluconate_transporter | 100 | 91.7 | 2.40E−289 | 1 |
| GCF_003261575.2 | transporter_f37_MKMCEHOJ_02530_Low-affinity_gluconate_transporter | 100 | 96.9 | 4.88E−305 | 0 |
| GCF_003287735.1 | transporter_f37_MKMCEHOJ_02530_Low-affinity_gluconate_transporter | 100 | 77.3 | 8.98E−250 | 0 |
| GCF_003363015.1 | transporter_f37_MKMCEHOJ_02530_Low-affinity_gluconate_transporter | 99.8 | 73 | 2.98E−234 | 0 |
| GCF_003367905.1 | transporter_i33_ABMIDDIK_01306_High-affinity_gluconate_transporter | 99.5 | 92.2 | 8.72E−277 | 0 |
| GCF_003386665.1 | transporter_f37_MKMCEHOJ_02530_Low-affinity_gluconate_transporter | 100 | 77.8 | 2.21E−250 | 0 |
| GCF_003397585.1 | transporter_i01_CAKGJPAO_00190_High-affinity_gluconate_transporter | 98.4 | 68.6 | 1.65E−197 | 0 |
| GCF_003429605.1 | transporter_f37_MKMCEHOJ_02530_Low-affinity_gluconate_transporter | 100 | 96.4 | 6.63E−303 | 0 |
| GCF_003516125.1 | transporter_f37_MKMCEHOJ_02530_Low-affinity_gluconate_transporter | 100 | 92.8 | 9.77E−289 | 1 |
| GCF_003516165.1 | transporter_f37_MKMCEHOJ_02505_High-affinity_gluconate_transporter | 100 | 87 | 1.75E−264 | 1 |
| GCF_003594915.1 | transporter_f37_MKMCEHOJ_02530_Low-affinity_gluconate_transporter | 100 | 96 | 3.14E−301 | 0 |
| GCF_003594935.2 | transporter_f37_MKMCEHOJ_02530_Low-affinity_gluconate_transporter | 100 | 95.7 | 1.28E−300 | 0 |
| GCF_003601925.1 | transporter_f37_MKMCEHOJ_02530_Low-affinity_gluconate_transporter | 100 | 92.8 | 3.06E−292 | 0 |
| GCF_003602095.1 | transporter_f37_MKMCEHOJ_02505_High-affinity_gluconate_transporter | 100 | 85.2 | 4.78E−262 | 0 |
| GCF_003610465.1 | transporter_f37_MKMCEHOJ_02530_Low-affinity_gluconate_transporter | 100 | 76.2 | 1.90E−244 | 0 |
| GCF_003610915.1 | transporter_f37_MKMCEHOJ_02505_High-affinity_gluconate_transporter | 100 | 85.4 | 8.28E−263 | 0 |
| GCF_003612015.1 | transporter_f37_MKMCEHOJ_02530_Low-affinity_gluconate_transporter | 100 | 80.7 | 2.40E−259 | 0 |
| GCF_003614975.1 | transporter_f37_MKMCEHOJ_02505_High-affinity_gluconate_transporter | 100 | 85.2 | 4.78E−262 | 0 |
| GCF_003634235.1 | transporter_f37_MKMCEHOJ_02530_Low-affinity_gluconate_transporter | 100 | 96.2 | 5.68E−304 | 1 |
| GCF_003710245.1 | transporter_f37_MKMCEHOJ_02530_Low-affinity_gluconate_transporter | 100 | 80.5 | 9.76E−259 | 0 |
| GCF_003795375.1 | transporter_f37_MKMCEHOJ_02530_Low-affinity_gluconate_transporter | 100 | 96.2 | 2.31E−303 | 0 |
| GCF_003812345.1 | transporter_f37_MKMCEHOJ_02530_Low-affinity_gluconate_transporter | 100 | 96.4 | 7.72E−302 | 1 |
| GCF_003812925.1 | transporter_f37_MKMCEHOJ_02530_Low-affinity_gluconate_transporter | 100 | 96.4 | 1.98E−304 | 1 |
| GCF_003846135.1 | transporter_f37_MKMCEHOJ_02530_Low-affinity_gluconate_transporter | 100 | 81.2 | 6.20E−262 | 0 |
| GCF_003935895.2 | transporter_f37_MKMCEHOJ_02530_Low-affinity_gluconate_transporter | 100 | 96 | 6.34E−301 | 0 |
| GCF_003938205.1 | transporter_f37_MKMCEHOJ_02530_Low-affinity_gluconate_transporter | 100 | 96.6 | 8.07E−304 | 1 |
| GCF_003951095.1 | transporter_i01_CAKGJPAO_00190_High-affinity_gluconate_transporter | 100 | 81.9 | 4.40E−239 | 0 |
| GCF_003952345.1 | transporter_f37_MKMCEHOJ_02530_Low-affinity_gluconate_transporter | 100 | 77.1 | 8.99E−243 | 0 |
| GCF_003952945.1 | transporter_i02_JFDANPEK_00220_Gnt-II_system_L-idonate_transporter | 100 | 78.1 | 1.60E−227 | 0 |

TABLE 27

| | organism. | | | gntK_ | gntK_ | gntK_ |
|---|---|---|---|---|---|---|
| accession | taxId | species | gntK_sseqid | qcovhsp | pident | evalue |
| GCF_003964805.1 | 2497436 | Enterobacter | gntK_Kp2H7_NOCLEBFK_02257_Thermoresistant_gluconokinase | 98.9 | 93.1 | 4.10E−116 |
| | | quasiroggenkampii | | | | |
| GCF_003992065.1 | 1502943 | Veillonella | gntK_i33_ABMIDDIK_01308_Xylulose_kinase | 97.1 | 63.5 | 8.10E−238 |
| | | seminalis | | | | |
| GCF_003992115.1 | 103892 | Veillonella ratti | gntK_i37_IIDCKEOG_02051_Xylulose_kinase | 97.1 | 63.3 | 3.40E−237 |
| GCF_004116975.1 | 183417 | Proteus hauseri | gntK_f37_MKMCEHOJ_02531_Thermoresistant_gluconokinase | 94 | 72.7 | 1.19E−87 |
| GCF_004168465.1 | 357233 | Siccibacter | gntK_f37_MKMCEHOJ_02531_Thermoresistant_gluconokinase | 100 | 81.5 | 2.71E−102 |
| | | turicensis | | | | |
| GCF_004331265.1 | 2529381 | Enterobacter | gntK_Kp2H7_NOCLEBFK_02257_Thermoresistant_gluconokinase | 98.9 | 94.2 | 4.45E−120 |
| | | wuhouensis | | | | |
| GCF_004331385.1 | 2529382 | Enterobacter | gntK_Kp2H7_NOCLEBFK_02257_Thermoresistant_gluconokinase | 98.9 | 94.2 | 3.00E−118 |
| | | quasihormaechei | | | | |
| GCF_004331415.1 | 2529380 | Kosakonia | gntK_Kp2H7_NOCLEBFK_02257_Thermoresistant_gluconokinase | 98.9 | 93.1 | 4.63E−118 |
| | | quasisacchari | | | | |
| GCF_004332295.1 | 2496842 | Alloscardovia | gntK_i02_JFDANPEK_00221_Gluconokinase | 95.4 | 74.7 | 8.45E−92 |
| | | theropitheci | | | | |
| GCF_004341685.1 | 86956 | Pectinatus | gntK_i33_ABMIDDIK_01308_Xylulose_kinase | 99.2 | 73.2 | 2.75E−287 |
| | | cerevisiiphilus | | | | |
| GCF_004353845.1 | 2546350 | Citrobacter | gntK_Kp2H7_NOCLEBFK_02257_Thermoresistant_gluconokinase | 99.4 | 92.6 | 8.97E−118 |
| | | arsenatis | | | | |
| GCF_004684345.1 | 2562449 | Citrobacter | gntK_f37_MKMCEHOJ_02531_Thermoresistant_gluconokinase | 98.9 | 93.1 | 3.68E−119 |
| | | tructae | | | | |
| GCF_004768745.1 | 458197 | Serratia | gntK_f37_MKMCEHOJ_02531_Thermoresistant_gluconokinase | 97.1 | 69.4 | 3.90E−84 |
| | | nematodiphila | | | | |
| GCF_004792415.1 | 470934 | Pantoea vagans | gntK_Kp2H7_NOCLEBFK_02257_Thermoresistant_gluconokinase | 96.1 | 74.3 | 9.52E−93 |
| GCF_004801125.1 | 67829 | Citrobacter | gntK_f37_MKMCEHOJ_02531_Thermoresistant_gluconokinase | 98.9 | 93.1 | 7.42E−119 |
| | | murliniae | | | | |
| GCF_005405725.1 | 1333651 | Bifidobacterium | gntK_i01_CAKGJPAO_00191_Gluconokinase | 94.6 | 87.4 | 5.80E−112 |
| | | moukalabense | | | | |
| GCF_005671395.1 | 2579935 | Jejubacter | gntK_Kp2H7_NOCLEBFK_02257_Thermoresistant_gluconokinase | 99.4 | 88.4 | 2.18E−113 |
| | | calystegiae | | | | |
| GCF_005860775.1 | 2582917 | Klebsiella indica | gntK_Kp2H7_NOCLEBFK_02257_Thermoresistant_gluconokinase | 99.4 | 94.3 | 6.12E−123 |
| GCF_006517625.1 | 2562439 | Mixta tenebrionis | gntK_Kp2H7_NOCLEBFK_02257_Thermoresistant_gluconokinase | 96.1 | 77.2 | 1.16E−93 |
| GCF_006711645.1 | 1259973 | Raoultella | gntK_Kp2H7_NOCLEBFK_02257_Thermoresistant_gluconokinase | 100 | 96.6 | 1.23E−124 |
| | | electrica | | | | |
| GCF_007035805.1 | 61645 | Enterobacter | gntK_Kp2H7_NOCLEBFK_02257_Thermoresistant_gluconokinase | 98.9 | 93.1 | 4.10E−116 |
| | | asburiae | | | | |
| GCF_007632255.1 | 548 | Klebsiella | gntK_Kp2H7_NOCLEBFK_02257_Thermoresistant_gluconokinase | 100 | 98.3 | 4.30E−125 |
| | | aerogenes | | | | |
| GCF_007681265.1 | 665913 | Mixta calida | gntK_f37_MKMCEHOJ_02531_Thermoresistant_gluconokinase | 96.6 | 75 | 3.92E−94 |
| GCF_008330085.1 | 283686 | Kosakonia | gntK_f37_MKMCEHOJ_02531_Thermoresistant_gluconokinase | 98.3 | 94.3 | 3.28E−120 |
| | | radicincitans | | | | |
| GCF_008364255.1 | 2703794 | Serratia nevei | gntK_Kp2H7_NOCLEBFK_02257_Thermoresistant_gluconokinase | 97.1 | 70 | 1.64E−83 |
| GCF_008364325.2 | 614 | Serratia | gntK_f37_MKMCEHOJ_02531_Thermoresistant_gluconokinase | 94.4 | 75.1 | 5.27E−90 |
| | | liquefaciens | | | | |
| GCF_008364555.1 | 2797505 | Enterobacter | gntK_Kp2H7_NOCLEBFK_02257_Thermoresistant_gluconokinase | 98.9 | 93.1 | 4.10E−116 |
| | | vonholyi | | | | |
| GCF_008364625.1 | 2797506 | Enterobacter | gntK_Kp2H7_NOCLEBFK_02257_Thermoresistant_gluconokinase | 98.9 | 93.1 | 4.10E−116 |
| | | dykesii | | | | |
| GCF_008692955.1 | 368603 | Morganella | gntK_Kp2H7_NOCLEBFK_02257_Thermoresistant_gluconokinase | 96.7 | 67.8 | 6.96E−84 |
| | | psychrotolerans | | | | |
| GCF_008693605.1 | 1639133 | Citrobacter | gntK_Kp2H7_NOCLEBFK_02257_Thermoresistant_gluconokinase | 99.4 | 92.6 | 8.97E−118 |
| | | portucalensis | | | | |
| GCF_008693645.1 | 67827 | Citrobacter | gntK_Kp2H7_NOCLEBFK_02257_Thermoresistant_gluconokinase | 99.4 | 92 | 8.61E−116 |
| | | werkmanii | | | | |
| GCF_008710095.1 | 2590031 | Affinibrenneria | gntK_Kp2H7_NOCLEBFK_02257_Thermoresistant_gluconokinase | 93.8 | 74.7 | 4.81E−88 |
| | | salicis | | | | |
| GCF_009036245.1 | 2071710 | Enterobacter | gntK_Kp2H7_NOCLEBFK_02257_Thermoresistant_gluconokinase | 98.9 | 93.6 | 2.47E−117 |
| | | sichuanensis | | | | |
| GCF_009176645.1 | 2478464 | Enterobacter | gntK_Kp2H7_NOCLEBFK_02257_Thermoresistant_gluconokinase | 98.9 | 94.8 | 4.45E−120 |
| | | oligotrophicus | | | | |
| GCF_009295515.1 | 1563158 | Erwinia | gntK_f37_MKMCEHOJ_02531_Thermoresistant_gluconokinase | 96.6 | 78.5 | 1.75E−97 |
| | | endophytica | | | | |

TABLE 28

| accession | transporter_sseqid | transporter_qcovhsp | transporter_pident | transporter_evalue | gut_microbes |
|---|---|---|---|---|---|
| GCF_003964805.1 | transporter_f37_MKMCEHOJ_02530_Low-affinity_gluconate_transporter | 100 | 96 | 3.14E−301 | 0 |
| GCF_003992065.1 | transporter_i33_ABMIDDIK_01306_High-affinity_gluconate_transporter | 100 | 73.9 | 1.15E−223 | 1 |
| GCF_003992115.1 | transporter_i33_ABMIDDIK_01306_High-affinity_gluconate_transporter | 100 | 73.4 | 3.83E−222 | 0 |
| GCF_004116975.1 | transporter_f37_MKMCEHOJ_02530_Low-affinity_gluconate_transporter | 100 | 75.5 | 2.10E−241 | 0 |
| GCF_004168465.1 | transporter_f37_MKMCEHOJ_02530_Low-affinity_gluconate_transporter | 100 | 92.6 | 1.45E−290 | 0 |
| GCF_004331265.1 | transporter_f37_MKMCEHOJ_02530_Low-affinity_gluconate_transporter | 100 | 95.7 | 1.28E−300 | 0 |
| GCF_004331385.1 | transporter_f37_MKMCEHOJ_02530_Low-affinity_gluconate_transporter | 100 | 96.2 | 1.56E−301 | 0 |
| GCF_004331415.1 | transporter_f37_MKMCEHOJ_02530_Low-affinity_gluconate_transporter | 100 | 93.3 | 4.76E−296 | 0 |
| GCF_004332295.1 | transporter_i02_JFDANPEK_00220_Gnt-II_system_L-idonate_transporter | 98 | 70.1 | 6.11E−200 | 0 |
| GCF_004341685.1 | transporter_i37_IIDCKEOG_02049_High-affinity_gluconate_transporter | 100 | 80.4 | 6.79E−246 | 0 |
| GCF_004353845.1 | transporter_f37_MKMCEHOJ_02530_Low-affinity_gluconate_transporter | 100 | 96.2 | 2.31E−303 | 0 |
| GCF_004684345.1 | transporter_f37_MKMCEHOJ_02530_Low-affinity_gluconate_transporter | 100 | 96.9 | 4.67E−303 | 0 |
| GCF_004768745.1 | transporter_f37_MKMCEHOJ_02505_High-affinity_gluconate_transporter | 100 | 87 | 1.75E−264 | 0 |
| GCF_004792415.1 | transporter_f37_MKMCEHOJ_02530_Low-affinity_gluconate_transporter | 100 | 80.9 | 7.22E−261 | 0 |
| GCF_004801125.1 | transporter_f37_MKMCEHOJ_02530_Low-affinity_gluconate_transporter | 100 | 96 | 3.83E−302 | 0 |
| GCF_005405725.1 | transporter_i02_JFDANPEK_00220_Gnt-II_system_L-idonate_transporter | 100 | 93.9 | 1.47E−281 | 0 |
| GCF_005671395.1 | transporter_f37_MKMCEHOJ_02530_Low-affinity_gluconate_transporter | 100 | 94.4 | 9.60E−296 | 0 |
| GCF_005860775.1 | transporter_f37_MKMCEHOJ_02530_Low-affinity_gluconate_transporter | 100 | 96.2 | 1.10E−301 | 0 |
| GCF_006517625.1 | transporter_f37_MKMCEHOJ_02530_Low-affinity_gluconate_transporter | 100 | 83.4 | 7.94E−265 | 0 |
| GCF_006711645.1 | transporter_f37_MKMCEHOJ_02530_Low-affinity_gluconate_transporter | 100 | 95.3 | 7.07E−298 | 0 |
| GCF_007035805.1 | transporter_f37_MKMCEHOJ_02530_Low-affinity_gluconate_transporter | 100 | 95.5 | 1.82E−300 | 1 |
| GCF_007632255.1 | transporter_f37_MKMCEHOJ_02530_Low-affinity_gluconate_transporter | 100 | 96 | 2.58E−300 | 1 |
| GCF_007681265.1 | transporter_f37_MKMCEHOJ_02530_Low-affinity_gluconate_transporter | 100 | 83.4 | 7.94E−265 | 0 |
| GCF_008330085.1 | transporter_f37_MKMCEHOJ_02530_Low-affinity_gluconate_transporter | 100 | 92.8 | 6.76E−296 | 0 |
| GCF_008364255.1 | transporter_f37_MKMCEHOJ_02505_High-affinity_gluconate_transporter | 100 | 87 | 5.01E−264 | 0 |
| GCF_008364325.2 | transporter_f37_MKMCEHOJ_02530_Low-affinity_gluconate_transporter | 99.8 | 72.1 | 1.89E−230 | 1 |
| GCF_008364555.1 | transporter_f37_MKMCEHOJ_02530_Low-affinity_gluconate_transporter | 100 | 96 | 3.14E−301 | 0 |
| GCF_008364625.1 | transporter_f37_MKMCEHOJ_02530_Low-affinity_gluconate_transporter | 100 | 95.7 | 1.28E−300 | 0 |
| GCF_008692955.1 | transporter_f37_MKMCEHOJ_02530_Low-affinity_gluconate_transporter | 100 | 76.2 | 2.57E−242 | 0 |
| GCF_008693605.1 | transporter_f37_MKMCEHOJ_02530_Low-affinity_gluconate_transporter | 100 | 96.2 | 1.63E−303 | 0 |
| GCF_008693645.1 | transporter_f37_MKMCEHOJ_02530_Low-affinity_gluconate_transporter | 100 | 96 | 1.56E−301 | 1 |
| GCF_008710095.1 | transporter_f37_MKMCEHOJ_02530_Low-affinity_gluconate_transporter | 100 | 72.9 | 1.45E−225 | 0 |
| GCF_009036245.1 | transporter_f37_MKMCEHOJ_02530_Low-affinity_gluconate_transporter | 100 | 95.3 | 4.27E−299 | 0 |
| GCF_009176645.1 | transporter_f37_MKMCEHOJ_02530_Low-affinity_gluconate_transporter | 100 | 96.2 | 1.63E−303 | 0 |
| GCF_009295515.1 | transporter_f37_MKMCEHOJ_02530_Low-affinity_gluconate_transporter | 100 | 81.8 | 3.58E−261 | 0 |

TABLE 29

| | organism. | | | gntK_ | gntK_ | gntK_ |
|---|---|---|---|---|---|---|
| accession | taxId | species | gntK_sseqid | qcovhsp | pident | evalue |
| GCF_009295665.1 | 2653932 | Citrobacter | gntK_f37_MKMCEHOJ_02531_Thermoresistant_gluconokinase | 98.9 | 97.7 | 9.41E−124 |
| | | telavivensis | | | | |
| GCF_009363135.1 | 551989 | Kosakonia arachidis | gntK_f37_MKMCEHOJ_02531_Thermoresistant_gluconokinase | 98.3 | 93.7 | 1.63E−120 |
| GCF_009646115.1 | 470933 | Pantoea eucalypti | gntK_Kp2H7_NOCLEBFK_02257_Thermoresistant_gluconokinase | 96.1 | 74.9 | 2.34E−93 |
| GCF_009648935.1 | 57706 | Citrobacter braakii | gntK_Kp2H7_NOCLEBFK_02257_Thermoresistant_gluconokinase | 99.4 | 92.6 | 8.97E−118 |
| GCF_009648975.1 | 244366 | Klebsiella variicola | gntK_Kp2H7_NOCLEBFK_02257_Thermoresistant_gluconokinase | 100 | 99.4 | 2.70E−128 |
| GCF_009649915.1 | 61648 | Kluyvera intermedia | gntK_Kp2H7_NOCLEBFK_02257_Thermoresistant_gluconokinase | 98.3 | 92 | 1.62E−118 |
| GCF_009728735.1 | 2052056 | Pantoea | gntK_Kp2H7_NOCLEBFK_02257_Thermoresistant_gluconokinase | 100 | 74.2 | 7.02E−95 |
| | | phytobeneficialis | | | | |
| GCF_009730055.1 | 631 | Yersinia intermedia | gntK_f37_MKMCEHOJ_02531_Thermoresistant_gluconokinase | 91.8 | 60.3 | 3.18E−70 |
| GCF_009817885.1 | 2597702 | Serratia | gntK_Kp2H7_NOCLEBFK_02257_Thermoresistant_gluconokinase | 94.9 | 73.1 | 1.26E−85 |
| | | rhizosphaerae | | | | |
| GCF_009831665.3 | 138074 | Serratia symbiotica | gntK_Kp2H7_NOCLEBFK_02257_Thermoresistant_gluconokinase | 93.7 | 70.7 | 2.21E−81 |
| GCF_009914055.1 | 1615494 | Mixta intestinalis | gntK_Kp2H7_NOCLEBFK_02257_Thermoresistant_gluconokinase | 96.1 | 77.2 | 1.73E−95 |
| GCF_009936155.1 | 394935 | Chromobacterium | gntK_f37_MKMCEHOJ_02531_Thermoresistant_gluconokinase | 94.3 | 62.4 | 6.09E−74 |
| | | haemolyticum | | | | |
| GCF_010667645.1 | 2661628 | Bifidobacterium | gntK_i01_CAKGJPAO_00191_Gluconokinase | 94.9 | 77.7 | 1.35E−94 |
| | | platyrrhinorum | | | | |
| GCF_011044475.1 | 66269 | Pantoea stewartii | gntK_f37_MKMCEHOJ_02531_Thermoresistant_gluconokinase | 96.6 | 74.4 | 2.76E−94 |
| GCF_011045835.1 | 626774 | Proteus terrae | gntK_Kp2H7_NOCLEBFK_02257_Thermoresistant_gluconokinase | 93.5 | 73.8 | 6.33E−88 |
| GCF_011065485.1 | 2703882 | Rahnella | gntK_Kp2H7_NOCLEBFK_02257_Thermoresistant_gluconokinase | 89.5 | 62.1 | 3.74E−67 |
| | | contaminans | | | | |
| GCF_011067265.1 | 1646377 | Rouxiella badensis | gntK_Kp2H7_NOCLEBFK_02257_Thermoresistant_gluconokinase | 87.4 | 60.8 | 2.50E−66 |
| GCF_011068345.1 | 565 | Atlantibacter | gntK_f37_MKMCEHOJ_02531_Thermoresistant_gluconokinase | 98.9 | 86.9 | 3.10E−109 |
| | | hermannii | | | | |
| GCF_011189495.1 | 1123042 | Photorhabdus | gntK_f37_MKMCEHOJ_02531_Thermoresistant_gluconokinase | 95.5 | 70.2 | 1.76E−88 |
| | | stackebrandtii | | | | |
| GCF_011189505.1 | 1004159 | Photorhabdus | gntK_f37_MKMCEHOJ_02531_Thermoresistant_gluconokinase | 95.5 | 69.6 | 1.44E−87 |
| | | tasmaniensis | | | | |
| GCF_011189575.1 | 471575 | Photorhabdus | gntK_f37_MKMCEHOJ_02531_Thermoresistant_gluconokinase | 95.5 | 69.6 | 1.01E−87 |
| | | cinerea | | | | |
| GCF_011601265.1 | 3044271 | lodobacter violacea | gntK_f37_MKMCEHOJ_02531_Thermoresistant_gluconokinase | 91.7 | 68.5 | 3.18E−82 |
| GCF_011617105.1 | 569 | Hafnia alvei | gntK_Kp2H7_NOCLEBFK_02257_Thermoresistant_gluconokinase | 98.9 | 68.2 | 2.89E−87 |
| GCF_012029655.1 | 577 | Raoultella terrigena | gntK_Kp2H7_NOCLEBFK_02257_Thermoresistant_gluconokinase | 100 | 96.6 | 1.23E−124 |
| GCF_012923785.1 | 395631 | Phytobacter | gntK_f37_MKMCEHOJ_02531_Thermoresistant_gluconokinase | 100 | 93.6 | 4.62E−119 |
| | | diazotrophicus | | | | |
| GCF_013234275.1 | 82977 | Buttiauxella agrestis | gntK_f37_MKMCEHOJ_02531_Thermoresistant_gluconokinase | 98.3 | 90.2 | 2.38E−113 |
| GCF_013282725.1 | 349967 | Yersinia mollaretii | gntK_f37_MKMCEHOJ_02531_Thermoresistant_gluconokinase | 94.1 | 60 | 1.11E−70 |
| GCF_013284055.1 | 2739061 | Deefgea piscis | gntK_Kp2H7_NOCLEBFK_02257_Thermoresistant_gluconokinase | 94.7 | 72.8 | 1.71E−84 |
| GCF_013374815.1 | 624 | Shigella sonnei | gntK_f37_MKMCEHOJ_02531_Thermoresistant_gluconokinase | 100 | 99.4 | 1.45E−127 |
| GCF_013403315.1 | 472694 | Pantoea brenneri | gntK_Kp2H7_NOCLEBFK_02257_Thermoresistant_gluconokinase | 96.6 | 75 | 1.42E−94 |
| GCF_013403565.1 | 2739434 | Chitinibacter | gntK_f37_MKMCEHOJ_02531_Thermoresistant_gluconokinase | 94.7 | 67.3 | 1.92E−76 |
| | | bivalviorum | | | | |
| GCF_013423785.1 | 1737446 | Chitinibacter | gntK_f37_MKMCEHOJ_02531_Thermoresistant_gluconokinase | 90.2 | 71.8 | 8.89E−78 |
| | | fontanus | | | | |
| GCF_013753875.1 | 2518128 | Candidatus | gntK_f37_MKMCEHOJ_02531_Thermoresistant_gluconokinase | 97.2 | 69.9 | 5.90E−89 |
| | | Pantoea persica | | | | |
| GCF_014156615.1 | 1592631 | Pantoea pleuroti | gntK_Kp2H7_NOCLEBFK_02257_Thermoresistant_gluconokinase | 96.1 | 74.3 | 6.71E−93 |
| GCF_014218705.1 | 2725494 | Kluyvera | gntK_Kp2H7_NOCLEBFK_02257_Thermoresistant_gluconokinase | 98.3 | 92 | 4.63E−118 |
| | | sichuanensis | | | | |

TABLE 30

| accession | transporter_sseqid | transporter_qcovhsp | transporter_pident | transporter_evalue | gut_microbes |
|---|---|---|---|---|---|
| GCF_009295665.1 | transporter_f37_MKMCEHOJ_02530_Low-affinity_gluconate_transporter | 100 | 96.2 | 8.07E−304 | 0 |
| GCF_009363135.1 | transporter_f37_MKMCEHOJ_02530_Low-affinity_gluconate_transporter | 100 | 93 | 6.76E−296 | 0 |
| GCF_009646115.1 | transporter_f37_MKMCEHOJ_02530_Low-affinity_gluconate_transporter | 100 | 80.9 | 7.22E−261 | 0 |
| GCF_009648935.1 | transporter_f37_MKMCEHOJ_02530_Low-affinity_gluconate_transporter | 100 | 96.6 | 1.40E−304 | 1 |
| GCF_009648975.1 | transporter_f37_MKMCEHOJ_02530_Low-affinity_gluconate_transporter | 100 | 96.6 | 2.31E−303 | 1 |
| GCF_009649915.1 | transporter_f37_MKMCEHOJ_02530_Low-affinity_gluconate_transporter | 100 | 92.8 | 7.53E−293 | 1 |
| GCF_009728735.1 | transporter_f37_MKMCEHOJ_02530_Low-affinity_gluconate_transporter | 100 | 80.5 | 1.61E−257 | 0 |
| GCF_009730055.1 | transporter_f37_MKMCEHOJ_02505_High-affinity_gluconate_transporter | 100 | 87 | 1.29E−266 | 1 |
| GCF_009817885.1 | transporter_f37_MKMCEHOJ_02530_Low-affinity_gluconate_transporter | 100 | 81.6 | 1.09E−257 | 0 |
| GCF_009831665.3 | transporter_f37_MKMCEHOJ_02505_High-affinity_gluconate_transporter | 100 | 82 | 2.79E−247 | 0 |
| GCF_009914055.1 | transporter_f37_MKMCEHOJ_02530_Low-affinity_gluconate_transporter | 100 | 83 | 1.31E−263 | 0 |
| GCF_009936155.1 | transporter_f37_MKMCEHOJ_02530_Low-affinity_gluconate_transporter | 99.1 | 74.4 | 1.09E−229 | 0 |
| GCF_010667645.1 | transporter_i02_JFDANPEK_00220_Gnt-II_system_L-idonate_transporter | 100 | 80.1 | 1.97E−235 | 0 |
| GCF_011044475.1 | transporter_f37_MKMCEHOJ_02530_Low-affinity_gluconate_transporter | 99.8 | 80.7 | 9.76E−259 | 0 |
| GCF_011045835.1 | transporter_f37_MKMCEHOJ_02530_Low-affinity_gluconate_transporter | 100 | 76.4 | 7.35E−249 | 0 |
| GCF_011065485.1 | transporter_f37_MKMCEHOJ_02505_High-affinity_gluconate_transporter | 100 | 84.2 | 9.18E−260 | 0 |
| GCF_011067265.1 | transporter_f37_MKMCEHOJ_02505_High-affinity_gluconate_transporter | 100 | 84.2 | 6.17E−258 | 0 |
| GCF_011068345.1 | transporter_f37_MKMCEHOJ_02530_Low-affinity_gluconate_transporter | 100 | 92.4 | 1.07E−292 | 0 |
| GCF_011189495.1 | transporter_f37_MKMCEHOJ_02530_Low-affinity_gluconate_transporter | 99.3 | 76.9 | 2.70E−244 | 0 |
| GCF_011189505.1 | transporter_f37_MKMCEHOJ_02530_Low-affinity_gluconate_transporter | 100 | 76.2 | 1.21E−247 | 0 |
| GCF_011189575.1 | transporter_f37_MKMCEHOJ_02530_Low-affinity_gluconate_transporter | 99.3 | 77.4 | 1.72E−247 | 0 |
| GCF_011601265.1 | transporter_f37_MKMCEHOJ_02530_Low-affinity_gluconate_transporter | 100 | 76.6 | 2.10E−241 | 0 |
| GCF_011617105.1 | transporter_f37_MKMCEHOJ_02530_Low-affinity_gluconate_transporter | 99.8 | 73.6 | 1.99E−232 | 1 |
| GCF_012029655.1 | transporter_f37_MKMCEHOJ_02530_Low-affinity_gluconate_transporter | 100 | 95.3 | 8.61E−299 | 0 |
| GCF_012923785.1 | transporter_f37_MKMCEHOJ_02530_Low-affinity_gluconate_transporter | 100 | 92.8 | 6.18E−292 | 0 |
| GCF_013234275.1 | transporter_f37_MKMCEHOJ_02530_Low-affinity_gluconate_transporter | 100 | 93 | 4.55E−294 | 0 |
| GCF_013282725.1 | transporter_f37_MKMCEHOJ_02505_High-affinity_gluconate_transporter | 100 | 86.1 | 1.43E−263 | 1 |
| GCF_013284055.1 | transporter_f37_MKMCEHOJ_02530_Low-affinity_gluconate_transporter | 100 | 75.8 | 1.13E−241 | 0 |
| GCF_013374815.1 | transporter_f37_MKMCEHOJ_02530_Low-affinity_gluconate_transporter | 100 | 99.8 | 1.67E−312 | 0 |
| GCF_013403315.1 | transporter_f37_MKMCEHOJ_02530_Low-affinity_gluconate_transporter | 100 | 80.7 | 1.39E−258 | 0 |
| GCF_013403565.1 | transporter_f37_MKMCEHOJ_02530_Low-affinity_gluconate_transporter | 100 | 77.5 | 4.04E−246 | 0 |
| GCF_013423785.1 | transporter_f37_MKMCEHOJ_02530_Low-affinity_gluconate_transporter | 100 | 76 | 1.56E−243 | 0 |
| GCF_013753875.1 | transporter_f37_MKMCEHOJ_02530_Low-affinity_gluconate_transporter | 100 | 67.2 | 5.55E−201 | 0 |
| GCF_014156615.1 | transporter_f37_MKMCEHOJ_02530_Low-affinity_gluconate_transporter | 100 | 80.9 | 2.40E−259 | 0 |
| GCF_014218705.1 | transporter_f37_MKMCEHOJ_02530_Low-affinity_gluconate_transporter | 99.8 | 93.3 | 7.25E−285 | 0 |

TABLE 31

| | organism. | | | gntK_ | gntK_ | gntK_ |
|---|---|---|---|---|---|---|
| accession | taxId | species | gntK_sseqid | qcovhsp | pident | evalue |
| GCF_014250475.1 | 2754044 | Selenomonas | gntK_i33_ABMIDDIK_01308_Xylulose_kinase | 99.2 | 67.9 | 6.97E−262 |
| | | timonae | | | | |
| GCF_014295015.1 | 628 | Xenorhabdus | gntK_Kp2H7_NOCLEBFK_02257_Thermoresistant_gluconokinase | 97.2 | 71.1 | 2.03E−87 |
| | | nematophila | | | | |
| GCF_014467235.1 | 333964 | Xenorhabdus indica | gntK_Kp2H7_NOCLEBFK_02257_Thermoresistant_gluconokinase | 96.6 | 66.9 | 9.60E−86 |
| GCF_014635685.1 | 1769422 | Galliscardovia | gntK_i01_CAKGJPAO_00191_Gluconokinase | 97.6 | 65.1 | 1.51E−78 |
| | | ingluviei | | | | |
| GCF_014644275.1 | 2047724 | Franconibacter | gntK_Kp2H7_NOCLEBFK_02257_Thermoresistant_gluconokinase | 99.4 | 83.8 | 6.15E−104 |
| | | daqui | | | | |
| GCF_014652175.1 | 990144 | Providencia | gntK_f37_MKMCEHOJ_02531_Thermoresistant_gluconokinase | 89.3 | 68.9 | 2.16E−77 |
| | | thailandensis | | | | |
| GCF_014653055.1 | 1398493 | Pseudocitrobacter | gntK_Kp2H7_NOCLEBFK_02257_Thermoresistant_gluconokinase | 99.4 | 91.5 | 9.74E−120 |
| | | faecalis | | | | |
| GCF_014898155.1 | 1765219 | Bifidobacterium | gntK_i01_CAKGJPAO_00191_Gluconokinase | 94.4 | 79.3 | 6.04E−99 |
| | | eulemuris | | | | |
| GCF_014898175.1 | 1603886 | Bifidobacterium | gntK_i01_CAKGJPAO_00191_Gluconokinase | 94.4 | 79.9 | 5.18E−100 |
| | | lemurum | | | | |
| GCF_014946725.1 | 2758443 | Mixta | gntK_f37_MKMCEHOJ_02531_Thermoresistant_gluconokinase | 97.2 | 73.4 | 1.31E−92 |
| | | mediterraneensis | | | | |
| GCF_015139575.1 | 1134687 | Klebsiella | gntK_Kp2H7_NOCLEBFK_02257_Thermoresistant_gluconokinase | 99.4 | 96.6 | 1.01E−123 |
| | | michiganensis | | | | |
| GCF_015163655.1 | 351672 | Xenorhabdus | gntK_Kp2H7_NOCLEBFK_02257_Thermoresistant_gluconokinase | 92.4 | 70.8 | 7.28E−85 |
| | | griffiniae | | | | |
| GCF_015167415.1 | 1646340 | Kosakonia | gntK_Kp2H7_NOCLEBFK_02257_Thermoresistant_gluconokinase | 98.9 | 93.1 | 1.55E−116 |
| | | pseudosacchari | | | | |
| GCF_015223195.1 | 2866724 | Chitinilyticum | gntK_f37_MKMCEHOJ_02531_Thermoresistant_gluconokinase | 95 | 70 | 2.26E−84 |
| | | piscinae | | | | |
| GCF_015644585.1 | 2787622 | Rahnella laticis | gntK_Kp2H7_NOCLEBFK_02257_Thermoresistant_gluconokinase | 89.5 | 62.1 | 3.74E−67 |
| GCF_015732185.1 | 349096 | Pectinatus haikarae | gntK_i33_ABMIDDIK_01308_Xylulose_kinase | 98.4 | 73.8 | 1.84E−285 |
| GCF_016026395.1 | 93378 | Edwardsiella | gntK_f37_MKMCEHOJ_02531_Thermoresistant_gluconokinase | 97.7 | 73.8 | 7.74E−89 |
| | | hoshinae | | | | |
| GCF_016026735.1 | 61652 | Serratia rubidaea | gntK_f37_MKMCEHOJ_02531_Thermoresistant_gluconokinase | 95.5 | 70.2 | 2.86E−84 |
| GCF_016306625.1 | 2763499 | Xenorhabdus | gntK_Kp2H7_NOCLEBFK_02257_Thermoresistant_gluconokinase | 93 | 71.5 | 3.79E−87 |
| | | lircayensis | | | | |
| GCF_016415625.1 | 570012 | Shimwellia | gntK_f37_MKMCEHOJ_02531_Thermoresistant_gluconokinase | 98.9 | 84.4 | 8.21E−109 |
| | | pseudoproteus | | | | |
| GCF_016415705.1 | 1463165 | Klebsiella | gntK_Kp2H7_NOCLEBFK_02257_Thermoresistant_gluconokinase | 100 | 98.9 | 7.74E−128 |
| | | quasipneumoniae | | | | |
| GCF_016632365.1 | 2799638 | Tenebrionibacter | gntK_Kp2H7_NOCLEBFK_02257_Thermoresistant_gluconokinase | 98.3 | 83 | 1.76E−103 |
| | | intestinalis | | | | |
| GCF_016756775.1 | 3051154 | Cedecea | gntK_f37_MKMCEHOJ_02531_Thermoresistant_gluconokinase | 98.3 | 90.2 | 3.79E−112 |
| | | sulfonylureivorans | | | | |
| GCF_016771845.1 | 2080838 | Lelliottia aquatilis | gntK_Kp2H7_NOCLEBFK_02257_Thermoresistant_gluconokinase | 98.9 | 93.1 | 2.03E−116 |
| GCF_016881405.1 | 570276 | Deefgea | gntK_f37_MKMCEHOJ_02531_Thermoresistant_gluconokinase | 94.7 | 70.4 | 2.24E−82 |
| | | chitinilytica | | | | |
| GCF_016893685.1 | 1748967 | Citrobacter cronae | gntK_Kp2H7_NOCLEBFK_02257_Thermoresistant_gluconokinase | 99.4 | 92 | 8.61E−116 |
| GCF_016904755.1 | 208962 | Escherichia albertii | gntK_f37_MKMCEHOJ_02531_Thermoresistant_gluconokinase | 98.9 | 98.9 | 1.06E−127 |
| GCF_016909495.1 | 1465635 | Pantoea coffeiphila | gntK_f37_MKMCEHOJ_02531_Thermoresistant_gluconokinase | 95.5 | 76.6 | 3.01E−96 |
| GCF_016937655.1 | 1071719 | Chromobacterium | gntK_f37_MKMCEHOJ_02531_Thermoresistant_gluconokinase | 94.3 | 62.4 | 1.23E−73 |
| | | alkanivorans | | | | |
| GCF_017309605.1 | 300181 | Serratia ureilytica | gntK_Kp2H7_NOCLEBFK_02257_Thermoresistant_gluconokinase | 93.9 | 73.2 | 1.49E−87 |
| GCF_017348915.1 | 2815358 | Leclercia | gntK_Kp2H7_NOCLEBFK_02257_Thermoresistant_gluconokinase | 98.9 | 93.6 | 1.49E−118 |
| | | pneumoniae | | | | |
| GCF_017743015.1 | 290110 | Xenorhabdus | gntK_Kp2H7_NOCLEBFK_02257_Thermoresistant_gluconokinase | 96.1 | 68.4 | 6.76E−86 |
| | | budapestensis | | | | |
| GCF_017875465.1 | 92490 | Erwinia toletana | gntK_f37_MKMCEHOJ_02531_Thermoresistant_gluconokinase | 96.6 | 73.3 | 7.55E−92 |
| GCF_018128425.1 | 67826 | Citrobacter sedlakii | gntK_f37_MKMCEHOJ_02531_Thermoresistant_gluconokinase | 98.9 | 94.8 | 1.05E−120 |
| GCF_018139045.1 | 2587529 | Klebsiella pasteurii | gntK_Kp2H7_NOCLEBFK_02257_Thermoresistant_gluconokinase | 99.4 | 96 | 5.87E−123 |

TABLE 32

| accession | transporter_sseqid | transporter_qcovhsp | transporter_pident | transporter_evalue | gut_microbes |
|---|---|---|---|---|---|
| GCF_014250475.1 | transporter_i33_ABMIDDIK_01306_High-affinity_gluconate_transporter | 100 | 77.7 | 1.57E−242 | 0 |
| GCF_014295015.1 | transporter_f37_MKMCEHOJ_02530_Low-affinity_gluconate_transporter | 100 | 78.2 | 5.18E−249 | 0 |
| GCF_014467235.1 | transporter_f37_MKMCEHOJ_02530_Low-affinity_gluconate_transporter | 100 | 76.9 | 9.93E−247 | 0 |
| GCF_014635685.1 | transporter_i02_JFDANPEK_00220_Gnt-II_system_L-idonate_transporter | 98.7 | 65.5 | 6.70E−190 | 0 |
| GCF_014644275.1 | transporter_f37_MKMCEHOJ_02530_Low-affinity_gluconate_transporter | 100 | 89.7 | 8.52E−283 | 0 |
| GCF_014652175.1 | transporter_f37_MKMCEHOJ_02530_Low-affinity_gluconate_transporter | 98.5 | 73.9 | 4.56E−240 | 0 |
| GCF_014653055.1 | transporter_f37_MKMCEHOJ_02530_Low-affinity_gluconate_transporter | 100 | 94.8 | 5.80E−297 | 0 |
| GCF_014898155.1 | transporter_i02_JFDANPEK_00220_Gnt-II_system_L-idonate_transporter | 99.3 | 82.3 | 8.18E−232 | 0 |
| GCF_014898175.1 | transporter_i01_CAKGJPAO_00190_High-affinity_gluconate_transporter | 99.8 | 81.2 | 2.51E−243 | 0 |
| GCF_014946725.1 | transporter_f37_MKMCEHOJ_02530_Low-affinity_gluconate_transporter | 100 | 79.6 | 9.30E−257 | 0 |
| GCF_015139575.1 | transporter_f37_MKMCEHOJ_02530_Low-affinity_gluconate_transporter | 100 | 96.6 | 9.84E−305 | 1 |
| GCF_015163655.1 | transporter_f37_MKMCEHOJ_02530_Low-affinity_gluconate_transporter | 100 | 75.7 | 7.74E−244 | 0 |
| GCF_015167415.1 | transporter_f37_MKMCEHOJ_02530_Low-affinity_gluconate_transporter | 100 | 92.8 | 1.36E−295 | 0 |
| GCF_015223195.1 | transporter_f37_MKMCEHOJ_02530_Low-affinity_gluconate_transporter | 99.3 | 78.3 | 1.21E−247 | 0 |
| GCF_015644585.1 | transporter_f37_MKMCEHOJ_02505_High-affinity_gluconate_transporter | 100 | 84.9 | 7.89E−261 | 0 |
| GCF_015732185.1 | transporter_i37_IIDCKEOG_02049_High-affinity_gluconate_transporter | 100 | 82 | 3.54E−248 | 0 |
| GCF_016026395.1 | transporter_f37_MKMCEHOJ_02530_Low-affinity_gluconate_transporter | 100 | 79.1 | 2.00E−253 | 0 |
| GCF_016026735.1 | transporter_f37_MKMCEHOJ_02530_Low-affinity_gluconate_transporter | 100 | 80.7 | 7.33E−256 | 0 |
| GCF_016306625.1 | transporter_f37_MKMCEHOJ_02530_Low-affinity_gluconate_transporter | 100 | 78.9 | 8.11E−253 | 0 |
| GCF_016415625.1 | transporter_f37_MKMCEHOJ_02530_Low-affinity_gluconate_transporter | 100 | 89.6 | 4.37E−282 | 0 |
| GCF_016415705.1 | transporter_f37_MKMCEHOJ_02530_Low-affinity_gluconate_transporter | 100 | 96.2 | 3.29E−303 | 1 |
| GCF_016632365.1 | transporter_f37_MKMCEHOJ_02530_Low-affinity_gluconate_transporter | 100 | 92.2 | 7.20E−291 | 0 |
| GCF_016756775.1 | transporter_Kp2H7_NOCLEBFK_02256_Low-affinity_gluconate_transporter | 97.5 | 95.5 | 3.45E−286 | 0 |
| GCF_016771845.1 | transporter_f37_MKMCEHOJ_02530_Low-affinity_gluconate_transporter | 100 | 95.9 | 1.31E−298 | 0 |
| GCF_016881405.1 | transporter_f37_MKMCEHOJ_02530_Low-affinity_gluconate_transporter | 100 | 77.3 | 8.99E−243 | 0 |
| GCF_016893685.1 | transporter_f37_MKMCEHOJ_02530_Low-affinity_gluconate_transporter | 100 | 96 | 1.56E−301 | 0 |
| GCF_016904755.1 | transporter_f37_MKMCEHOJ_02530_Low-affinity_gluconate_transporter | 100 | 98.9 | 3.22E−310 | 1 |
| GCF_016909495.1 | transporter_f37_MKMCEHOJ_02530_Low-affinity_gluconate_transporter | 100 | 82.5 | 9.24E−264 | 0 |
| GCF_016937655.1 | transporter_f37_MKMCEHOJ_02530_Low-affinity_gluconate_transporter | 99.1 | 74.6 | 5.70E−232 | 0 |
| GCF_017309605.1 | transporter_f37_MKMCEHOJ_02530_Low-affinity_gluconate_transporter | 99.8 | 71.4 | 2.96E−227 | 0 |
| GCF_017348915.1 | transporter_f37_MKMCEHOJ_02530_Low-affinity_gluconate_transporter | 100 | 95.5 | 1.82E−300 | 0 |
| GCF_017743015.1 | transporter_f37_MKMCEHOJ_02530_Low-affinity_gluconate_transporter | 100 | 76.6 | 4.24E−248 | 0 |
| GCF_017875465.1 | transporter_f37_MKMCEHOJ_02530_Low-affinity_gluconate_transporter | 100 | 83.2 | 8.33E−267 | 0 |
| GCF_018128425.1 | transporter_f37_MKMCEHOJ_02530_Low-affinity_gluconate_transporter | 100 | 97.1 | 4.18E−306 | 1 |
| GCF_018139045.1 | transporter_f37_MKMCEHOJ_02530_Low-affinity_gluconate_transporter | 100 | 96.4 | 1.63E−303 | 0 |

TABLE 33

| | organism. | | | gntK_ | gntK_ | gntK_ |
|---|---|---|---|---|---|---|
| accession | taxId | species | gntK_sseqid | qcovhsp | pident | evalue |
| GCF_018288855.1 | 58169 | Rahnella inusitata | gntK_Kp2H7_NOCLEBFK_02257_Thermoresistant_gluconokinase | 89.5 | 61.4 | 1.07E−66 |
| GCF_018448885.1 | 1004165 | Photorhabdus | gntK_Kp2H7_NOCLEBFK_02257_Thermoresistant_gluconokinase | 96.1 | 70.3 | 3.32E−84 |
| | | caribbeanensis | | | | |
| GCF_018448965.1 | 1004166 | Photorhabdus | gntK_Kp2H7_NOCLEBFK_02257_Thermoresistant_gluconokinase | 96.1 | 70.9 | 6.70E−84 |
| | | hainanensis | | | | |
| GCF_018494035.1 | 1544696 | Rosenbergiella | gntK_Kp2H7_NOCLEBFK_02257_Thermoresistant_gluconokinase | 97.2 | 73.7 | 4.36E−93 |
| | | australiborealis | | | | |
| GCF_018494065.1 | 2726987 | Rosenbergiella | gntK_Kp2H7_NOCLEBFK_02257_Thermoresistant_gluconokinase | 97.2 | 72.5 | 4.16E−91 |
| | | gaditana | | | | |
| GCF_018597345.1 | 2838947 | Enterobacter | gntK_Kp2H7_NOCLEBFK_02257_Thermoresistant_gluconokinase | 98.9 | 93.6 | 2.47E−117 |
| | | quasimori | | | | |
| GCF_019047765.1 | 1563222 | Citrobacter | gntK_Kp2H7_NOCLEBFK_02257_Thermoresistant_gluconokinase | 99.4 | 92 | 1.49E−116 |
| | | pasteurii | | | | |
| GCF_019048385.1 | 549 | Pantoea | gntK_Kp2H7_NOCLEBFK_02257_Thermoresistant_gluconokinase | 96.1 | 74.3 | 9.52E−93 |
| | | agglomerans | | | | |
| GCF_019048625.1 | 158836 | Enterobacter | gntK_Kp2H7_NOCLEBFK_02257_Thermoresistant_gluconokinase | 98.9 | 94.2 | 3.00E−118 |
| | | hormaechei | | | | |
| GCF_019049655.1 | 2816249 | Rahnella rivi | gntK_Kp2H7_NOCLEBFK_02257_Thermoresistant_gluconokinase | 89.5 | 61.4 | 1.07E−66 |
| GCF_019049675.1 | 2816248 | Rahnella | gntK_Kp2H7_NOCLEBFK_02257_Thermoresistant_gluconokinase | 93.6 | 60 | 2.64E−67 |
| | | bonaserana | | | | |
| GCF_019049695.1 | 2816244 | Rahnella | gntK_f37_MKMCEHOJ_02531_Thermoresistant_gluconokinase | 94.2 | 60.2 | 4.44E−68 |
| | | perminowiae | | | | |
| GCF_019090985.1 | 171438 | Photorhabdus | gntK_Kp2H7_NOCLEBFK_02257_Thermoresistant_gluconokinase | 96.1 | 70.3 | 2.23E−82 |
| | | akhurstii | | | | |
| GCF_019132875.1 | 2853256 | Erwinia | gntK_Kp2H7_NOCLEBFK_02257_Thermoresistant_gluconokinase | 92.7 | 78 | 1.76E−90 |
| | | phyllosphaerae | | | | |
| GCF_019140855.1 | 2815733 | Tenebrionicola | gntK_Kp2H7_NOCLEBFK_02257_Thermoresistant_gluconokinase | 98.3 | 83 | 1.76E−103 |
| | | larvae | | | | |
| GCF_019331805.1 | 2834432 | Bifidobacterium | gntK_i01_CAKGJPAO_00191_Gluconokinase | 93.9 | 79.8 | 9.54E−96 |
| | | pongonis | | | | |
| GCF_019355955.1 | 61646 | Lelliottia amnigena | gntK_Kp2H7_NOCLEBFK_02257_Thermoresistant_gluconokinase | 98.9 | 93.6 | 2.47E−117 |
| GCF_019665745.1 | 69218 | Enterobacter | gntK_f37_MKMCEHOJ_02531_Thermoresistant_gluconokinase | 98.9 | 94.8 | 2.49E−119 |
| | | cancerogenus | | | | |
| GCF_019665765.1 | 2865840 | Deefgea tanakiae | gntK_f37_MKMCEHOJ_02531_Thermoresistant_gluconokinase | 90.6 | 72.9 | 1.11E−82 |
| GCF_019844095.1 | 55211 | Erwinia persicina | gntK_Kp2H7_NOCLEBFK_02257_Thermoresistant_gluconokinase | 96.1 | 77.3 | 7.65E−97 |
| GCF_019890955.1 | 59814 | Pantoea dispersa | gntK_Kp2H7_NOCLEBFK_02257_Thermoresistant_gluconokinase | 100 | 77.5 | 8.96E−98 |
| GCF_020042625.1 | 881260 | Enterobacter | gntK_Kp2H7_NOCLEBFK_02257_Thermoresistant_gluconokinase | 98.9 | 93.6 | 2.47E−117 |
| | | bugandensis | | | | |
| GCF_020079945.1 | 472693 | Pantoea eucrina | gntK_Kp2H7_NOCLEBFK_02257_Thermoresistant_gluconokinase | 99.4 | 73.6 | 1.21E−93 |
| GCF_020097475.1 | 564 | Escherichia | gntK_f37_MKMCEHOJ_02531_Thermoresistant_gluconokinase | 100 | 99.4 | 1.45E−127 |
| | | fergusonii | | | | |
| GCF_020150375.1 | 546367 | Hafnia paralvei | gntK_f37_MKMCEHOJ_02531_Thermoresistant_gluconokinase | 96.1 | 72.1 | 8.71E−89 |
| GCF_020268605.1 | 2879119 | Arsenophonus | gntK_f37_MKMCEHOJ_02531_Thermoresistant_gluconokinase | 95 | 67.3 | 5.24E−81 |
| | | apicola | | | | |
| GCF_020342335.1 | 1619313 | Duffyella | gntK_f37_MKMCEHOJ_02531_Thermoresistant_gluconokinase | 95.6 | 74.4 | 2.10E−94 |
| | | gerundensis | | | | |
| GCF_020381325.1 | 333965 | Providencia | gntK_Kp2H7_NOCLEBFK_02257_Thermoresistant_gluconokinase | 96.1 | 69.9 | 1.15E−82 |
| | | vermicola | | | | |
| GCF_020387775.1 | 2862867 | Photorhabdus | gntK_Kp2H7_NOCLEBFK_02257_Thermoresistant_gluconokinase | 96.1 | 71.5 | 4.96E−86 |
| | | antumapuensis | | | | |
| GCF_020525665.1 | 2026240 | Klebsiella | gntK_Kp2H7_NOCLEBFK_02257_Thermoresistant_gluconokinase | 100 | 99.4 | 2.70E−128 |
| | | quasivariicola | | | | |
| GCF_020526085.1 | 2489010 | Klebsiella africana | gntK_Kp2H7_NOCLEBFK_02257_Thermoresistant_gluconokinase | 100 | 99.4 | 2.70E−128 |
| GCF_020531905.1 | 2875502 | Deefgea salmonis | gntK_Kp2H7_NOCLEBFK_02257_Thermoresistant_gluconokinase | 94.7 | 71 | 6.95E−84 |
| GCF_020541885.1 | 28026 | Bifidobacterium | gntK_i02_JFDANPEK_00221_Gluconokinase | 95 | 96.5 | 1.92E−119 |
| | | pseudocatenulatum | | | | |
| GCF_020544465.1 | 1778675 | Chromobacterium | gntK_f37_MKMCEHOJ_02531_Thermoresistant_gluconokinase | 94.3 | 63.6 | 3.03E−74 |
| | | rhizoryzae | | | | |
| GCF_020683125.1 | 55212 | Erwinia rhapontici | gntK_Kp2H7_NOCLEBFK_02257_Thermoresistant_gluconokinase | 95.5 | 77.8 | 5.39E−97 |

TABLE 34

| accession | transporter_sseqid | transporter_qcovhsp | transporter_pident | transporter_evalue | gut_microbes |
|---|---|---|---|---|---|
| GCF_018288855.1 | transporter_f37_MKMCEHOJ_02505_High-affinity_gluconate_transporter | 100 | 84.7 | 1.30E−259 | 0 |
| GCF_018448885.1 | transporter_f37_MKMCEHOJ_02530_Low-affinity_gluconate_transporter | 100 | 77.5 | 7.35E−249 | 0 |
| GCF_018448965.1 | transporter_f37_MKMCEHOJ_02530_Low-affinity_gluconate_transporter | 100 | 77.5 | 2.57E−249 | 0 |
| GCF_018494035.1 | transporter_f37_MKMCEHOJ_02530_Low-affinity_gluconate_transporter | 100 | 78.5 | 8.03E−251 | 0 |
| GCF_018494065.1 | transporter_f37_MKMCEHOJ_02530_Low-affinity_gluconate_transporter | 100 | 79.1 | 5.94E−253 | 0 |
| GCF_018597345.1 | transporter_f37_MKMCEHOJ_02530_Low-affinity_gluconate_transporter | 100 | 95.7 | 1.28E−300 | 0 |
| GCF_019047765.1 | transporter_f37_MKMCEHOJ_02530_Low-affinity_gluconate_transporter | 100 | 96.9 | 8.07E−304 | 0 |
| GCF_019048385.1 | transporter_f37_MKMCEHOJ_02530_Low-affinity_gluconate_transporter | 100 | 80.9 | 2.40E−259 | 1 |
| GCF_019048625.1 | transporter_f37_MKMCEHOJ_02530_Low-affinity_gluconate_transporter | 100 | 96 | 3.14E−301 | 0 |
| GCF_019049655.1 | transporter_f37_MKMCEHOJ_02505_High-affinity_gluconate_transporter | 100 | 85.4 | 1.94E−261 | 0 |
| GCF_019049675.1 | transporter_f37_MKMCEHOJ_02505_High-affinity_gluconate_transporter | 100 | 85.4 | 8.28E−263 | 0 |
| GCF_019049695.1 | transporter_f37_MKMCEHOJ_02505_High-affinity_gluconate_transporter | 100 | 84.9 | 1.94E−261 | 0 |
| GCF_019090985.1 | transporter_f37_MKMCEHOJ_02530_Low-affinity_gluconate_transporter | 100 | 77.8 | 2.21E−250 | 0 |
| GCF_019132875.1 | transporter_f37_MKMCEHOJ_02530_Low-affinity_gluconate_transporter | 100 | 82.7 | 2.52E−261 | 0 |
| GCF_019140855.1 | transporter_f37_MKMCEHOJ_02530_Low-affinity_gluconate_transporter | 100 | 92.2 | 7.20E−291 | 0 |
| GCF_019331805.1 | transporter_i02_JFDANPEK_00220_Gnt-II_system_L-idonate_transporter | 100 | 80.1 | 1.54E−239 | 0 |
| GCF_019355955.1 | transporter_f37_MKMCEHOJ_02530_Low-affinity_gluconate_transporter | 100 | 95.1 | 1.22E−298 | 0 |
| GCF_019665745.1 | transporter_f37_MKMCEHOJ_02530_Low-affinity_gluconate_transporter | 100 | 95.7 | 3.83E−302 | 1 |
| GCF_019665765.1 | transporter_f37_MKMCEHOJ_02530_Low-affinity_gluconate_transporter | 100 | 76.6 | 4.46E−243 | 0 |
| GCF_019844095.1 | transporter_f37_MKMCEHOJ_02530_Low-affinity_gluconate_transporter | 100 | 83.4 | 1.95E−265 | 0 |
| GCF_019890955.1 | transporter_f37_MKMCEHOJ_02530_Low-affinity_gluconate_transporter | 100 | 79.8 | 6.24E−255 | 0 |
| GCF_020042625.1 | transporter_f37_MKMCEHOJ_02530_Low-affinity_gluconate_transporter | 100 | 96 | 3.14E−301 | 0 |
| GCF_020079945.1 | transporter_f37_MKMCEHOJ_02530_Low-affinity_gluconate_transporter | 100 | 79.8 | 3.10E−255 | 0 |
| GCF_020097475.1 | transporter_f37_MKMCEHOJ_02530_Low-affinity_gluconate_transporter | 100 | 99.1 | 7.9E−311 | 1 |
| GCF_020150375.1 | transporter_f37_MKMCEHOJ_02530_Low-affinity_gluconate_transporter | 99.3 | 79.2 | 1.34E−251 | 0 |
| GCF_020268605.1 | transporter_f37_MKMCEHOJ_02530_Low-affinity_gluconate_transporter | 96.9 | 69.5 | 5.34E−218 | 0 |
| GCF_020342335.1 | transporter_f37_MKMCEHOJ_02530_Low-affinity_gluconate_transporter | 100 | 81.2 | 7.22E−261 | 0 |
| GCF_020381325.1 | transporter_f37_MKMCEHOJ_02530_Low-affinity_gluconate_transporter | 98.4 | 73.9 | 5.61E−236 | 0 |
| GCF_020387775.1 | transporter_f37_MKMCEHOJ_02530_Low-affinity_gluconate_transporter | 100 | 77.8 | 4.46E−250 | 0 |
| GCF_020525665.1 | transporter_f37_MKMCEHOJ_02530_Low-affinity_gluconate_transporter | 100 | 97.1 | 2.82E−304 | 0 |
| GCF_020526085.1 | transporter_f37_MKMCEHOJ_02530_Low-affinity_gluconate_transporter | 100 | 96.6 | 2.31E−303 | 0 |
| GCF_020531905.1 | transporter_f37_MKMCEHOJ_02530_Low-affinity_gluconate_transporter | 100 | 76.1 | 3.95E−242 | 0 |
| GCF_020541885.1 | transporter_i02_JFDANPEK_00220_Gnt-II_system_L-idonate_transporter | 100 | 95.7 | 1.38E−286 | 1 |
| GCF_020544465.1 | transporter_f37_MKMCEHOJ_02530_Low-affinity_gluconate_transporter | 99.1 | 74.6 | 9.39E−231 | 0 |
| GCF_020683125.1 | transporter_f37_MKMCEHOJ_02530_Low-affinity_gluconate_transporter | 100 | 83.2 | 3.39E−266 | 0 |

TABLE 35

| | organism. | | | gntK_ | gntK_ | gntK_ |
|---|---|---|---|---|---|---|
| accession | taxId | species | gntK_sseqid | qcovhsp | pident | evalue |
| GCF_021083665.1 | 69220 | Lelliottia | gntK_Kp2H7_NOCLEBFK_02257_Thermoresistant_gluconokinase | 98.9 | 94.2 | 1.22E−117 |
| | | nimipressuralis | | | | |
| GCF_021129195.1 | 467180 | Chromobacterium | gntK_f37_MKMCEHOJ_02531_Thermoresistant_gluconokinase | 94.3 | 63.6 | 2.25E−76 |
| | | aquaticum | | | | |
| GCF_021172065.1 | 2891570 | Pseudocitrobacter | gntK_Kp2H7_NOCLEBFK_02257_Thermoresistant_gluconokinase | 99.4 | 92.6 | 1.18E−120 |
| | | corydidari | | | | |
| GCF_021276285.1 | 1510570 | Rahnella victoriana | gntK_Kp2H7_NOCLEBFK_02257_Thermoresistant_gluconokinase | 89.5 | 62.1 | 2.64E−67 |
| GCF_021278985.1 | 1218085 | Citrobacter | gntK_f37_MKMCEHOJ_02531_Thermoresistant_gluconokinase | 98.9 | 95.4 | 6.36E−122 |
| | | rodentium | | | | |
| GCF_021462285.1 | 42906 | Serratia entomophila | gntK_f37_MKMCEHOJ_02531_Thermoresistant_gluconokinase | 94.4 | 74 | 1.06E−89 |
| GCF_021733145.1 | 1263550 | Edwardsiella | gntK_f37_MKMCEHOJ_02531_Thermoresistant_gluconokinase | 97.7 | 72.1 | 1.82E−87 |
| | | piscicida | | | | |
| GCF_022014715.1 | 539813 | Enterobacter mori | gntK_Kp2H7_NOCLEBFK_02257_Thermoresistant_gluconokinase | 98.9 | 93.6 | 2.47E−117 |
| GCF_022171985.1 | 2899785 | Huaxiibacter | gntK_Kp2H7_NOCLEBFK_02257_Thermoresistant_gluconokinase | 98.9 | 91.9 | 5.82E−116 |
| | | chinensis | | | | |
| GCF_022354085.1 | 622 | Shigella dysenteriae | gntK_f37_MKMCEHOJ_02531_Thermoresistant_gluconokinase | 100 | 99.4 | 1.45E−127 |
| GCF_022369495.1 | 102862 | Proteus penneri | gntK_Kp2H7_NOCLEBFK_02257_Thermoresistant_gluconokinase | 93.5 | 73.3 | 2.99E−86 |
| GCF_022592395.1 | 158849 | Moellerella | gntK_f37_MKMCEHOJ_02531_Thermoresistant_gluconokinase | 96.6 | 71.5 | 3.25E−86 |
| | | wisconsensis | | | | |
| GCF_022602085.1 | 1544695 | Rosenbergiella | gntK_Kp2H7_NOCLEBFK_02257_Thermoresistant_gluconokinase | 97.2 | 72.5 | 2.93E−91 |
| | | collisarenosi | | | | |
| GCF_022602565.1 | 2921185 | Rosenbergiella | gntK_Kp2H7_NOCLEBFK_02257_Thermoresistant_gluconokinase | 97.2 | 71.9 | 5.91E−91 |
| | | metrosideri | | | | |
| GCF_022637475.1 | 2703885 | Rahnella aceris | gntK_Kp2H7_NOCLEBFK_02257_Thermoresistant_gluconokinase | 89.5 | 61.4 | 1.07E−66 |
| GCF_022637515.1 | 82989 | Buttiauxella | gntK_f37_MKMCEHOJ_02531_Thermoresistant_gluconokinase | 98.3 | 92 | 1.23E−115 |
| | | ferragutiae | | | | |
| GCF_022637595.1 | 575 | Raoultella planticola | gntK_Kp2H7_NOCLEBFK_02257_Thermoresistant_gluconokinase | 100 | 96.6 | 1.23E−124 |
| GCF_022647325.1 | 470932 | Pantoea deleyi | gntK_Kp2H7_NOCLEBFK_02257_Thermoresistant_gluconokinase | 96.6 | 74.4 | 1.16E−93 |
| GCF_022647505.1 | 1324864 | [Pantoea] | gntK_Kp2H7_NOCLEBFK_02257_Thermoresistant_gluconokinase | 95.5 | 77.2 | 3.46E−93 |
| | | beijingensis | | | | |
| GCF_023108895.1 | 1208607 | Photorhabdus | gntK_Kp2H7_NOCLEBFK_02257_Thermoresistant_gluconokinase | 96.1 | 72.1 | 1.22E−86 |
| | | noenieputensis | | | | |
| GCF_023195735.1 | 51288 | Kluyvera ascorbata | gntK_Kp2H7_NOCLEBFK_02257_Thermoresistant_gluconokinase | 98.3 | 93.1 | 4.83E−120 |
| GCF_023282985.1 | 2824122 | Serratia silvae | gntK_f37_MKMCEHOJ_02531_Thermoresistant_gluconokinase | 96.4 | 60.5 | 3.11E−72 |
| GCF_023517775.1 | 2872648 | Mixta hanseatica | gntK_f37_MKMCEHOJ_02531_Thermoresistant_gluconokinase | 96.6 | 74.4 | 2.26E−93 |
| GCF_023613945.1 | 1544694 | Rosenbergiella | gntK_Kp2H7_NOCLEBFK_02257_Thermoresistant_gluconokinase | 97.2 | 72.5 | 1.45E−91 |
| | | epipactidis | | | | |
| GCF_024023655.1 | 2894201 | Citrobacter | gntK_Kp2H7_NOCLEBFK_02257_Thermoresistant_gluconokinase | 99.4 | 92.6 | 8.97E−118 |
| | | meridianamericanus | | | | |
| GCF_024169515.1 | 68334 | Erwinia aphidicola | gntK_Kp2H7_NOCLEBFK_02257_Thermoresistant_gluconokinase | 96.1 | 77.9 | 4.63E−98 |
| GCF_024224615.1 | 255519 | Atlantibacter | gntK_f37_MKMCEHOJ_02531_Thermoresistant_gluconokinase | 98.9 | 85.1 | 4.41E−109 |
| | | subterranea | | | | |
| GCF_024764775.1 | 2926520 | Scandinavium | gntK_Kp2H7_NOCLEBFK_02257_Thermoresistant_gluconokinase | 99.4 | 93.1 | 1.62E−120 |
| | | manionii | | | | |
| GCF_024764795.1 | 2926519 | Scandinavium | gntK_Kp2H7_NOCLEBFK_02257_Thermoresistant_gluconokinase | 98.9 | 92 | 1.97E−119 |
| | | hiltneri | | | | |
| GCF_024764965.1 | 2926521 | Scandinavium | gntK_Kp2H7_NOCLEBFK_02257_Thermoresistant_gluconokinase | 99.4 | 93.1 | 2.30E−120 |
| | | tedordense | | | | |
| GCF_025215115.1 | 2925397 | Dryocola | gntK_f37_MKMCEHOJ_02531_Thermoresistant_gluconokinase | 98.3 | 90.2 | 2.90E−114 |
| | | boscaweniae | | | | |
| GCF_025215155.1 | 2925396 | Dryocola | gntK_f37_MKMCEHOJ_02531_Thermoresistant_gluconokinase | 98.3 | 90.2 | 3.79E−112 |
| | | clanedunensis | | | | |
| GCF_025384885.1 | 768034 | Photorhabdus kleinii | gntK_Kp2H7_NOCLEBFK_02257_Thermoresistant_gluconokinase | 96.1 | 70.9 | 2.86E−85 |
| GCF_025384895.1 | 230088 | Photorhabdus kayaii | gntK_Kp2H7_NOCLEBFK_02257_Thermoresistant_gluconokinase | 96.1 | 72.1 | 1.22E−86 |
| GCF_025527015.1 | 2978220 | Winslowiella | gntK_f37_MKMCEHOJ_02531_Thermoresistant_gluconokinase | 96.6 | 74.4 | 1.86E−92 |
| | | arboricola | | | | |

TABLE 36

| accession | transporter_sseqid | transporter_qcovhsp | transporter_pident | transporter_evalue | gut_microbes |
|---|---|---|---|---|---|
| GCF_021083665.1 | transporter_f37_MKMCEHOJ_02530_Low-affinity_gluconate_transporter | 100 | 95.5 | 2.58E−300 | 0 |
| GCF_021129195.1 | transporter_f37_MKMCEHOJ_02530_Low-affinity_gluconate_transporter | 100 | 74.2 | 7.68E−230 | 0 |
| GCF_021172065.1 | transporter_f37_MKMCEHOJ_02530_Low-affinity_gluconate_transporter | 100 | 94.8 | 5.80E−297 | 0 |
| GCF_021276285.1 | transporter_f37_MKMCEHOJ_02505_High-affinity_gluconate_transporter | 100 | 85.2 | 4.78E−262 | 0 |
| GCF_021278985.1 | transporter_f37_MKMCEHOJ_02530_Low-affinity_gluconate_transporter | 100 | 96.4 | 4.88E−305 | 0 |
| GCF_021462285.1 | transporter_f37_MKMCEHOJ_02530_Low-affinity_gluconate_transporter | 99.3 | 72.4 | 5.41E−230 | 0 |
| GCF_021733145.1 | transporter_f37_MKMCEHOJ_02530_Low-affinity_gluconate_transporter | 100 | 78.7 | 1.34E−251 | 0 |
| GCF_022014715.1 | transporter_f37_MKMCEHOJ_02530_Low-affinity_gluconate_transporter | 100 | 95.7 | 1.28E−300 | 1 |
| GCF_022171985.1 | transporter_f37_MKMCEHOJ_02530_Low-affinity_gluconate_transporter | 100 | 95.7 | 1.10E−301 | 0 |
| GCF_022354085.1 | transporter_f37_MKMCEHOJ_02530_Low-affinity_gluconate_transporter | 100 | 99.8 | 1.67E−312 | 0 |
| GCF_022369495.1 | transporter_f37_MKMCEHOJ_02530_Low-affinity_gluconate_transporter | 100 | 77.1 | 1.81E−249 | 1 |
| GCF_022592395.1 | transporter_f37_MKMCEHOJ_02530_Low-affinity_gluconate_transporter | 100 | 74.8 | 3.14E−243 | 1 |
| GCF_022602085.1 | transporter_f37_MKMCEHOJ_02530_Low-affinity_gluconate_transporter | 100 | 79.1 | 1.79E−254 | 0 |
| GCF_022602565.1 | transporter_f37_MKMCEHOJ_02530_Low-affinity_gluconate_transporter | 100 | 78.9 | 5.11E−254 | 0 |
| GCF_022637475.1 | transporter_f37_MKMCEHOJ_02505_High-affinity_gluconate_transporter | 100 | 84.5 | 7.52E−259 | 0 |
| GCF_022637515.1 | transporter_f37_MKMCEHOJ_02530_Low-affinity_gluconate_transporter | 100 | 92.8 | 1.30E−293 | 0 |
| GCF_022637595.1 | transporter_f37_MKMCEHOJ_02530_Low-affinity_gluconate_transporter | 100 | 96 | 1.22E−298 | 0 |
| GCF_022647325.1 | transporter_f37_MKMCEHOJ_02530_Low-affinity_gluconate_transporter | 100 | 80.9 | 7.22E−261 | 0 |
| GCF_022647505.1 | transporter_f37_MKMCEHOJ_02530_Low-affinity_gluconate_transporter | 100 | 81.8 | 1.02E−260 | 0 |
| GCF_023108895.1 | transporter_f37_MKMCEHOJ_02530_Low-affinity_gluconate_transporter | 99.3 | 77.4 | 1.48E−248 | 0 |
| GCF_023195735.1 | transporter_f37_MKMCEHOJ_02530_Low-affinity_gluconate_transporter | 100 | 93 | 5.30E−293 | 1 |
| GCF_023282985.1 | transporter_f37_MKMCEHOJ_02505_High-affinity_gluconate_transporter | 100 | 86.5 | 7.45E−266 | 0 |
| GCF_023517775.1 | transporter_f37_MKMCEHOJ_02530_Low-affinity_gluconate_transporter | 100 | 82.3 | 8.33E−267 | 0 |
| GCF_023613945.1 | transporter_f37_MKMCEHOJ_02530_Low-affinity_gluconate_transporter | 100 | 78.9 | 2.95E−253 | 0 |
| GCF_024023655.1 | transporter_f37_MKMCEHOJ_02530_Low-affinity_gluconate_transporter | 99.8 | 96.7 | 1.19E−294 | 0 |
| GCF_024169515.1 | transporter_f37_MKMCEHOJ_02530_Low-affinity_gluconate_transporter | 100 | 82.3 | 7.94E−265 | 0 |
| GCF_024224615.1 | transporter_f37_MKMCEHOJ_02530_Low-affinity_gluconate_transporter | 100 | 91.5 | 2.40E−289 | 0 |
| GCF_024764775.1 | transporter_f37_MKMCEHOJ_02530_Low-affinity_gluconate_transporter | 100 | 95.5 | 6.06E−299 | 0 |
| GCF_024764795.1 | transporter_f37_MKMCEHOJ_02530_Low-affinity_gluconate_transporter | 100 | 95.7 | 1.82E−300 | 0 |
| GCF_024764965.1 | transporter_f37_MKMCEHOJ_02530_Low-affinity_gluconate_transporter | 100 | 95.7 | 2.58E−300 | 0 |
| GCF_025215115.1 | transporter_f37_MKMCEHOJ_02530_Low-affinity_gluconate_transporter | 100 | 94.4 | 3.90E−295 | 0 |
| GCF_025215155.1 | transporter_f37_MKMCEHOJ_02530_Low-affinity_gluconate_transporter | 100 | 93.5 | 1.59E−294 | 0 |
| GCF_025384885.1 | transporter_f37_MKMCEHOJ_02530_Low-affinity_gluconate_transporter | 100 | 77.5 | 3.14E−250 | 0 |
| GCF_025384895.1 | transporter_f37_MKMCEHOJ_02530_Low-affinity_gluconate_transporter | 99.3 | 77.6 | 1.81E−249 | 0 |
| GCF_025527015.1 | transporter_f37_MKMCEHOJ_02530_Low-affinity_gluconate_transporter | 100 | 83.4 | 3.55E−268 | 0 |

TABLE 37

| | organism. | | | gntK_ | gntK_ | gntK_ |
|---|---|---|---|---|---|---|
| accession | taxId | species | gntK_sseqid | qcovhsp | pident | evalue |
| GCF_025564065.1 | 2926469 | Silvania | gntK_Kp2H7_NOCLEBFK_02257_Thermoresistant_gluconokinase | 98.9 | 93.1 | 6.06E−118 |
| | | hatchlandensis | | | | |
| GCF_025564085.1 | 2926470 | Silvania confinis | gntK_Kp2H7_NOCLEBFK_02257_Thermoresistant_gluconokinase | 98.9 | 92.5 | 1.22E−117 |
| GCF_025566025.1 | 2926467 | Leclercia tamurae | gntK_Kp2H7_NOCLEBFK_02257_Thermoresistant_gluconokinase | 98.9 | 94.2 | 1.49E−118 |
| GCF_025567065.1 | 2981791 | Megasphaera | gntK_i33_ABMIDDIK_01308_Xylulose_kinase | 99.8 | 88.5 | 0 |
| | | butyrica | | | | |
| GCF_025758125.1 | 208223 | Kosakonia cowanii | gntK_f37_MKMCEHOJ_02531_Thermoresistant_gluconokinase | 98.9 | 90.9 | 2.48E−115 |
| GCF_025914095.1 | 1505757 | Siccibacter colletis | gntK_f37_MKMCEHOJ_02531_Thermoresistant_gluconokinase | 100 | 82.7 | 1.40E−104 |
| GCF_026153155.1 | 2991723 | Photorhabdus | gntK_f37_MKMCEHOJ_02531_Thermoresistant_gluconokinase | 95.5 | 70.8 | 6.80E−86 |
| | | aballayi | | | | |
| GCF_026154845.1 | 2703793 | Serratia | gntK_Kp2H7_NOCLEBFK_02257_Thermoresistant_gluconokinase | 93.9 | 73.2 | 1.49E−87 |
| | | bockelmannii | | | | |
| GCF_026241935.1 | 580 | Kluyvera | gntK_f37_MKMCEHOJ_02531_Thermoresistant_gluconokinase | 98.3 | 91.4 | 4.47E−118 |
| | | cryocrescens | | | | |
| GCF_026344075.1 | 2994648 | Enterobacter | gntK_Kp2H7_NOCLEBFK_02257_Thermoresistant_gluconokinase | 98.9 | 93.1 | 4.10E−116 |
| | | nematophilus | | | | |
| GCF_027886705.1 | 1005665 | Kosakonia | gntK_Kp2H7_NOCLEBFK_02257_Thermoresistant_gluconokinase | 98.9 | 93.7 | 1.89E−117 |
| | | oryzendophytica | | | | |
| GCF_029277985.1 | 588 | Providencia stuartii | gntK_f37_MKMCEHOJ_02531_Thermoresistant_gluconokinase | 95 | 64.3 | 2.35E−77 |
| GCF_029876145.1 | 2608867 | Escherichia ruysiae | gntK_f37_MKMCEHOJ_02531_Thermoresistant_gluconokinase | 100 | 98.9 | 1.69E−126 |
| GCF_030026565.1 | 413502 | Cronobacter | gntK_Kp2H7_NOCLEBFK_02257_Thermoresistant_gluconokinase | 99.4 | 82.2 | 1.57E−104 |
| | | turicensis | | | | |
| GCF_030053895.1 | 379893 | Trabulsiella | gntK_Kp2H7_NOCLEBFK_02257_Thermoresistant_gluconokinase | 99.4 | 93.1 | 3.13E−118 |
| | | odontotermitis | | | | |
| GCF_030286765.1 | 2050967 | Proteus faecis | gntK_Kp2H7_NOCLEBFK_02257_Thermoresistant_gluconokinase | 93.5 | 73.8 | 2.57E−87 |
| GCF_030294585.1 | 133448 | Citrobacter youngae | gntK_Kp2H7_NOCLEBFK_02257_Thermoresistant_gluconokinase | 99.4 | 93.1 | 3.13E−118 |
| GCF_030406165.1 | 2996112 | Enterobacter | gntK_Kp2H7_NOCLEBFK_02257_Thermoresistant_gluconokinase | 98.9 | 93.1 | 4.10E−116 |
| | | pseudoroggenkampii | | | | |
| GCF_900021175.1 | 1755099 | Pseudenterobacter | gntK_f37_MKMCEHOJ_02531_Thermoresistant_gluconokinase | 98.9 | 93.6 | 5.02E−119 |
| | | timonensis | | | | |
| GCF_900094795.1 | 1005667 | Kosakonia | gntK_f37_MKMCEHOJ_02531_Thermoresistant_gluconokinase | 98.3 | 93.7 | 2.31E−120 |
| | | oryziphila | | | | |
| GCF_900095855.1 | 1852379 | Colibacter | gntK_i33_ABMIDDIK_01308_Xylulose_kinase | 99.6 | 82.8 | 0 |
| | | massiliensis | | | | |
| GCF_900103535.1 | 349095 | Megasphaera | gntK_i33_ABMIDDIK_01308_Xylulose_kinase | 99.4 | 76.2 | 1.34E−299 |
| | | paucivorans | | | | |
| GCF_900109225.1 | 84035 | Propionispira | gntK_i33_ABMIDDIK_01308_Xylulose_kinase | 98.3 | 67.2 | 1.56E−265 |
| | | arboris | | | | |
| GCF_900111105.1 | 988801 | Rosenbergiella | gntK_Kp2H7_NOCLEBFK_02257_Thermoresistant_gluconokinase | 97.2 | 71.3 | 1.97E−89 |
| | | nectarea | | | | |
| GCF_900113945.1 | 351675 | Xenorhabdus | gntK_Kp2H7_NOCLEBFK_02257_Thermoresistant_gluconokinase | 91.9 | 73.1 | 3.95E−87 |
| | | mauleonii | | | | |
| GCF_900115195.1 | 53341 | Xenorhabdus | gntK_Kp2H7_NOCLEBFK_02257_Thermoresistant_gluconokinase | 86.8 | 71.9 | 9.96E−86 |
| | | japonica | | | | |
| GCF_900155355.1 | 290109 | Xenorhabdus innexi | gntK_f37_MKMCEHOJ_02531_Thermoresistant_gluconokinase | 97.2 | 68.8 | 1.61E−86 |
| GCF_900167325.1 | 1122219 | Megasphaera | gntK_i33_ABMIDDIK_01308_Xylulose_kinase | 99.6 | 84.2 | 0 |
| | | cerevisiae | | | | |
| GCF_900178525.1 | 1673718 | Massilibacteroides | gntK_f37_MKMCEHOJ_02531_Thermoresistant_gluconokinase | 92.2 | 61.4 | 6.03E−73 |
| | | vaginae | | | | |
| GCF_900186025.1 | 82995 | Serratia grimesii | gntK_Kp2H7_NOCLEBFK_02257_Thermoresistant_gluconokinase | 97.1 | 69.4 | 6.66E−83 |
| GCF_900187015.1 | 61651 | Serratia ficaria | gntK_f37_MKMCEHOJ_02531_Thermoresistant_gluconokinase | 94.4 | 73.4 | 4.32E−89 |
| GCF_900187035.1 | 158847 | Megamonas | gntK_i33_ABMIDDIK_01308_Xylulose_kinase | 99.4 | 66.6 | 2.92E−260 |
| | | hypermegale | | | | |
| GCF_900451195.1 | 537 | lodobacter | gntK_f37_MKMCEHOJ_02531_Thermoresistant_gluconokinase | 91.7 | 68.5 | 3.18E−82 |
| | | fluviatilis | | | | |
| GCF_900455105.1 | 158850 | Providencia | gntK_f37_MKMCEHOJ_02531_Thermoresistant_gluconokinase | 96.1 | 65.9 | 8.21E−78 |
| | | rustigianii | | | | |
| GCF_900460315.1 | 103891 | Veillonella criceti | gntK_i37_IIDCKEOG_02051_Xylulose_kinase | 97.7 | 62.9 | 2.54E−239 |

TABLE 38

| accession | transporter_sseqid | transporter_qcovhsp | transporter_pident | transporter_evalue | gut_microbes |
|---|---|---|---|---|---|
| GCF_025564065.1 | transporter_f37_MKMCEHOJ_02530_Low-affinity_gluconate_transporter | 100 | 94.4 | 1.22E−298 | 0 |
| GCF_025564085.1 | transporter_f37_MKMCEHOJ_02530_Low-affinity_gluconate_transporter | 100 | 94.6 | 2.87E−297 | 0 |
| GCF_025566025.1 | transporter_f37_MKMCEHOJ_02530_Low-affinity_gluconate_transporter | 100 | 94.8 | 1.17E−296 | 0 |
| GCF_025567065.1 | transporter_i33_ABMIDDIK_01306_High-affinity_gluconate_transporter | 99.5 | 92.2 | 8.72E−277 | 0 |
| GCF_025758125.1 | transporter_f37_MKMCEHOJ_02530_Low-affinity_gluconate_transporter | 100 | 91.7 | 1.02E−290 | 0 |
| GCF_025914095.1 | transporter_f37_MKMCEHOJ_02530_Low-affinity_gluconate_transporter | 99.8 | 93.3 | 1.07E−292 | 0 |
| GCF_026153155.1 | transporter_f37_MKMCEHOJ_02530_Low-affinity_gluconate_transporter | 100 | 78 | 1.56E−250 | 0 |
| GCF_026154845.1 | transporter_f37_MKMCEHOJ_02530_Low-affinity_gluconate_transporter | 99.8 | 71.4 | 2.96E−227 | 0 |
| GCF_026241935.1 | transporter_f37_MKMCEHOJ_02530_Low-affinity_gluconate_transporter | 100 | 93.5 | 5.80E−297 | 0 |
| GCF_026344075.1 | transporter_f37_MKMCEHOJ_02530_Low-affinity_gluconate_transporter | 100 | 96 | 3.14E−301 | 0 |
| GCF_027886705.1 | transporter_f37_MKMCEHOJ_02530_Low-affinity_gluconate_transporter | 100 | 92.4 | 2.63E−293 | 0 |
| GCF_029277985.1 | transporter_f37_MKMCEHOJ_02530_Low-affinity_gluconate_transporter | 98.5 | 74.4 | 9.66E−242 | 1 |
| GCF_029876145.1 | transporter_f37_MKMCEHOJ_02530_Low-affinity_gluconate_transporter | 100 | 99.3 | 9.62E−312 | 0 |
| GCF_030026565.1 | transporter_f37_MKMCEHOJ_02530_Low-affinity_gluconate_transporter | 100 | 91.9 | 1.33E−286 | 0 |
| GCF_030053895.1 | transporter_f37_MKMCEHOJ_02530_Low-affinity_gluconate_transporter | 100 | 94.4 | 1.49E−299 | 0 |
| GCF_030286765.1 | transporter_f37_MKMCEHOJ_02530_Low-affinity_gluconate_transporter | 100 | 77.3 | 1.10E−250 | 0 |
| GCF_030294585.1 | transporter_f37_MKMCEHOJ_02530_Low-affinity_gluconate_transporter | 100 | 96.6 | 4.67E−303 | 1 |
| GCF_030406165.1 | transporter_f37_MKMCEHOJ_02530_Low-affinity_gluconate_transporter | 100 | 95.7 | 1.28E−300 | 0 |
| GCF_900021175.1 | transporter_f37_MKMCEHOJ_02530_Low-affinity_gluconate_transporter | 100 | 95.1 | 1.05E−299 | 0 |
| GCF_900094795.1 | transporter_f37_MKMCEHOJ_02530_Low-affinity_gluconate_transporter | 100 | 93 | 2.75E−295 | 0 |
| GCF_900095855.1 | transporter_i33_ABMIDDIK_01306_High-affinity_gluconate_transporter | 99.8 | 85.6 | 4.26E−254 | 1 |
| GCF_900103535.1 | transporter_i33_ABMIDDIK_01306_High-affinity_gluconate_transporter | 100 | 81.3 | 2.76E−245 | 0 |
| GCF_900109225.1 | transporter_i33_ABMIDDIK_01306_High-affinity_gluconate_transporter | 100 | 74.5 | 2.32E−223 | 0 |
| GCF_900111105.1 | transporter_f37_MKMCEHOJ_02530_Low-affinity_gluconate_transporter | 100 | 78.5 | 3.99E−251 | 0 |
| GCF_900113945.1 | transporter_f37_MKMCEHOJ_02530_Low-affinity_gluconate_transporter | 100 | 77.3 | 1.04E−248 | 0 |
| GCF_900115195.1 | transporter_f37_MKMCEHOJ_02530_Low-affinity_gluconate_transporter | 100 | 77.3 | 1.21E−247 | 0 |
| GCF_900155355.1 | transporter_f37_MKMCEHOJ_02530_Low-affinity_gluconate_transporter | 100 | 78.4 | 3.83E−251 | 0 |
| GCF_900167325.1 | transporter_i33_ABMIDDIK_01306_High-affinity_gluconate_transporter | 100 | 91.1 | 8.72E−277 | 0 |
| GCF_900178525.1 | transporter_i13_AFPGDEAI_02973_Gnt-II_system_L-idonate_transporter | 100 | 71.5 | 1.06E−207 | 0 |
| GCF_900186025.1 | transporter_f37_MKMCEHOJ_02505_High-affinity_gluconate_transporter | 100 | 86.1 | 2.76E−261 | 0 |
| GCF_900187015.1 | transporter_f37_MKMCEHOJ_02530_Low-affinity_gluconate_transporter | 99.3 | 72.9 | 5.70E−232 | 0 |
| GCF_900187035.1 | transporter_i37_IIDCKEOG_02049_High-affinity_gluconate_transporter | 99.3 | 73.4 | 8.95E−221 | 1 |
| GCF_900451195.1 | transporter_f37_MKMCEHOJ_02530_Low-affinity_gluconate_transporter | 100 | 76.2 | 2.10E−241 | 0 |
| GCF_900455105.1 | transporter_f37_MKMCEHOJ_02530_Low-affinity_gluconate_transporter | 98.5 | 74.2 | 6.47E−240 | 1 |
| GCF_900460315.1 | transporter_i33_ABMIDDIK_01306_High-affinity_gluconate_transporter | 100 | 73.4 | 3.13E−221 | 0 |

TABLE 39

| | organism. | | | gntK_ | gntK_ | gntK_ |
|---|---|---|---|---|---|---|
| accession | taxId | species | gntK_sseqid | qcovhsp | pident | evalue |
| GCF_900475855.1 | 333962 | Providencia | gntK_f37_MKMCEHOJ_02531_Thermoresistant_gluconokinase | 96.1 | 69.9 | 8.64E−80 |
| | | heimbachae | | | | |
| GCF_900635955.1 | 158823 | Cedecea lapagei | gntK_Kp2H7_NOCLEBFK_02257_Thermoresistant_gluconokinase | 98.9 | 89.7 | 1.81E−115 |
| GCF_900638135.1 | 137545 | Serratia | gntK_f37_MKMCEHOJ_02531_Thermoresistant_gluconokinase | 94.4 | 75.1 | 2.62E−90 |
| | | quinivorans | | | | |
| GCF_900698925.1 | 1922217 | Candidatus | gntK_Kp2H7_NOCLEBFK_02257_Thermoresistant_gluconokinase | 94 | 65.3 | 7.35E−80 |
| | | Erwinia haradaeae | | | | |
| GCF_901421005.1 | 54291 | Raoultella | gntK_Kp2H7_NOCLEBFK_02257_Thermoresistant_gluconokinase | 100 | 96.6 | 1.23E−124 |
| | | ornithinolytica | | | | |
| GCF_901563875.1 | 2587528 | Klebsiella | gntK_Kp2H7_NOCLEBFK_02257_Thermoresistant_gluconokinase | 100 | 96 | 7.14E−124 |
| | | spallanzanii | | | | |
| GCF_902164675.1 | 2058152 | Klebsiella grimontii | gntK_Kp2H7_NOCLEBFK_02257_Thermoresistant_gluconokinase | 99.4 | 96 | 5.87E−123 |
| GCF_902164725.1 | 566 | Pseudescherichia | gntK_Kp2H7_NOCLEBFK_02257_Thermoresistant_gluconokinase | 100 | 93.6 | 6.80E−119 |
| | | vulneris | | | | |
| GCF_902374985.1 | 1485952 | Phytobacter | gntK_Kp2H7_NOCLEBFK_02257_Thermoresistant_gluconokinase | 100 | 92.5 | 4.59E−117 |
| | | massiliensis | | | | |
| GCF_902386985.1 | 472695 | Pantoea septica | gntK_Kp2H7_NOCLEBFK_02257_Thermoresistant_gluconokinase | 97.2 | 74 | 4.72E−93 |
| GCF_902387845.1 | 582 | Morganella | gntK_Kp2H7_NOCLEBFK_02257_Thermoresistant_gluconokinase | 97.8 | 67.6 | 3.45E−84 |
| | | morganii | | | | |
| GCF_902500225.1 | 2831590 | Buttiauxella | gntK_f37_MKMCEHOJ_02531_Thermoresistant_gluconokinase | 98.3 | 89.7 | 9.69E−113 |
| | | massiliensis | | | | |
| GCF_902709585.1 | 1499973 | Escherichia | gntK_f37_MKMCEHOJ_02531_Thermoresistant_gluconokinase | 100 | 99.4 | 1.45E−127 |
| | | marmotae | | | | |
| GCF_905331265.2 | 550 | Enterobacter | gntK_Kp2H7_NOCLEBFK_02257_Thermoresistant_gluconokinase | 98.9 | 93.6 | 7.08E−117 |
| | | cloacae | | | | |
| GCF_943193155.1 | 2941341 | Veillonella | gntK_i37_IIDCKEOG_02051_Xylulose_kinase | 97.7 | 63.1 | 7.25E−239 |
| | | intestinalis | | | | |
| GCF_943590815.1 | 2488306 | Pseudocitrobacter | gntK_f37_MKMCEHOJ_02531_Thermoresistant_gluconokinase | 98.3 | 92 | 3.83E−119 |
| | | vendiensis | | | | |
| GCF_949794035.1 | 28151 | Serratia | gntK_f37_MKMCEHOJ_02531_Thermoresistant_gluconokinase | 97.1 | 71.2 | 2.88E−86 |
| | | proteamaculans | | | | |
| GCF_959600285.1 | 464322 | Veillonella magna | gntK_i37_IIDCKEOG_02051_Xylulose_kinase | 98.5 | 64.2 | 9.10E−247 |

TABLE 40

| | | trans- | trans- | trans- | |
|---|---|---|---|---|---|
| | | porter_ | porter_ | porter_ | gut_ |
| accession | transporter_sseqid | qcovhsp | pident | evalue | microbes |
| GCF_900475855.1 | transporter_f37_MKMCEHOJ_02530_Low-affinity_gluconate_transporter | 98.5 | 74.4 | 1.51E−238 | 0 |
| GCF_900635955.1 | transporter_f37_MKMCEHOJ_02530_Low-affinity_gluconate_transporter | 100 | 92.4 | 4.84E−289 | 0 |
| GCF_900638135.1 | transporter_f37_MKMCEHOJ_02530_Low-affinity_gluconate_transporter | 99.8 | 72.5 | 8.91E−229 | 0 |
| GCF_900698925.1 | transporter_f37_MKMCEHOJ_02530_Low-affinity_gluconate_transporter | 99.1 | 69.7 | 1.11E−215 | 0 |
| GCF_901421005.1 | transporter_f37_MKMCEHOJ_02530_Low-affinity_gluconate_transporter | 100 | 96 | 8.61E−299 | 1 |
| GCF_901563875.1 | transporter_f37_MKMCEHOJ_02530_Low-affinity_gluconate_transporter | 100 | 96.6 | 9.84E−305 | 0 |
| GCF_902164675.1 | transporter_f37_MKMCEHOJ_02530_Low-affinity_gluconate_transporter | 100 | 96.4 | 1.63E−303 | 0 |
| GCF_902164725.1 | transporter_f37_MKMCEHOJ_02530_Low-affinity_gluconate_transporter | 100 | 93.9 | 9.18E−294 | 0 |
| GCF_902374985.1 | transporter_f37_MKMCEHOJ_02530_Low-affinity_gluconate_transporter | 100 | 93.3 | 2.16E−292 | 1 |
| GCF_902386985.1 | transporter_f37_MKMCEHOJ_02530_Low-affinity_gluconate_transporter | 100 | 80.3 | 2.40E−259 | 1 |
| GCF_902387845.1 | transporter_f37_MKMCEHOJ_02530_Low-affinity_gluconate_transporter | 100 | 77.5 | 1.56E−243 | 1 |
| GCF_902500225.1 | transporter_f37_MKMCEHOJ_02530_Low-affinity_gluconate_transporter | 100 | 92.6 | 3.73E−293 | 0 |
| GCF_902709585.1 | transporter_f37_MKMCEHOJ_02530_Low-affinity_gluconate_transporter | 100 | 99.3 | 9.62E−312 | 0 |
| GCF_905331265.2 | transporter_f37_MKMCEHOJ_02530_Low-affinity_gluconate_transporter | 100 | 96.2 | 7.72E−302 | 1 |
| GCF_943193155.1 | transporter_i33_ABMIDDIK_01306_High-affinity_gluconate_transporter | 100 | 73.2 | 1.80E−220 | 0 |
| GCF_943590815.1 | transporter_f37_MKMCEHOJ_02530_Low-affinity_gluconate_transporter | 100 | 94.8 | 5.80E−297 | 0 |
| GCF_949794035.1 | transporter_f37_MKMCEHOJ_02505_High-affinity_gluconate_transporter | 100 | 86.3 | 8.28E−263 | 0 |
| GCF_959600285.1 | transporter_i33_ABMIDDIK_01306_High-affinity_gluconate_transporter | 100 | 72.9 | 6.65E−223 | 1 |

Furthermore, Taxonomy information of 204, 938 intestinal bacterial genomes reported by A. Almeida et al., 2020, Nature Biotechnology (https://www.nature.com/articles/s41587-020-0603-3) was obtained. To assess whether the bacterial species analytically identified as gluconate-metabolizing bacteria this time were included in intestinal bacteria, the lists of bacteria indicated in Table 13 to 40 were compared with the obtained Taxonomy information used as an intestinal bacterial list.

As a result, from the match of species names with the intestinal bacterial list, it was suggested that 10 species (gad) and 68 species (gnt) were detected from the intestine, respectively. The 10 species (gad) and 68 species (gnt) are bacteria whose item “gut_microbes” is “1” in Tables 13 to 40.

That is, from the results of FIGS. 19A to 19D and the above, as bacteria that can suppress the intestinal colonization of bacteria that cause diseases such as Kp-2H7 by competing in gluconate consumption, whose gluconate amount in the middle bar graph in FIGS. 19A to 19D is 100 μM or less and at least one of the 70 species (gad) and 403 species (gnt) are preferable, and bacteria whose gluconate amount in the middle bar graph in FIGS. 19A to 19D is 100 μM or less and at least one of the 10 species (gad) and 68 species (gnt) are more preferable, and bacteria whose gluconate amount in the middle bar graph in FIGS. 19A to 19D is 100 μM or less are particularly preferable.

### Example 12

### Suppressive Effect of F18Mix or F13Mix on Intestinal Colonization of Kp2H7 Strain

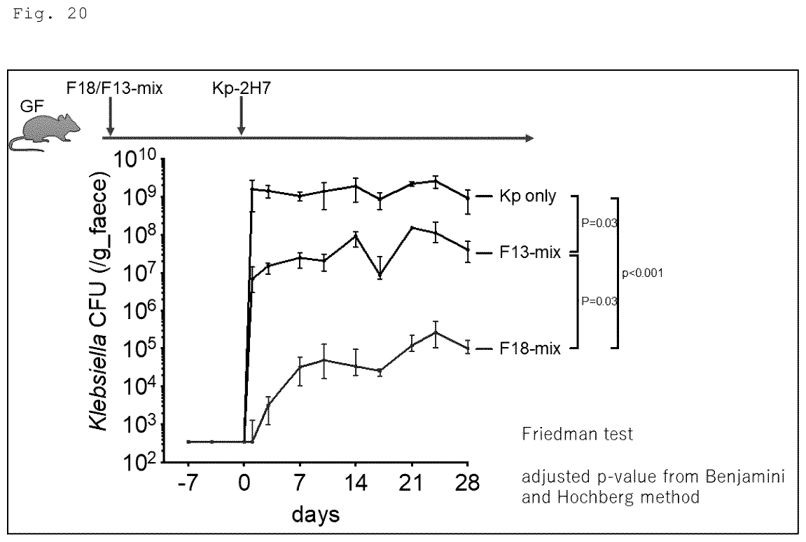

Germ-free mice were divided into 3 groups (4 mice in each group): a group administered with 18 intestinal bacterial strains (F18-mix) isolated from a fecal sample of healthy individual #F, a group administered with 13 strains (F13-mix), and a group administered with nothing (Kp only). For the first two groups, F18mix or F13mix was administered immediately after the start of the experiment. Kp2H7 strain was administered to each of the three groups two weeks after the experiment began, and the bacterial amount of Kp2H7 strain in feces was analyzed over time. The obtained results are shown in FIG. 20.

As is clear from the results shown in FIG. 20, it was confirmed that the increase in the bacterial amount of Kp2H7 strain was greatly suppressed when F18mix was administered in particular, compared to the group administered with nothing.

As described above, according to the present invention, by suppressing colonization etc. of gluconate-utilizing bacteria such as drug-resistant bacteria and proinflammatory bacteria in the intestinal tract, it is also possible to treat or prevent diseases caused by these bacteria.

Therefore, the present invention is extremely useful in the development, treatment, improvement, and prevention of infectious diseases caused by the above-mentioned bacteria.

### Accession Number

(1) Identification label: f1_42H6
(2) Accession number: NITE BP-03147
(3) Deposit date: Mar. 2, 2020
(4) Depositary institution: National Institute of Technology and Evaluation, Patent Microorganisms Depositary
(1) Identification label: f12_42H4
(2) Accession number: NITE BP-03148
(3) Deposit date: Mar. 2, 2020
(4) Depositary institution: National Institute of Technology and Evaluation, Patent Microorganisms Depositary
(1) Identification label: f17_42I7
(2) Accession number: NITE BP-03149
(3) Deposit date: Mar. 2, 2020
(4) Depositary institution: National Institute of Technology and Evaluation, Patent Microorganisms Depositary
(1) Identification label: f18_42I2
(2) Accession number: NITE BP-03150
(3) Deposit date: Mar. 2, 2020
(4) Depositary institution: National Institute of Technology and Evaluation, Patent Microorganisms Depositary
(1) Identification label: f19_43G2
(2) Accession number: NITE BP-03151
(3) Deposit date: Mar. 2, 2020
(4) Depositary institution: National Institute of Technology and Evaluation, Patent Microorganisms Depositary
(1) Identification label: f20_43G1
(2) Accession number: NITE BP-03152
(3) Deposit date: Mar. 2, 2020
(4) Depositary institution: National Institute of Technology and Evaluation, Patent Microorganisms Depositary
(1) Identification label: f21_42A8
(2) Accession number: NITE BP-03153
(3) Deposit date: Mar. 2, 2020
(4) Depositary institution: National Institute of Technology and Evaluation, Patent Microorganisms Depositary
(1) Identification label: f22_43C3
(2) Accession number: NITE BP-03154
(3) Deposit date: Mar. 2, 2020
(4) Depositary institution: National Institute of Technology and Evaluation, Patent Microorganisms Depositary
(1) Identification label: f23_42K4
(2) Accession number: NITE BP-03155
(3) Deposit date: Mar. 2, 2020
(4) Depositary institution: National Institute of Technology and Evaluation, Patent Microorganisms Depositary
(1) Identification label: f24_42I4
(2) Accession number: NITE BP-03156
(3) Deposit date: Mar. 2, 2020
(4) Depositary institution: National Institute of Technology and Evaluation, Patent Microorganisms Depositary
(1) Identification label: f26_42K2
(2) Accession number: NITE BP-03157
(3) Deposit date: Mar. 2, 2020
(4) Depositary institution: National Institute of Technology and Evaluation, Patent Microorganisms Depositary
(1) Identification label: f28_43A3
(2) Accession number: NITE BP-03158
(3) Deposit date: Mar. 2, 2020
(4) Depositary institution: National Institute of Technology and Evaluation, Patent Microorganisms Depositary
(1) Identification label: f30_43A5
(2) Accession number: NITE BP-03159
(3) Deposit date: Mar. 2, 2020
(4) Depositary institution: National Institute of Technology and Evaluation, Patent Microorganisms Depositary
(1) Identification label: f31_43J5
(2) Accession number: NITE BP-03160
(3) Deposit date: Mar. 2, 2020
(4) Depositary institution: National Institute of Technology and Evaluation, Patent Microorganisms Depositary
(1) Identification label: f32_42A7
(2) Accession number: NITE BP-03161
(3) Deposit date: Mar. 2, 2020
(4) Depositary institution: National Institute of Technology and Evaluation, Patent Microorganisms Depositary
(1) Identification label: f33_43N2
(2) Accession number: NITE BP-03162
(3) Deposit date: Mar. 2, 2020
(4) Depositary institution: National Institute of Technology and Evaluation, Patent Microorganisms Depositary
(1) Identification label: f35_42L8
(2) Accession number: NITE BP-03163
(3) Deposit date: Mar. 2, 2020
(4) Depositary institution: National Institute of Technology and Evaluation, Patent Microorganisms Depositary
(1) Identification label: f37_42G1
(2) Accession number: NITE BP-03164
(3) Deposit date: Mar. 2, 2020
(4) Depositary institution: National Institute of Technology and Evaluation, Patent Microorganisms Depositary
(1) Identification label: f10_43J3
(2) Accession number: NITE BP-03800
(3) Deposit date: Jan. 12, 2023
(4) Depositary institution: National Institute of Technology and Evaluation, Patent Microorganisms Depositary
(1) Identification label: f29_43J8
(2) Accession number: NITE BP-03801
(3) Deposit date: Jan. 12, 2023
(4) Depositary institution: National Institute of Technology and Evaluation, Patent Microorganisms Depositary

1. An antibacterial composition comprising at least one gluconate-utilizing first bacterial strain as an active agent, wherein
the composition exhibits antibacterial activity against a gluconate-utilizing second bacterial strain.

2. The composition according to claim 1, wherein
the first bacterial strain comprises a gene encoding a gluconate transporter and at least one gene selected from the group consisting of a gene encoding gluconate kinase and a gene encoding gluconate dehydratase, and
the gene encoding the gluconate transporter and the at least one selected gene are located in the same gene cluster.

3. The composition according to claim 1, wherein the first bacterial strain comprises a gene encoding gluconate dehydratase and a gene encoding a gluconate transporter.

4. The composition according to claim 1, wherein the composition is ingested by a subject in combination with a food containing gluconate at a low concentration.

5. The composition according to claim 4, wherein the composition is ingested by a subject simultaneously with the food or separately from the food.

6. The composition according to claim 1, wherein the second bacterial strain is pathogenic.

7. The composition according to claim 6, wherein the composition is a pharmaceutical composition.

8. A method for screening a bacterial strain having antibacterial activity against a gluconate-utilizing bacterial strain, comprising:
detecting gluconate utilization ability of a test bacterial strain; and
determining that the test bacterial strain has antibacterial activity when the gluconate utilization ability is detected.

9. A method for screening a bacterial strain having antibacterial activity against a gluconate-utilizing bacterial strain, comprising:
detecting a gene encoding gluconate transporter and at least one gene selected from the group consisting of a gene encoding gluconate kinase and a gene encoding gluconate dehydratase;
identifying the gene cluster corresponding to each detected gene; and
determining that the test bacterial strain has antibacterial activity when the detected genes are located in the same gene cluster.

10. (canceled)

11. (canceled)

12. The composition according to claim 1, wherein the first bacterial strain is selected a group consisting of:
a bacterial strain having a nucleotide sequence having at least 99% identity with the nucleotide sequence of SEQ ID NO: 17,
a bacterial strain having a nucleotide sequence having at least 99% identity with the nucleotide sequence of SEQ ID NO: 18,
a bacterial strain having a nucleotide sequence having at least 99% identity with the nucleotide sequence of SEQ ID NO: 20,
a bacterial strain having a nucleotide sequence having at least 99% identity with the nucleotide sequence of SEQ ID NO: 21,
a bacterial strain having a nucleotide sequence having at least 99% identity with the nucleotide sequence of SEQ ID NO: 22,
a bacterial strain having a nucleotide sequence having at least 99% identity with the nucleotide sequence of SEQ ID NO: 23,
a bacterial strain having a nucleotide sequence having at least 99% identity with the nucleotide sequence of SEQ ID NO: 32, and
a bacterial strain having a nucleotide sequence having at least 99% identity with the nucleotide sequence of SEQ ID NO: 37.

13. The composition according to claim 12, wherein the composition comprises eight gluconate-utilizing first bacterial strains.

14. The composition according to claim 13, wherein the gluconate-utilizing first bacterial strains are:
a bacterial strain having a nucleotide sequence having at least 99% identity with the nucleotide sequence of SEQ ID NO: 17,
a bacterial strain having a nucleotide sequence having at least 99% identity with the nucleotide sequence of SEQ ID NO: 18,
a bacterial strain having a nucleotide sequence having at least 99% identity with the nucleotide sequence of SEQ ID NO: 20,
a bacterial strain having a nucleotide sequence having at least 99% identity with the nucleotide sequence of SEQ ID NO: 21,
a bacterial strain having a nucleotide sequence having at least 99% identity with the nucleotide sequence of SEQ ID NO: 22,
a bacterial strain having a nucleotide sequence having at least 99% identity with the nucleotide sequence of SEQ ID NO: 23,
a bacterial strain having a nucleotide sequence having at least 99% identity with the nucleotide sequence of SEQ ID NO: 32, and
a bacterial strain having a nucleotide sequence having at least 99% identity with the nucleotide sequence of SEQ ID NO: 37.

15. The composition according to claim 6, wherein the second bacterial strain is Enterobacteriaceae.
